(19) United States
(12) Patent Application Publication
(10) Pub. No.: US 2022/0324933 A1
(43) Pub. Date: Oct. 13, 2022

(54) IL-2 COMPOSITIONS AND METHODS OF USE THEREOF

(71) Applicant: PROVIVA THERAPEUTICS (HONG KONG) LIMITED, Admiralty, Hong Kong (CN)

(72) Inventor: Zijuan LI, Shanghai (CN)

(21) Appl. No.: 17/626,365

(22) PCT Filed: Jul. 10, 2020
(86) PCT No.: PCT/US2020/041543

§ 371 Date: Jan. 11, 2022

Related U.S. Application Data
(60) Provisional application No. 62/873,399, filed on Jul. 12, 2019.
(60) Provisional application No. 62/908,782, filed on Oct. 1, 2019.

(51) Int. Cl. C07K 14/55; C07K 14/715; C07K 16/24

(52) U.S. Cl. CPC C07K 14/55; C07K 14/7155; C07K 16/246; C07K 2319/30; C07K 2319/32; C07K 2317/31; A61K 38/00

(57) ABSTRACT

Provided are activatable proprotein homodimers, comprising at least two separate polypeptide chains, each chain comprising an IL-2 protein a cleavable linker, and an IL-2 binding protein, among other optional features, and related pharmaceutical compositions and methods of use thereof.

### CROSS-REFERENCE TO RELATED APPLICATIONS

This application claims the benefit under 35 U.S.C. § 119(e) to U.S. Provisional Application No. 62/908,782, filed Oct. 1, 2019; and U.S. Provisional Application No. 62/873,399, filed Jul. 12, 2019, each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is PRVA_003_02WO_ST25.txt. The text file is about 952 KB, created on Jul. 9, 2020, and is being submitted electronically via EFS-Web.

### BACKGROUND

### Technical Field

The present disclosure relates to an activatable proprotein homodimer comprising at least two separate polypeptide chains, each chain comprising an IL-2 protein, a cleavable linker, and an IL-2 binding protein, among other optional features, and related pharmaceutical compositions and methods of use thereof.

### Description of the Related Art

Interleukin-2 (IL-2) immunotherapy has proven utility in the treatment of cancers such as malignant melanoma and renal cell cancer, and chronic infections such as HIV infections.

However, there are certain problems associated with most IL-2 therapies. For example, current forms of IL-2 therapy have a short half-life in circulation and predominantly expand immunosuppressive regulatory T cells, or T_{regs} (see, for example, Arenas-Ramirez et al., Trends in Immunology. 36: 763-777, 2015). Also, the effects of IL-2 therapy are predominantly systemic, rather than being localized to target tissues, resulting in many severe side effects such as breathing problems, nausea, low blood pressure, loss of appetite, confusion, serious infections, seizures, allergic reactions, heart problems, renal failure, and vascular leak syndrome. Nonetheless, IL-2 therapy can be effective, and there is an unmet need in the art to overcome these and other drawbacks.

Embodiments of the present disclosure address these problems and more by providing an activatable proprotein comprising IL-2 that can be activated within a disease tissue, for example, a cancer tissue or tumor.

### BRIEF SUMMARY

Embodiments of the present disclosure include an activatable proprotein homodimer, comprising a first polypeptide and a second polypeptide, wherein:

(a) the first polypeptide and the second polypeptide comprise, in an N- to C-terminal orientation, or a C- to N-terminal orientation, a binding moiety, a first linker, an IL-2 protein, a second linker, and an IL-2 binding protein; or

(b) the first polypeptide and the second polypeptide comprise, in an N- to C-terminal orientation, or a C- to N-terminal orientation, a binding moiety, a first linker, an IL-2 binding protein, a second linker, and an IL-2 protein,

wherein the binding moiety of the first polypeptide binds to the binding moiety of the second polypeptide, wherein the IL-2 protein of the first polypeptide binds to the IL-2 binding protein of the second polypeptide, and wherein the IL-2 binding protein of the first polypeptide binds to the IL-2 protein of the second polypeptide, wherein said (collective) binding masks a binding site of IL-2 protein(s) that otherwise binds to an IL-2Rβ/γc and/or IL-2Rα/β/γc chain present on the surface of an immune cell in vitro or in vivo, and wherein at least one of the first or the second linker is a cleavable linker; or

(c) the first and the second polypeptide comprise, in an N- to C-terminal orientation, or a C- to N-terminal orientation, an IL-2 protein, a first linker, an IL-2 binding protein, a second linker, and an affinity purification tag; or

(d) the first and the second polypeptide comprise, in an N- to C-terminal orientation, or a C- to N-terminal orientation, an IL-2 binding protein, a first linker, an IL-2 protein, a second linker, and an affinity purification tag,

wherein the IL-2 protein of the first polypeptide binds to the IL-2 binding protein of the second polypeptide, and wherein the IL-2 binding protein of the first polypeptide binds to the IL-2 protein of the second polypeptide, wherein said (collective) binding masks a binding site of IL-2 protein(s) that otherwise binds to an IL-2Rβ/γc and/or IL-2Rα/β/γc chain present on the surface of an immune cell in vitro or in vivo, and wherein the first linker is a cleavable linker.

In some embodiments, the first and second IL-2 proteins comprise, consist, or consist essentially of an amino acid sequence that is at least 80, 85, 90, 95, 98, or 100% identical to an amino acid sequence selected from Table S1, optionally amino acids 21-153 of SEQ ID NO: 1 (full-length wild-type human IL-2), optionally comprising a C145X (X is any amino acid) or a C145S substitution as defined by SEQ ID NO: 1. In some embodiments, the first and second IL-2 proteins comprise, consist, or consist essentially of an amino acid sequence that is at least 80, 85, 90, 95, 98, or 100% identical to SEQ ID NO: 2 (mature human IL-2 with C125S substitution), optionally wherein the IL-2 protein retains the S125 residue as defined by SEQ ID NO: 2. In some embodiments, the first and second IL-2 proteins comprise one or more substitutions selected from K35C, R38C, T41C, F42C, E61C, and V69C as defined by SEQ ID NO: 2.

In some embodiments, the first IL-2 protein forms a disulfide bond with the second IL-2 binding protein, and wherein the second IL-2 protein forms a disulfide bond with the first IL-2 binding protein, optionally via one or more of the cysteines in claim 4 and one or more cysteines in the first and second IL-2 binding proteins(s).

In some embodiments, the first and second IL-2 proteins comprise one or more amino acid substitutions at position 69, 74, and/or 128 as defined by SEQ ID NO: 2, optionally wherein the one or more amino acid substitutions are selected from V69A, Q74P, and I128T as defined by SEQ ID NO: 2. In some embodiments, the first and second IL-2 proteins comprise one or more amino acid substitutions at position T3, R38, F42, Y45, E61, E62, E68, and/or L72 as defined by SEQ ID NO: 2, optionally wherein the one or more amino acid substitutions are selected from T3A; R38A and R38K; F42A, F42G, F42S, F42T, F42Q, F42E, F42N, F42D, F42R, F42K, and F42I; Y45A, Y45G, Y45S, Y45T, Y45Q, Y45E, Y45N, Y45D, Y45R, and Y45K; E61S; E62A and E62L; E68A and E68V; and L72A, L72G, L72S, L72T, L72Q, L72E, L72N, L72D, L72R, and L72K, including combinations thereof, optionally a combination selected from F42A, Y45A, and L72G; R38K, F42Q, Y45N, E62L, and E68V; R38K, F42Q, Y45E, and E68V; R38A, F42I, Y45N, E62L, and E68V; R38K, F42K, Y45R, E62L, and E68V; R38K, F42I, Y45E, and E68V; and R38A, F42A, Y45A, and E62A.

In some embodiments, the first and second IL-2 proteins comprise, consist, or consist essentially of an amino acid sequence that is at least 80, 85, 90, 95, 98, or 100% identical to SEQ ID NO: 3 (mature human IL-2 “D10” variant), optionally wherein the IL-2 protein retains any one or more of the Q74H, L80F, R81D, L85V, I86V, and/or I92F substitutions as defined by SEQ ID NO: 3.

In some embodiments, the first and second IL-2 binding proteins comprise a first and second IL-2Rα protein, or a first and second antibody or antigen binding fragment thereof that specifically binds to the IL-2 protein(s), optionally a bi-specific antibody or antigen binding fragment thereof.

In some embodiments, the first and second IL-2Rα proteins comprise, consist, or consist essentially of an amino acid sequence that is at least 80, 85, 90, 95, 98, or 100% to an amino acid sequence selected from Table S2, optionally amino acids 22-187 of SEQ ID NO: 4 (full-length wild-type human IL-2Rα). In some embodiments, the first and second IL-2Rα proteins comprise one or more cysteine substitutions selected from D4C, D6C, N27C, K38C, S39C, L42C, Y43C, I118C, and H120C as defined by SEQ ID NO: 6 (human IL-2Rα Sushi 1 to Sushi 2 domain), and/or a K38S substitution. In some embodiments, the first IL-2Rα protein forms a disulfide bond with the second IL-2 protein, and wherein the second IL-2Rα protein forms a disulfide bond with the first IL-2 protein, optionally via one or more of the cysteines in claim 11 and one or more cysteines in the IL-2 protein, optionally one or more of the cysteines in claim 4, optionally one or more cysteine pairs selected from IL2-K35C and IL2Rα-D4C, IL2-R38C and IL2Rα-D6C, IL2-R38C and IL2Rα-H120C, IL2-T41C and IL2Rα-I118C, IL2-F42C and IL2Rα-N27C, IL2-E61C and IL2Rα-K38C, IL2-E61C and IL2Rα-S39C, and IL2-V69C and IL2Rα-L42C, wherein disulfide binding between the IL-2 protein and the IL-2Rα protein masks the binding site of the IL-2 protein that preferentially binds to the IL-2Rα/β/γc chain expressed on T_{regs}. In some embodiments, first and second IL-2Rα proteins comprise an alanine substitution at position 49 and/or 68 as defined by SEQ ID NO: 6.

In some embodiments, the first and second antibody or antigen binding fragment thereof that specifically binds to the IL-2 protein is selected from one or more of a whole antibody, Fab, Fab′, F(ab′)2, monospecific Fab2, bispecific Fab2, FV, single chain Fv (scFv), scFV-Fc, nanobody, diabody, camelid, and a minibody, optionally wherein the antibody is NARA1 or an antigen binding fragment thereof. In some embodiments, the binding moieties of (a) and/or (b) do not bind to the IL-2 protein or the IL-2 binding protein. In some embodiments, the binding moieties of (a) and/or (b) bind to the IL-2 protein. In some embodiments, the binding moieties of the first polypeptide and the second polypeptide of (a) and/or (b) bind together, optionally homodimerize, via at least one non-covalent interaction. In some embodiments, the binding moieties of the first polypeptide and the second polypeptide of (a) and/or (b) bind together, optionally homodimerize, via at least one covalent bond. In some embodiments, the at least one covalent bond comprises at least one disulfide bond.

In some embodiments, the binding moieties of the first polypeptide and the second polypeptide of (a) and/or (b) are selected from Table M1. In some embodiments, the binding moieties of the first polypeptide and the second polypeptide of (a) or (b) comprise an antigen binding domain of an immunoglobulin, including antigen binding fragments and variants thereof. In some embodiments, the binding moieties of the first polypeptide and the second polypeptide of (a) and/or (b) comprise a CH1, CH2, CH3, CH1CH3, CH2CH3, CH1CH2CH3, and/or CL domain of an immunoglobulin, including fragments and variants thereof. In some embodiments, the binding moieties of the first polypeptide and the second polypeptide of (a) and/or (b) comprise, in an N- to C-terminal orientation: (1) an antigen binding domain of an immunoglobulin, including antigen binding fragments and variants thereof; and (2) a CH1, CH2, CH3, CH1CH3, CH2CH3, CH1CH2CH3, and/or CL domain of an immunoglobulin, including fragments and variants thereof. In some embodiments, the antigen binding domain comprises a VH or VL domain of an immunoglobulin, including antigen binding fragments and variants thereof. In some embodiments, the binding moieties of the first polypeptide and the second polypeptide of (a) and/or (b) do not bind to an antigen. In some embodiments, the binding moieties of the first polypeptide and the second polypeptide of (a) and/or (b) comprise a CH2CH3 domain of an immunoglobulin. In some embodiments, the immunoglobulin is from an immunoglobulin class selected from IgG1, IgG2, IgG3, IgG4, IgA, IgD, IgE, and IgM. In some embodiments, the binding moieties of the first polypeptide and the second polypeptide of (a) and/or (b) comprise a leucine zipper peptide.

In some embodiments, the affinity purification tag of (c) and/or (d) is selected from a polyhistidine tag (optionally hexahistidine tag), a VSV-G tag, a universal tag, a Strep-tag, an S-tag, an S1-tag, a Phe-tag, a Cys-tag, an Asp-tag, an Arg-tag, a Myc epitope tag, a KT3 epitope tag, an HSV epitope tag, a histidine affinity tag, a hemagglutinin (HA) tag, a FLAG epitope tag, an E2 epitope tag, a V5-tag, a T7-tag, an AU5 epitope tag, and an AU1 epitope tag.

In some embodiments, the cleavable linker comprises a protease cleavage site, optionally wherein the cleavable linker is selected from Table S3. In some embodiments, the protease cleavage site is cleavable by a protease selected from one or more of a metalloprotease, a serine protease, a cysteine protease, and an aspartic acid protease. In some embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B. In some embodiments, the first linker and/or the second linker are about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16 ,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

In some embodiments, the first linker of (a) and/or (b) is a cleavable linker, and wherein the second linker of (a) and/or (b) is a non-cleavable linker. In some embodiments, cleavage, optionally protease cleavage, of the first linker of (a) and/or (b) exposes the binding site(s) of the first and/or second IL-2 proteins that bind to the IL-2Rβ/γc chain present on the surface of the immune cell in vitro or in vivo. In some embodiments, the first linker of (a) and/or (b) is a non-cleavable linker, and wherein the second linker of (a) and/or (b) is a cleavable linker. In some embodiments, cleavage, optionally protease cleavage, of the second linker of (a) and/or (b) exposes the binding site(s) of the first and/or second IL-2 proteins that bind to the IL-2Rβ/γc chain present on the surface of the immune cell in vitro or in vivo. In some embodiments, cleavage, optionally protease cleavage, of the first linker of (c) and/or (d) exposes the binding site(s) of the first and/or second IL-2 proteins that bind to the IL-2Rβ/γc chain present on the surface of the immune cell in vitro or in vivo. In some embodiments, the immune cell is selected from one or more of a T cell, a B cell, a natural killer cell, a monocyte, and a macrophage.

In some embodiments, the first polypeptide and the second polypeptide of (a) comprise, in an N- to C-terminal orientation, the binding moiety, the first linker, the IL-2 protein, the second linker, and the IL-2 binding protein. In some embodiments, the first polypeptide and the second polypeptide of (a) comprise, in an N- to C-terminal orientation, the IL-2 binding protein, the first linker, the IL-2 protein, the second linker, and the binding moiety. In some embodiments, the first polypeptide and the second polypeptide of (b) comprise, in an N- to C-terminal orientation, the binding moiety, the first linker, the IL-2 binding protein, the second linker, and the IL-2 protein. In some embodiments, the first polypeptide and the second polypeptide of (b) comprise, in an N- to C-terminal orientation, the IL-2 protein, the first linker, the IL-2 binding protein, the second linker, and the binding moiety. In some embodiments, the first polypeptide and the second polypeptide of (c) comprise, in an N- to C-terminal orientation, the IL-2 protein, the first linker, the IL-2 binding protein, the second linker, and the affinity purification tag. In some embodiments, the first polypeptide and the second polypeptide of (d) comprise, in an N- to C-terminal orientation, the IL-2 binding protein, the first linker, the IL-2 protein, the second linker, and the affinity purification tag.

In some embodiments, the first polypeptide and the second polypeptide comprise, consist, or consist essentially of an amino acid sequence that is at least 80, 85, 90, 95, 98, or 100% identical to a sequence selected from Table S4, optionally wherein the TEV protease cleavage site is replaced with cleavage site cleavable by a human protease, optionally a cleavable linker selected from Table S3.

In some embodiments, the activatable proprotein is substantially in homodimeric form in a physiological solution, or under physiological conditions, optionally in vivo conditions.

Also included are recombinant nucleic acid molecules encoding an activatable proprotein homodimer described herein, vectors comprising the recombinant nucleic acid molecules described herein, and host cells comprising the recombinant nucleic acid molecules or the vectors described herein.

Also included are methods of producing an activatable proprotein, comprising culturing a host cell described herein under culture conditions suitable for the expression of the activatable proprotein homodimer, and isolating the activatable proprotein from the culture.

Also included are pharmaceutical compositions, comprising an activatable proprotein homodimer described herein, and a pharmaceutically acceptable carrier.

Certain embodiments include methods of treating disease in a subject, and/or a method of enhancing an immune response in a subject, comprising administering to the subject a therapeutically effective amount of the pharmaceutical composition described herein.

In some embodiments, the disease is selected from one or more of a cancer, a viral infection, and an immune disorder. In some embodiments, the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, following administration, the activatable proprotein homodimer is activated through protease cleavage in a cell or tissue, optionally a cancer cell or cancer tissue, which exposes the binding site(s) of the first and/or second IL-2 proteins that bind to the IL-2Rβ/γc chain present on the surface of the immune cell in vitro or in vivo, and thereby generates an activated protein. In some embodiments, the activated protein binds via the IL-2 protein to the IL-2Rβ/γc chain present on the surface of an immune cell in vitro or in vivo. In some embodiments, the immune cell is selected from one or more of a T cell, a B cell, a natural killer cell, a monocyte, and a macrophage. In some embodiments, binding between the IL-2 protein(s) and the IL-2 binding protein(s) (optionally disulfide binding between the IL-2 protein(s) and the IL-2Rα protein(s)) in the activated protein masks the binding site of the IL-2 protein(s) that binds to the IL-2Rα/β/γc chain expressed on T_{regs}, and thereby interferes with binding of the activated protein to T_{regs}.

In some embodiments, administration and activation of the activatable proprotein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response. In some embodiments, administration and activation of the activatable proprotein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

In some embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

In some embodiments, the immune disorder is selected from one or more of type 1 diabetes, vasculitis, and an immunodeficiency.

In some embodiments, the pharmaceutical composition is administered to the subject by parenteral administration. In some embodiments, the parenteral administration is intravenous administration.

Also included is the use of a pharmaceutical composition described herein in the preparation of a medicament for treating a disease in a subject, and/or for enhancing an immune response in a subject. Particular embodiments include a pharmaceutical composition described herein for use in treating a disease in a subject, and/or for enhancing an immune response in a subject.

### BRIEF DESCRIPTION OF THE DRAWINGS

FIG. 1A shows the protein topology of human interleukin 2 (IL-2) and human interleukin 2 receptor alpha chain (IL-2Rα).

FIG. 1B shows the quaternary structure of IL-2 in complex with its receptors IL-2Rα (CD25), IL-2Rβ (CD122) and the common gamma chain (CD132) (PDB: 2ERJ).

FIG. 2A illustrates fusion of the C-terminus of IL-2 to the N-terminus of IL-2Rα with a cleavable/non-cleavable linker. An optional His-tag is added at the C-terminus of IL-2Rα to facilitate purification. A schematic homodimer structure is presented. IL-2 in this fusion protein is not able to bind to and signal through IL-2Rβ/γc receptors. IL-2 activity can be recovered after protease cleavage between IL-2 and IL-2Rα.

FIG. 2B illustrates a diagram of the protein sequence motifs and configurations for proteins described in FIG. 2A.

FIG. 2C illustrates fusion of the C-terminus of Fc to the N-terminus of IL-2 with a cleavable/non-cleavable linker and fusion of the C-terminus of IL-2 to the N-terminus of IL-2Rα with a cleavable/non-cleavable linker. IL-2 in this fusion protein is not able to bind to and signal through IL-2Rβ/γc receptors. Partial activity can be restored after protease cleavage between Fc and IL-2, and full activity can be recovered after protease cleavage between IL-2 and IL-2Rα or after protease cleavage between IL-2/IL-2Rα and Fc/IL-2.

FIG. 2D illustrates a diagram of the protein sequence motifs and configurations for proteins described in FIG. 2C.

FIG. 2E illustrates fusion of the C-terminus of IL-2 to the N-terminus of IL-2Rα with a cleavable/non-cleavable linker and fusion of the C-terminus of IL-2Rα to the N-terminus of Fc with a cleavable/non-cleavable linker. IL-2 in this fusion protein is not able to bind to and signal through IL-2Rβ/γc receptors. Partial activity can be restored after protease cleavage between Fc and IL-2, and full activity can be recovered after protease cleavage between IL-2 and IL-2Rα or after protease cleavage between IL-2/IL-2Rα and Fc/IL-2Rα.

FIG. 2F illustrates a diagram of the protein sequence motifs and configurations for proteins described in FIG. 2E.

FIG. 3A illustrates fusion of the C-terminus of IL-2Rα to the N-terminus of IL-2 with a cleavable/non-cleavable linker. An optional His-tag is added at the C-terminus of IL-2Rα to facilitate purification. A predicted homodimer structure is presented. IL-2 in this fusion protein is not able to bind to and signal through IL-2Rβ/γc receptors. IL-2 activity can be recovered after protease cleavage between IL-2 and IL-2Rα.

FIG. 3B illustrates a diagram of the protein sequence motifs and configurations for proteins described in FIG. 3A.

FIG. 3C illustrates fusion of the C-terminus of Fc to the N-terminus of IL-2Rα with a cleavable/non-cleavable linker and fusion of the C-terminus of IL-2Rα to the N-terminus of IL-2 with a cleavable/non-cleavable linker. IL-2 in this fusion protein is not able to bind to and signal through IL-2Rβ/γc receptors. Partial activity can be restored after protease cleavage between Fc and IL-2, and full activity can be recovered after protease cleavage between IL-2 and IL-2Rα or after protease cleavage between IL-2/IL-2Rα and Fc/IL-2Rα.

FIG. 3D illustrates a diagram of the protein sequence motifs and configurations for proteins described in FIG. 3C.

FIG. 3E illustrates fusion of the C-terminus of IL-2Rα to the N-terminus of IL-2 with a cleavable/non-cleavable linker and fusion of the C-terminus of IL-2 to the N-terminus of Fc with a cleavable/non-cleavable linker. IL-2 in this fusion protein is not able to bind to and signal through IL-2Rβ/γc receptors. Partial activity can be restored after protease cleavage between Fc and IL-2, and full activity can be recovered after protease cleavage between IL-2 and IL-2Rα or after protease cleavage between IL-2/IL-2Rα and Fc/IL-2.

FIG. 3F illustrates a diagram of the protein sequence motifs and configurations for proteins described in FIG. 3E.

FIG. 4A shows a schematic diagram of activation of an “IL-2-linker-IL-2Rα-linker-His6” activatable proprotein through protease cleavage of the substrate linker sequences between IL-2 and IL-2Rα.

FIG. 4B shows a schematic diagram of activation of an “Fc-linker-IL-2-linker-IL-2Rα ” activatable proprotein through protease cleavage of the substrate linker sequences between IL-2 and IL-2Rα.

FIG. 4C shows a schematic diagram of activation of an “IL-2-linker-IL-2Rα-linker-Fc” activatable proprotein through protease cleavage of the substrate linker sequences between IL-2 and IL-2Rα.

FIG. 4D shows a schematic diagram of activation of an “IL-2-linker-IL-2Rα-linker-Fc” activatable proprotein through protease cleavage of the substrate linker sequences between IL-2/IL-2Rα and IL-2Rα/Fc.

FIG. 4E shows a schematic diagram of partial activation of an “IL-2-linker-IL-2Rα-linker-Fc” activatable proprotein through protease cleavage of the substrate linker sequences between IL-2Rα and Fc.

FIG. 5A illustrates fusion of the C-terminus of a binding moiety to the N-terminus of an IL-2 protein via a cleavable/non-cleavable linker, and fusion of the C-terminus of an IL-2 protein to the N-terminus of an IL-2 binding protein via a cleavable/non-cleavable linker.

FIG. 5B illustrates fusion of the C-terminus of an IL-2 protein to the N-terminus of an IL-2 binding protein via a cleavable/non-cleavable linker, and fusion of the C-terminus of an IL-2 binding protein to the N-terminus of a binding moiety via a cleavable/non-cleavable linker.

FIG. 5C illustrates fusion of the C-terminus of a binding moiety to the N-terminus of an IL-2 binding protein via a cleavable/non-cleavable linker, and fusion of the C-terminus of an IL-2 binding protein to the N-terminus of an IL-2 protein via a cleavable/non-cleavable linker.

FIG. 5D illustrates fusion of the C-terminus of an IL-2 binding protein to the N-terminus of an IL-2 protein via a cleavable/non-cleavable linker, and fusion of the C-terminus of an IL-2 protein to the N-terminus of a binding moiety via a cleavable/non-cleavable linker.

FIGS. 6A-6C show SDS-PAGE results of purified proteins and cleavage of IL-2 fusion proteins. 6A shows non-reducing SDS-PAGE results, 6B shows reducing SDS-PAGE results and 6C shows cleavage results. “M” on the figures represents the protein standard marker. On FIG. 6C, “1” represents proteins before TEV cleavage and “2” represents proteins after TEV cleavage.

FIGS. 7A-7J illustrate representative HPLC analysis results of purified proteins.

FIGS. 8A-8L and FIGS. 9A-9E illustrate the activity of IL-2 fusion proteins on M-07e proliferation as determined by a colorimetric assay (Cell Counting Kit-8 (CCK-8)).

FIGS. 10A-10C show SDS-PAGE results of purified proteins and cleavage of IL-2 fusion proteins. 10A shows non-reducing SDS-PAGE results, 10B shows reducing SDS-PAGE results and 10C shows cleavage results. “M” on the figures represents the protein standard marker. On FIG. 10C, “1” represents proteins before TEV cleavage and “2” represents proteins after TEV cleavage.

FIGS. 11A-11F illustrate representative HPLC analysis results of purified proteins.

FIGS. 12A-12F illustrate the activity of IL-2 fusion proteins on M-07e proliferation determined by a colorimetric assay (Cell Counting Kit-8 (CCK-8)).

FIGS. 13A-13C show SDS-PAGE results of purified proteins and cleavage of IL-2 fusion proteins. 13A shows non-reducing SDS-PAGE results, 13B shows reducing SDS-PAGE results and 13C shows cleavage results. “M” on the figures represents the protein standard marker. On FIG. 13C, “1” represents proteins before uPA cleavage and “2” represents proteins after uPA cleavage.

FIGS. 14A-14D illustrate representative HPLC analysis results of purified proteins.

FIGS. 15A-15E illustrate activity of IL-2 fusion proteins on M-07e proliferation determined by a colorimetric assay (Cell Counting Kit-8 (CCK-8)).

FIGS. 16A-16C show SDS-PAGE results of purified proteins and cleavage of IL-2 fusion proteins. 16A shows non-reducing SDS-PAGE results, 16B shows reducing SDS-PAGE results and 16C shows cleavage results. “M” on the figures represents the protein standard marker. On FIG. 16C, “1” represents proteins before TEV or uPA cleavage and “2” represents proteins after TEV or uPA cleavage. (P1773-P1778 cleaved by TV; P1779-P1785 cleaved by uPA.)

FIGS. 17A-17D illustrate representative HPLC analysis results of purified proteins.

FIG. 18A-18N illustrate the activity of IL-2 fusion proteins on M-07e proliferation determined by a colorimetric assay (Cell Counting Kit-8 (CCK-8)).

FIGS. 19A-19D show SDS-PAGE results of purified proteins and cleavage of IL-2 fusion proteins. 19A shows non-reducing SDS-PAGE results, 19B shows reducing SDS-PAGE results, 19C shows cleavage results with single protease and 19D shows cleavage results with double proteases. “M” on the figures represents the protein standard marker. On FIG. 19C, “1” represents proteins before protease cleavage, “2” represents proteins after uPA cleavage, “3” represents proteins after MMP-2 cleavage and “4” represents proteins after matriptase cleavage. On FIG. 19D, “1” represents proteins before protease cleavage, “2” represents proteins after uPA cleavage, “3” represents proteins after MMP-2 cleavage and “4” represents proteins after double cleavage with uPA and MMP-2.

FIGS. 20A-20D illustrate representative HPLC analysis results of purified proteins.

FIGS. 21A-21Q illustrate the activity of IL-2 fusion proteins on M-07e proliferation determined by a colorimetric assay (Cell Counting Kit-8 (CCK-8)).

FIGS. 22A-22C show SDS-PAGE results of purified proteins and cleavage of IL-2 fusion proteins. 22A shows non-reducing SDS-PAGE results, 22B shows reducing SDS-PAGE results and 22C shows cleavage results. “M” on the figures represents the protein standard marker. On FIG. 22C, “1” represents proteins before TEV cleavage and “2” represents proteins after TEV cleavage.

FIGS. 23A-23D illustrate representative HPLC analysis results of purified proteins.

FIGS. 24A-24D illustrate the activity of IL-2 fusion proteins on M-07e proliferation determined by a colorimetric assay (Cell Counting Kit-8 (CCK-8)).

FIGS. 25A-25C show SDS-PAGE results of purified proteins and cleavage of IL-2 fusion proteins. 25A shows non-reducing SDS-PAGE results, 25B shows reducing SDS-PAGE results, 25C shows cleavage results. “M” on the figures represents the protein standard marker. On FIG. 25C, “1” represents proteins before protease cleavage, “2” represents proteins after MMP-2 cleavage, “3” represents proteins after uPA cleavage and “4” represents proteins after matriptase cleavage.

FIGS. 26A-26D illustrate representative HPLC analysis results of purified proteins.

FIGS. 27A-27D show SDS-PAGE and HPLC results. binding shows non-reducing SDS-PAGE results, binding shows reducing SDS-PAGE results, binding shows cleavage results, and binding shows HPLC analysis results. “M” on the figures represents the protein standard marker. On FIG. 27C, “1” represents proteins before TEV cleavage, “2” represents proteins after TEV cleavage.

FIG. 28 illustrates the activity of IL-2 fusion proteins on M-07e proliferation determined by a colorimetric assay (Cell Counting Kit-8 (CCK-8)).

FIGS. 29A-29B show SD S-PAGE results of purified proteins. 29A shows non-reducing SD S-PAGE results and 29B shows reducing SDS-PAGE results. “M” on the figures represents the protein standard marker.

FIG. 30 shows MMP-2 cleavage results. “M” on the figures represents the protein standard marker. “1” represents proteins before MMP-2 cleavage and “2” represents proteins after MMP-2 cleavage.

FIGS. 31A-31J illustrate representative HPLC analysis results of purified proteins.

FIGS. 32A-32M illustrate the activity of IL-2 proproteins on M-07e proliferation as determined by a colorimetric assay (Cell Counting Kit-8 (CCK-8)).

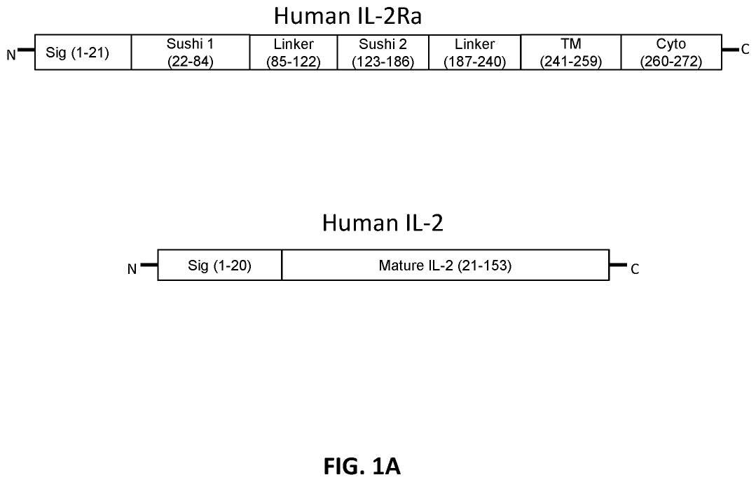

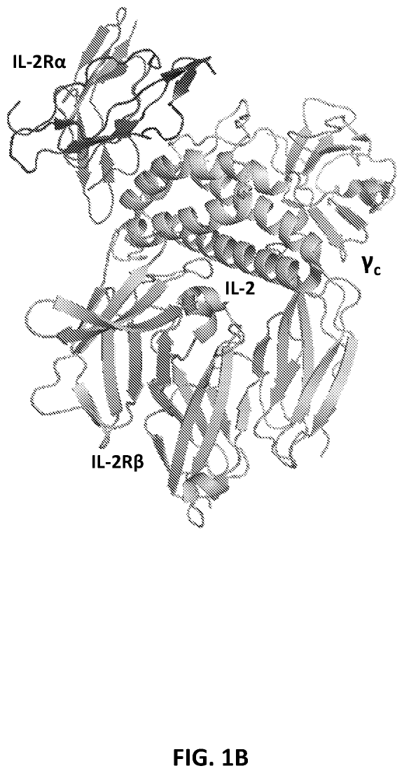

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “activatable proprotein,” “activatable prodrug”, “prodrug” or “proprotein” are used interchangeably herein and refer to an activatable proprotein comprising at least a masking moiety and an active domain, or derivatives/ variants therefrom, as described herein. In one embodiment, the proprotein may also comprise one or more protein domains

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (e.g., activatable proprotein) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₅₀ of an agent (e.g., activatable proprotein) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as an activatable proprotein can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18% , 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., activatable proprotein) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98% , 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60,70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., activatable proprotein) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., activatable proprotein, activated protein) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Activatable Proproteins

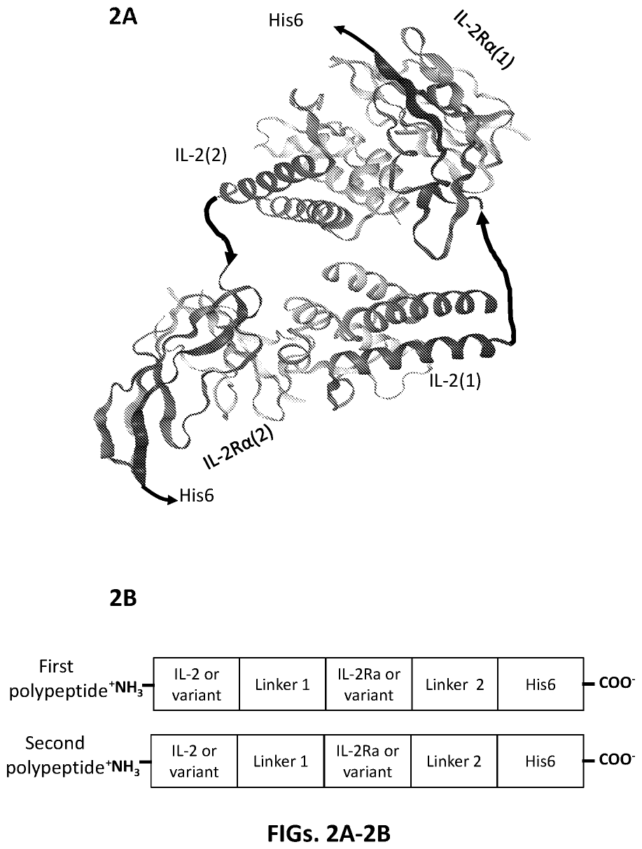

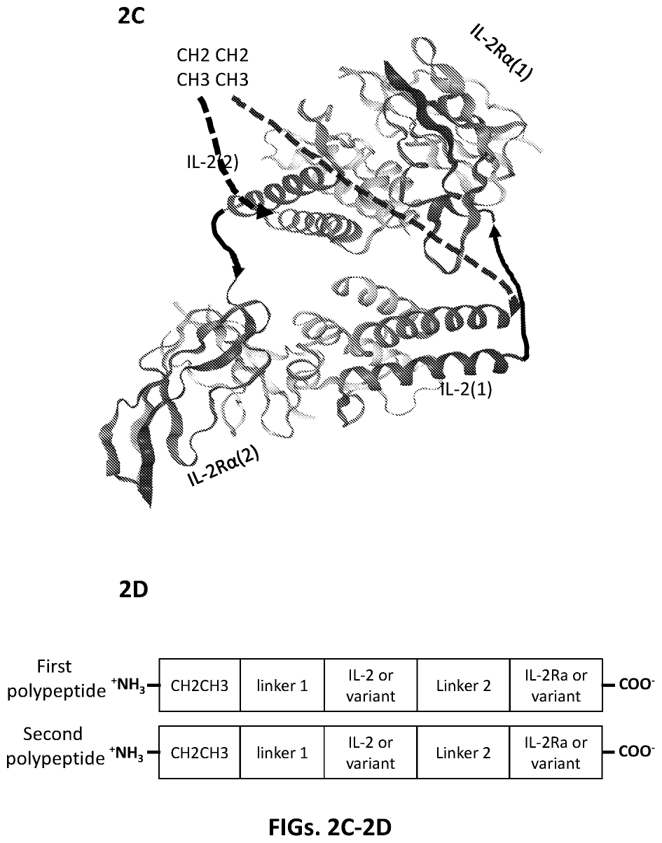

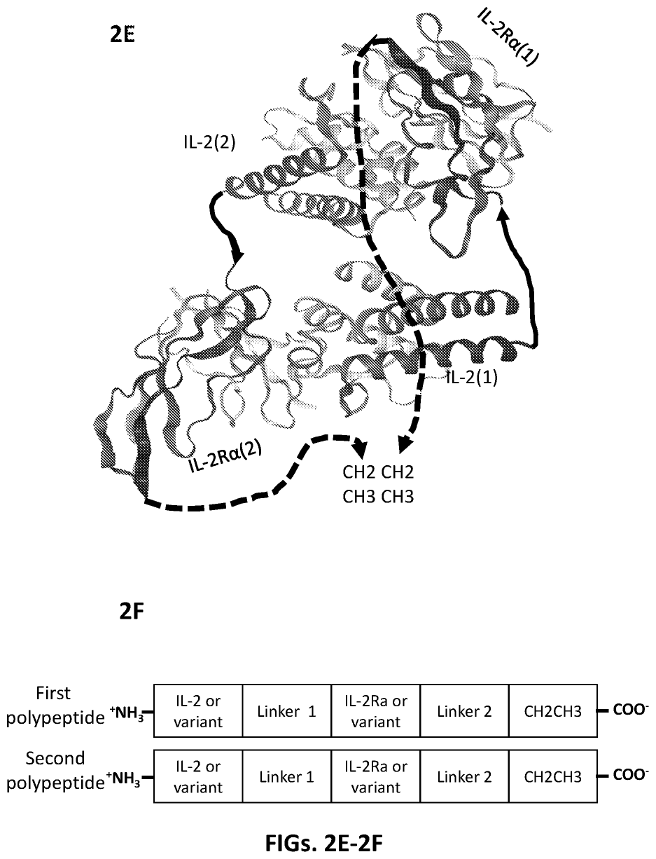

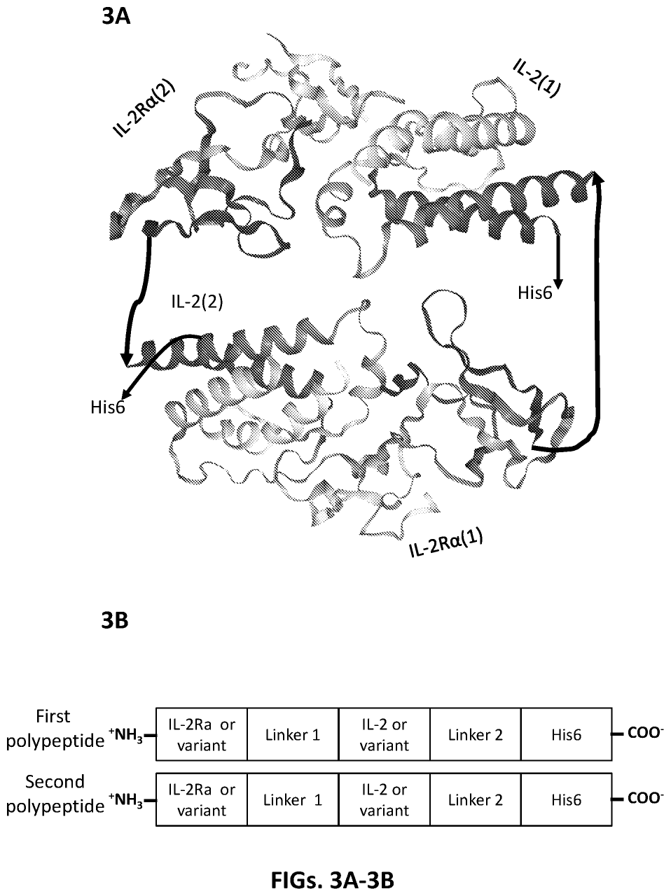

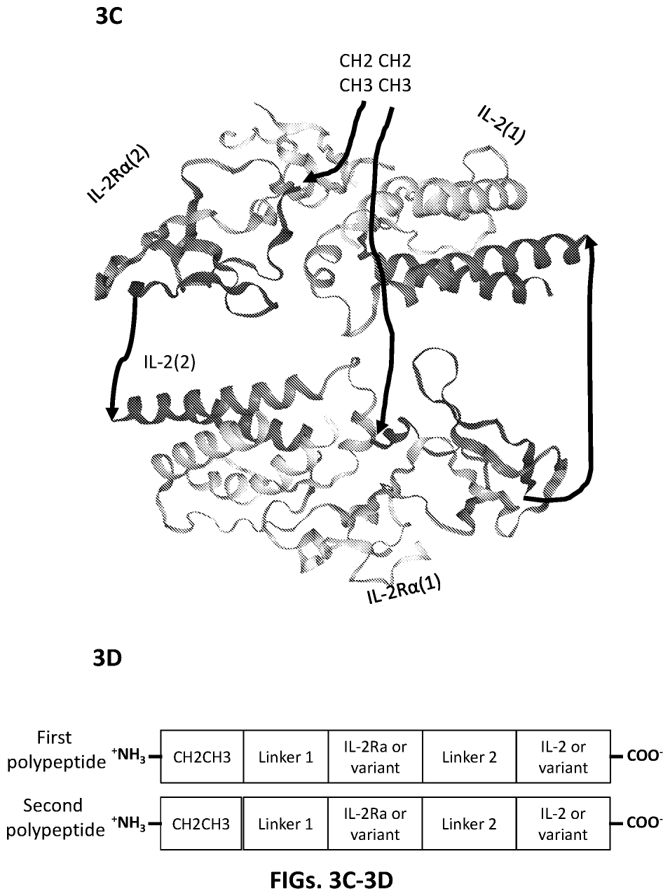

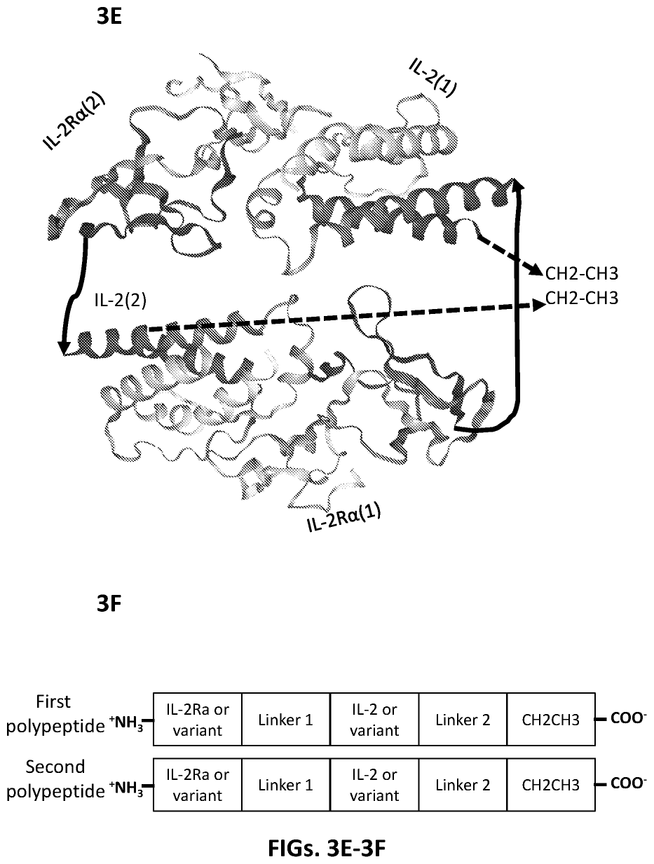

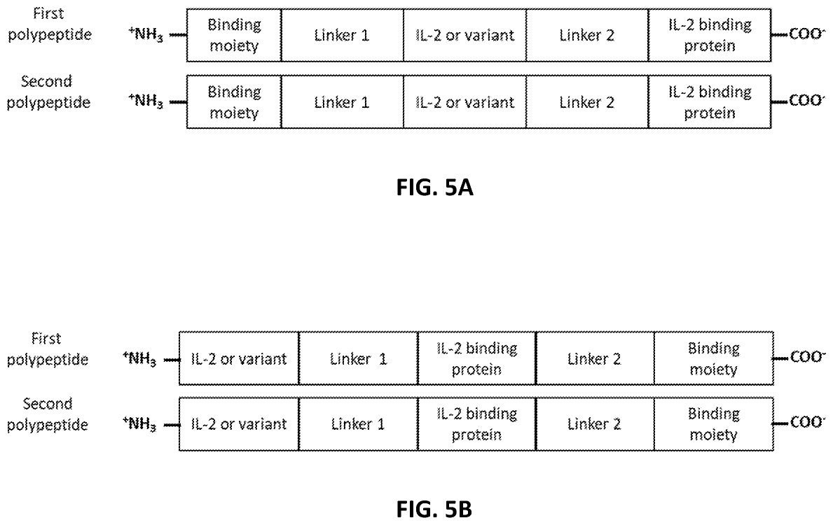

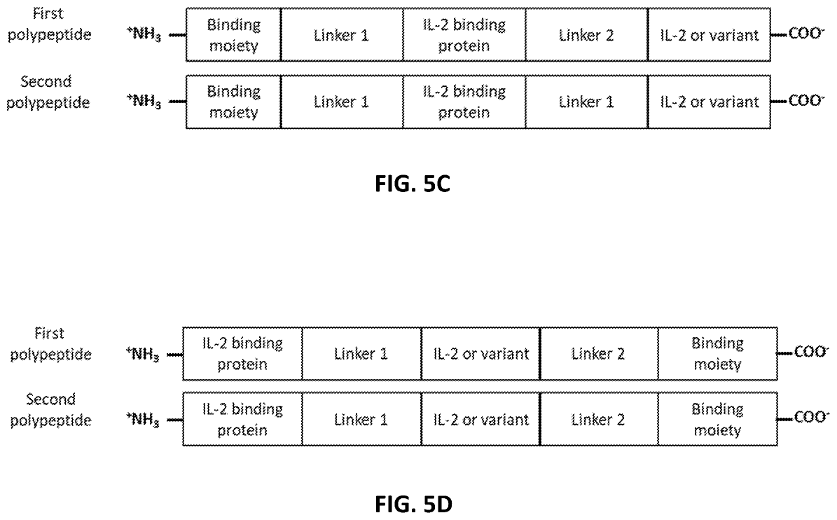

Embodiments of the present disclosure relate to activatable proprotein homodimers, or prodrugs, comprising two IL-2 proteins that remains relatively inactive in the proprotein form, and which can be activated upon contact with the appropriate environment. The activatable proproteins described herein comprise at least two separate but otherwise identical (or substantially identical) polypeptide chains, which bind together via non-covalent interactions and/or certain covalent bonds, for example, disulfide bonds, but not via peptide or amide bonds. Generally, each polypeptide chain comprises an IL-2 protein, an IL-2 binding protein such as an IL-2Rα protein, and a cleavable linker. Here, the IL-2 protein of the first polypeptide binds to the IL-2 binding protein of the second polypeptide, and the IL-2 protein of the second polypeptide binds to the IL-2 binding protein of the first polypeptide, to form a relatively stable homodimer in which these binding interactions block or sterically hinder the IL-2 proteins in each chain from interacting with or binding to their cognate receptor(s) on a cell (see, for example, FIGS. 2A and 2C). In some instances, each polypeptide chain comprises a purification tag at the N- or C-terminus, which is separated from the rest of the polypeptide by a linker (see, for example, FIG. 2B and FIG. 3B). In some instances, each polypeptide chain comprises a binding domain (for example, an Fc domain or a fragment thereof) at the N- or C-terminus, which is separated from the rest of the polypeptide by a linker (see, for example, FIGS. 5A-5D), and which binds to the binding domain on the other polypeptide chain to further stabilize the proprotein homodimer (see, for example, FIGS. 2C, 2E, 3C, and 3D). As noted above, at least one of the linkers is a cleavable linker, which upon cleavage in a target cell or tissue restores IL-2 activity by opening the homodimer and exposing at least one active or binding site of the IL-2 proteins. Such allows the IL-2 portions of the now activated protein(s) to interact with or bind to certain of their cognate receptor(s), for example, IL-2Rβ/γc and/or IL-2Rα/β/γc receptor chains on an immune cell, and thereby effect downstream immune cell-signaling pathways.

The activatable proproteins described herein address many of the drawbacks of standard IL-2 therapies in the treatment of cancer, infectious diseases, and other diseases, including high initial serum Cₘₐₓ, which causes over-activation of the immune system, preferential activation of regulatory T cells expressing IL-2Rα/β/γc receptor chains relative to immune cells expressing IL-2Rβ/γc receptor chains, short PK because of the otherwise small molecular size of IL-2 and/or catabolism by the large number of immune cells that express IL-2 receptors, poor accumulation in the target tissues (e.g., cancers, tumors) because of the short PK and/or ineffective tumor targeting, and undesirable accumulation and immune activation in normal tissues.

Embodiments of the present disclosure thus include an activatable proprotein homodimer (complex), comprising a first polypeptide (chain) and a second polypeptide (chain),

wherein the first polypeptide and the second polypeptide comprise, in an N- to C-terminal orientation, or a C- to N-terminal orientation, a binding moiety, a first linker, an IL-2 protein, a second linker, and an IL-2 binding protein;

or wherein the first polypeptide and the second polypeptide comprise, in an N- to C-terminal orientation, or a C- to N-terminal orientation, a binding moiety, a first linker, an IL-2 binding protein, a second linker, and an IL-2 protein,

wherein the binding moiety of the first polypeptide binds to the binding moiety of the second polypeptide, wherein the IL-2 protein of the first polypeptide binds to the IL-2 binding protein of the second polypeptide, and wherein the IL-2 binding protein of the first polypeptide binds to the IL-2 protein of the second polypeptide, wherein said (collective) binding masks a binding site of IL-2 protein(s) that otherwise binds to an IL-2Rβ/γc and/or IL-2Rα/β/γc chain present on the surface of an immune cell in vitro or in vivo, and wherein at least one of the first or the second linker is a cleavable linker.

Also included is an activatable proprotein homodimer (complex), comprising a first polypeptide (chain) and a second polypeptide (chain),

wherein the first and the second polypeptide comprise, in an N- to C-terminal orientation, or a C- to N-terminal orientation, an IL-2 protein, a first linker, an IL-2 binding protein, a second linker, and optionally an affinity purification tag;

or wherein the first and the second polypeptide comprise, in an N- to C-terminal orientation, or a C- to N-terminal orientation, an IL-2 binding protein, a first linker, an IL-2 protein, a second linker, and optionally an affinity purification tag,

wherein the IL-2 protein of the first polypeptide binds to the IL-2 binding protein of the second polypeptide, and wherein the IL-2 binding protein of the first polypeptide binds to the IL-2 protein of the second polypeptide, wherein said (collective) binding masks a binding site of IL-2 protein(s) that otherwise binds to an IL-2Rβ/γc and/or IL-2Rα/β/γc chain present on the surface of an immune cell in vitro or in vivo, and wherein the first linker is a cleavable linker.

As noted above, the IL-2 protein(s) and the IL-2 binding protein(s) interact or bind together, for example, via non-covalent interactions or certain covalent bonds (e.g., disulfide bonds). In some instances, the binding of the IL-2 protein(s) to the IL-2 binding protein(s), for example, IL-2Rα protein(s), sterically blocks or hinders binding of the IL-2 protein(s) to their cognate IL-2Rα/β/γc receptor chains expressed on regulatory T-cells (T_{regs}). In some instances, that binding and steric hindrance is preserved in the activated form of the protein, and can provide the advantage of minimizing the activation of immunosuppressive T_{regs}, and reducing the consumption of the proprotein and the active protein alike. Exemplary IL-2 proteins and IL-2 binding proteins are described elsewhere herein.

In some instances, the binding moieties of the first and second polypeptides dimerize together via at least one non-covalent interaction, at least one covalent bond (for example, at least one disulfide bond), or any combination of non-covalent interactions and covalent bonds, to further stabilize the activatable proprotein and/or to further mask the binding of the IL-2 proteins to their cognate receptors, for example, IL-2Rα/β/γc and/or IL-2Rβ/γc receptor chains. Typically, however, binding moieties of the first and second polypeptide do not bind together or dimerize via a peptide or amide bond. In some embodiments, the binding moieties bind together as a heterodimer, that is, a heterodimer composed of two different binding moieties. In some embodiments, the binding moieties bind together as a homodimer, that is, a homodimer composed of two identical or nearly identical binding moieties. Thus, the binding moieties of the first and second polypeptides can be the same (or substantially the same) or different. In most instances, the binding moieties of the first and second polypeptides are the same, and do not bind to the IL-2 protein, or the IL-2 binding protein. However, in some instances, one or both of the binding moieties can bind to the IL-2 protein and/or the IL-2 binding protein. Exemplary binding moieties are described herein.

As noted above, at least one of the linkers comprises a cleavable linker, for example, a linker cleavable by a protease. In some instances, one linker comprises a cleavable linker and the other linker is a stable (e.g., physiologically stable) linker. In some instances, both linkers comprise cleavable linkers. In some instances, the protease is expressed in target tissues or cells, for example, cancer tissues or cancer cells. Cleavage of the linker in that context releases a masking moiety, removes the steric hindrance of the IL-2 protein, and allows selective activation of the IL-2 protein in diseased tissues or cells, relative to normal or healthy tissues or cells. Such selective and localized activation not only reduces needless consumption of administered IL-2, thereby increasing its half-life, but also enhances tissue penetration and reduces undesirable systemic effects of IL-2, among other advantages. Exemplary linkers are described herein.

In some embodiments, the homodimeric binding between the first and second polypeptides allosterically inhibits the binding of the IL-2 proteins to their target, for example, cognate IL-2Rβ/γc and/or IL-2Rα/β/γc receptor chains on the surface of an immune cell. In these and related embodiments, the activatable proprotein shows no binding or substantially no binding to its target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding to its target, as compared to the binding of the active domain or the IL-2 protein alone, optionally for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater, optionally as measured in vivo or in a Target Displacement in vitro assay available in the art.

The various components of each polypeptide chain can be fused in any orientation. For example, in some embodiments, the first polypeptide and the second polypeptide of comprise, in an N- to C-terminal orientation, the binding moiety, the first linker, the IL-2 protein, the second linker, and the IL-2 binding protein. In some embodiments, the first polypeptide and the second polypeptide of comprise, in an N- to C-terminal orientation, the IL-2 binding protein, the first linker, the IL-2 protein, the second linker, and the binding moiety. In certain embodiments, the first polypeptide and the second polypeptide comprise, in an N- to C-terminal orientation, the binding moiety, the first linker, the IL-2 binding protein, the second linker, and the IL-2 protein. In some embodiments, the first polypeptide and the second polypeptide comprise, in an N- to C-terminal orientation, the IL-2 protein, the first linker, the IL-2 binding protein, the second linker, and the binding moiety. In particular embodiments, the first polypeptide and the second polypeptide comprise, in an N- to C-terminal orientation, the IL-2 protein, the first linker, the IL-2 binding protein, the second linker, and the affinity purification tag. In some embodiments, the first polypeptide and the second polypeptide of (d) comprise, in an N- to C-terminal orientation, the IL-2 binding protein, the first linker, the IL-2 protein, the second linker, and the affinity purification tag. Other possible orientations will be apparent to persons skilled in the art.

Certain activatable proproteins are composed only of two of the foregoing protein chains, that is, they are composed only of a first polypeptide and a second polypeptide, as described herein. In some instances, however, certain activatable proproteins comprise multiple chains, for example, where the first and second polypeptide chains form a “core structure” upon which additional or higher-order structures can be built, the various core structures being optionally bound together via additional protein binding domains.

The individual components of the activatable proproteins are described in greater detail herein.

IL-2 Proteins. The activatable proproteins described herein comprise at least one “IL-2 protein” (or Interleukin-2 protein), including human IL-2 proteins. IL-2 is a cytokine signals through the IL-2 receptor (IL-2R), a complex composed of up to three chains, termed the α (CD25), β (CD122) and γc (CD132) chains. 1L-2 is produced by T-cells in response to antigenic or mitogenic stimulation, and is required for T-cell proliferation and other activities crucial to regulation of the immune response. IL-2 can stimulate B-cells, monocytes, lymphokine-activated killer cells, natural killer cells, and glioma cells, among other immune cells.

IL-2 is a 15-16 kDA protein composed of a signal peptide (residues 1-20) and an active mature protein (residues 21-153). Exemplary human IL-2 amino acid sequences are provided in Table S1 below.

TABLE S1
Exemplary IL-2 Peptides

| | | SEQ ID |
|---|---|---|
| Name | Sequence | NO: |
| Human IL-2 | MYRMQLLSCIALSLALVTNSAPTSSSTKKTQLQLEHLLLDLQMILNGINNYK | 1 |
| FL | NPKLTRMLTFKFYMPKKATELKHLQCLEEELKPLEEVLNLAQSKNFHLRPRD | |
| Precursor | LISNINVIVLELKGSETTFMCEYADETATIVEFLNRWITFCQSIISTLT | |
| Human IL-2 | APTSSSTKKTQLQLEHLLLDLQMILNGINNYKNPKLTRMLTFKFYMPKKATE | 2 |
| mature | LKHLQCLEEELKPLEEVLNLAQSKNFHLRPRDLISNINVIVLELKGSETTFM | |
| form C125S | CEYADETATIVEFLNRWITFSQSIISTLT | |
| Human IL-2 | APTSSSTKKTQLQLEHLLLDLQMILNGINNYKNPKLTRMLTFKFYMPKKATE | 3 |
| mature | LKHLQCLEEELKPLEEVLNLAHSKNFHFDPRDVVSNINVFVLELKGSETTFM | |
| form (D10) | CEYADETATIVEFLNRWITFCQSIISTLT | |
| Q74H, L80F, | | |
| R81D, L85V, | | |
| I86V, and | | |
| I92F | | |
| Human IL-2 | APTSSSTKKTQLQLEHLLLDLQMILNGINNYKNPKLTRMLTFKFYMPKKATE | 7 |
| mature | LKHLQCLEEELKPLEEALNLAPSKNFHLRPRDLISNINVIVLELKGSETTFM | |
| form with | CEYADETATIVEFLNRWITFCQSTISTLT | |
| V69A, Q74P | | |
| and I128T | | |
| Human IL-2 | APTSSSTKKTQLQLEHLLLDLQMILNGINNYKNPKLTRMLTAKFAMPKKATE | 8 |
| mature | LKHLQCLEEELKPLEEVLNGAQSKNFHLRPRDLISNINVIVLELKGSETTFM | |
| form with | CEYADETATIVEFLNRWITFCQSIISTLT | |
| F42A, Y45A | | |
| and L72G | | |
| Human IL-2 | APTSSSTKKTQLQLEHLLLDLQMILNGINNYKNPKLTKMLTQKFNMPKKATE | 9 |
| mature | LKHLQCLEELLKPLEVVLNLAQSKNFHLRPRDLISNINVIVLELKGSETTFM | |
| form with | CEYADETATIVEFLNRWITFCQSIISTLT | |
| R38K, | | |
| F42Q, | | |
| Y45N, E62L | | |
| and E68V | | |
| Human IL-2 | APTSSSTKKTQLQLEHLLLDLQMILNGINNYKNPKLTKMLTQKFEMPKKATE | 10 |
| mature | LKHLQCLEEELKPLEVVLNLAQSKNFHLRPRDLISNINVIVLELKGSETTFM | |
| form with | CEYADETATIVEFLNRWITFCQSIISTLT | |
| R38K, | | |
| F42Q, Y45E | | |
| and E68V | | |
| Human IL-2 | APTSSSTKKTQLQLEHLLLDLQMILNGINNYKNPKLTAMLTIKFNMPKKATE | 11 |
| mature | LKHLQCLEELLKPLEVVLNLAQSKNFHLRPRDLISNINVIVLELKGSETTFM | |
| form with | CEYADETATIVEFLNRWITFCQSIISTLT | |
| R38A, | | |
| F42I, | | |
| Y45N, E62L | | |
| and E68V | | |
| Human IL-2 | APTSSSTKKTQLQLEHLLLDLQMILNGINNYKNPKLTKMLTKKFRMPKKATE | 12 |
| mature | LKHLQCLEELLKPLEVVLNLAQSKNFHLRPRDLISNINVIVLELKGSETTFM | |
| form with | CEYADETATIVEFLNRWITFCQSIISTLT | |
| R38K, | | |
| F42K, | | |
| Y45R, E62L | | |
| and E68V | | |
| Human IL-2 | APTSSSTKKTQLQLEHLLLDLQMILNGINNYKNPKLTKMLTIKFEMPKKATE | 13 |
| mature | LKHLQCLEEELKPLEVVLNLAQSKNFHLRPRDLISNINVIVLELKGSETTFM | |
| form with | CEYADETATIVEFLNRWITFCQSIISTLT | |
| R38K, | | |
| F42I, Y45E | | |
| and E68V | | |
| Human IL-2 | APTSSSTKKTQLQLEHLLLDLQMILNGINNYKNPKLTAMLTAKFAMPKKATE | 14 |
| mature | LKHLQCLEEALKPLEEVLNLAQSKNFHLRPRDLISNINVIVLELKGSETTFM | |
| form with | CEYADETATIVEFLNRWITFCQSIISTLT | |
| R38A, | | |
| F42A, Y45A | | |
| and E62A | | |
| Human IL-2 | APASSSTKKTQLQLEHLLLDLQMILNGINNYKNPKLTRMLTFKFYMPKKATE | 15 |
| mature | LKHLQCLEEELKPLEEVLNLAQSKNFHLRPRDLISNINVIVLELKGSETTFM | |
| form with | CEYADETATIVEFLNRWITFCQSIISTLT | |
| T3A | | |
| Human IL-2 | APTSSSTKKTQLQLEHLLLDLQMILNGINNYKNPKLTRMLTAKFYMPKKATE | 16 |
| mature | LKHLQCLEEELKPLEEVLNLAQSKNFHLRPRDLISNINVIVLELKGSETTFM | |
| form with | CEYADETATIVEFLNRWITFCQSIISTLT | |
| F42A | | |
| Human IL-2 | APTSSSTKKTQLQLEHLLLDLQMILNGINNYKNPKLTRMLTFKFAMPKKATE | 17 |
| mature | LKHLQCLEEELKPLEEVLNLAQSKNFHLRPRDLISNINVIVLELKGSETTFM | |
| form with | CEYADETATIVEFLNRWITFCQSIISTLT | |
| Y45A | | |
| Human IL-2 | APTSSSTKKTQLQLEHLLLDLQMILNGINNYKNPKLTRMLTAKFAMPKKATE | 18 |
| mature | LKHLQCLEEELKPLEEVLNLAQSKNFHLRPRDLISNINVIVLELKGSETTFM | |
| form with | CEYADETATIVEFLNRWITFCQSIISTLT | |
| F42A and | | |
| Y45A | | |
| Human IL-2 | APTSSSTKKTQLQLEHLLLDLQMILNGINNYKNPKLTRMLTFKFYMPKKATE | 19 |
| mature | LKHLQCLESELKPLEEVLNLAQSKNFHLRPRDLISNINVIVLELKGSETTFM | |
| form with | CEYADETATIVEFLNRWITFCQSIISTLT | |
| E61S | | |

Thus, in certain embodiments, an IL-2 protein comprises, consists, or consists essentially of an amino acid sequence selected from Table S1, or an active variant or fragment thereof that is at least 80, 85, 90, 95, 98, or 100% identical to a sequence selected from Table S1. In some embodiments, an “active” IL-2 protein or fragment or variant is characterized, for example, by its ability to bind to an IL-2Rβ/γc and/or IL-2Rα/β/γc receptor chain present on the surface of an immune cell in vitro or in vivo, and stimulate downstream signaling activities, absent steric hindrance by the masking moieties described herein. Examples of downstream signaling activities include IL-2 mediated signaling via one or more of the JAK-STAT, PI3K/Akt/mTOR, and MAPK/ERK pathways, including combinations thereof. Altogether, IL-2 signaling stimulates an array of downstream pathways leading to responses that have a significant role in the development, function, and survival of CD4 T cells, CD8 T cells, NK cells, NKT cells, macrophages, and intestinal intraepithelial lymphocytes, among others.

In particular embodiments, the IL-2 protein is a mature form of IL-2, or an active variant or fragment thereof, which comprises, consists, or consists essentially of an amino acid sequence that is at least 80, 85, 90, 95, 98, or 100% identical to amino acids 21-153 of SEQ ID NO: 1. In some embodiments, the IL-2 protein comprises a C145X substitution, as defined by SEQ ID NO: 1, wherein X is any amino acid. In specific embodiments, the IL-2 protein comprises a C145S substitution as defined by SEQ ID NO: 1.

Certain IL-2 proteins comprise, consist, or consist essentially of an amino acid sequence that is at least 80, 85, 90, 95, 98, or 100% identical to SEQ ID NO: 2 (mature human IL-2 with C125S substitution). In some embodiments, an active variant or fragment of SEQ ID NO: 2 retains the S125 residue as defined therein.

Certain IL-2 proteins comprise one or more defined amino acid substitutions relative to the exemplary amino acid sequences in Table S1. For example, some IL-2 proteins comprise one or more amino acid substitutions selected from K35C, R38C, T41C, F42C, E61C, and V69C as defined by SEQ ID NO: 2. In some embodiments, the IL-2 protein forms a disulfide bond with the IL-2 binding protein (e.g., IL-2Rα) via one or more of the cysteine substitutions selected from K35C, R38C, T41C, F42C, E61C, and V69C. Certain IL-2 proteins comprise one or more amino acid substitutions at position 69, 74, and/or 128 as defined by SEQ ID NO: 2, including combinations thereof and including, for example, wherein the one or more amino acid substitutions are selected from V69A, Q74P, and I128T as defined by SEQ ID NO: 2. Some IL-2 proteins comprise one or more amino acid substitutions at position R38, F42, Y45, E62, E68, and/or L72 as defined by SEQ ID NO: 2, including combinations thereof and including, for example, wherein the one or more amino acid substitutions are selected from R38A and R38K; F42A, F42G, F42S, F42T, F42Q, F42E, F42N, F42D, F42R, F42K, and F42I; Y45A, Y45G, Y45S, Y45T, Y45Q, Y45E, Y45N, Y45D, Y45R, and Y45K; E62A and E62L; E68A and E68V; and L72A, L72G, L72S, L72T, L72Q, L72E, L72N, L72D, L72R, and L72K, including combinations thereof. Specific examples include where the IL-2 protein comprises one or a combination of amino acid substitutions selected from F42A, Y45A, and L72G; R38K, F42Q, Y45N, E62L, and E68V; R38K, F42Q, Y45E, and E68V; R38A, F42I, Y45N, E62L, and E68V; R38K, F42K, Y45R, E62L, and E68V; R38K, F42I, Y45E, and E68V; and R38A, F42A, Y45A, and E62A. Some IL-2 proteins comprise one or a combination of amino acid substitutions at T3 and/or E61 as defined by SEQ ID NO: 2, for example, T3A and/or E61S. Thus, an IL-2 protein can comprise any one or more of the foregoing amino acid substitutions, including combinations thereof.

It will be appreciated that any one or more of the foregoing IL-2 proteins can be combined with any of the other components described herein, for example, IL-2 bindings proteins such as IL-2Rα proteins, masking moieties including binding moieties and linkers, and other optional protein domains, to generate one or more activatable proproteins or larger, multi-chain structures comprising the same.

IL-2 Binding Proteins. The activatable proproteins described herein comprise at least one “IL-2 binding protein”. Examples of IL-2 binding proteins include IL-2Rα proteins, including human IL-2Rα proteins, and antibodies and antigen binding fragments thereof that bind to an IL-2 protein described herein.

In particular embodiments, the IL-2 binding protein is a human IL-2Rα protein, or a variant or fragment thereof that binds to an IL-2 protein. Exemplary human IL-2Rα amino acid sequences are provided in Table S2 below.

TABLE S2
Exemplary IL-2Rα Proteins

| | | SEQ ID |
|---|---|---|
| Name | Sequence | NO: |
| Human IL- | MDSYLLMWGLLTFIMVPGCQAELCDDDPPEIPHATFKAMAYKEGTMLNCECK | 4 |
| 2Rα FL | RGFRRIKSGSLYMLCTGNSSHSSWDNQCQCTSSATRNTTKQVTPQPEEQKER | |
| | KTTEMQSPMQPVDQASLPGHCREPPPWENEATERIYHFVVGQMVYYQCVQGY | |
| | RALHRGPAESVCKMTHGKTRWTQPQLICTGEMETSQFPGEEKPQASPEGRPE | |
| | SETSCLVTTTDFQIQTEMAATMETSIFTTEYQVAVAGCVFLLISVLLLSGLT | |
| | WQRRQRKSRRTI | |
| Human IL- | ELCDDDPPEIPHATFKAMAYKEGTMLNCECKRGFRRIKSGSLYMLCTGNSSH | 5 |
| 2Rα-ECD | SSWDNQCQCTSSATRNTTKQVTPQPEEQKERKTTEMQSPMQPVDQASLPGHC | |
| (22-240) | REPPPWENEATERIYHFVVGQMVYYQCVQGYRALHRGPAESVCKMTHGKTRW | |
| | TQPQLICTGEMETSQFPGEEKPQASPEGRPESETSCLVTTTDFQIQTEMAAT | |
| | METSIFTTEYQ | |
| Human IL- | ELCDDDPPEIPHATFKAMAYKEGTMLNCECKRGFRRIKSGSLYMLCTGNSSH | 6 |
| 2Rα-sushi | SSWDNQCQCTSSATRNTTKQVTPQPEEQKERKTTEMQSPMQPVDQASLPGHC | |
| (22-187) | REPPPWENEATERIYHFVVGQMVYYQCVQGYRALHRGPAESVCKMTHGKTRW | |
| | TQPQLICTGE | |
| Human IL- | ELCDDDPPEIPHATFKAMAYKEGTMLNCECKRGFRRISSGSLYMLCTGNSSH | 20 |
| 2Rα-sushi | SSWDNQCQCTSSATRNTTKQVTPQPEEQKERKTTEMQSPMQPVDQASLPGHC | |
| (22-187) | REPPPWENEATERIYHFVVGQMVYYQCVQGYRALHRGPAESVCKMTHGKTRW | |
| with K38S | TQPQLICTGE | |

Thus, in certain embodiments, an IL-2Rα protein comprises, consists, or consists essentially of an amino acid sequence selected from Table S2, or an active variant or fragment thereof that is at least 80, 85, 90, 95, 98, or 100% identical to a sequence selected from Table S2, and which binds to an IL-2 protein. In some embodiments, the IL-2Rα protein comprises, consists, or consists essentially of an amino acid sequence that is at least 80, 85, 90, 95, 98, or 100% to amino acids 22-187 or 22-240 of SEQ ID NO: 4 (full-length wild-type human IL-2Rα).

Certain IL-2Rα proteins comprise one or more defined amino acid substitutions relative to the exemplary amino acid sequences in Table S2. For example, in some instances the IL-2Rα protein comprises one or more cysteine substitutions selected from D4C, D6C, N27C, K38C, S39C, L42C, Y43C, I118C, and H120C as defined by SEQ ID NO: 6 (human IL-2Rα Sushi 1 to Sushi 2 domain). In some instances, the IL-2Rα protein comprises an alanine substitution at position 49 and/or 68 as defined by SEQ ID NO: 6. In some embodiments, the IL-2Rα protein comprises a K38S substitution as defined by SEQ ID NO:6. Thus, an IL-2Rα protein can comprise any one or more of the foregoing amino acid substitutions, including combinations thereof.

In certain of these and related embodiments, the IL-2Rα protein forms at least one disulfide bond with the IL-2 protein via one or more of the foregoing cysteines and one or more cysteines in the IL-2 protein. In specific embodiments, the IL-2Rα and IL-2 protein form disulfide at least one disulfide bond between one or more cysteine pairs selected from IL2-K35C and IL2Rα-D4C, IL2-R38C and IL2Rα-D6C, IL2-R38C and IL2Rα-H120C, IL2-T41C and IL2Rα-I118C, IL2-F42C and IL2Rα-N27C, IL2-E61C and IL2Rα-K38C, IL2-E61C and IL2Rα-S39C, and IL2-V69C and IL2Rα-L42C. In particular embodiments, as noted above, the binding (for example, disulfide binding) between the IL-2 protein and the IL-2Rα protein masks or sterically hinders the binding site of the IL-2 protein that preferentially binds to the IL-2Rα/β/γc chain expressed on T_{regs}. In some instances, the active or activated form of the protein, following cleavage of at least one linker and release of the corresponding masking moiety, retains the binding between the IL-2 protein and the IL-2Rα protein, and thus does not preferentially bind to the IL-2Rα/β/γc chain expressed on T_{regs}.

As noted above, in certain embodiments, the IL-2 binding protein comprises an antibody or antigen binding fragment thereof that specifically binds to the IL-2 protein. Examples include a whole antibody, Fab, Fab′, F(ab′)2, monospecific Fab2, bispecific Fab2, FV, single chain Fv (scFv), scFV-Fc, nanobody, diabody, camelid, and a minibody. In specific embodiments, the antibody is NARA1 or an antigen binding fragment thereof (see, for example, Arenas-Ramirez et al., Science Translational Medicine. 8: 367ra166, 2016; and U.S. Application No. 2019/0016797, herein incorporated by reference). In particular embodiments, and likewise to above, the binding (for example, disulfide binding) between the IL-2 protein and the anti-IL-2 antibody (or antigen binding fragment thereof) masks or sterically hinders the binding site of the IL-2 protein that preferentially binds to the IL-2Rα/β/γc chain expressed on T_{regs}. In some instances, the active or activated form of the protein, following cleavage of at least one linker and release of the corresponding masking moiety, retains the binding between the IL-2 protein and the IL-2Rα protein, and thus does not preferentially bind to the IL-2Rα/β/γc chain expressed on T_{regs}.

As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target, such as an immune checkpoint molecule, through at least one epitope recognition site, located in the variable region of the immunoglobulin molecule. The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} sequence from antibodies that bind to a target molecule.

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, an IL-2 protein or an epitope or complex thereof, with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for an IL-2 protein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as a polypeptide or antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff /Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of an antibody for an IL-2 protein or epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv (scFV) antibodies are contemplated. For example, Kappa bodies (Ill et al., Prot. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (scFv) polypeptide is a covalently linked VH::VL heterodimer which is expressed from a gene fusion including VH- and VL-encoding genes linked by a peptide-encoding linker. Huston et al. (PNAS USA. 85(16):5879-5883, 1988). A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, the antibodies or antigen-binding fragments described herein are in the form of a “diabody.” Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen-binding site: antigen-binding sites are formed by the association of the first domain of one polypeptide within the multimer with the second domain of another polypeptide within the multimer (WO94/13804). A dAb fragment of an antibody consists of a VH domain (Ward et al., Nature 341:544-546, 1989). Diabodies and other multivalent or multispecific fragments can be constructed, for example, by gene fusion (see WO94/13804; and Holliger et al., PNAS USA. 90:6444-6448, 1993)).

Minibodies comprising a scFv joined to a CH3 domain are also included (see Hu et al., Cancer Res. 56:3055-3061, 1996). See also Ward et al., Nature. 341:544-546, 1989; Bird et al., Science. 242:423-426, 1988; Huston et al., PNAS USA. 85:5879-5883, 1988); PCT/US92/09965; WO94/13804; and Reiter et al., Nature Biotech. 14:1239-1245, 1996.

Where bispecific antibodies are to be used, these may be conventional bispecific antibodies, which can be manufactured in a variety of ways (Holliger and Winter, Current Opinion Biotechnol. 4:446-449, 1993), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al., Protein Eng., 9:616-621, 1996).

In certain embodiments, the antibodies or antigen-binding fragments described herein are in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies and antigen-binding fragments described herein are in the form of a nanobody. Minibodies are encoded by single genes and are efficiently produced in almost all prokaryotic and eukaryotic hosts, for example, E. coli (see U.S. Pat. No. 6,765,087), molds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells. Also included are heavy chain dimers, such as antibodies from camelids and sharks. Camelid and shark antibodies comprise a homodimeric pair of two chains of V-like and C-like domains (neither has a light chain). Since the VH region of a heavy chain dimer IgG in a camelid does not have to make hydrophobic interactions with a light chain, the region in the heavy chain that normally contacts a light chain is changed to hydrophilic amino acid residues in a camelid. VH domains of heavy-chain dimer IgGs are called VHH domains. Shark Ig-NARs comprise a homodimer of one variable domain (termed a V-NAR domain) and five C-like constant domains (C-NAR domains).

In camelids, the diversity of antibody repertoire is determined by the complementary determining regions (CDR) 1, 2, and 3 in the VH or VHH regions. The CDR3 in the camel VHH region is characterized by its relatively long length averaging 16 amino acids (Muyldermans et al., 1994, Protein Engineering 7(9): 1129). This is in contrast to CDR3 regions of antibodies of many other species. For example, the CDR3 of mouse VH has an average of 9 amino acids. Libraries of camelid-derived antibody variable regions, which maintain the in vivo diversity of the variable regions of a camelid, can be made by, for example, the methods disclosed in U.S. Patent Application Ser. No. 20050037421, published Feb. 17, 2005

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

It will be appreciated that any one or more of the foregoing IL-2 binding proteins can be combined with any of the other components described herein, for example, IL-2 proteins, masking moieties including binding moieties and linkers, and other optional protein domains, to generate one or more activatable proproteins or larger, multi-chain structures comprising the same.

Binding Moieties. As noted above, the activatable proprotein homodimers described herein comprise a first polypeptide and a second polypeptide, each of which comprises a “binding moiety”. The binding moiety facilitates and further stabilizes the binding interaction between the first and second polypeptides. In some embodiments, the binding moieties do not bind to the IL-2 protein or the IL-2 binding protein.

General examples of binding moieties are provided in Table M1 below.

TABLE M1
Exemplary Binding Moieties

Short peptide
Leucine zipper peptide
VH
VL
VH-CH1
VL-CL
VH-CL
VL-CH1
CH3
CH2CH3
Fab-CH3
Fab-CH2CH3
Antigen binding domain-CH3
Antigen binding domain-CH2CH3
CH3 variant
CH2CH3 variant
Fab-CH3 variant
Fab-CH2CH3 variant
Antigen binding domain-CH3 variant
Antigen binding domain-CH2CH3 variant

Thus, in certain embodiments, a binding moiety is selected from Table M1.

In particular embodiments, a binding moiety comprises an antigen binding domain of an immunoglobulin, including antigen binding fragments and variants thereof, such as a VL domain and/or a VH domain. In some embodiments, an antigen binding domain does not bind to an antigen, for example, a human antigen. In some embodiments, an antigen binding domain binds to an antigen, for example, a human antigen.

In some embodiments, a binding moiety comprises a constant domain of an immunoglobulin, or a fragment or variant thereof. For example, in certain embodiments a binding moiety comprises a CH1, CH2, CH3, CH1CH3, CH2CH3, CH1CH2CH3, and/or CL domain of an immunoglobulin, including fragments and variants thereof, and combinations thereof. In some instances, the light chain (CL) is a lambda or kappa chain. In some embodiments, the constant domains present in binding moiety of an activatable proprotein homodimer provided herein is glycosylated. In some embodiments, the glycosylation is N-glycosylation. In some embodiments, the glycosylation is O-glycosylation.

In specific embodiments, a binding moiety comprises, in an N- to C- terminal orientation: (1) an antigen binding domain of an immunoglobulin, including antigen binding fragments and variants thereof; and (2) an immunoglobulin constant domain, including fragments and variants thereof, for example, a CH1, CH2, CH3, CH1CH3, CH2CH3, CH1CH2CH3, and/or CL domain of an immunoglobulin, including combinations thereof. In specific embodiments, a binding moiety comprises, consists, or consists essentially of a CH2CH3 domain of an immunoglobulin.

The immunoglobulin domains used herein (antigen binding domains, constant domains) optionally comprise IgG domains. However, certain embodiments comprise alternate immunoglobulins such as IgM, IgA, IgD, and IgE. Furthermore, all possible isotypes of the various immunoglobulins are also encompassed within the current embodiments. Thus, IgG1, IgG2, IgG3, etc., are all possible molecules in the binding domains. In addition to choice in selection of the type of immunoglobulin and isotype, certain embodiments comprise various hinge regions (or functional equivalents thereof). Such hinge regions provide flexibility between the different domains of the proproteins described herein. In some embodiments, the immunoglobulin portion of the binding domain (or larger masking moiety) is from an immunoglobulin class selected from IgG1, IgG2, IgG3, IgG4, IgD, IgA, and IgM.

Linkers. As noted above, in certain embodiments, each polypeptide comprises at least one or two linkers, or peptide linkers. In some embodiments, at least one of the linkers is a cleavable linker, for example, a cleavable linker that comprises a protease cleavage site. In some embodiments, at least one of the linkers is a non-cleavable linker, that is, a physiologically-stable linker.

In some embodiments, the first linker and/or the second linker are about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length. In particular embodiments, the first linker is a cleavable linker, and the second linker is a non-cleavable linker. In some embodiments, the first linker is a non-cleavable linker, and the second linker is a cleavable linker. In some embodiments, both linkers are cleavable linkers. In some embodiments, a cleavable linker comprises at least one protease cleavage site.

Suitable protease cleavages sites and self-cleaving peptides are known to the skilled person (see, e.g., Ryan et al., J. Gener. Virol. 78:699-722, 1997; and Scymczak et al., Nature Biotech. 5:589-594, 2004). In some embodiments, the protease cleavage site is cleavable by a protease selected from one or more of a metalloprotease, a serine protease, a cysteine protease, and an aspartic acid protease. In particular embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

Examples of cleavable linkers are provided in Table S3 below.

TABLE S3
Exemplary cleavable linkers

| Name | Sequence | SEQ ID NO: |
|---|---|---|
| CTMX | GSLSGRSDNHGS | 21 |
| PS1 | GSLGGSGRSANAGS | 22 |
| PS2 | GGSLSGRSANAGGS | 23 |
| PS3 | GPLGLAGRSANAGS | 24 |
| PS4 | PLGLSGRSANAGPA | 25 |
| PS5 | PLGLAGRSANAGPA | 26 |
| PS6 | GPLGLSGRSANAGPASG | 27 |
| PS7 | GPLGLAGRSANAGPASG | 28 |
| PS8 | SGPLGLAGRSANAGPAS | 29 |
| PS9 | SGPASGRSANAPLGLAG | 30 |
| PS10 | GSGPASGRSANAPLGLAGS | 31 |
| PS11 | GPLGLAGRSANPGPASG | 32 |
| PS12 | GPLGLAGRSDNHGPASG | 33 |
| PS13 | GPLGLAGRSDNPGPASG | 34 |
| PS14 | GPLGLAGRSENPGPASG | 35 |
| PS15 | GPLGLAGRSDNLGPASG | 36 |
| PS16 | GPLGLAGRNAQVGPASG | 37 |
| PS17 | GSLSGRSDNAGS | 38 |
| PS18 | GSLSGRSDNDGS | 39 |
| PS19 | GSLSGRSDNEGS | 40 |
| PS20 | GSLSGRSDNFGS | 41 |
| PS21 | GSLSGRSDNGGS | 42 |
| PS22 | GSLSGRSDNIGS | 43 |
| PS23 | GSLSGRSDNKGS | 44 |
| PS24 | GSLSGRSDNLGS | 45 |
| PS25 | GSLSGRSDNMGS | 46 |
| PS26 | GSLSGRSDNNGS | 47 |
| PS27 | GSLSGRSDNPGS | 48 |
| PS28 | GSLSGRSDNQGS | 49 |
| PS29 | GSLSGRSDNRGS | 50 |
| PS30 | GSLSGRSDNSGS | 51 |
| PS31 | GSLSGRSDNTGS | 52 |
| PS32 | GSLSGRSDNVGS | 53 |
| PS33 | GSLSGRSDNWGS | 54 |
| PS34 | GSLSGRSDNYGS | 55 |
| PS35 | GSLSGRSANDGS | 56 |
| PS36 | GSLSGRSANEGS | 57 |
| PS37 | GSLSGRSANFGS | 58 |
| PS38 | GSLSGRSANGSS | 59 |
| PS39 | GSLSGRSANHGS | 60 |
| PS40 | GSLSGRSANIGS | 61 |
| PS41 | GSLSGRSANKGS | 62 |
| PS42 | GSLSGRSANLGS | 63 |
| PS43 | GSLSGRSANMGS | 64 |
| PS44 | GSLSGRSANNGS | 65 |
| PS45 | GSLSGRSANPGS | 66 |
| PS46 | GSLSGRSANQSS | 67 |
| PS47 | GSLSGRSANRGS | 68 |
| PS48 | GSLSGRSANSGS | 69 |
| PS49 | GSLSGRSANTGS | 70 |
| PS50 | GSLSGRSANVGS | 71 |
| PS51 | GSLSGRSANWGS | 72 |
| PS52 | GSLSGRSANYSS | 73 |
| PS12b | GPLGLAGRSDNHSG | 74 |
| PS53 | PLGLAGSGRSDNR | 75 |
| PS54 | PLGLAGSGRSDNRGS | 76 |
| PS55 | GSPLGLAGSGRSDNRGS | 77 |
| PS103 | PLGLAGSGRSDNRGA | 78 |
| PS104 | PLGLAGSGRSDNQGA | 79 |
| PS105 | PLGLAGSGRSDNYGA | 80 |
| PS106 | GPLGLAGSGRSDNQG | 81 |
| PS107 | GSPLGLAGSGRSDNQGA | 82 |
| PS108 | GGSPLGLAGSGRSDNQGGA | 83 |
| PS109 | GGGSPLGLAGSGRSDNQGGGA | 84 |
| PS110 | GGSGSPLGLAGSGRSDNQGGGGA | 85 |
| PS111 | GGSGGSPLGLAGSGRSDNQGGSGGA | 86 |
| PS112 | GPLGLAGSGRSDNRG | 87 |
| PS113 | GSPLGLAGSGRSDNRGA | 88 |
| PS114 | GGSPLGLAGSGRSDNRGGA | 89 |
| PS115 | GGGSPLGLAGSGRSDNRGGGA | 90 |
| PS116 | GGSGSPLGLAGSGRSDNRGGGGA | 91 |
| PS117 | GGSGGSPLGLAGSGRSDNRGGSGGA | 92 |
| PS118 | GGSGGSPLGLAGSGRSDNHGGSGGA | 93 |
| | PLGLAGSGRSDNQGA | 94 |
| | GPLGLAGSGRSDNQGA | 95 |
| | GGPLGLAGSGRSDNQGA | 96 |
| | GGSPLGLAGSGRSDNHGGA | 97 |
| | GGSPLGLAGSGRSDNQGGA | 98 |
| | GGSPLGLAGSGRSDNRGGA | 99 |
| | GGSPLGLAGSGRSDNTGGA | 100 |
| | GGGSGRSDNHGGA | 101 |
| | GGGGSGRSDNHGGA | 102 |
| | GGGGSSGRSDNHGGGA | 103 |
| | GGSGGSSGRSDNHGGGGA | 104 |
| | GGSGGSSGRSDNHGGSGGA | 105 |
| | GGSGRSDNHGGA | 106 |
| | GGSGGSGRSDNHGGA | 107 |
| | GSPLGLAGS | 108 |
| | GGSPLGLAGGS | 109 |
| | GGSPLGLAGGGS | 110 |
| | GGGSPLGLAGGGS | 111 |
| | GGGSPLGLAGGGGS | 112 |
| | GGSGGGPLGLAGGGA | 113 |
| | GGSGGSGRSDNHGGA | 114 |
| | SGRSDNA | 442 |
| | SGRSDND | 443 |
| | SGRSDNE | 444 |
| | SGRSDNF | 445 |
| | SGRSDNG | 446 |
| | SGRSDNI | 447 |
| | SGRSDNK | 448 |
| | SGRSDNL | 449 |
| | SGRSDNM | 450 |
| | SGRSDNN | 451 |
| | SGRSDNP | 452 |
| | SGRSDNQ | 453 |
| | SGRSDNR | 454 |
| | SGRSDNS | 455 |
| | SGRSDNT | 456 |
| | SGRSDNV | 457 |
| | SGRSDNW | 458 |
| | SGRSDNY | 459 |
| | SGRSAND | 460 |
| | SGRSANE | 461 |
| | SGRSANF | 462 |
| | SGRSANG | 463 |
| | SGRSANH | 464 |
| | SGRSANI | 465 |
| | SGRSANK | 466 |
| | SGRSANL | 467 |
| | SGRSANM | 468 |
| | SGRSANN | 469 |
| | SGRSANP | 470 |
| | SGRSANQ | 471 |
| | SGRSANR | 472 |
| | SGRSANS | 473 |
| | SGRSANT | 474 |
| | SGRSANV | 475 |
| | SGRSANW | 476 |
| | SGRSANY | 477 |

Thus, in certain embodiment, a cleavable linker is selected from Table S3. Additional examples of cleavable linkers include an amino acid sequence cleaved by a serine protease such as thrombin, chymotrypsin, trypsin, elastase, kallikrein, or subtilisin. Illustrative examples of thrombin-cleavable amino acid sequences include, but are not limited to: -Gly-Arg-Gly-Asp-(SEQ ID NO:115), -Gly-Gly-Arg-, -Gly-Arg-Gly-Asp-Asn-Pro-(SEQ ID NO: 116), -Gly-Arg-Gly-Asp-Ser-(SEQ ID NO: 117), -Gly-Arg-Gly-Asp-Ser-Pro-Lys-(SEQ ID NO: 118), -Gly-Pro-Arg-, -Val-Pro-Arg-, and -Phe-Val-Arg-. Illustrative examples of elastase-cleavable amino acid sequences include, but are not limited to: -Ala-Ala-Ala-, -Ala-Ala-Pro-Val-(SEQ ID NO: 119), -Ala-Ala-Pro-Leu-(SEQ ID NO: 120), -Ala-Ala-Pro-Phe-(SEQ ID NO: 121), -Ala-Ala-Pro-Ala-(SEQ ID NO: 122), and -Ala-Tyr-Leu-Val-(SEQ ID NO: 123).

Cleavable linkers also include amino acid sequences that can be cleaved by a matrix metalloproteinase such as collagenase, stromelysin, and gelatinase. Illustrative examples of matrix metalloproteinase-cleavable amino acid sequences include, but are not limited to: -Gly-Pro-Y-Gly-Pro-Z-(SEQ ID NO: 124), -Gly-Pro-, Leu-Gly-Pro-Z-(SEQ ID NO: 125), -Gly-Pro-Ile-Gly-Pro-Z- (SEQ ID NO: 126), and -Ala-Pro-Gly-Leu-Z-(SEQ ID NO: 127), where Y and Z are amino acids. Illustrative examples of collagenase-cleavable amino acid sequences include, but are not limited to: -Pro-Leu-Gly-Pro-D-Arg-Z-(SEQ ID NO: 128), -Pro- Leu-Gly-Leu-Leu-Gly-Z-(SEQ ID NO: 129), -Pro-Gln-Gly-Ile-Ala-Gly-Trp-(SEQ ID NO: 130), -Pro-Leu-Gly-Cys(Me)-His-(SEQ ID NO: 131), -Pro-Leu-Gly-Leu-Tyr-Ala-(SEQ ID NO: 132), -Pro-Leu-Ala-Leu-Trp-Ala-Arg-(SEQ ID NO: 133), and -Pro-Leu-Ala-Tyr-Trp-Ala-Arg-(SEQ ID NO: 134), where Z is an amino acid. An illustrative example of a stromelysin-cleavable amino acid sequence is -Pro-Tyr-Ala-Tyr-Tyr-Met-Arg- (SEQ ID NO: 135); and an example of a gelatinase-cleavable amino acid sequence is -Pro-Leu-Gly-Met-Tyr-Ser-Arg-(SEQ ID NO: 136).

Cleavable linkers also include amino acid sequences that can be cleaved by an angiotensin converting enzyme, such as, for example, -Asp-Lys-Pro-, -Gly-Asp-Lys-Pro-(SEQ ID NO: 137), and -Gly-Ser-Asp-Lys-Pro- (SEQ ID NO: 138). Cleavable linkers also include amino acid sequences that can be degraded by cathepsin B, such as, for example, Val-Cit, Ala-Leu-Ala-Leu-(SEQ ID NO: 139), Gly-Phe-Leu-Gly-(SEQ ID NO: 140) and Phe-Lys.

In particular embodiments, a cleavable linker has a half life at pH 7.4, 25° C., for example, at physiological pH, human body temperature (e.g., in vivo, in serum, in a given tissue), of about or less than about 30 minutes, 1 hour, 2 hours, 3 hours, 4 hours, 5 hours, 6 hours, 12 hours, 18 hours, 24 hours, 36 hours, 48 hours, 72 hours, or 96 hours, or any intervening half-life.

Typically, at least one of the first or second linker is a non-cleavable linker. Exemplary non-cleavable linkers include those disclosed in Maratea et al., Gene 40:39-46, 1985; Murphy et al., PNAS USA. 83:8258-8262, 1986; U.S. Pat. Nos. 4,935,233 and 4,751,180. Particular non-cleavable linker sequences contain Gly, Ser, and/or Asn residues. Other near neutral amino acids, such as Thr and Ala may also be employed in the peptide linker sequence, if desired.

Certain exemplary non-cleavable linkers include Gly, Ser and/or Asn-containing linkers, as follows: [G]ₓ, [S]ₓ, [N]ₓ, [GS]ₓ, [GGS]ₓ, [GSS]ₓ, [GSGS]ₓ (SEQ ID NO: 141), [GGSG]ₓ (SEQ ID NO: 142), [GGGS]ₓ (SEQ ID NO: 143), [GGGGS]ₓ (SEQ ID NO: 144), [GN]ₓ, [GGN]ₓ, [GNN]ₓ, [GNGN]ₓ (SEQ ID NO: 145), [GGNG]ₓ (SEQ ID NO: 146), [GGGN]ₓ (SEQ ID NO: 147), [GGGGN]ₓ (SEQ ID NO: 148) linkers, where x is 1, 2, 3, 4, 5, 6, 7, 8, 9, 10, 11, 12, 13, 14, 15, 16, 17, 18, 19, or 20 or more. Other combinations of these and related amino acids will be apparent to persons skilled in the art.

Additional examples of non-cleavable linkers include the following amino acid sequences: Gly-Gly-Gly-Gly-Ser-Gly-Gly-Gly-Gly-Ser-Gly-Gly-Gly-Gly-Ser-(SEQ ID NO: 149); Gly-Ser-Gly-Gly-Gly-Gly-Ser-Gly-Gly-Gly-Gly-Ser-Gly-Gly-Gly-Gly-Ser-Gly-Gly-Gly-Gly-Ser-(SEQ ID NO: 150); Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-(SEQ ID NO: 151); Asp-Ala-Ala-Ala-Lys-Glu-Ala-Ala-Ala-Lys-Asp-Ala-Ala-Ala-Arg-Glu-Ala-Ala-Ala-Arg-Asp-Ala-Ala-Ala-Lys-(SEQ ID NO: 152); and Asn-Val-Asp-His-Lys-Pro-Ser-Asn-Thr-Lys-Val-Asp-Lys-Arg-(SEQ ID NO: 153).

Further non-limiting examples of non-cleavable linkers include DGGGS (SEQ ID NO: 154); TGEKP (SEQ ID NO: 155) (see, e.g., Liu et al., PNAS. 94:5525-5530, 1997); GGRR (SEQ ID NO: 156) (Pomerantz et al. 1995); (GGGGS)ₙ (SEQ ID NO: 144) (Kim et al., PNAS. 93:1156-1160, 1996); EGKSSGSGSESKVD (SEQ ID NO: 157) (Chaudhary et al., PNAS. 87:1066-1070, 1990); KESGSVSSEQLAQFRSLD (SEQ ID NO: 158) (Bird et al., Science. 242:423-426, 1988), GGRRGGGS (SEQ ID NO: 159); LRQRDGERP (SEQ ID NO: 160); LRQKDGGGSERP (SEQ ID NO: 161); LRQKd(GGGS)₂ ERP (SEQ ID NO: 162). In specific embodiments, the linker comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

In some embodiment, a linker comprises an immunoglobulin (Ig)/antibody hinge region or fragment thereof, for example, a hinge region obtained or derived from an IgG1 antibody. In some embodiments, the term Ig “hinge” region refers to a polypeptide comprising an amino acid sequence that shares sequence identity, or similarity, with a portion of a naturally-occurring Ig hinge region sequence, which optionally includes the cysteine residues at which the disulfide bonds link the two heavy chains of the immunoglobulin. Sequence similarity of the hinge region linkers of the present invention with naturally-occurring immunoglobulin hinge region amino acid sequences can range from at least 50% to about 75-80%, and typically greater than about 90%.

In some embodiments, the linker comprises a spacer element and a cleavable element so as to make the cleavable element more accessible to the enzyme responsible for cleavage.

It will be appreciated that any one or more of the foregoing linkers can be combined with any one or more of the binding moieties, IL-2 proteins, IL-2 binding proteins, and/or purification tags described herein, to form an activatable proprotein homodimer of the disclosure.

Affinity Purification Tags. In certain embodiments, the first and second polypeptides comprise at least one affinity purification tag. Exemplary affinity purification tags including a polyhistidine tag (optionally hexahistidine tag), a VSV-G tag (YTDIEMNRLGK; SEQ ID NO:163), a universal tag (HTTPHH; SEQ ID NO:164), a Strep-tag (WSHPQFEK; SEQ ID NO:165) or AWAHPQPGG; SEQ ID NO:166), an S-tag (KETAAAKFERQHMDS; SEQ ID NO:167), an S1-tag (NANNPDWDF; SEQ ID NO:168), a Phe-tag (composed, for example, of about 3, 4, 5, 6, 7, 8, 9, 10, 11, or 12 Phe residues), a Cys-tag (composed, for example, of about 3, 4, 5, 6, 7, 8, 9, 10, 11, or 12 Cys residues), an Asp-tag (composed, for example, of about 3, 4, 5, 6, 7, 8, 9, 10, 11, or 12 Asp residues), an Arg-tag (composed, for example, of about 3, 4, 5, 6, 7, 8, 9, 10, 11, or 12 Arg residues), a Myc epitope tag (CEQKLISEEDL, SEQ ID NO:169), a KT3 epitope tag (KPPTPPPEPET, SEQ ID NO:170), an HSV epitope tag (QPELAPED; SEQ ID NO:171), a histidine affinity tag (KDHLIHNVHKEFHAHAHNK; SEQ ID NO:172), a hemagglutinin (HA) tag, a FLAG epitope tag (DYKDDDK; SEQ ID NO:173), an E2 epitope tag (SSTSSDFRDR; SEQ ID NO:174), a V5-tag (GKPIPNPLLGLDST; SEQ ID NO:175), a T7-tag (MASMTGGQQMG; SEQ ID NO:176), an AU5 epitope tag (TDFYLK; SEQ ID NO:177), and an AU1 epitope tag (DTYRYI; SEQ ID NO:178).

Additional Domains. Certain activatable proproteins comprise one or more additional domains, for example, binding domains. In some embodiments, each of polypeptides in an activatable proprotein further comprise a protein domain A at one free terminus and/or a protein domain B at the other free terminus.

In some embodiments, the protein domains A and B are the same or different. In particular embodiments, the protein domains A and B are selected from one or more of cell receptor targeting moieties optionally bi-specific targeting moieties, antigen binding domains optionally bi-specific antigen binding domains, cell membrane receptor extracellular domains (ECDs), Fc domains, human serum albumin (HSA), Fc binding domains, HSA binding domains, cytokines, chemokines, and soluble protein ligands

In some embodiments, the one or more additional protein domains can be used to form complexes of two, three, four, five, or more activatable proproteins, which are bound to together via the additional domain(s).

Illustrative examples of activatable proproteins and certain of their expected cleavage products are provided in Table S4 below (see also the Examples).

TABLE S4
Exemplary Activatable Proproteins

| | | |
|---|---|---|
| | | SEQ ID |
| Name | Sequence | NO: |

P1522 Activatable Proprotein

| | | |
|---|---|---|
| Chains 1 | APTSSSTKKTQLQLEHLLLDLQMILNGINNYKNPKLTRMLTFKFYMPKKATEL | 179 |
| and 2: | KHLQCLEEELKPLEEALNLAPSKNFHLRPRDLISNINVIVLELKGSETTFMCE | |
| L5R- | YADETATIVEFLNRWITFSQSTISTLTGGGGSELCDDDPPEIPHATFKAMAYK | |
| GGGGS- | EGTMLNCECKRGFRRIKSGSLYMLCTGNSSHSSWDNQCQCTSSATRNTTKQVT | |
| his6 | PQPEEQKERKTTEMQSPMQPVDQASLPGHCREPPPWENEATERIYHFVVGQMV | |
| | YYQCVQGYRALHRGPAESVCKMTHGKTRWTQPQLICTGEGGGGSHHHHHH | |

P1523 Activatable Proprotein

| | | |
|---|---|---|
| Chains 1 | EPKSSDKTHTCPPCPAPELLGGPSVFLFPPKPKDTLMISRTPEVTCVVVDVSH | 180 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GSENLYFQ | SDIAVEWESNGQPENNYKTTPPVLDSDGSFFLYSKLTVDKSRWQQGNVFSCSV | |
| G-GS-L5R | MHEALHNHYTQKSLSLSPGKGSENLYFQGGSAPTSSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGGGSELCDDDPPEIPHATFKAMAYKEGTMLNCECKRGFRRIKSGSLY | |
| | MLCTGNSSHSSWDNQCQCTSSATRNTTKQVTPQPEEQKERKTTEMQSPMQPVD | |
| | QASLPGHCREPPPWENEATERIYHFVVGQMVYYQCVQGYRALHRGPAESVCKM | |
| | THGKTRWTQPQLICTGE | |

P1523 Cleaved proteins (Complete TEV cleavage)

| | | |
|---|---|---|
| Chains 1 | EPKSSDKTHTCPPCPAPELLGGPSVFLFPPKPKDTLMISRTPEVTCVVVDVSH | 181 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GSENLYFQ | SDIAVEWESNGQPENNYKTTPPVLDSDGSFFLYSKLTVDKSRWQQGNVFSCSV | |
| | MHEALHNHYTQKSLSLSPGKGSENLYFQ | |
| Chains 3 | GGSAPTSSSTKKTQLQLEHLLLDLQMILNGINNYKNPKLTRMLTFKFYMPKKA | 182 |
| and 4: | TELKHLQCLEEELKPLEEALNLAPSKNFHLRPRDLISNINVIVLELKGSETTF | |
| G-GS-L5R | MCEYADETATIVEFLNRWITFSQSTISTLTGGGGSELCDDDPPEIPHATFKAM | |
| | AYKEGTMLNCECKRGFRRIKSGSLYMLCTGNSSHSSWDNQCQCTSSATRNTTK | |
| | QVTPQPEEQKERKTTEMQSPMQPVDQASLPGHCREPPPWENEATERIYHFVVG | |
| | QMVYYQCVQGYRALHRGPAESVCKMTHGKTRWTQPQLICTGE | |

P1523 Cleaved proteins (Partial TEV cleavage)

| | | |
|---|---|---|
| Chain 1: | EPKSSDKTHTCPPCPAPELLGGPSVFLFPPKPKDTLMISRTPEVTCVVVDVSH | 183 |
| Fc- | EDPEVKFNWYVDGVEVHNAKTKPREEQYNSTYRVVSVLTVLHQDWLNGKEYKC | |
| GSENLYFQ | KVSNKALPAPIEKTISKAKGQPREPQVYTLPPSREEMTKNQVSLTCLVKGFYP | |
| G-GS-L5R | SDIAVEWESNGQPENNYKTTPPVLDSDGSFFLYSKLTVDKSRWQQGNVFSCSV | |
| | MHEALHNHYTQKSLSLSPGKGSENLYFQGGSAPTSSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGGGSELCDDDPPEIPHATFKAMAYKEGTMLNCECKRGFRRIKSGSLY | |
| | MLCTGNSSHSSWDNQCQCTSSATRNTTKQVTPQPEEQKERKTTEMQSPMQPVD | |
| | QASLPGHCREPPPWENEATERIYHFVVGQMVYYQCVQGYRALHRGPAESVCKM | |
| | THGKTRWTQPQLICTGE | |
| Chain 2: | EPKSSDKTHTCPPCPAPELLGGPSVFLFPPKPKDTLMISRTPEVTCVVVDVSH | 184 |
| Fc- | EDPEVKFNWYVDGVEVHNAKTKPREEQYNSTYRVVSVLTVLHQDWLNGKEYKC | |
| GSENLYFQ | KVSNKALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGKGSENLYFQ | |
| Chain 3: | GGSAPTSSSTKKTQLQLEHLLLDLQMILNGINNYKNPKLTRMLTFKFYMPKKA | 185 |
| G-GS-L5R | TELKHLQCLEEELKPLEEALNLAPSKNFHLRPRDLISNINVIVLELKGSETTF | |
| | MCEYADETATIVEFLNRWITFSQSTISTLTGGGGSELCDDDPPEIPHATFKAM | |
| | AYKEGTMLNCECKRGFRRIKSGSLYMLCTGNSSHSSWDNQCQCTSSATRNTTK | |
| | QVTPQPEEQKERKTTEMQSPMQPVDQASLPGHCREPPPWENEATERIYHFVVG | |
| | QMVYYQCVQGYRALHRGPAESVCKMTHGKTRWTQPQLICTGE | |

P1524 Activatable Proprotein

| | | |
|---|---|---|
| Chains 1 | APTSSSTKKTQLQLEHLLLDLQMILNGINNYKNPKLTRMLTFKFYMPKKATEL | 186 |
| and 2: | KHLQCLEEELKPLEEALNLAPSKNFHLRPRDLISNINVIVLELKGSETTFMCE | |
| L5R-GS- | YADETATIVEFLNRWITFSQSTISTLTGGGGSELCDDDPPEIPHATFKAMAYK | |
| TEV-Fc | EGTMLNCECKRGFRRIKSGSLYMLCTGNSSHSSWDNQCQCTSSATRNTTKQVT | |
| | PQPEEQKERKTTEMQSPMQPVDQASLPGHCREPPPWENEATERIYHFVVGQMV | |
| | YYQCVQGYRALHRGPAESVCKMTHGKTRWTQPQLICTGEGSENLYFQGEPKSS | |
| | DKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIAV | |
| | EWESNGQPENNYKTTPPVLDSDGSFFLYSKLTVDKSRWQQGNVFSCSVMHEAL | |
| | HNHYTQKSLSLSPGK | |

P1525 Activatable Proprotein

| | | |
|---|---|---|
| Chains 1 | APTSSSTKKTQLQLEHLLLDLQMILNGINNYKNPKLTRMLTFKFYMPKKATEL | 187 |
| and 2: | KHLQCLEEELKPLEEALNLAPSKNFHLRPRDLISNINVIVLELKGSETTFMCE | |
| L10R- | YADETATIVEFLNRWITFSQSTISTLTGGGGSGGGGSELCDDDPPEIPHATFK | |
| GGGGS- | AMAYKEGTMLNCECKRGFRRIKSGSLYMLCTGNSSHSSWDNQCQCTSSATRNT | |
| his6 | TKQVTPQPEEQKERKTTEMQSPMQPVDQASLPGHCREPPPWENEATERIYHFV | |
| | VGQMVYYQCVQGYRALHRGPAESVCKMTHGKTRWTQPQLICTGEGGGGSHHHH | |
| | HH | |

P1526 Activatable Proprotein

| | | |
|---|---|---|
| Chains 1 | EPKSSDKTHTCPPCPAPELLGGPSVFLFPPKPKDTLMISRTPEVTCVVVDVSH | 188 |
| and 2: | EDPEVKFNWYVDGVEVHNAKTKPREEQYNSTYRVVSVLTVLHQDWLNGKEYKC | |
| Fc-GS- | KVSNKALPAPIEKTISKAKGQPREPQVYTLPPSREEMTKNQVSLTCLVKGFYP | |
| TEV-GS- | SDIAVEWESNGQPENNYKTTPPVLDSDGSFFLYSKLTVDKSRWQQGNVFSCSV | |
| L10R | MHEALHNHYTQKSLSLSPGKGSENLYFQGGSAPTSSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGGGSGGGGSELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |

P1527 Activatable Proprotein

| | | |
|---|---|---|
| Chains 1 | APTSSSTKKTQLQLEHLLLDLQMILNGINNYKNPKLTRMLTFKFYMPKKATEL | 189 |
| and 2: | KHLQCLEEELKPLEEALNLAPSKNFHLRPRDLISNINVIVLELKGSETTFMCE | |
| L10R-GS- | YADETATIVEFLNRWITFSQSTISTLTGGGGSGGGGSELCDDDPPEIPHATFK | |
| TEV-Fc | AMAYKEGTMLNCECKRGFRRIKSGSLYMLCTGNSSHSSWDNQCQCTSSATRNT | |
| | TKQVTPQPEEQKERKTTEMQSPMQPVDQASLPGHCREPPPWENEATERIYHFV | |
| | VGQMVYYQCVQGYRALHRGPAESVCKMTHGKTRWTQPQLICTGEGSENLYFQG | |
| | EPKSSDKTHTCPPCPAPELLGGPSVFLFPPKPKDTLMISRTPEVTCVVVDVSH | |
| | EDPEVKFNWYVDGVEVHNAKTKPREEQYNSTYRVVSVLTVLHQDWLNGKEYKC | |
| | KVSNKALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGK | |

P1528 Activatable Proprotein

| | | |
|---|---|---|
| Chains 1 | ELCDDDPPEIPHATFKAMAYKEGTMLNCECKRGFRRIKSGSLYMLCTGNSSHS | 190 |
| and 2: | SWDNQCQCTSSATRNTTKQVTPQPEEQKERKTTEMQSPMQPVDQASLPGHCRE | |
| R5L- | PPPWENEATERIYHFVVGQMVYYQCVQGYRALHRGPAESVCKMTHGKTRWTQP | |
| GGGGS- | QLICTGEGGGGSAPTSSSTKKTQLQLEHLLLDLQMILNGINNYKNPKLTRMLT | |
| his6 | FKFYMPKKATELKHLQCLEEELKPLEEALNLAPSKNFHLRPRDLISNINVIVL | |
| | ELKGSETTFMCEYADETATIVEFLNRWITFSQSTISTLTGGGGSHHHHHH | |

P1529 Activatable Proprotein

| | | |
|---|---|---|
| Chains 1 | EPKSSDKTHTCPPCPAPELLGGPSVFLFPPKPKDTLMISRTPEVTCVVVDVSH | 191 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GSTEV- | SDIAVEWESNGQPENNYKTTPPVLDSDGSFFLYSKLTVDKSRWQQGNVFSCSV | |
| GS-R5L | MHEALHNHYTQKSLSLSPGKGSENLYFQGGSELCDDDPPEIPHATFKAMAYKE | |
| | GTMLNCECKRGFRRIKSGSLYMLCTGNSSHSSWDNQCQCTSSATRNTTKQVTP | |
| | QPEEQKERKTTEMQSPMQPVDQASLPGHCREPPPWENEATERIYHFVVGQMVY | |
| | YQCVQGYRALHRGPAESVCKMTHGKTRWTQPQLICTGEGGGGSAPTSSSTKKT | |
| | QLQLEHLLLDLQMILNGINNYKNPKLTRMLTFKFYMPKKATELKHLQCLEEEL | |
| | KPLEEALNLAPSKNFHLRPRDLISNINVIVLELKGSETTFMCEYADETATIVE | |
| | FLNRWITFSQSTISTLT | |

P1530 Activatable Proprotein

| | | |
|---|---|---|
| Chains 1 | ELCDDDPPEIPHATFKAMAYKEGTMLNCECKRGFRRIKSGSLYMLCTGNSSHS | 192 |
| and 2: | SWDNQCQCTSSATRNTTKQVTPQPEEQKERKTTEMQSPMQPVDQASLPGHCRE | |
| R5L-GS- | PPPWENEATERIYHFVVGQMVYYQCVQGYRALHRGPAESVCKMTHGKTRWTQP | |
| TEV-Fc | QLICTGEGGGGSAPTSSSTKKTQLQLEHLLLDLQMILNGINNYKNPKLTRMLT | |
| | FKFYMPKKATELKHLQCLEEELKPLEEALNLAPSKNFHLRPRDLISNINVIVL | |
| | ELKGSETTFMCEYADETATIVEFLNRWITFSQSTISTLTGSENLYFQGEPKSS | |
| | DKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIAV | |
| | EWESNGQPENNYKTTPPVLDSDGSFFLYSKLTVDKSRWQQGNVFSCSVMHEAL | |
| | HNHYTQKSLSLSPGK | |

P1531 Activatable Proprotein

| | | |
|---|---|---|
| Chains 1 | ELCDDDPPEIPHATFKAMAYKEGTMLNCECKRGFRRIKSGSLYMLCTGNSSHS | 193 |
| and 2: | SWDNQCQCTSSATRNTTKQVTPQPEEQKERKTTEMQSPMQPVDQASLPGHCRE | |
| R10L- | PPPWENEATERIYHFVVGQMVYYQCVQGYRALHRGPAESVCKMTHGKTRWTQP | |
| GGGGS- | QLICTGEGGGGSGGGGSAPTSSSTKKTQLQLEHLLLDLQMILNGINNYKNPKL | |
| his6 | TRMLTFKFYMPKKATELKHLQCLEEELKPLEEALNLAPSKNFHLRPRDLISNI | |
| | NVIVLELKGSETTFMCEYADETATIVEFLNRWITFSQSTISTLTGGGGSHHHH | |
| | HH | |

P1532 Activatable Proprotein

| | | |
|---|---|---|
| Chains 1 | EPKSSDKTHTCPPCPAPELLGGPSVFLFPPKPKDTLMISRTPEVTCVVVDVSH | 194 |
| and 2: | EDPEVKFNWYVDGVEVHNAKTKPREEQYNSTYRVVSVLTVLHQDWLNGKEYKC | |
| Fc-GS- | KVSNKALPAPIEKTISKAKGQPREPQVYTLPPSREEMTKNQVSLTCLVKGFYP | |
| TEV-GS- | SDIAVEWESNGQPENNYKTTPPVLDSDGSFFLYSKLTVDKSRWQQGNVFSCSV | |
| R10L | MHEALHNHYTQKSLSLSPGKGSENLYFQGGSELCDDDPPEIPHATFKAMAYKE | |
| | GTMLNCECKRGFRRIKSGSLYMLCTGNSSHSSWDNQCQCTSSATRNTTKQVTP | |
| | QPEEQKERKTTEMQSPMQPVDQASLPGHCREPPPWENEATERIYHFVVGQMVY | |
| | YQCVQGYRALHRGPAESVCKMTHGKTRWTQPQLICTGEGGGGSGGGGSAPTSS | |
| | STKKTQLQLEHLLLDLQMILNGINNYKNPKLTRMLTFKFYMPKKATELKHLQC | |
| | LEEELKPLEEALNLAPSKNFHLRPRDLISNINVIVLELKGSETTFMCEYADET | |
| | ATIVEFLNRWITFSQSTISTLT | |

P1533 Activatable Proprotein

| | | |
|---|---|---|
| Chains 1 | ELCDDDPPEIPHATFKAMAYKEGTMLNCECKRGFRRIKSGSLYMLCTGNSSHS | 195 |
| and 2: | SWDNQCQCTSSATRNTTKQVTPQPEEQKERKTTEMQSPMQPVDQASLPGHCRE | |
| R10L-GS- | PPPWENEATERIYHFVVGQMVYYQCVQGYRALHRGPAESVCKMTHGKTRWTQP | |
| TEV-Fc | QLICTGEGGGGSGGGGSAPTSSSTKKTQLQLEHLLLDLQMILNGINNYKNPKL | |
| | TRMLTFKFYMPKKATELKHLQCLEEELKPLEEALNLAPSKNFHLRPRDLISNI | |
| | NVIVLELKGSETTFMCEYADETATIVEFLNRWITFSQSTISTLTGSENLYFQG | |
| | EPKSSDKTHTCPPCPAPELLGGPSVFLFPPKPKDTLMISRTPEVTCVVVDVSH | |
| | EDPEVKFNWYVDGVEVHNAKTKPREEQYNSTYRVVSVLTVLHQDWLNGKEYKC | |
| | KVSNKALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGK | |

P1630 Activatable Proprotein

| | | |
|---|---|---|
| Chains 1 | APTSSSTKKTQLQLEHLLLDLQMILNGINNYKNPKLTRMLTFKFYMPKKATEL | 196 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSENLYFQGGSELCDDDPPEIPHATF | |
| GSENLYFQ | KAMAYKEGTMLNCECKRGFRRIKSGSLYMLCTGNSSHSSWDNQCQCTSSATRN | |
| GGS-IL- | TTKQVTPQPEEQKERKTTEMQSPMQPVDQASLPGHCREPPPWENEATERIYHF | |
| 2Rα- | VVGQMVYYQCVQGYRALHRGPAESVCKMTHGKTRWTQPQLICTGEGGGGSHHH | |
| GGGGSHHH | HHH | |
| HHH | | |

P1630 Cleaved proteins (Complete TEV cleavage)

| | | |
|---|---|---|
| Chain 1: | APTSSSTKKTQLQLEHLLLDLQMILNGINNYKNPKLTRMLTFKFYMPKKATEL | 197 |
| IL-2-T3- | KHLQCLEEELKPLEEALNLAPSKNFHLRPRDLISNINVIVLELKGSETTFMCE | |
| GSENLYFQ | YADETATIVEFLNRWITFSQSTISTLTGSENLYFQ | |
| Chain 2: | GGSELCDDDPPEIPHATFKAMAYKEGTMLNCECKRGFRRIKSGSLYMLCTGNS | 198 |
| GGS-IL- | SHSSWDNQCQCTSSATRNTTKQVTPQPEEQKERKTTEMQSPMQPVDQASLPGH | |
| 2Rα- | CREPPPWENEATERIYHFVVGQMVYYQCVQGYRALHRGPAESVCKMTHGKTRW | |
| GGGGSHHH | TQPQLICTGEGGGGSHHHHHH | |
| HHH | | |

P1630 Cleaved proteins (Partial TEV cleavage)

| | | |
|---|---|---|
| Chain 1: | APTSSSTKKTQLQLEHLLLDLQMILNGINNYKNPKLTRMLTFKFYMPKKATEL | 199 |
| IL-2-T3- | KHLQCLEEELKPLEEALNLAPSKNFHLRPRDLISNINVIVLELKGSETTFMCE | |
| GSENLYFQ | YADETATIVEFLNRWITFSQSTISTLTGSENLYFQGGSELCDDDPPEIPHATF | |
| GGS-IL- | KAMAYKEGTMLNCECKRGFRRIKSGSLYMLCTGNSSHSSWDNQCQCTSSATRN | |
| 2Rα- | TTKQVTPQPEEQKERKTTEMQSPMQPVDQASLPGHCREPPPWENEATERIYHF | |
| GGGGSHHH | VVGQMVYYQCVQGYRALHRGPAESVCKMTHGKTRWTQPQLICTGEGGGGSHHH | |
| HHH | HHH | |
| Chain 2: | APTSSSTKKTQLQLEHLLLDLQMILNGINNYKNPKLTRMLTFKFYMPKKATEL | 200 |
| IL-2-T3- | KHLQCLEEELKPLEEALNLAPSKNFHLRPRDLISNINVIVLELKGSETTFMCE | |
| GSENLYFQ | YADETATIVEFLNRWITFSQSTISTLTGSENLYFQ | |
| Chain 3: | GGSELCDDDPPEIPHATFKAMAYKEGTMLNCECKRGFRRIKSGSLYMLCTGNS | 201 |
| GGS-IL- | SHSSWDNQCQCTSSATRNTTKQVTPQPEEQKERKTTEMQSPMQPVDQASLPGH | |
| 2Rα- | CREPPPWENEATERIYHFVVGQMVYYQCVQGYRALHRGPAESVCKMTHGKTRW | |
| GGGGSHHH | TQPQLICTGEGGGGSHHHHHH | |
| HHH | | |

P1631 Activatable Proprotein

| | | |
|---|---|---|
| Chains 1 | EPKSSDKTHTCPPCPAPELLGGPSVFLFPPKPKDTLMISRTPEVTCVVVDVSH | 202 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GGGGSGGG | SDIAVEWESNGQPENNYKTTPPVLDSDGSFFLYSKLTVDKSRWQQGNVFSCSV | |
| GS-IL-2- | MHEALHNHYTQKSLSLSPGKGGGGSGGGGSAPTSSSTKKTQLQLEHLLLDLQM | |
| T3- | ILNGINNYKNPKLTRMLTFKFYMPKKATELKHLQCLEEELKPLEEALNLAPSK | |
| GSENLYFQ | NFHLRPRDLISNINVIVLELKGSETTFMCEYADETATIVEFLNRWITFSQSTI | |
| GGS-IL- | STLTGSENLYFQGGSELCDDDPPEIPHATFKAMAYKEGTMLNCECKRGFRRIK | |
| 2Rα | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |

P1632 Activatable Proprotein

| | | |
|---|---|---|
| Chains 1 | APTSSSTKKTQLQLEHLLLDLQMILNGINNYKNPKLTRMLTFKFYMPKKATEL | 203 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSENLYFQGGSELCDDDPPEIPHATF | |
| GSENLYFQ | KAMAYKEGTMLNCECKRGFRRIKSGSLYMLCTGNSSHSSWDNQCQCTSSATRN | |
| GGS-IL- | TTKQVTPQPEEQKERKTTEMQSPMQPVDQASLPGHCREPPPWENEATERIYHF | |
| 2Rα- | VVGQMVYYQCVQGYRALHRGPAESVCKMTHGKTRWTQPQLICTGEGGGGSGGG | |
| GGGGSGGG | GSEPKSSDKTHTCPPCPAPELLGGPSVFLFPPKPKDTLMISRTPEVTCVVVDV | |
| GS-Fc | SHEDPEVKFNWYVDGVEVHNAKTKPREEQYNSTYRVVSVLTVLHQDWLNGKEY | |
| | KCKVSNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVFSC | |
| | SVMHEALHNHYTQKSLSLSPGK | |

P1633 Activatable Proprotein

| | | |
|---|---|---|
| Chains 1 | ELCDDDPPEIPHATFKAMAYKEGTMLNCECKRGFRRIKSGSLYMLCTGNSSHS | 204 |
| and 2: | SWDNQCQCTSSATRNTTKQVTPQPEEQKERKTTEMQSPMQPVDQASLPGHCRE | |
| II-2Rα- | PPPWENEATERIYHFVVGQMVYYQCVQGYRALHRGPAESVCKMTHGKTRWTQP | |
| GSENLYFQ | QLICTGEGSENLYFQGGSAPTSSSTKKTQLQLEHLLLDLQMILNGINNYKNPK | |
| GGS-IL- | LTRMLTFKFYMPKKATELKHLQCLEEELKPLEEALNLAPSKNFHLRPRDLISN | |
| 2-T3- | INVIVLELKGSETTFMCEYADETATIVEFLNRWITFSQSTISTLTGGGGSHHH | |
| GGGGSHHH | HHH | |
| HHH | | |

P1634 Activatable Proprotein

| | | |
|---|---|---|
| Chains 1 | EPKSSDKTHTCPPCPAPELLGGPSVFLFPPKPKDTLMISRTPEVTCVVVDVSH | 205 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GGGGSGGG | SDIAVEWESNGQPENNYKTTPPVLDSDGSFFLYSKLTVDKSRWQQGNVFSCSV | |
| GS-IL- | MHEALHNHYTQKSLSLSPGKGGGGSGGGGSELCDDDPPEIPHATFKAMAYKEG | |
| 2Rα- | TMLNCECKRGFRRIKSGSLYMLCTGNSSHSSWDNQCQCTSSATRNTTKQVTPQ | |
| GSENLYFQ | PEEQKERKTTEMQSPMQPVDQASLPGHCREPPPWENEATERIYHFVVGQMVYY | |
| GGS-IL- | QCVQGYRALHRGPAESVCKMTHGKTRWTQPQLICTGEGSENLYFQGGSAPTSS | |
| 2-T3 | STKKTQLQLEHLLLDLQMILNGINNYKNPKLTRMLTFKFYMPKKATELKHLQC | |
| | LEEELKPLEEALNLAPSKNFHLRPRDLISNINVIVLELKGSETTFMCEYADET | |
| | ATIVEFLNRWITFSQSTISTLT | |

P1635 Activatable Proprotein

| | | |
|---|---|---|
| Chains 1 | ELCDDDPPEIPHATFKAMAYKEGTMLNCECKRGFRRIKSGSLYMLCTGNSSHS | 206 |
| and 2: | SWDNQCQCTSSATRNTTKQVTPQPEEQKERKTTEMQSPMQPVDQASLPGHCRE | |
| IL-2Rα- | PPPWENEATERIYHFVVGQMVYYQCVQGYRALHRGPAESVCKMTHGKTRWTQP | |
| GSENLYFQ | QLICTGEGSENLYFQGGSAPTSSSTKKTQLQLEHLLLDLQMILNGINNYKNPK | |
| GGS-IL- | LTRMLTFKFYMPKKATELKHLQCLEEELKPLEEALNLAPSKNFHLRPRDLISN | |
| 2-T3- | INVIVLELKGSETTFMCEYADETATIVEFLNRWITFSQSTISTLTGGGGSGGG | |
| GGGGSGGG | GSEPKSSDKTHTCPPCPAPELLGGPSVFLFPPKPKDTLMISRTPEVTCVVVDV | |
| GS-Fc | SHEDPEVKFNWYVDGVEVHNAKTKPREEQYNSTYRVVSVLTVLHQDWLNGKEY | |
| | KCKVSNKALPAPIEKTISKAKGQPREPQVYTLPPSREEMTKNQVSLTCLVKGF | |
| | YPSDIAVEWESNGQPENNYKTTPPVLDSDGSFFLYSKLTVDKSRWQQGNVFSC | |
| | SVMHEALHNHYTQKSLSLSPGK | |

P1664 Activatable Proprotein

| | | |
|---|---|---|
| Chains 1 | APTSSSTKKTQLQLEHLLLDLQMILNGINNYKNPKLTRMLTFKFYMPKKATEL | 207 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSGSENLYFQGGSGSELCDDDPPEIP | |
| GSGSENLY | HATFKAMAYKEGTMLNCECKRGFRRIKSGSLYMLCTGNSSHSSWDNQCQCTSS | |
| FQGGSGS- | ATRNTTKQVTPQPEEQKERKTTEMQSPMQPVDQASLPGHCREPPPWENEATER | |
| IL-2Rα- | IYHFVVGQMVYYQCVQGYRALHRGPAESVCKMTHGKTRWTQPQLICTGEGGGG | |
| GGGGSHHH | SHHHHHH | |
| HHH | | |

P1665 Activatable Proprotein

| | | |
|---|---|---|
| Chains 1 | EPKSSDKTHTCPPCPAPELLGGPSVFLFPPKPKDTLMISRTPEVTCVVVDVSH | 208 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GGGGSGGG | SDIAVEWESNGQPENNYKTTPPVLDSDGSFFLYSKLTVDKSRWQQGNVFSCSV | |
| GS-IL-2- | MHEALHNHYTQKSLSLSPGKGGGGSGGGGSAPTSSSTKKTQLQLEHLLLDLQM | |
| T3- | ILNGINNYKNPKLTRMLTFKFYMPKKATELKHLQCLEEELKPLEEALNLAPSK | |
| GSGSENLY | NFHLRPRDLISNINVIVLELKGSETTFMCEYADETATIVEFLNRWITFSQSTI | |
| FQGGSGS- | STLTGSGSENLYFQGGSGSELCDDDPPEIPHATFKAMAYKEGTMLNCECKRGF | |
| IL-2Rα | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1666 Activatable Proprotein

| | | |
|---|---|---|
| Chains 1 | APTSSSTKKTQLQLEHLLLDLQMILNGINNYKNPKLTRMLTFKFYMPKKATEL | 209 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSGSENLYFQGGSGSELCDDDPPEIP | |
| GSGSENLY | HATFKAMAYKEGTMLNCECKRGFRRIKSGSLYMLCTGNSSHSSWDNQCQCTSS | |
| FQGGSGS- | ATRNTTKQVTPQPEEQKERKTTEMQSPMQPVDQASLPGHCREPPPWENEATER | |
| IL-2Rα- | IYHFVVGQMVYYQCVQGYRALHRGPAESVCKMTHGKTRWTQPQLICTGEGGGG | |
| GGGGSGGG | SGGGGSEPKSSDKTHTCPPCPAPELLGGPSVFLFPPKPKDTLMISRTPEVTCV | |
| GS-Fc | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSRWQQGN | |
| | VFSCSVMHEALHNHYTQKSLSLSPGK | |

P1667 Activatable Proprotein

| | | |
|---|---|---|
| Chains 1 | APTSSSTKKTQLQLEHLLLDLQMILNGINNYKNPKLTRMLTFKFYMPKKATEL | 210 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSGSGSENLYFQGGSGSGSELCDDDP | |
| GSGSGSEN | PEIPHATFKAMAYKEGTMLNCECKRGFRRIKSGSLYMLCTGNSSHSSWDNQCQ | |
| LYFQGGSG | CTSSATRNTTKQVTPQPEEQKERKTTEMQSPMQPVDQASLPGHCREPPPWENE | |
| SGS-IL- | ATERIYHFVVGQMVYYQCVQGYRALHRGPAESVCKMTHGKTRWTQPQLICTGE | |
| 2Rα- | GGGGSHHHHHH | |
| GGGGSHHH | | |
| HHH | | |

P1668 Activatable Proprotein

| | | |
|---|---|---|
| Chains 1 | EPKSSDKTHTCPPCPAPELLGGPSVFLFPPKPKDTLMISRTPEVTCVVVDVSH | 211 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| GGGGSGGG | SDIAVEWESNGQPENNYKTTPPVLDSDGSFFLYSKLTVDKSRWQQGNVFSCSV | |
| GS-IL-2- | MHEALHNHYTQKSLSLSPGKGGGGSGGGGSAPTSSSTKKTQLQLEHLLLDLQM | |
| T3- | ILNGINNYKNPKLTRMLTFKFYMPKKATELKHLQCLEEELKPLEEALNLAPSK | |
| GSGSGS3E | NFHLRPRDLISNINVIVLELKGSETTFMCEYADETATIVEFLNRWITFSQSTI | |
| NLYFQGGS | STLTGSGSGSENLYFQGGSGSGSELCDDDPPEIPHATFKAMAYKEGTMLNCEC | |
| GSGS-IL- | KRGFRRIKSGSLYMLCTGNSSHSSWDNQCQCTSSATRNTTKQVTPQPEEQKER | |
| 2Rα | KTTEMQSPMQPVDQASLPGHCREPPPWENEATERIYHFVVGQMVYYQCVQGYR | |
| | ALHRGPAESVCKMTHGKTRWTQPQLICTGE | |

P1669 Activatable Proprotein

| | | |
|---|---|---|
| Chains 1 | APTSSSTKKTQLQLEHLLLDLQMILNGINNYKNPKLTRMLTFKFYMPKKATEL | 212 |
| and 2: | KHLQCLEEELKPLEEALNLAPSKNFHLRPRDLISNINVIVLELKGSETTFMCE | |
| IL-2-T3- | YADETATIVEFLNRWITFSQSTISTLTGSGSGSENLYFQGGSGSGSELCDDDP | |
| GSGSGSEN | PEIPHATFKAMAYKEGTMLNCECKRGFRRIKSGSLYMLCTGNSSHSSWDNQCQ | |
| LYFQGGSG | CTSSATRNTTKQVTPQPEEQKERKTTEMQSPMQPVDQASLPGHCREPPPWENE | |
| SGS-IL- | ATERIYHFVVGQMVYYQCVQGYRALHRGPAESVCKMTHGKTRWTQPQLICTGE | |
| 2Rα- | GGGGSGGGGSEPKSSDKTHTCPPCPAPELLGGPSVFLFPPKPKDTLMISRTPE | |
| GGGGSGGG | VTCVVVDVSHEDPEVKFNWYVDGVEVHNAKTKPREEQYNSTYRVVSVLTVLHQ | |
| GS-Fc | DWLNGKEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1719 Activatable Proprotein

| | | |
|---|---|---|
| Chains 1 | APASSSTKKTQLQLEHLLLDLQMILNGINNYKNPKLTRMLTFKFYMPKKATEL | 213 |
| and 2: | KHLQCLECELKPLEEVLNLAQSKNFHLRPRDLISNINVIVLELKGSETTFMCE | |
| L_T3A_E61C- | YADETATIVEFLNRWITFSQSIISTLTPLGLAGSGRSDNQGA | |
| PLGLAGSG | ELCDDDPPEIPHATFKAMAYKEGTMLNCECKRGFRRICSGSLYMLCTGNSSHS | |
| RSDNQGA- | SWDNQCQCTSSATRNTTKQVTPQPEEQKERKTTEMQSPMQPVDQASLPGHCRE | |
| R_K38C- | PPPWENEATERIYHFVVGQMVYYQCVQGYRALHRGPAESVCKMTHGKTRWTQP | |
| GGGGS- | QLICTGEGGGGSHHHHHH | |
| his6 | | |

P1719 Cleaved proteins (Complete uPA/Matriptase cleavage)

| | | |
|---|---|---|
| Chain 1: | APASSSTKKTQLQLEHLLLDLQMILNGINNYKNPKLTRMLTFKFYMPKKATEL | 214 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGR | |
| R | | |
| Chain 2: | SDNQGAELCDDDPPEIPHATFKAMAYKEGTMLNCECKRGFRRICSGSLYMLCT | 215 |
| SDNQGA- | GNSSHSSWDNQCQCTSSATRNTTKQVTPQPEEQKERKTTEMQSPMQPVDQASL | |
| R_K38C- | PGHCREPPPWENEATERIYHFVVGQMVYYQCVQGYRALHRGPAESVCKMTHGK | |
| GGGGS- | TRWTQPQLICTGEGGGGSHHHHHH | |
| his6 | | |

P1719 Cleaved proteins (Partial uPA/Matriptase cleavage)

| | | |
|---|---|---|
| Chain 1: | APASSSTKKTQLQLEHLLLDLQMILNGINNYKNPKLTRMLTFKFYMPKKATEL | 216 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGRSDNQGA | |
| RSDNQGA- | ELCDDDPPEIPHATFKAMAYKEGTMLNCECKRGFRRICSGSLYMLCTGNSSHS | |
| R_K38C- | SWDNQCQCTSSATRNTTKQVTPQPEEQKERKTTEMQSPMQPVDQASLPGHCRE | |
| GGGGS- | PPPWENEATERIYHFVVGQMVYYQCVQGYRALHRGPAESVCKMTHGKTRWTQP | |
| his6 | QLICTGEGGGGSHHHHHH | |
| Chain 2: | APASSSTKKTQLQLEHLLLDLQMILNGINNYKNPKLTRMLTFKFYMPKKATEL | 217 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGR | |
| R | | |
| Chain 3: | SDNQGAELCDDDPPEIPHATFKAMAYKEGTMLNCECKRGFRRICSGSLYMLCT | 218 |
| SDNQGA- | GNSSHSSWDNQCQCTSSATRNTTKQVTPQPEEQKERKTTEMQSPMQPVDQASL | |
| R_K38C- | PGHCREPPPWENEATERIYHFVVGQMVYYQCVQGYRALHRGPAESVCKMTHGK | |
| GGGGS- | TRWTQPQLICTGEGGGGSHHHHHH | |
| his6 | | |

P1719 Cleaved proteins (Complete MMP-2/MMP-9 cleavage)

| | | |
|---|---|---|
| Chain 1: | APASSSTKKTQLQLEHLLLDLQMILNGINNYKNPKLTRMLTFKFYMPKKATEL | 219 |
| L_T3A_E61C- | KHLQCLECELKPLEEVLNLAQSKNFHLRPRDLISNINVIVLELKGSETTFMCE | |
| PLG | YADETATIVEFLNRWITFSQSIISTLTPLG | |
| Chain 2: | LAGSGRSDNQGAELCDDDPPEIPHATFKAMAYKEGTMLNCECKRGFRRICSGS | 220 |
| LAGSGRSD | LYMLCTGNSSHSSWDNQCQCTSSATRNTTKQVTPQPEEQKERKTTEMQSPMQP | |
| NQGA- | VDQASLPGHCREPPPWENEATERIYHFVVGQMVYYQCVQGYRALHRGPAESVC | |
| R_K38C- | KMTHGKTRWTQPQLICTGEGGGGSHHHHHH | |
| GGGGS- | | |
| his6 | | |

P1719 Cleaved proteins (Partial MMP-2/MMP-9 cleavage)

| | | |
|---|---|---|
| Chain 1: | APASSSTKKTQLQLEHLLLDLQMILNGINNYKNPKLTRMLTFKFYMPKKATEL | 221 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGRSDNQGA | |
| RSDNQGA- | ELCDDDPPEIPHATFKAMAYKEGTMLNCECKRGFRRICSGSLYMLCTGNSSHS | |
| R_K38C- | SWDNQCQCTSSATRNTTKQVTPQPEEQKERKTTEMQSPMQPVDQASLPGHCRE | |
| GGGGS- | PPPWENEATERIYHFVVGQMVYYQCVQGYRALHRGPAESVCKMTHGKTRWTQP | |
| his6 | QLICTGEGGGGSHHHHHH | |
| Chain 2: | APASSSTKKTQLQLEHLLLDLQMILNGINNYKNPKLTRMLTFKFYMPKKATEL | 222 |
| L_T3A_E61C- | KHLQCLECELKPLEEVLNLAQSKNFHLRPRDLISNINVIVLELKGSETTFMCE | |
| PLG | YADETATIVEFLNRWITFSQSIISTLT | |
| | PLG | |
| Chain 3: | LAGSGRSDNQGA | 223 |
| LAGSGRSD | ELCDDDPPEIPHATFKAMAYKEGTMLNCECKRGFRRICSGSLYMLCTGNSSHS | |
| NQGA- | SWDNQCQCTSSATRNTTKQVTPQPEEQKERKTTEMQSPMQPVDQASLPGHCRE | |
| R_K38C- | PPPWENEATERIYHFVVGQMVYYQCVQGYRALHRGPAESVCKMTHGKTRWTQP | |
| GGGGS- | QLICTGE | |
| his6 | GGGGSHHHHHH | |

P1719 Cleaved proteins (Complete Legumain cleavage)

| | | |
|---|---|---|
| Chain 1: | APASSSTKKTQLQLEHLLLDLQMILNGINNYKNPKLTRMLTFKFYMPKKATEL | 224 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGRSDN | |
| RSDN | | |
| Chain 2: | QGAELCDDDPPEIPHATFKAMAYKEGTMLNCECKRGFRRICSGSLYMLCTGNS | 225 |
| QGA- | SHSSWDNQCQCTSSATRNTTKQVTPQPEEQKERKTTEMQSPMQPVDQASLPGH | |
| R_K38C- | CREPPPWENEATERIYHFVVGQMVYYQCVQGYRALHRGPAESVCKMTHGKTRW | |
| GGGGS- | TQPQLICTGEGGGGSHHHHHH | |
| his6 | | |

P1719 Cleaved proteins (Partial Legumain cleavage)

| | | |
|---|---|---|
| Chain 1: | APASSSTKKTQLQLEHLLLDLQMILNGINNYKNPKLTRMLTFKFYMPKKATEL | 226 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGRSDNQGA | |
| RSDNQGA- | ELCDDDPPEIPHATFKAMAYKEGTMLNCECKRGFRRICSGSLYMLCTGNSSHS | |
| R_K38C- | SWDNQCQCTSSATRNTTKQVTPQPEEQKERKTTEMQSPMQPVDQASLPGHCRE | |
| GGGGS- | PPPWENEATERIYHFVVGQMVYYQCVQGYRALHRGPAESVCKMTHGKTRWTQP | |
| his6 | QLICTGEGGGGSHHHHHH | |
| Chain 2: | APASSSTKKTQLQLEHLLLDLQMILNGINNYKNPKLTRMLTFKFYMPKKATEL | 227 |
| L_T3A_E61C- | KHLQCLECELKPLEEVLNLAQSKNFHLRPRDLISNINVIVLELKGSETTFMCE | |
| PLGLAGSG | YADETATIVEFLNRWITFSQSIISTLTPLGLAGSGRSDN | |
| RSDN | | |
| Chain 3: | QGAELCDDDPPEIPHATFKAMAYKEGTMLNCECKRGFRRICSGSLYMLCTGNS | 228 |
| QGA- | SHSSWDNQCQCTSSATRNTTKQVTPQPEEQKERKTTEMQSPMQPVDQASLPGH | |
| R_K38C- | CREPPPWENEATERIYHFVVGQMVYYQCVQGYRALHRGPAESVCKMTHGKTRW | |
| GGGGS- | TQPQLICTGEGGGGSHHHHHH | |
| his6 | | |

P1720 Activatable Proprotein

| | | |
|---|---|---|
| Chains 1 | EPKSSDKTHTCPPCPAPELLGGPSVFLFPPKPKDTLMISRTPEVTCVVVDVSH | 229 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_T3A_E61C- | MHEALHNHYTQKSLSLSPGGGGGSGGGGSAPASSSTKKTQLQLEHLLLDLQMI | |
| GPLGLAGS | LNGINNYKNPKLTRMLTFKFYMPKKATELKHLQCLECELKPLEEVLNLAQSKN | |
| GRSDNQGA- | FHLRPRDLISNINVIVLELKGSETTFMCEYADETATIVEFLNRWITFSQSIIS | |
| R_K38C | TLTGPLGLAGSGRSDNQGAELCDDDPPEIPHATFKAMAYKEGTMLNCECKRGF | |
| | RRICSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1721 Activatable Proprotein

| | | |
|---|---|---|
| Chains 1 | APASSSTKKTQLQLEHLLLDLQMILNGINNYKNPKLTRMLTFKFYMPKKATEL | 230 |
| and 2: | KHLQCLECELKPLEEVLNLAQSKNFHLRPRDLISNINVIVLELKGSETTFMCE | |
| L_T3A_E61C- | YADETATIVEFLNRWITFSQSIISTLTPLGLAGSGRSDNQGA | |
| PLGLAGSG | ELCDDDPPEIPHATFKAMAYKEGTMLNCECKRGFRRICSGSLYMLCTGNSSHS | |
| RSDNQGA- | SWDNQCQCTSSATRNTTKQVTPQPEEQKERKTTEMQSPMQPVDQASLPGHCRE | |
| R_K38C- | PPPWENEATERIYHFVVGQMVYYQCVQGYRALHRGPAESVCKMTHGKTRWTQP | |
| stableLinker-Fc | QLICTGEGGGGSGGGGSEPKSSDKTHTCPPCPAPELLGGPSVFLFPPKPKDTL | |
| | MISRTPEVTCVVVDVSHEDPEVKFNWYVDGVEVHNAKTKPREEQYNSTYRVVS | |
| | VLTVLHQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFFLYSKL | |
| | TVDKSRWQQGNVFSCSVMHEALHNHYTQKSLSLSPGK | |

P1722 Activatable Proprotein

| | | |
|---|---|---|
| Chains 1 | APASSSTKKTQLQLEHLLLDLQMILNGINNYKNPKLTRMLTFKFYMPKKATEL | 231 |
| and 2: | KHLQCLECELKPLEEVLNLAQSKNFHLRPRDLISNINVIVLELKGSETTFMCE | |
| L_T3A_E61C- | YADETATIVEFLNRWITFSQSIISTLTGPLGLAGSGRSDNQGAELCDDDPPEI | |
| GPLGLAGS | PHATFKAMAYKEGTMLNCECKRGFRRICSGSLYMLCTGNSSHSSWDNQCQCTS | |
| GRSDNQGA- | SATRNTTKQVTPQPEEQKERKTTEMQSPMQPVDQASLPGHCREPPPWENEATE | |
| R_K38C- | RIYHFVVGQMVYYQCVQGYRALHRGPAESVCKMTHGKTRWTQPQLICTGEGGG | |
| GGGGS- | GSHHHHHH | |
| his6 | | |

P1723 Activatable Proprotein

| | | |
|---|---|---|
| Chains 1 | EPKSSDKTHTCPPCPAPELLGGPSVFLFPPKPKDTLMISRTPEVTCVVVDVSH | 232 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_T3A_E61C- | MHEALHNHYTQKSLSLSPGGGGGSGGGGSAPASSSTKKTQLQLEHLLLDLQMI | |
| GGPLGLAG | LNGINNYKNPKLTRMLTFKFYMPKKATELKHLQCLECELKPLEEVLNLAQSKN | |
| SGRSDNQG | FHLRPRDLISNINVIVLELKGSETTFMCEYADETATIVEFLNRWITFSQSIIS | |
| A-R_K38C | TLTGGPLGLAGSGRSDNQGAELCDDDPPEIPHATFKAMAYKEGTMLNCECKRG | |
| | FRRICSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P1724 Activatable Proprotein

| | | |
|---|---|---|
| Chains 1 | APASSSTKKTQLQLEHLLLDLQMILNGINNYKNPKLTRMLTFKFYMPKKATEL | 233 |
| and 2: | KHLQCLECELKPLEEVLNLAQSKNFHLRPRDLISNINVIVLELKGSETTFMCE | |
| L_T3A_E61C- | YADETATIVEFLNRWITFSQSIISTLTGPLGLAGSGRSDNQGAELCDDDPPEI | |
| GPLGLAGS | PHATFKAMAYKEGTMLNCECKRGFRRICSGSLYMLCTGNSSHSSWDNQCQCTS | |
| GRSDNQGA- | SATRNTTKQVTPQPEEQKERKTTEMQSPMQPVDQASLPGHCREPPPWENEATE | |
| R_K38C- | RIYHFVVGQMVYYQCVQGYRALHRGPAESVCKMTHGKTRWTQPQLICTGEGGG | |
| stableLinker-Fc | GSGGGGSEPKSSDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRWQQG | |
| | NVFSCSVMHEALHNHYTQKSLSLSPGK | |

P1725 Activatable Proprotein

| | | |
|---|---|---|
| Chains 1 | EPKSSDKTHTCPPCPAPELLGGPSVFLFPPKPKDTLMISRTPEVTCVVVDVSH | 234 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| R- | MHEALHNHYTQKSLSLSPGGGGGSGGGGSELCDDDPPEIPHATFKAMAYKEGT | |
| PLGLAGSG | MLNCECKRGFRRIKSGSLYMLCTGNSSHSSWDNQCQCTSSATRNTTKQVTPQP | |
| RSDNQGA- | EEQKERKTTEMQSPMQPVDQASLPGHCREPPPWENEATERIYHFVVGQMVYYQ | |
| L | CVQGYRALHRGPAESVCKMTHGKTRWTQPQLICTGEPLGLAGSGRSDNQGAAP | |
| | TSSSTKKTQLQLEHLLLDLQMILNGINNYKNPKLTRMLTFKFYMPKKATELKH | |
| | LQCLEEELKPLEEVLNLAQSKNFHLRPRDLISNINVIVLELKGSETTFMCEYA | |
| | DETATIVEFLNRWITFSQSIISTLT | |

P1726 Activatable Proprotein

| | | |
|---|---|---|
| Chains 1 | EPKSSDKTHTCPPCPAPELLGGPSVFLFPPKPKDTLMISRTPEVTCVVVDVSH | 235 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker-R- | SDIAVEWESNGQPENNYKTTPPVLDSDGSFFLYSKLTVDKSRWQQGNVFSCSV | |
| PLGLAGSG | MHEALHNHYTQKSLSLSPGGGGGSGGGGSELCDDDPPEIPHATFKAMAYKEGT | |
| RSDNQGA- | MLNCECKRGFRRIKSGSLYMLCTGNSSHSSWDNQCQCTSSATRNTTKQVTPQP | |
| L_T3A | EEQKERKTTEMQSPMQPVDQASLPGHCREPPPWENEATERIYHFVVGQMVYYQ | |
| | CVQGYRALHRGPAESVCKMTHGKTRWTQPQLICTGEPLGLAGSGRSDNQGAAP | |
| | ASSSTKKTQLQLEHLLLDLQMILNGINNYKNPKLTRMLTFKFYMPKKATELKH | |
| | LQCLEEELKPLEEVLNLAQSKNFHLRPRDLISNINVIVLELKGSETTFMCEYA | |
| | DETATIVEFLNRWITFSQSIISTLT | |

P1727 Activatable Proprotein

| | | |
|---|---|---|
| Chains 1 | EPKSSDKTHTCPPCPAPELLGGPSVFLFPPKPKDTLMISRTPEVTCVVVDVSH | 236 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| R_D04C- | MHEALHNHYTQKSLSLSPGGGGGSGGGGSELCCDDPPEIPHATFKAMAYKEGT | |
| PLGLAGSG | MLNCECKRGFRRIKSGSLYMLCTGNSSHSSWDNQCQCTSSATRNTTKQVTPQP | |
| RSDNQGA- | EEQKERKTTEMQSPMQPVDQASLPGHCREPPPWENEATERIYHFVVGQMVYYQ | |
| L_K35C_T3A | CVQGYRALHRGPAESVCKMTHGKTRWTQPQLICTGEPLGLAGSGRSDNQGAAP | |
| | ASSSTKKTQLQLEHLLLDLQMILNGINNYKNPCLTRMLTFKFYMPKKATELKH | |
| | LQCLEEELKPLEEVLNLAQSKNFHLRPRDLISNINVIVLELKGSETTFMCEYA | |
| | DETATIVEFLNRWITFSQSIISTLT | |

P1728 Activatable Proprotein

| | | |
|---|---|---|
| Chains 1 | EPKSSDKTHTCPPCPAPELLGGPSVFLFPPKPKDTLMISRTPEVTCVVVDVSH | 237 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| R_H120C- | MHEALHNHYTQKSLSLSPGGGGGSGGGGSELCDDDPPEIPHATFKAMAYKEGT | |
| PLGLAGSG | MLNCECKRGFRRIKSGSLYMLCTGNSSHSSWDNQCQCTSSATRNTTKQVTPQP | |
| RSDNQGA- | EEQKERKTTEMQSPMQPVDQASLPGHCREPPPWENEATERIYCFVVGQMVYYQ | |
| L_R38C_T3A | CVQGYRALHRGPAESVCKMTHGKTRWTQPQLICTGEPLGLAGSGRSDNQGAAP | |
| | ASSSTKKTQLQLEHLLLDLQMILNGINNYKNPKLTCMLTFKFYMPKKATELKH | |
| | LQCLEEELKPLEEVLNLAQSKNFHLRPRDLISNINVIVLELKGSETTFMCEYA | |
| | DETATIVEFLNRWITFSQSIISTLT | |

P1729 Activatable Proprotein

| | | |
|---|---|---|
| Chains 1 | EPKSSDKTHTCPPCPAPELLGGPSVFLFPPKPKDTLMISRTPEVTCVVVDVSH | 238 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| R_K38C- | MHEALHNHYTQKSLSLSPGGGGGSGGGGSELCDDDPPEIPHATFKAMAYKEGT | |
| PLGLAGSG | MLNCECKRGFRRICSGSLYMLCTGNSSHSSWDNQCQCTSSATRNTTKQVTPQP | |
| RSDNQGA- | EEQKERKTTEMQSPMQPVDQASLPGHCREPPPWENEATERIYHFVVGQMVYYQ | |
| L_E61C_T3A | CVQGYRALHRGPAESVCKMTHGKTRWTQPQLICTGEPLGLAGSGRSDNQGAAP | |
| | ASSSTKKTQLQLEHLLLDLQMILNGINNYKNPKLTRMLTFKFYMPKKATELKH | |
| | LQCLECELKPLEEVLNLAQSKNFHLRPRDLISNINVIVLELKGSETTFMCEYA | |
| | DETATIVEFLNRWITFSQSIISTLT | |

P1730 Activatable Proprotein

| | | |
|---|---|---|
| Chains 1 | EPKSSDKTHTCPPCPAPELLGGPSVFLFPPKPKDTLMISRTPEVTCVVVDVSH | 239 |
| and 2: | EDPEVKFNWYVDGVEVHNAKTKPREEQYNSTYRVVSVLTVLHQDWLNGKEYKC | |
| Fc_delK- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| R_S39C- | MHEALHNHYTQKSLSLSPGGGGGSGGGGSELCDDDPPEIPHATFKAMAYKEGT | |
| PLGLAGSG | MLNCECKRGFRRIKCGSLYMLCTGNSSHSSWDNQCQCTSSATRNTTKQVTPQP | |
| RSDNQGA- | EEQKERKTTEMQSPMQPVDQASLPGHCREPPPWENEATERIYHFVVGQMVYYQ | |
| L_E61C_T3A | CVQGYRALHRGPAESVCKMTHGKTRWTQPQLICTGEPLGLAGSGRSDNQGAAP | |
| | ASSSTKKTQLQLEHLLLDLQMILNGINNYKNPKLTRMLTFKFYMPKKATELKH | |
| | LQCLECELKPLEEVLNLAQSKNFHLRPRDLISNINVIVLELKGSETTFMCEYA | |
| | DETATIVEFLNRWITFSQSIISTLT | |

P1773 Activatable Proprotein

| | | |
|---|---|---|
| Chains 1 | ELCDDDPPEIPHATFKAMAYKEGTMLNCECKRGFRRIKSGSLYMLCTGNSSHS | 240 |
| and 2: | SWDNQCQCTSSATRNTTKQVTPQPEEQKERKTTEMQSPMQPVDQASLPGHCRE | |
| R- | PPPWENEATERIYHFVVGQMVYYQCVQGYRALHRGPAESVCKMTHGKTRWTQP | |
| GSGSENLY | QLICTGEGSGSENLYFQGGSGSAPTSSSTKKTQLQLEHLLLDLQMILNGINNY | |
| FQGGSGS- | KNPKLTRMLTFKFYMPKKATELKHLQCLEEELKPLEEALNLAPSKNFHLRPRD | |
| L-GGGGS- | LISNINVIVLELKGSETTFMCEYADETATIVEFLNRWITFSQSTISTLTGGGG | |
| his6 | SHHHHHH | |

P1774 Activatable Proprotein

| | | |
|---|---|---|
| Chains 1 | EPKSSDKTHTCPPCPAPELLGGPSVFLFPPKPKDTLMISRTPEVTCVVVDVSH | 241 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stable | SDIAVEWESNGQPENNYKTTPPVLDSDGSFFLYSKLTVDKSRWQQGNVFSCSV | |
| Linker- | MHEALHNHYTQKSLSLSPGKGGGGSGGGGSELCDDDPPEIPHATFKAMAYKEG | |
| R- | TMLNCECKRGFRRIKSGSLYMLCTGNSSHSSWDNQCQCTSSATRNTTKQVTPQ | |
| GSGSENLY | PEEQKERKTTEMQSPMQPVDQASLPGHCREPPPWENEATERIYHFVVGQMVYY | |
| FQGGSGS- | QCVQGYRALHRGPAESVCKMTHGKTRWTQPQLICTGEGSGSENLYFQGGSGSA | |
| L | PTSSSTKKTQLQLEHLLLDLQMILNGINNYKNPKLTRMLTFKFYMPKKATELK | |
| | HLQCLEEELKPLEEALNLAPSKNFHLRPRDLISNINVIVLELKGSETTFMCEY | |
| | ADETATIVEFLNRWITFSQSTISTLT | |

P1775 Activatable Proprotein

| | | |
|---|---|---|
| Chains 1 | ELCDDDPPEIPHATFKAMAYKEGTMLNCECKRGFRRIKSGSLYMLCTGNSSHS | 242 |
| and 2: | SWDNQCQCTSSATRNTTKQVTPQPEEQKERKTTEMQSPMQPVDQASLPGHCRE | |
| R- | PPPWENEATERIYHFVVGQMVYYQCVQGYRALHRGPAESVCKMTHGKTRWTQP | |
| GSGSENLY | QLICTGEGSGSENLYFQGGSGSAPTSSSTKKTQLQLEHLLLDLQMILNGINNY | |
| FQGGSGS- | KNPKLTRMLTFKFYMPKKATELKHLQCLEEELKPLEEALNLAPSKNFHLRPRD | |
| L-stable | LISNINVIVLELKGSETTFMCEYADETATIVEFLNRWITFSQSTISTLTGGGG | |
| Linker- | SGGGGSEPKSSDKTHTCPPCPAPELLGGPSVFLFPPKPKDTLMISRTPEVTCV | |
| Fc | VVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSRWQQGN | |
| | VFSCSVMHEALHNHYTQKSLSLSPGK | |

P1776 Activatable Proprotein

| | | |
|---|---|---|
| Chains 1 | ELCDDDPPEIPHATFKAMAYKEGTMLNCECKRGFRRIKSGSLYMLCTGNSSHS | 243 |
| and 2: | SWDNQCQCTSSATRNTTKQVTPQPEEQKERKTTEMQSPMQPVDQASLPGHCRE | |
| R- | PPPWENEATERIYHFVVGQMVYYQCVQGYRALHRGPAESVCKMTHGKTRWTQP | |
| GSGSGSEN | QLICTGEGSGSGSENLYFQGGSGSGSAPTSSSTKKTQLQLEHLLLDLQMILNG | |
| LYFQGGSG | INNYKNPKLTRMLTFKFYMPKKATELKHLQCLEEELKPLEEALNLAPSKNFHL | |
| SGS-L- | RPRDLISNINVIVLELKGSETTFMCEYADETATIVEFLNRWITFSQSTISTLT | |
| GGGGS- | GGGGSHHHHHH | |
| his6 | | |

P1777 Activatable Proprotein

| | | |
|---|---|---|
| Chains 1 | EPKSSDKTHTCPPCPAPELLGGPSVFLFPPKPKDTLMISRTPEVTCVVVDVSH | 244 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stable | SDIAVEWESNGQPENNYKTTPPVLDSDGSFFLYSKLTVDKSRWQQGNVFSCSV | |
| Linker- | MHEALHNHYTQKSLSLSPGKGGGGSGGGGSELCDDDPPEIPHATFKAMAYKEG | |
| R- | TMLNCECKRGFRRIKSGSLYMLCTGNSSHSSWDNQCQCTSSATRNTTKQVTPQ | |
| GSGSGSEN | PEEQKERKTTEMQSPMQPVDQASLPGHCREPPPWENEATERIYHFVVGQMVYY | |
| LYFQGGSG | QCVQGYRALHRGPAESVCKMTHGKTRWTQPQLICTGEGSGSGSENLYFQGGSG | |
| SGS-L | SGSAPTSSSTKKTQLQLEHLLLDLQMILNGINNYKNPKLTRMLTFKFYMPKKA | |
| | TELKHLQCLEEELKPLEEALNLAPSKNFHLRPRDLISNINVIVLELKGSETTF | |
| | MCEYADETATIVEFLNRWITFSQSTISTLT | |

P1778 Activatable Proprotein

| | | |
|---|---|---|
| Chains 1 | ELCDDDPPEIPHATFKAMAYKEGTMLNCECKRGFRRIKSGSLYMLCTGNSSHS | 245 |
| and 2: | SWDNQCQCTSSATRNTTKQVTPQPEEQKERKTTEMQSPMQPVDQASLPGHCRE | |
| R- | PPPWENEATERIYHFVVGQMVYYQCVQGYRALHRGPAESVCKMTHGKTRWTQP | |
| GSGSGSEN | QLICTGEGSGSGSENLYFQGGSGSGSAPTSSSTKKTQLQLEHLLLDLQMILNG | |
| LYFQGGSG | INNYKNPKLTRMLTFKFYMPKKATELKHLQCLEEELKPLEEALNLAPSKNFHL | |
| SGS-L- | RPRDLISNINVIVLELKGSETTFMCEYADETATIVEFLNRWITFSQSTISTLT | |
| stable | GGGGSGGGGSEPKSSDKTHTCPPCPAPELLGGPSVFLFPPKPKDTLMISRTPE | |
| Linker- | VTCVVVDVSHEDPEVKFNWYVDGVEVHNAKTKPREEQYNSTYRVVSVLTVLHQ | |
| Fc | DWLNGKEYKCKVSNKALPAPIEKTISKAKGQPREPQVYTLPPSREEMTKNQVS | |
| (stable | LTCLVKGFYPSDIAVEWESNGQPENNYKTTPPVLDSDGSFFLYSKLTVDKSRW | |
| linker: | QQGNVFSCSVMHEALHNHYTQKSLSLSPGK | |
| GGGGSGGG | | |
| GSEPKSSD | | |
| KTHT) | | |

P1779 Activatable Proprotein

| | | |
|---|---|---|
| Chains 1 | APASSSTKKTQLQLEHLLLDLQMILNGINNYKNPKLTRMLTFKFYMPKKATEL | 246 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNHGGAELCDDDP | |
| GGSPLGLA | PEIPHATFKAMAYKEGTMLNCECKRGFRRIKSGSLYMLCTGNSSHSSWDNQCQ | |
| GSGRSDNH | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGGGSGGGGSEPKSSDKTHTCPPCPAPEAAGGPSVFLFPPKPKDTLMISRTPE | |
| Fc(IgG1 | VTCVVVDVSHEDPEVKFNWYVDGVEVHNAKTKPREEQYNSTYRVVSVLTVLHQ | |
| AAA) | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1779 Cleaved proteins (Complete uPA/Matriptase cleavage)

| | | |
|---|---|---|
| Chains 1 | APASSSTKKTQLQLEHLLLDLQMILNGINNYKNPKLTRMLTFKFYMPKKATEL | 247 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGR | |
| GGSPLGLA | | |
| GSGR | | |
| Chains 3 | SDNHGGAELCDDDPPEIPHATFKAMAYKEGTMLNCECKRGFRRIKSGSLYMLC | 248 |
| and 4: | TGNSSHSSWDNQCQCTSSATRNTTKQVTPQPEEQKERKTTEMQSPMQPVDQAS | |
| SDNHGGA- | LPGHCREPPPWENEATERIYHFVVGQMVYYQCVQGYRALHRGPAESVCKMTHG | |
| R-stable | KTRWTQPQLICTGEGGGGSGGGGSEPKSSDKTHTCPPCPAPEAAGGPSVFLFP | |
| Linker- | PKPKDTLMISRTPEVTCVVVDVSHEDPEVKFNWYVDGVEVHNAKTKPREEQYN | |
| Fc | STYRVVSVLTVLHQDWLNGKEYKCKVSNKALAAPIEKTISKAKGQPREPQVYT | |
| | LPPSRDELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |

P1779 Cleaved proteins (Partial uPA/Matriptase cleavage)

| | | |
|---|---|---|
| Chain 1: | APASSSTKKTQLQLEHLLLDLQMILNGINNYKNPKLTRMLTFKFYMPKKATEL | 249 |
| L_T3A- | KHLQCLEEELKPLEEALNLAPSKNFHLRPRDLISNINVIVLELKGSETTFMCE | |
| GGSPLGLA | YADETATIVEFLNRWITFSQSTISTLTGGSPLGLAGSGRSDNHGGAELCDDDP | |
| GSGRSDNH | PEIPHATFKAMAYKEGTMLNCECKRGFRRIKSGSLYMLCTGNSSHSSWDNQCQ | |
| GGA-R- | CTSSATRNTTKQVTPQPEEQKERKTTEMQSPMQPVDQASLPGHCREPPPWENE | |
| stableLinker-Fc | ATERIYHFVVGQMVYYQCVQGYRALHRGPAESVCKMTHGKTRWTQPQLICTGE | |
| | GGGGSGGGGSEPKSSDKTHTCPPCPAPEAAGGPSVFLFPPKPKDTLMISRTPE | |
| | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |
| Chain 2: | APASSSTKKTQLQLEHLLLDLQMILNGINNYKNPKLTRMLTFKFYMPKKATEL | 250 |
| L_T3A- | KHLQCLEEELKPLEEALNLAPSKNFHLRPRDLISNINVIVLELKGSETTFMCE | |
| GGSPLGLA | YADETATIVEFLNRWITFSQSTISTLTGGSPLGLAGSGR | |
| GSGR | | |
| Chain 3: | SDNHGGAELCDDDPPEIPHATFKAMAYKEGTMLNCECKRGFRRIKSGSLYMLC | 251 |
| SDNHGGA- | TGNSSHSSWDNQCQCTSSATRNTTKQVTPQPEEQKERKTTEMQSPMQPVDQAS | |
| R-stable | LPGHCREPPPWENEATERIYHFVVGQMVYYQCVQGYRALHRGPAESVCKMTHG | |
| Linker- | KTRWTQPQLICTGEGGGGSGGGGSEPKSSDKTHTCPPCPAPEAAGGPSVFLFP | |
| Fc | PKPKDTLMISRTPEVTCVVVDVSHEDPEVKFNWYVDGVEVHNAKTKPREEQYN | |
| | STYRVVSVLTVLHQDWLNGKEYKCKVSNKALAAPIEKTISKAKGQPREPQVYT | |
| | LPPSRDELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |

P1779 Cleaved proteins (Complete MMP-2/MMP-9 cleavage)

| | | |
|---|---|---|
| Chains 1 | APASSSTKKTQLQLEHLLLDLQMILNGINNYKNPKLTRMLTFKFYMPKKATEL | 252 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLG | |
| GGSPLG | | |
| Chains 3 | LAGSGRSDNHGGAELCDDDPPEIPHATFKAMAYKEGTMLNCECKRGFRRIKSG | 253 |
| and 4: | SLYMLCTGNSSHSSWDNQCQCTSSATRNTTKQVTPQPEEQKERKTTEMQSPMQ | |
| LAGSGRSD | PVDQASLPGHCREPPPWENEATERIYHFVVGQMVYYQCVQGYRALHRGPAESV | |
| NHGGA-R- | CKMTHGKTRWTQPQLICTGEGGGGSGGGGSEPKSSDKTHTCPPCPAPEAAGGP | |
| stable | SVFLFPPKPKDTLMISRTPEVTCVVVDVSHEDPEVKFNWYVDGVEVHNAKTKP | |
| Linker- | REEQYNSTYRVVSVLTVLHQDWLNGKEYKCKVSNKALAAPIEKTISKAKGQPR | |
| Fc | EPQVYTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |

P1779 Cleaved proteins (Partial MMP-2/MMP-9 cleavage)

| | | |
|---|---|---|
| Chain 1: | APASSSTKKTQLQLEHLLLDLQMILNGINNYKNPKLTRMLTFKFYMPKKATEL | 254 |
| L_T3A- | KHLQCLEEELKPLEEALNLAPSKNFHLRPRDLISNINVIVLELKGSETTFMCE | |
| GGSPLGLA | YADETATIVEFLNRWITFSQSTISTLTGGSPLGLAGSGRSDNHGGAELCDDDP | |
| GSGRSDNH | PEIPHATFKAMAYKEGTMLNCECKRGFRRIKSGSLYMLCTGNSSHSSWDNQCQ | |
| GGA-R- | CTSSATRNTTKQVTPQPEEQKERKTTEMQSPMQPVDQASLPGHCREPPPWENE | |
| stableLinker-Fc | ATERIYHFVVGQMVYYQCVQGYRALHRGPAESVCKMTHGKTRWTQPQLICTGE | |
| | GGGGSGGGGSEPKSSDKTHTCPPCPAPEAAGGPSVFLFPPKPKDTLMISRTPE | |
| | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |
| Chain 2: | APASSSTKKTQLQLEHLLLDLQMILNGINNYKNPKLTRMLTFKFYMPKKATEL | 255 |
| L_T3A- | KHLQCLEEELKPLEEALNLAPSKNFHLRPRDLISNINVIVLELKGSETTFMCE | |
| GGSPLG | YADETATIVEFLNRWITFSQSTISTLTGGSPLG | |
| Chain 3: | LAGSGRSDNHGGAELCDDDPPEIPHATFKAMAYKEGTMLNCECKRGFRRIKSG | 256 |
| LAGSGRSD | SLYMLCTGNSSHSSWDNQCQCTSSATRNTTKQVTPQPEEQKERKTTEMQSPMQ | |
| NHGGA-R- | PVDQASLPGHCREPPPWENEATERIYHFVVGQMVYYQCVQGYRALHRGPAESV | |
| stable | CKMTHGKTRWTQPQLICTGEGGGGSGGGGSEPKSSDKTHTCPPCPAPEAAGGP | |
| Linker- | SVFLFPPKPKDTLMISRTPEVTCVVVDVSHEDPEVKFNWYVDGVEVHNAKTKP | |
| Fc | REEQYNSTYRVVSVLTVLHQDWLNGKEYKCKVSNKALAAPIEKTISKAKGQPR | |
| | EPQVYTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSPGK | |

P1779 Cleaved proteins (Complete Legumain cleavage)

| | | |
|---|---|---|
| Chains 1 | APASSSTKKTQLQLEHLLLDLQMILNGINNYKNPKLTRMLTFKFYMPKKATEL | 257 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDN | |
| GGSPLGLA | | |
| GSGRSDN | | |
| Chains 3 | HGGAELCDDDPPEIPHATFKAMAYKEGTMLNCECKRGFRRIKSGSLYMLCTGN | 258 |
| and 4: | SSHSSWDNQCQCTSSATRNTTKQVTPQPEEQKERKTTEMQSPMQPVDQASLPG | |
| HGGA-R- | HCREPPPWENEATERIYHFVVGQMVYYQCVQGYRALHRGPAESVCKMTHGKTR | |
| stable | WTQPQLICTGEGGGGSGGGGSEPKSSDKTHTCPPCPAPEAAGGPSVFLFPPKP | |
| Linker- | KDTLMISRTPEVTCVVVDVSHEDPEVKFNWYVDGVEVHNAKTKPREEQYNSTY | |
| Fc | RVVSVLTVLHQDWLNGKEYKCKVSNKALAAPIEKTISKAKGQPREPQVYTLPP | |
| | SRDELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |

P1779 Cleaved proteins (Partial Legumain cleavage)

| | | |
|---|---|---|
| Chain 1: | APASSSTKKTQLQLEHLLLDLQMILNGINNYKNPKLTRMLTFKFYMPKKATEL | 259 |
| L_T3A- | KHLQCLEEELKPLEEALNLAPSKNFHLRPRDLISNINVIVLELKGSETTFMCE | |
| GGSPLGLA | YADETATIVEFLNRWITFSQSTISTLTGGSPLGLAGSGRSDNHGGAELCDDDP | |
| GSGRSDNH | PEIPHATFKAMAYKEGTMLNCECKRGFRRIKSGSLYMLCTGNSSHSSWDNQCQ | |
| GGA-R- | CTSSATRNTTKQVTPQPEEQKERKTTEMQSPMQPVDQASLPGHCREPPPWENE | |
| stableLinker-Fc | ATERIYHFVVGQMVYYQCVQGYRALHRGPAESVCKMTHGKTRWTQPQLICTGE | |
| | GGGGSGGGGSEPKSSDKTHTCPPCPAPEAAGGPSVFLFPPKPKDTLMISRTPE | |
| | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |
| Chain 2: | APASSSTKKTQLQLEHLLLDLQMILNGINNYKNPKLTRMLTFKFYMPKKATEL | 260 |
| L_T3A- | KHLQCLEEELKPLEEALNLAPSKNFHLRPRDLISNINVIVLELKGSETTFMCE | |
| GGSPLGLA | YADETATIVEFLNRWITFSQSTISTLTGGSPLGLAGSGRSDN | |
| GSGRSDN | | |
| Chain 3: | HGGAELCDDDPPEIPHATFKAMAYKEGTMLNCECKRGFRRIKSGSLYMLCTGN | 261 |
| HGGA-R- | SSHSSWDNQCQCTSSATRNTTKQVTPQPEEQKERKTTEMQSPMQPVDQASLPG | |
| stable | HCREPPPWENEATERIYHFVVGQMVYYQCVQGYRALHRGPAESVCKMTHGKTR | |
| Linker- | WTQPQLICTGEGGGGSGGGGSEPKSSDKTHTCPPCPAPEAAGGPSVFLFPPKP | |
| Fc | KDTLMISRTPEVTCVVVDVSHEDPEVKFNWYVDGVEVHNAKTKPREEQYNSTY | |
| | RVVSVLTVLHQDWLNGKEYKCKVSNKALAAPIEKTISKAKGQPREPQVYTLPP | |
| | SRDELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |

P1780 Activatable Proprotein

| | | |
|---|---|---|
| Chains 1 | APASSSTKKTQLQLEHLLLDLQMILNGINNYKNPKLTRMLTFKFYMPKKATEL | 262 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNHGGAELCDDDP | |
| GGSPLGLA | PEIPHATFKAMAYKEGTMLNCECKRGFRRIKSGSLYMLCTGNSSHSSWDNQCQ | |
| GSGRSDNH | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGSGGGGSEPKSSDKTHTCPPCPAPEAAGGPSVFLFPPKPKDTLMISRTPEVT | |
| Fc(IgG1_AAA) | CVVVDVSHEDPEVKFNWYVDGVEVHNAKTKPREEQYNSTYRVVSVLTVLHQDW | |
| | LNGKEYKCKVSNKALAAPIEKTISKAKGQPREPQVYTLPPSRDELTKNQVSLT | |
| | CLVKGFYPSDIAVEWESNGQPENNYKTTPPVLDSDGSFFLYSKLTVDKSRWQQ | |
| | GNVFSCSVMHEALHNHYTQKSLSLSPGK | |

P1781 Activatable Proprotein

| | | |
|---|---|---|
| Chains 1 | APASSSTKKTQLQLEHLLLDLQMILNGINNYKNPKLTRMLTFKFYMPKKATEL | 263 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNHGGAELCDDDP | |
| GGSPLGLA | PEIPHATFKAMAYKEGTMLNCECKRGFRRIKSGSLYMLCTGNSSHSSWDNQCQ | |
| GSGRSDNH | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | SGGGGSEPKSSDKTHTCPPCPAPEAAGGPSVFLFPPKPKDTLMISRTPEVTCV | |
| Fc(IgG1_AAA) | VVDVSHEDPEVKFNWYVDGVEVHNAKTKPREEQYNSTYRVVSVLTVLHQDWLN | |
| | GKEYKCKVSNKALAAPIEKTISKAKGQPREPQVYTLPPSRDELTKNQVSLTCL | |
| | VKGFYPSDIAVEWESNGQPENNYKTTPPVLDSDGSFFLYSKLTVDKSRWQQGN | |
| | VFSCSVMHEALHNHYTQKSLSLSPGK | |

P1782 Activatable Proprotein

| | | |
|---|---|---|
| Chains 1 | APASSSTKKTQLQLEHLLLDLQMILNGINNYKNPKLTRMLTFKFYMPKKATEL | 264 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNHGGAELCDDDP | |
| GGSPLGLA | PEIPHATFKAMAYKEGTMLNCECKRGFRRIKSGSLYMLCTGNSSHSSWDNQCQ | |
| GSGRSDNH | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGGSEPKSSDKTHTCPPCPAPEAAGGPSVFLFPPKPKDTLMISRTPEVTCVVV | |
| Fc(IgG1_AAA) | DVSHEDPEVKFNWYVDGVEVHNAKTKPREEQYNSTYRVVSVLTVLHQDWLNGK | |
| | EYKCKVSNKALAAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPGK | |

P1783 Activatable Proprotein

| | | |
|---|---|---|
| Chains 1 | APASSSTKKTQLQLEHLLLDLQMILNGINNYKNPKLTRMLTFKFYMPKKATEL | 265 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNQGGAELCDDDP | |
| GGSPLGLA | PEIPHATFKAMAYKEGTMLNCECKRGFRRIKSGSLYMLCTGNSSHSSWDNQCQ | |
| GSGRSDNQ | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGGGSGGGGSEPKSSDKTHTCPPCPAPEAAGGPSVFLFPPKPKDTLMISRTPE | |
| Fc(IgG1_AAA) | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1784 Activatable Proprotein

| | | |
|---|---|---|
| Chains 1 | APASSSTKKTQLQLEHLLLDLQMILNGINNYKNPKLTRMLTFKFYMPKKATEL | 266 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNRGGAELCDDDP | |
| GGSPLGLA | PEIPHATFKAMAYKEGTMLNCECKRGFRRIKSGSLYMLCTGNSSHSSWDNQCQ | |
| GSGRSDNR | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLi | GGGGSGGGGSEPKSSDKTHTCPPCPAPEAAGGPSVFLFPPKPKDTLMISRTPE | |
| nker- | VTCVVVDVSHEDPEVKFNWYVDGVEVHNAKTKPREEQYNSTYRVVSVLTVLHQ | |
| Fc(IgG1 | DWLNGKEYKCKVSNKALAAPIEKTISKAKGQPREPQVYTLPPSRDELTKNQVS | |
| AAA) | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1785 Activatable Proprotein

| | | |
|---|---|---|
| Chains 1 | APASSSTKKTQLQLEHLLLDLQMILNGINNYKNPKLTRMLTFKFYMPKKATEL | 267 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSPLGLAGSGRSDNTGGAELCDDDP | |
| GGSPLGLA | PEIPHATFKAMAYKEGTMLNCECKRGFRRIKSGSLYMLCTGNSSHSSWDNQCQ | |
| GSGRSDNT | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGGGSGGGGSEPKSSDKTHTCPPCPAPEAAGGPSVFLFPPKPKDTLMISRTPE | |
| Fc(IgG1_AAA) | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1786 Activatable Proprotein

| | | |
|---|---|---|
| Chains 1 | APASSSTKKTQLQLEHLLLDLQMILNGINNYKNPKLTRMLTFKFYMPKKATEL | 268 |
| and 2: | KHLQCLEEELKPLEEALNLAPSKNFHLRPRDLISNINVIVLELKGSETTFMCE | |
| L_T3A- | YADETATIVEFLNRWITFSQSTISTLTGGSGGSGGSGGSGGSGGGAELCDDDP | |
| GGSGGSGG | PEIPHATFKAMAYKEGTMLNCECKRGFRRIKSGSLYMLCTGNSSHSSWDNQCQ | |
| SGGSGGSG | CTSSATRNTTKQVTPQPEEQKERKTTEMQSPMQPVDQASLPGHCREPPPWENE | |
| GGA-R- | ATERIYHFVVGQMVYYQCVQGYRALHRGPAESVCKMTHGKTRWTQPQLICTGE | |
| stableLinker- | GGGGSGGGGSEPKSSDKTHTCPPCPAPEAAGGPSVFLFPPKPKDTLMISRTPE | |
| Fc(IgG1_AAA) | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |

P1834 Activatable Proprotein

| | | |
|---|---|---|
| Chains 1 | EPKSSDKTHTCPPCPAPEAAGGPSVFLFPPKPKDTLMISRTPEVTCVVVDVSH | 269 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGSAPASSSTKKTQLQLEHLLLDLQMIL | |
| S-L_T3A- | NGINNYKNPKLTRMLTFKFYMPKKATELKHLQCLEEELKPLEEALNLAPSKNF | |
| GGG- | HLRPRDLISNINVIVLELKGSETTFMCEYADETATIVEFLNRWITFSQSTIST | |
| SGRSDNH- | LTGGGSGRSDNHGGAELCDDDPPEIPHATFKAMAYKEGTMLNCECKRGFRRIK | |
| GGA-R | SGSLYMLCTGNSSHSSWDNQCQCTSSATRNTTKQVTPQPEEQKERKTTEMQSP | |
| 9/13 | MQPVDQASLPGHCREPPPWENEATERIYHFVVGQMVYYQCVQGYRALHRGPAE | |
| | SVCKMTHGKTRWTQPQLICTGE | |

P1834 Cleaved proteins (Complete uPA/Matriptase cleavage)

| | | |
|---|---|---|
| Chains 1 | EPKSSDKTHTCPPCPAPEAAGGPSVFLFPPKPKDTLMISRTPEVTCVVVDVSH | 270 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGSAPASSSTKKTQLQLEHLLLDLQMIL | |
| S-L_T3A- | NGINNYKNPKLTRMLTFKFYMPKKATELKHLQCLEEELKPLEEALNLAPSKNF | |
| GGG-SGR | HLRPRDLISNINVIVLELKGSETTFMCEYADETATIVEFLNRWITFSQSTIST | |
| | LTGGGSGR | |
| Chains 3 | SDNHGGAELCDDDPPEIPHATFKAMAYKEGTMLNCECKRGFRRIKSGSLYMLC | 271 |
| and 4: | TGNSSHSSWDNQCQCTSSATRNTTKQVTPQPEEQKERKTTEMQSPMQPVDQAS | |
| SDNH- | LPGHCREPPPWENEATERIYHFVVGQMVYYQCVQGYRALHRGPAESVCKMTHG | |
| GGA-R | KTRWTQPQLICTGE | |

P1834 Cleaved proteins (Partial uPA/Matriptase cleavage)

| | | |
|---|---|---|
| Chain 1: | EPKSSDKTHTCPPCPAPEAAGGPSVFLFPPKPKDTLMISRTPEVTCVVVDVSH | 272 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDNH- | HLRPRDLISNINVIVLELKGSETTFMCEYADETATIVEFLNRWITFSQSTIST | |
| GGA-R | LTGGGSGRSDNHGGAELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |
| Chain 2: | EPKSSDKTHTCPPCPAPEAAGGPSVFLFPPKPKDTLMISRTPEVTCVVVDVSH | 273 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG-SGR | NGINNYKNPKLTRMLTFKFYMPKKATELKHLQCLEEELKPLEEALNLAPSKNF | |
| | HLRPRDLISNINVIVLELKGSETTFMCEYADETATIVEFLNRWITFSQSTIST | |
| | LTGGGSGR | |
| Chain 3: | SDNHGGAELCDDDPPEIPHATFKAMAYKEGTMLNCECKRGFRRIKSGSLYMLC | 274 |
| SDNH- | TGNSSHSSWDNQCQCTSSATRNTTKQVTPQPEEQKERKTTEMQSPMQPVDQAS | |
| GGA-R | LPGHCREPPPWENEATERIYHFVVGQMVYYQCVQGYRALHRGPAESVCKMTHG | |
| | KTRWTQPQLICTGE | |

P1834 Cleaved proteins (Complete MMP-2/MMP-9 cleavage)

| | | |
|---|---|---|
| Chains 1 | EPKSSDKTHTCPPCPAPEAAGGPSVFLFPPKPKDTLMISRTPEVTCVVVDVSH | 275 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS-PLG | SDIAVEWESNGQPENNYKTTPPVLDSDGSFFLYSKLTVDKSRWQQGNVFSCSV | |
| | MHEALHNHYTQKSLSLSPGGSPLG | |
| Chains 3 | LAGSAPASSSTKKTQLQLEHLLLDLQMILNGINNYKNPKLTRMLTFKFYMPKK | 276 |
| and 4: | ATELKHLQCLEEELKPLEEALNLAPSKNFHLRPRDLISNINVIVLELKGSETT | |
| LAG-S- | FMCEYADETATIVEFLNRWITFSQSTISTLTGGGSGRSDNHGGAELCDDDPPE | |
| L_T3A- | IPHATFKAMAYKEGTMLNCECKRGFRRIKSGSLYMLCTGNSSHSSWDNQCQCT | |
| GGG- | SSATRNTTKQVTPQPEEQKERKTTEMQSPMQPVDQASLPGHCREPPPWENEAT | |
| SGRSDNH- | ERIYHFVVGQMVYYQCVQGYRALHRGPAESVCKMTHGKTRWTQPQLICTGE | |
| GGA-R | | |

P1834 Cleaved proteins (Partial MMP-2/MMP-9 cleavage)

| | | |
|---|---|---|
| Chain 1: | EPKSSDKTHTCPPCPAPEAAGGPSVFLFPPKPKDTLMISRTPEVTCVVVDVSH | 277 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDNH- | HLRPRDLISNINVIVLELKGSETTFMCEYADETATIVEFLNRWITFSQSTIST | |
| GGA-R | LTGGGSGRSDNHGGAELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |
| Chain 2: | EPKSSDKTHTCPPCPAPEAAGGPSVFLFPPKPKDTLMISRTPEVTCVVVDVSH | 278 |
| Fc(G1_AAA_C217S)_delK- | EDPEVKFNWYVDGVEVHNAKTKPREEQYNSTYRVVSVLTVLHQDWLNGKEYKC | |
| GS-PLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 279 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDNHGGAELCDDDPPE | |
| GGG- | IPHATFKAMAYKEGTMLNCECKRGFRRIKSGSLYMLCTGNSSHSSWDNQCQCT | |
| SGRSDNH- | SSATRNTTKQVTPQPEEQKERKTTEMQSPMQPVDQASLPGHCREPPPWENEAT | |
| GGA-R | ERIYHFVVGQMVYYQCVQGYRALHRGPAESVCKMTHGKTRWTQPQLICTGE | |

P1834 Cleaved proteins (Complete Legumain cleavage)

| | | |
|---|---|---|
| Chains 1 | EPKSSDKTHTCPPCPAPEAAGGPSVFLFPPKPKDTLMISRTPEVTCVVVDVSH | 280 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGSAPASSSTKKTQLQLEHLLLDLQMIL | |
| S-L_T3A- | NGINNYKNPKLTRMLTFKFYMPKKATELKHLQCLEEELKPLEEALNLAPSKNF | |
| GGG- | HLRPRDLISNINVIVLELKGSETTFMCEYADETATIVEFLNRWITFSQSTIST | |
| SGRSDN | LTGGGSGRSDN | |
| Chains 3 | HGGAELCDDDPPEIPHATFKAMAYKEGTMLNCECKRGFRRIKSGSLYMLCTGN | 281 |
| and 4: | SSHSSWDNQCQCTSSATRNTTKQVTPQPEEQKERKTTEMQSPMQPVDQASLPG | |
| H-GGA-R | HCREPPPWENEATERIYHFVVGQMVYYQCVQGYRALHRGPAESVCKMTHGKTR | |
| | WTQPQLICTGE | |

P1834 Cleaved proteins (Partial Legumain cleavage)

| | | |
|---|---|---|
| Chain 1: | EPKSSDKTHTCPPCPAPEAAGGPSVFLFPPKPKDTLMISRTPEVTCVVVDVSH | 282 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDNH- | HLRPRDLISNINVIVLELKGSETTFMCEYADETATIVEFLNRWITFSQSTIST | |
| GGA-R | LTGGGSGRSDNHGGAELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |
| Chain 2: | EPKSSDKTHTCPPCPAPEAAGGPSVFLFPPKPKDTLMISRTPEVTCVVVDVSH | 283 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDN | HLRPRDLISNINVIVLELKGSETTFMCEYADETATIVEFLNRWITFSQSTIST | |
| | LTGGGSGRSDN | |
| Chain 3: | HGGAELCDDDPPEIPHATFKAMAYKEGTMLNCECKRGFRRIKSGSLYMLCTGN | 284 |
| H-GGA-R | SSHSSWDNQCQCTSSATRNTTKQVTPQPEEQKERKTTEMQSPMQPVDQASLPG | |
| | HCREPPPWENEATERIYHFVVGQMVYYQCVQGYRALHRGPAESVCKMTHGKTR | |
| | WTQPQLICTGE | |

P1834 Cleaved proteins (Complete uPA/Matriptase cleavage,
complete MMP-2/MMP-9 cleavage)

| | | |
|---|---|---|
| Chains 1 | EPKSSDKTHTCPPCPAPEAAGGPSVFLFPPKPKDTLMISRTPEVTCVVVDVSH | 285 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS-PLG | SDIAVEWESNGQPENNYKTTPPVLDSDGSFFLYSKLTVDKSRWQQGNVFSCSV | |
| | MHEALHNHYTQKSLSLSPGGSPLG | |
| Chains 3 | LAGSAPASSSTKKTQLQLEHLLLDLQMILNGINNYKNPKLTRMLTFKFYMPKK | 286 |
| and 4: | ATELKHLQCLEEELKPLEEALNLAPSKNFHLRPRDLISNINVIVLELKGSETT | |
| LAG-S- | FMCEYADETATIVEFLNRWITFSQSTISTLTGGGSGR | |
| L_T3A- | | |
| GGG-SGR | | |
| Chains 5 | SDNHGGAELCDDDPPEIPHATFKAMAYKEGTMLNCECKRGFRRIKSGSLYMLC | 287 |
| and 6: | TGNSSHSSWDNQCQCTSSATRNTTKQVTPQPEEQKERKTTEMQSPMQPVDQAS | |
| SDNH- | LPGHCREPPPWENEATERIYHFVVGQMVYYQCVQGYRALHRGPAESVCKMTHG | |
| GGA-R | KTRWTQPQLICTGE | |

P1834 Cleaved proteins (Complete uPA/Matriptase cleavage,
partial MMP-2/MMP-9 cleavage)

| | | |
|---|---|---|
| Chains 1 | EPKSSDKTHTCPPCPAPEAAGGPSVFLFPPKPKDTLMISRTPEVTCVVVDVSH | 288 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS-PLG | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 289 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDNHGGAELCDDDPPE | |
| GGG- | IPHATFKAMAYKEGTMLNCECKRGFRRIKSGSLYMLCTGNSSHSSWDNQCQCT | |
| SGRSDNH- | SSATRNTTKQVTPQPEEQKERKTTEMQSPMQPVDQASLPGHCREPPPWENEAT | |
| GGA-R | ERIYHFVVGQMVYYQCVQGYRALHRGPAESVCKMTHGKTRWTQPQLICTGE | |
| Chain 4: | LAGSAPASSSTKKTQLQLEHLLLDLQMILNGINNYKNPKLTRMLTFKFYMPKK | 290 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGR | |
| GGG- SGR | | |
| Chain 5: | SDNHGGAELCDDDPPEIPHATFKAMAYKEGTMLNCECKRGFRRIKSGSLYMLC | 291 |
| SDNH- | TGNSSHSSWDNQCQCTSSATRNTTKQVTPQPEEQKERKTTEMQSPMQPVDQAS | |
| GGA-R | LPGHCREPPPWENEATERIYHFVVGQMVYYQCVQGYRALHRGPAESVCKMTHG | |
| | KTRWTQPQLICTGE | |

P1834 Cleaved proteins (Partial uPA/Matriptase cleavage,
complete MMP-2/MMP-9 cleavage)

| | | |
|---|---|---|
| Chain 1: | EPKSSDKTHTCPPCPAPEAAGGPSVFLFPPKPKDTLMISRTPEVTCVVVDVSH | 292 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG-SGP | NGINNYKNPKLTRMLTFKFYMPKKATELKHLQCLEEELKPLEEALNLAPSKNF | |
| | HLRPRDLISNINVIVLELKGSETTFMCEYADETATIVEFLNRWITFSQSTIST | |
| | LTGGGSGR | |
| Chain 2: | EPKSSDKTHTCPPCPAPEAAGGPSVFLFPPKPKDTLMISRTPEVTCVVVDVSH | 293 |
| Fc(G1_AAA_C217S)_delK- | EDPEVKFNWYVDGVEVHNAKTKPREEQYNSTYRVVSVLTVLHQDWLNGKEYKC | |
| GS-PLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 294 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGR | |
| GGG- SGP | | |
| Chains 4 | SDNHGGAELCDDDPPEIPHATFKAMAYKEGTMLNCECKRGFRRIKSGSLYMLC | 295 |
| and 5: | TGNSSHSSWDNQCQCTSSATRNTTKQVTPQPEEQKERKTTEMQSPMQPVDQAS | |
| SDNH- | LPGHCREPPPWENEATERIYHFVVGQMVYYQCVQGYRALHRGPAESVCKMTHG | |
| GGA-R | KTRWTQPQLICTGE | |

P1834 Cleaved proteins (Partial uPA/Matriptase cleavage,
partial MMP-2/MMP-9 cleavage)

| | | |
|---|---|---|
| Chain 1: | EPKSSDKTHTCPPCPAPEAAGGPSVFLFPPKPKDTLMISRTPEVTCVVVDVSH | 296 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDNH- | HLRPRDLISNINVIVLELKGSETTFMCEYADETATIVEFLNRWITFSQSTIST | |
| GGA-R | LTGGGSGRSDNHGGAELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |
| Chain 2: | EPKSSDKTHTCPPCPAPEAAGGPSVFLFPPKPKDTLMISRTPEVTCVVVDVSH | 297 |
| Fc(G1_AAA_C217S)_delK- | EDPEVKFNWYVDGVEVHNAKTKPREEQYNSTYRVVSVLTVLHQDWLNGKEYKC | |
| GS-PLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 298 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDNHGGAELCDDDPPE | |
| GGG- | IPHATFKAMAYKEGTMLNCECKRGFRRIKSGSLYMLCTGNSSHSSWDNQCQCT | |
| SGRSDNH- | SSATRNTTKQVTPQPEEQKERKTTEMQSPMQPVDQASLPGHCREPPPWENEAT | |
| GGA-R | ERIYHFVVGQMVYYQCVQGYRALHRGPAESVCKMTHGKTRWTQPQLICTGE | |
| Chain 4: | EPKSSDKTHTCPPCPAPEAAGGPSVFLFPPKPKDTLMISRTPEVTCVVVDVSH | 299 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG-SGR | NGINNYKNPKLTRMLTFKFYMPKKATELKHLQCLEEELKPLEEALNLAPSKNF | |
| | HLRPRDLISNINVIVLELKGSETTFMCEYADETATIVEFLNRWITFSQSTIST | |
| | LTGGGSGR | |
| Chain 5: | SDNHGGAELCDDDPPEIPHATFKAMAYKEGTMLNCECKRGFRRIKSGSLYMLC | 300 |
| SDNH- | TGNSSHSSWDNQCQCTSSATRNTTKQVTPQPEEQKERKTTEMQSPMQPVDQAS | |
| GGA-R | LPGHCREPPPWENEATERIYHFVVGQMVYYQCVQGYRALHRGPAESVCKMTHG | |
| | KTRWTQPQLICTGE | |
| Chain 6: | LAGSAPASSSTKKTQLQLEHLLLDLQMILNGINNYKNPKLTRMLTFKFYMPKK | 301 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGR | |
| GGG-SGR | | |

P1834 Cleaved proteins (Complete uPA/Matriptase cleavage,
complete Legumain cleavage)

| | | |
|---|---|---|
| Chains 1 | EPKSSDKTHTCPPCPAPEAAGGPSVFLFPPKPKDTLMISRTPEVTCVVVDVSH | 302 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS-PLG | SDIAVEWESNGQPENNYKTTPPVLDSDGSFFLYSKLTVDKSRWQQGNVFSCSV | |
| | MHEALHNHYTQKSLSLSPGGSPLG | |
| Chains 3 | LAGSAPASSSTKKTQLQLEHLLLDLQMILNGINNYKNPKLTRMLTFKFYMPKK | 303 |
| and 4: | ATELKHLQCLEEELKPLEEALNLAPSKNFHLRPRDLISNINVIVLELKGSETT | |
| LAG-S- | FMCEYADETATIVEFLNRWITFSQSTISTLTGGGSGRSDN | |
| L_T3A- | | |
| GGG- | | |
| SGRSDN | | |
| Chains 5 | HGGAELCDDDPPEIPHATFKAMAYKEGTMLNCECKRGFRRIKSGSLYMLCTGN | 304 |
| and 6: | SSHSSWDNQCQCTSSATRNTTKQVTPQPEEQKERKTTEMQSPMQPVDQASLPG | |
| H-GGA-R | HCREPPPWENEATERIYHFVVGQMVYYQCVQGYRALHRGPAESVCKMTHGKTR | |
| | WTQPQLICTGE | |

P1834 Cleaved proteins (Complete uPA/Matriptase cleavage,
partial Legumain cleavage)

| | | |
|---|---|---|
| Chains 1 | EPKSSDKTHTCPPCPAPEAAGGPSVFLFPPKPKDTLMISRTPEVTCVVVDVSH | 305 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| GS-PLG | | |
| Chain 3: | LAGSAPASSSTKKTQLQLEHLLLDLQMILNGINNYKNPKLTRMLTFKFYMPKK | 306 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDNHGGAELCDDDPPE | |
| GGG- | IPHATFKAMAYKEGTMLNCECKRGFRRIKSGSLYMLCTGNSSHSSWDNQCQCT | |
| SGRSDNH- | SSATRNTTKQVTPQPEEQKERKTTEMQSPMQPVDQASLPGHCREPPPWENEAT | |
| GGA-R | ERIYHFVVGQMVYYQCVQGYRALHRGPAESVCKMTHGKTRWTQPQLICTGE | |
| Chain 4: | LAGSAPASSSTKKTQLQLEHLLLDLQMILNGINNYKNPKLTRMLTFKFYMPKK | 307 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDN | |
| GGG- | | |
| SGRSDN | | |
| Chain 5: | HGGAELCDDDPPEIPHATFKAMAYKEGTMLNCECKRGFRRIKSGSLYMLCTGN | 308 |
| H-GGA-R | SSHSSWDNQCQCTSSATRNTTKQVTPQPEEQKERKTTEMQSPMQPVDQASLPG | |
| | HCREPPPWENEATERIYHFVVGQMVYYQCVQGYRALHRGPAESVCKMTHGKTR | |
| | WTQPQLICTGE | |

P1834 Cleaved proteins (Partial uPA/Matriptase cleavage,
complete Legumain cleavage)

| | | |
|---|---|---|
| Chain 1: | EPKSSDKTHTCPPCPAPEAAGGPSVFLFPPKPKDTLMISRTPEVTCVVVDVSH | 309 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDN | HLRPRDLISNINVIVLELKGSETTFMCEYADETATIVEFLNRWITFSQSTIST | |
| | LTGGGSGRSDN | |
| Chain 2: | EPKSSDKTHTCPPCPAPEAAGGPSVFLFPPKPKDTLMISRTPEVTCVVVDVSH | 310 |
| Fc(G1_AAA_C217S)_delK- | EDPEVKFNWYVDGVEVHNAKTKPREEQYNSTYRVVSVLTVLHQDWLNGKEYKC | |
| GS-PLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 311 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDN | |
| GGG- | | |
| SGRSDN | | |
| Chains 4 | HGGAELCDDDPPEIPHATFKAMAYKEGTMLNCECKRGFRRIKSGSLYMLCTGN | 312 |
| and 5: | SSHSSWDNQCQCTSSATRNTTKQVTPQPEEQKERKTTEMQSPMQPVDQASLPG | |
| H-GGA-R | HCREPPPWENEATERIYHFVVGQMVYYQCVQGYRALHRGPAESVCKMTHGKTR | |
| | WTQPQLICTGE | |

P1834 Cleaved proteins (Partial uPA/Matriptase cleavage,
partial Legumain cleavage)

| | | |
|---|---|---|
| Chain 1: | EPKSSDKTHTCPPCPAPEAAGGPSVFLFPPKPKDTLMISRTPEVTCVVVDVSH | 313 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDNH- | HLRPRDLISNINVIVLELKGSETTFMCEYADETATIVEFLNRWITFSQSTIST | |
| GGA-R | LTGGGSGRSDNHGGAELCDDDPPEIPHATFKAMAYKEGTMLNCECKRGFRRIK | |
| | SGSLYMLCTGNSSHSSWDNQCQCTSSATRNTTKQVTPQPEEQKERKTTEMQSP | |
| | MQPVDQASLPGHCREPPPWENEATERIYHFVVGQMVYYQCVQGYRALHRGPAE | |
| | SVCKMTHGKTRWTQPQLICTGE | |
| Chain 2: | EPKSSDKTHTCPPCPAPEAAGGPSVFLFPPKPKDTLMISRTPEVTCVVVDVSH | 314 |
| Fc(G1_AAA_C217S)_delK- | EDPEVKFNWYVDGVEVHNAKTKPREEQYNSTYRVVSVLTVLHQDWLNGKEYKC | |
| GS-PLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGSPLG | |
| Chain 3: | LAGSAPASSSTKKTQLQLEHLLLDLQMILNGINNYKNPKLTRMLTFKFYMPKK | 315 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDNHGGAELCDDDPPE | |
| GGG- | IPHATFKAMAYKEGTMLNCECKRGFRRIKSGSLYMLCTGNSSHSSWDNQCQCT | |
| SGRSDNH- | SSATRNTTKQVTPQPEEQKERKTTEMQSPMQPVDQASLPGHCREPPPWENEAT | |
| GGA-R | ERIYHFVVGQMVYYQCVQGYRALHRGPAESVCKMTHGKTRWTQPQLICTGE | |
| Chain 4: | EPKSSDKTHTCPPCPAPEAAGGPSVFLFPPKPKDTLMISRTPEVTCVVVDVSH | 316 |
| Fc(G1_AAA_C217S)_delK- | EDPEVKFNWYVDGVEVHNAKTKPREEQYNSTYRVVSVLTVLHQDWLNGKEYKC | |
| GS- | KVSNKALAAPIEKTISKAKGQPREPQVYTLPPSRDELTKNQVSLTCLVKGFYP | |
| PLGLAG- | SDIAVEWESNGQPENNYKTTPPVLDSDGSFFLYSKLTVDKSRWQQGNVFSCSV | |
| S-L_T3A- | MHEALHNHYTQKSLSLSPGGSPLGLAGSAPASSSTKKTQLQLEHLLLDLQMIL | |
| GGG- | NGINNYKNPKLTRMLTFKFYMPKKATELKHLQCLEEELKPLEEALNLAPSKNF | |
| SGRSDN | HLRPRDLISNINVIVLELKGSETTFMCEYADETATIVEFLNRWITFSQSTIST | |
| | LTGGGSGRSDN | |
| Chain 5: | HGGAELCDDDPPEIPHATFKAMAYKEGTMLNCECKRGFRRIKSGSLYMLCTGN | 317 |
| H-GGA-R | SSHSSWDNQCQCTSSATRNTTKQVTPQPEEQKERKTTEMQSPMQPVDQASLPG | |
| | HCREPPPWENEATERIYHFVVGQMVYYQCVQGYRALHRGPAESVCKMTHGKTR | |
| | WTQPQLICTGE | |
| Chain 6: | LAGSAPASSSTKKTQLQLEHLLLDLQMILNGINNYKNPKLTRMLTFKFYMPKK | 318 |
| LAG-S- | ATELKHLQCLEEELKPLEEALNLAPSKNFHLRPRDLISNINVIVLELKGSETT | |
| L_T3A- | FMCEYADETATIVEFLNRWITFSQSTISTLTGGGSGRSDN | |
| GGG- | | |
| SGRSDN | | |

P1835 Activatable Proprotein

| | | |
|---|---|---|
| Chains 1 | EPKSSDKTHTCPPCPAPEAAGGPSVFLFPPKPKDTLMISRTPEVTCVVVDVSH | 319 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGG- | TLTGGGSGRSDNHGGAELCDDDPPEIPHATFKAMAYKEGTMLNCECKRGFRRI | |
| SGRSDNH- | KSGSLYMLCTGNSSHSSWDNQCQCTSSATRNTTKQVTPQPEEQKERKTTEMQS | |
| GGA-R | PMQPVDQASLPGHCREPPPWENEATERIYHFVVGQMVYYQCVQGYRALHRGPA | |
| 10/13 | ESVCKMTHGKTRWTQPQLICTGE | |

P1836 Activatable Proprotein

| | | |
|---|---|---|
| Chains 1 | EPKSSDKTHTCPPCPAPEAAGGPSVFLFPPKPKDTLMISRTPEVTCVVVDVSH | 320 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGS- | SDIAVEWESNGQPENNYKTTPPVLDSDGSFFLYSKLTVDKSRWQQGNVFSCSV | |
| PLGLAG- | MHEALHNHYTQKSLSLSPGGGSPLGLAGGSAPASSSTKKTQLQLEHLLLDLQM | |
| GS- | ILNGINNYKNPKLTRMLTFKFYMPKKATELKHLQCLEEELKPLEEALNLAPSK | |
| L_T3A- | NFHLRPRDLISNINVIVLELKGSETTFMCEYADETATIVEFLNRWITFSQSTI | |
| GGG- | STLTGGGSGRSDNHGGAELCDDDPPEIPHATFKAMAYKEGTMLNCECKRGFRR | |
| SGRSDNH- | IKSGSLYMLCTGNSSHSSWDNQCQCTSSATRNTTKQVTPQPEEQKERKTTEMQ | |
| GGA-R | SPMQPVDQASLPGHCREPPPWENEATERIYHFVVGQMVYYQCVQGYRALHRGP | |
| 11/13 | AESVCKMTHGKTRWTQPQLICTGE | |

P1837 Activatable Proprotein

| | | |
|---|---|---|
| Chains 1 | EPKSSDKTHTCPPCPAPEAAGGPSVFLFPPKPKDTLMISRTPEVTCVVVDVSH | 321 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGS- | SDIAVEWESNGQPENNYKTTPPVLDSDGSFFLYSKLTVDKSRWQQGNVFSCSV | |
| PLGLAG- | MHEALHNHYTQKSLSLSPGGGSPLGLAGGGSAPASSSTKKTQLQLEHLLLDLQ | |
| GGS- | MILNGINNYKNPKLTRMLTFKFYMPKKATELKHLQCLEEELKPLEEALNLAPS | |
| L_T3A- | KNFHLRPRDLISNINVIVLELKGSETTFMCEYADETATIVEFLNRWITFSQST | |
| GGG- | ISTLTGGGSGRSDNHGGAELCDDDPPEIPHATFKAMAYKEGTMLNCECKRGFR | |
| SGRSDNH- | RIKSGSLYMLCTGNSSHSSWDNQCQCTSSATRNTTKQVTPQPEEQKERKTTEM | |
| GGA-R | QSPMQPVDQASLPGHCREPPPWENEATERIYHFVVGQMVYYQCVQGYRALHRG | |
| 12/13 | PAESVCKMTHGKTRWTQPQLICTGE | |

P1838 Activatable Proprotein

| | | |
|---|---|---|
| Chains 1 | EPKSSDKTHTCPPCPAPEAAGGPSVFLFPPKPKDTLMISRTPEVTCVVVDVSH | 322 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| PLGLAG- | MHEALHNHYTQKSLSLSPGGGGSPLGLAGGGSAPASSSTKKTQLQLEHLLLDL | |
| GGS- | QMILNGINNYKNPKLTRMLTFKFYMPKKATELKHLQCLEEELKPLEEALNLAP | |
| L_T3A- | SKNFHLRPRDLISNINVIVLELKGSETTFMCEYADETATIVEFLNRWITFSQS | |
| GGG- | TISTLTGGGSGRSDNHGGAELCDDDPPEIPHATFKAMAYKEGTMLNCECKRGF | |
| SGRSDNH- | RRIKSGSLYMLCTGNSSHSSWDNQCQCTSSATRNTTKQVTPQPEEQKERKTTE | |
| GGA-R | MQSPMQPVDQASLPGHCREPPPWENEATERIYHFVVGQMVYYQCVQGYRALHR | |
| 13/13 | GPAESVCKMTHGKTRWTQPQLICTGE | |

P1839 Activatable Proprotein

| | | |
|---|---|---|
| Chains 1 | EPKSSDKTHTCPPCPAPEAAGGPSVFLFPPKPKDTLMISRTPEVTCVVVDVSH | 323 |
| and 2: | EDPEVKFNWYVDGVEVHNAKTKPREEQYNSTYRVVSVLTVLHQDWLNGKEYKC | |
| Fc(G1_AA | KVSNKALAAPIEKTISKAKGQPREPQVYTLPPSRDELTKNQVSLTCLVKGFYP | |
| A_C217S) | SDIAVEWESNGQPENNYKTTPPVLDSDGSFFLYSKLTVDKSRWQQGNVFSCSV | |
| _delK- | MHEALHNHYTQKSLSLSPGGGGSPLGLAGGGGSAPASSSTKKTQLQLEHLLLD | |
| GGGS- | LQMILNGINNYKNPKLTRMLTFKFYMPKKATELKHLQCLEEELKPLEEALNLA | |
| PLGLAG- | PSKNFHLRPRDLISNINVIVLELKGSETTFMCEYADETATIVEFLNRWITFSQ | |
| GGGS- | STISTLTGGGSGRSDNHGGAELCDDDPPEIPHATFKAMAYKEGTMLNCECKRG | |
| L_T3A- | FRRIKSGSLYMLCTGNSSHSSWDNQCQCTSSATRNTTKQVTPQPEEQKERKTT | |
| GGG- | EMQSPMQPVDQASLPGHCREPPPWENEATERIYHFVVGQMVYYQCVQGYRALH | |
| SGRSDNH- | RGPAESVCKMTHGKTRWTQPQLICTGE | |
| GGA-R | | |
| 14/13 | | |

P1840 Activatable Proprotein

| | | |
|---|---|---|
| Chains 1 | EPKSSDKTHTCPPCPAPEAAGGPSVFLFPPKPKDTLMISRTPEVTCVVVDVSH | 324 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGGG- | TLTGGGGSGRSDNHGGAELCDDDPPEIPHATFKAMAYKEGTMLNCECKRGFRR | |
| SGRSDNH- | IKSGSLYMLCTGNSSHSSWDNQCQCTSSATRNTTKQVTPQPEEQKERKTTEMQ | |
| GGA-R | SPMQPVDQASLPGHCREPPPWENEATERIYHFVVGQMVYYQCVQGYRALHRGP | |
| 10/14 | AESVCKMTHGKTRWTQPQLICTGE | |

P1841 Activatable Proprotein

| | | |
|---|---|---|
| Chains 1 | EPKSSDKTHTCPPCPAPEAAGGPSVFLFPPKPKDTLMISRTPEVTCVVVDVSH | 325 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGGG- | TLTGGGGSGRSDNHGGGAELCDDDPPEIPHATFKAMAYKEGTMLNCECKRGFR | |
| SGRSDNH- | RIKSGSLYMLCTGNSSHSSWDNQCQCTSSATRNTTKQVTPQPEEQKERKTTEM | |
| GGGA-R | QSPMQPVDQASLPGHCREPPPWENEATERIYHFVVGQMVYYQCVQGYRALHRG | |
| 10/15 | PAESVCKMTHGKTRWTQPQLICTGE | |

P1842 Activatable Proprotein

| | | |
|---|---|---|
| Chains 1 | EPKSSDKTHTCPPCPAPEAAGGPSVFLFPPKPKDTLMISRTPEVTCVVVDVSH | 326 |
| and 2: | EDPEVKFNWYVDGVEVHNAKTKPREEQYNSTYRVVSVLTVLHQDWLNGKEYKC | |
| Fc(G1_AA | KVSNKALAAPIEKTISKAKGQPREPQVYTLPPSRDELTKNQVSLTCLVKGFYP | |
| A_C217S) | SDIAVEWESNGQPENNYKTTPPVLDSDGSFFLYSKLTVDKSRWQQGNVFSCSV | |
| _delK- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| PLGLAG- | FHLRPRDLISNINVIVLELKGSETTFMCEYADETATIVEFLNRWITFSQSTIS | |
| GS- | TLTGGGGSSGRSDNHGGGAELCDDDPPEIPHATFKAMAYKEGTMLNCECKRGF | |
| L_T3A- | RRIKSGSLYMLCTGNSSHSSWDNQCQCTSSATRNTTKQVTPQPEEQKERKTTE | |
| GGGGS- | MQSPMQPVDQASLPGHCREPPPWENEATERIYHFVVGQMVYYQCVQGYRALHR | |
| SGRSDNH- | GPAESVCKMTHGKTRWTQPQLICTGE | |
| GGGA-R | | |
| 10/16 | | |

P1843 Activatable Proprotein

| | | |
|---|---|---|
| Chains 1 | EPKSSDKTHTCPPCPAPEAAGGPSVFLFPPKPKDTLMISRTPEVTCVVVDVSH | 327 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGGGS- | TLTGGGGSSGRSDNHGGGGAELCDDDPPEIPHATFKAMAYKEGTMLNCECKRG | |
| SGRSDNH- | FRRIKSGSLYMLCTGNSSHSSWDNQCQCTSSATRNTTKQVTPQPEEQKERKTT | |
| GGGGA-R | EMQSPMQPVDQASLPGHCREPPPWENEATERIYHFVVGQMVYYQCVQGYRALH | |
| 10/17 | RGPAESVCKMTHGKTRWTQPQLICTGE | |

P1844 Activatable Proprotein

| | | |
|---|---|---|
| Chains 1 | EPKSSDKTHTCPPCPAPEAAGGPSVFLFPPKPKDTLMISRTPEVTCVVVDVSH | 328 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGSGGS- | TLTGGSGGSSGRSDNHGGGGAELCDDDPPEIPHATFKAMAYKEGTMLNCECKR | |
| SGRSDNH- | GFRRIKSGSLYMLCTGNSSHSSWDNQCQCTSSATRNTTKQVTPQPEEQKERKT | |
| GGGGA-R | TEMQSPMQPVDQASLPGHCREPPPWENEATERIYHFVVGQMVYYQCVQGYRAL | |
| 10/18 | HRGPAESVCKMTHGKTRWTQPQLICTGE | |

P1845 Activatable Proprotein

| | | |
|---|---|---|
| Chains 1 | EPKSSDKTHTCPPCPAPEAAGGPSVFLFPPKPKDTLMISRTPEVTCVVVDVSH | 329 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGSGGS- | TLTGGSGGSSGRSDNHGGSGGAELCDDDPPEIPHATFKAMAYKEGTMLNCECK | |
| SGRSDNH- | RGFRRIKSGSLYMLCTGNSSHSSWDNQCQCTSSATRNTTKQVTPQPEEQKERK | |
| GGSGGA-R | TTEMQSPMQPVDQASLPGHCREPPPWENEATERIYHFVVGQMVYYQCVQGYRA | |
| 10/19 | LHRGPAESVCKMTHGKTRWTQPQLICTGE | |

P1846 Activatable Proprotein

| | | |
|---|---|---|
| Chains 1 | EPKSSDKTHTCPPCPAPEAAGGPSVFLFPPKPKDTLMISRTPEVTCVVVDVSH | 330 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEALNLAPSKN | |
| L_T3A- | FHLRPRDLISNINVIVLELKGSETTFMCEYADETATIVEFLNRWITFSQSTIS | |
| GGSGGS- | TLTGGSGGSSGRSDNHGGSGGAELCDDDPPEIPHATFKAMAYKEGTMLNCECK | |
| SGRSDNH- | RGFRRIKSGSLYMLCTGASSHSSWDNQCQCTSSATRATTKQVTPQPEEQKERK | |
| GGSGGA- | TTEMQSPMQPVDQASLPGHCREPPPWENEATERIYHFVVGQMVYYQCVQGYRA | |
| R_N49A_N68A | LHRGPAESVCKMTHGKTRWTQPQLICTGE | |
| 10/19 | | |

P1847 Activatable Proprotein

| | | |
|---|---|---|
| Chains 1 | EPKSSDKTHTCPPCPAPEAAGGPSVFLFPPKPKDTLMISRTPEVTCVVVDVSH | 331 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GS- | SDIAVEWESNGQPENNYKTTPPVLDSDGSFFLYSKLTVDKSRWQQGNVFSCSV | |
| PLGLAG- | MHEALHNHYTQKSLSLSPGGSPLGLAGGSAPASSSTKKTQLQLEHLLLDLQMI | |
| GS- | LNGINNYKNPKLTRMLTFKFYMPKKATELKHLQCLEEELKPLEEVLNLAHSKN | |
| IL2_D10_T3A- | FHFDPRDVVSNINVFVLELKGSETTFMCEYADETATIVEFLNRWITFSQSIIS | |
| GGSGGS- | TLTGGSGGSSGRSDNHGGSGGAELCDDDPPEIPHATFKAMAYKEGTMLNCECK | |
| SGRSDNH- | RGFRRIKSGSLYMLCTGNSSHSSWDNQCQCTSSATRNTTKQVTPQPEEQKERK | |
| GGSGGA-R | TTEMQSPMQPVDQASLPGHCREPPPWENEATERIYHFVVGQMVYYQCVQGYRA | |
| 10/18 | LHRGPAESVCKMTHGKTRWTQPQLICTGE | |

P1848 Activatable Proprotein

| | | |
|---|---|---|
| Chains 1 | EPKSSDKTHTCPPCPAPEAAGGPSVFLFPPKPKDTLMISRTPEVTCVVVDVSH | 332 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGS- | SDIAVEWESNGQPENNYKTTPPVLDSDGSFFLYSKLTVDKSRWQQGNVFSCSV | |
| PLGMLS- | MHEALHNHYTQKSLSLSPGGGSPLGMLSGGSAPASSSTKKTQLQLEHLLLDLQ | |
| GGS- | MILNGINNYKNPKLTRMLTFKFYMPKKATELKHLQCLEEELKPLEEALNLAPS | |
| L_T3A- | KNFHLRPRDLISNINVIVLELKGSETTFMCEYADETATIVEFLNRWITFSQST | |
| GGGGS- | ISTLTGGGGSSGRSDNHGGGAELCDDDPPEIPHATFKAMAYKEGTMLNCECKR | |
| SGRSDNH- | GFRRIKSGSLYMLCTGNSSHSSWDNQCQCTSSATRNTTKQVTPQPEEQKERKT | |
| GGGA-R | TEMQSPMQPVDQASLPGHCREPPPWENEATERIYHFVVGQMVYYQCVQGYRAL | |
| 12/16 | HRGPAESVCKMTHGKTRWTQPQLICTGE | |

P1849 Activatable Proprotein

| | | |
|---|---|---|
| Chains 1 | EPKSSDKTHTCPPCPAPEAAGGPSVFLFPPKPKDTLMISRTPEVTCVVVDVSH | 333 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGS- | SDIAVEWESNGQPENNYKTTPPVLDSDGSFFLYSKLTVDKSRWQQGNVFSCSV | |
| PLGIAG- | MHEALHNHYTQKSLSLSPGGGSPLGIAGGGSAPASSSTKKTQLQLEHLLLDLQ | |
| GGS- | MILNGINNYKNPKLTRMLTFKFYMPKKATELKHLQCLEEELKPLEEALNLAPS | |
| L_T3A- | KNFHLRPRDLISNINVIVLELKGSETTFMCEYADETATIVEFLNRWITFSQST | |
| GGGGS- | ISTLTGGGGSSGRSDNHGGGAELCDDDPPEIPHATFKAMAYKEGTMLNCECKR | |
| SGRSDNH- | GFRRIKSGSLYMLCTGNSSHSSWDNQCQCTSSATRNTTKQVTPQPEEQKERKT | |
| GGGA-R | TEMQSPMQPVDQASLPGHCREPPPWENEATERIYHFVVGQMVYYQCVQGYRAL | |
| 12/16 | HRGPAESVCKMTHGKTRWTQPQLICTGE | |

P1850 Activatable Proprotein

| | | |
|---|---|---|
| Chains 1 | EPKSSDKTHTCPPCPAPEAAGGPSVFLFPPKPKDTLMISRTPEVTCVVVDVSH | 334 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGS- | SDIAVEWESNGQPENNYKTTPPVLDSDGSFFLYSKLTVDKSRWQQGNVFSCSV | |
| PVGLLA- | MHEALHNHYTQKSLSLSPGGGSPVGLLAGGSAPASSSTKKTQLQLEHLLLDLQ | |
| GGS- | MILNGINNYKNPKLTRMLTFKFYMPKKATELKHLQCLEEELKPLEEALNLAPS | |
| L_T3A- | KNFHLRPRDLISNINVIVLELKGSETTFMCEYADETATIVEFLNRWITFSQST | |
| GGGGS- | ISTLTGGGGSSGRSDNHGGGAELCDDDPPEIPHATFKAMAYKEGTMLNCECKR | |
| SGRSDNH- | GFRRIKSGSLYMLCTGNSSHSSWDNQCQCTSSATRNTTKQVTPQPEEQKERKT | |
| GGGA-R | TEMQSPMQPVDQASLPGHCREPPPWENEATERIYHFVVGQMVYYQCVQGYRAL | |
| 12/16 | HRGPAESVCKMTHGKTRWTQPQLICTGE | |

P1946 Activatable Proprotein

| | | |
|---|---|---|
| Chains 1 | EPKSSDKTHTCPPCPAPELLGGPSVFLFPPKPKDTLMISRTPEVTCVVVDVSH | 335 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_wt_T3A- | MHEALHNHYTQKSLSLSPGKGGGGSGGGGSAPASSSTKKTQLQLEHLLLDLQM | |
| GSGSENLY | ILNGINNYKNPKLTRMLTFKFYMPKKATELKHLQCLEEELKPLEEVLNLAQSK | |
| FQGGSGS- | NFHLRPRDLISNINVIVLELKGSETTFMCEYADETATIVEFLNRWITFSQSII | |
| R | STLTGSGSENLYFQGGSGSELCDDDPPEIPHATFKAMAYKEGTMLNCECKRGF | |
| | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1946 Cleaved proteins (Complete TEV cleavage)

| | | |
|---|---|---|
| Chains 1 | EPKSSDKTHTCPPCPAPELLGGPSVFLFPPKPKDTLMISRTPEVTCVVVDVSH | 336 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_wt_T3A- | MHEALHNHYTQKSLSLSPGKGGGGSGGGGSAPASSSTKKTQLQLEHLLLDLQM | |
| GSGSENLY | ILNGINNYKNPKLTRMLTFKFYMPKKATELKHLQCLEEELKPLEEVLNLAQSK | |
| FQ | NFHLRPRDLISNINVIVLELKGSETTFMCEYADETATIVEFLNRWITFSQSII | |
| | STLTGSGSENLYFQ | |
| Chains 3 | GGSGSELCDDDPPEIPHATFKAMAYKEGTMLNCECKRGFRRIKSGSLYMLCTG | 337 |
| and 4: | NSSHSSWDNQCQCTSSATRNTTKQVTPQPEEQKERKTTEMQSPMQPVDQASLP | |
| GGSGS-R | GHCREPPPWENEATERIYHFVVGQMVYYQCVQGYRALHRGPAESVCKMTHGKT | |
| | RWTQPQLICTGE | |

P1946 Cleaved proteins (Partial TEV cleavage)

| | | |
|---|---|---|
| Chain 1: | EPKSSDKTHTCPPCPAPELLGGPSVFLFPPKPKDTLMISRTPEVTCVVVDVSH | 338 |
| Fc- | EDPEVKFNWYVDGVEVHNAKTKPREEQYNSTYRVVSVLTVLHQDWLNGKEYKC | |
| stableLinker- | KVSNKALPAPIEKTISKAKGQPREPQVYTLPPSREEMTKNQVSLTCLVKGFYP | |
| L_wt_T3A- | SDIAVEWESNGQPENNYKTTPPVLDSDGSFFLYSKLTVDKSRWQQGNVFSCSV | |
| GSGSENLY | MHEALHNHYTQKSLSLSPGKGGGGSGGGGSAPASSSTKKTQLQLEHLLLDLQM | |
| FQGGSGS- | ILNGINNYKNPKLTRMLTFKFYMPKKATELKHLQCLEEELKPLEEVLNLAQSK | |
| R | NFHLRPRDLISNINVIVLELKGSETTFMCEYADETATIVEFLNRWITFSQSII | |
| | STLTGSGSENLYFQGGSGSELCDDDPPEIPHATFKAMAYKEGTMLNCECKRGF | |
| | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |
| Chain 2: | EPKSSDKTHTCPPCPAPELLGGPSVFLFPPKPKDTLMISRTPEVTCVVVDVSH | 339 |
| Fc- | EDPEVKFNWYVDGVEVHNAKTKPREEQYNSTYRVVSVLTVLHQDWLNGKEYKC | |
| stableLinker- | KVSNKALPAPIEKTISKAKGQPREPQVYTLPPSREEMTKNQVSLTCLVKGFYP | |
| L_wt_T3A- | SDIAVEWESNGQPENNYKTTPPVLDSDGSFFLYSKLTVDKSRWQQGNVFSCSV | |
| GSGSENLY | MHEALHNHYTQKSLSLSPGKGGGGSGGGGSAPASSSTKKTQLQLEHLLLDLQM | |
| FQ | ILNGINNYKNPKLTRMLTFKFYMPKKATELKHLQCLEEELKPLEEVLNLAQSK | |
| | NFHLRPRDLISNINVIVLELKGSETTFMCEYADETATIVEFLNRWITFSQSII | |
| | STLTGSGSENLYFQ | |
| Chain 3: | GGSGSELCDDDPPEIPHATFKAMAYKEGTMLNCECKRGFRRIKSGSLYMLCTG | 340 |
| GGSGS-R | NSSHSSWDNQCQCTSSATRNTTKQVTPQPEEQKERKTTEMQSPMQPVDQASLP | |
| | GHCREPPPWENEATERIYHFVVGQMVYYQCVQGYRALHRGPAESVCKMTHGKT | |
| | RWTQPQLICTGE | |

P1947 Activatable Proprotein

| | | |
|---|---|---|
| Chains 1 | EPKSSDKTHTCPPCPAPELLGGPSVFLFPPKPKDTLMISRTPEVTCVVVDVSH | 341 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_F42A_T3A- | MHEALHNHYTQKSLSLSPGKGGGGSGGGGSAPASSSTKKTQLQLEHLLLDLQM | |
| GSGSTEVG | ILNGINNYKNPKLTRMLTAKFYMPKKATELKHLQCLEEELKPLEEVLNLAQSK | |
| SGS-R | NFHLRPRDLISNINVIVLELKGSETTFMCEYADETATIVEFLNRWITFSQSII | |
| | STLTGSGSENLYFQGGSGSELCDDDPPEIPHATFKAMAYKEGTMLNCECKRGF | |
| | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1948 Activatable Proprotein

| | | |
|---|---|---|
| Chains 1 | EPKSSDKTHTCPPCPAPELLGGPSVFLFPPKPKDTLMISRTPEVTCVVVDVSH | 342 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_Y45A_T3A- | MHEALHNHYTQKSLSLSPGKGGGGSGGGGSAPASSSTKKTQLQLEHLLLDLQM | |
| GSGSTEVG | ILNGINNYKNPKLTRMLTFKFAMPKKATELKHLQCLEEELKPLEEVLNLAQSK | |
| SGS-R | NFHLRPRDLISNINVIVLELKGSETTFMCEYADETATIVEFLNRWITFSQSII | |
| | STLTGSGSENLYFQGGSGSELCDDDPPEIPHATFKAMAYKEGTMLNCECKRGF | |
| | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1949 Activatable Proprotein

| | | |
|---|---|---|
| Chains 1 | EPKSSDKTHTCPPCPAPELLGGPSVFLFPPKPKDTLMISRTPEVTCVVVDVSH | 343 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_F42A_Y45A_T3A- | MHEALHNHYTQKSLSLSPGKGGGGSGGGGSAPASSSTKKTQLQLEHLLLDLQM | |
| GSGSTEVG | ILNGINNYKNPKLTRMLTAKFAMPKKATELKHLQCLEEELKPLEEVLNLAQSK | |
| SGS-R | NFHLRPRDLISNINVIVLELKGSETTFMCEYADETATIVEFLNRWITFSQSII | |
| | STLTGSGSENLYFQGGSGSELCDDDPPEIPHATFKAMAYKEGTMLNCECKRGF | |
| | RRIKSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P14501950 Activatable Proprotein

| | | |
|---|---|---|
| Chains 1 | DIVMTQTPLSLSVTPGQPASISCRASKSVSTSAYSYMHWYLQKPGQSPQLLIY | 344 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 345 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKY | |
| GGSPLGLA | EPKSSDKTHTCPPCPAPEAAGGPSVFLFPPKPKDTLMISRTPEVTCVVVDVSH | |
| GGGS- | EDPEVKFNWYVDGVEVHNAKTKPREEQYNSTYRVVSVLTVLHQDWLNGKEYKC | |
| L_T3A- | KVSNKALAAPIEKTISKAKGQPREPQVYTLPPSRDELTKNQVSLTCLVKGFYP | |
| GGSGGGSG | SDIAVEWESNGQPENNYKTTPPVLDSDGSFFLYSKLTVDKSRWQQGNVFSCSV | |
| GGSGGGA- | MHEALHNHYTQKSLSLSPGGGSPLGLAGGGSAPASSSTKKTQLQLEHLLLDLQ | |
| R 12/15 | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGGSGGGSGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P14501950 Cleaved proteins (Complete MMP-2/MMP-9 cleavage)

| | | |
|---|---|---|
| Chains 1 | DIVMTQTPLSLSVTPGQPASISCRASKSVSTSAYSYMHWYLQKPGQSPQLLIY | 346 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 347 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKYEPKSSDKTHTCPPCPAPEAAGGPSVFLFPPKPKDTLMISRTPE | |
| GGSPLG | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGGGSPLG | |
| Chains 5 | LAGGGSAPASSSTKKTQLQLEHLLLDLQMILNGINNYKNPKLTRMLTFKFYMP | 348 |
| and 6: | KKATELKHLQCLEEELKPLEEALNLAPSKNFHLRPRDLISNINVIVLELKGSE | |
| LAGGGS- | TTFMCEYADETATIVEFLNRWITFSQSTISTLTGGSGGGSGGGSGGGAELCDD | |
| L_T3A- | DPPEIPHATFKAMAYKEGTMLNCECKRGFRRIKSGSLYMLCTGNSSHSSWDNQ | |
| GGSGGGSG | CQCTSSATRNTTKQVTPQPEEQKERKTTEMQSPMQPVDQASLPGHCREPPPWE | |
| GGSGGGA- | NEATERIYHFVVGQMVYYQCVQGYRALHRGPAESVCKMTHGKTRWTQPQLICT | |
| R | GE | |

P14501950 Cleaved proteins (Partial MMP-2/MMP-9 cleavage)

| | | |
|---|---|---|
| Chains 1 | DIVMTQTPLSLSVTPGQPASISCRASKSVSTSAYSYMHWYLQKPGQSPQLLIY | 349 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 350 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSPLGLA | KVDKRVESKYEPKSSDKTHTCPPCPAPEAAGGPSVFLFPPKPKDTLMISRTPE | |
| GGGS- | VTCVVVDVSHEDPEVKFNWYVDGVEVHNAKTKPREEQYNSTYRVVSVLTVLHQ | |
| L_T3A- | DWLNGKEYKCKVSNKALAAPIEKTISKAKGQPREPQVYTLPPSRDELTKNQVS | |
| GGSGGGSG | LTCLVKGFYPSDIAVEWESNGQPENNYKTTPPVLDSDGSFFLYSKLTVDKSRW | |
| GGSGGGA- | QQGNVFSCSVMHEALHNHYTQKSLSLSPGGGSPLGLAGGGSAPASSSTKKTQL | |
| R | QLEHLLLDLQMILNGINNYKNPKLTRMLTFKFYMPKKATELKHLQCLEEELKP | |
| | LEEALNLAPSKNFHLRPRDLISNINVIVLELKGSETTFMCEYADETATIVEFL | |
| | NRWITFSQSTISTLTGGSGGGSGGGSGGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |
| Chain 4: | EVQLVQSGAEVKKPGESLKISCKGSGYTFTENIIHWVRQMPGKGLEWIGWFHP | 351 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSPLG | KVDKRVESKY | |
| | EPKSSDKTHTCPPCPAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKC | |
| | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSPLG | |
| Chain 5: | LAGGGSAPASSSTKKTQLQLEHLLLDLQMILNGINNYKNPKLTRMLTFKFYMP | 352 |
| LAGGGS- | KKATELKHLQCLEEELKPLEEALNLAPSKNFHLRPRDLISNINVIVLELKGSE | |
| L_T3A- | TTFMCEYADETATIVEFLNRWITFSQSTISTLTGGSGGGSGGGSGGGAELCDD | |
| GGSGGGSG | DPPEIPHATFKAMAYKEGTMLNCECKRGFRRIKSGSLYMLCTGNSSHSSWDNQ | |
| GGSGGGA- | CQCTSSATRNTTKQVTPQPEEQKERKTTEMQSPMQPVDQASLPGHCREPPPWE | |
| R | NEATERIYHFVVGQMVYYQCVQGYRALHRGPAESVCKMTHGKTRWTQPQLICT | |
| | GE | |

P14501951 Activatable Proprotein

| | | |
|---|---|---|
| Chains 1 | DIVMTQTPLSLSVTPGQPASISCRASKSVSTSAYSYMHWYLQKPGQSPQLLIY | 353 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 354 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKY | |
| GGSGRSDN | EPKSSDKTHTCPPCPAPEAAGGPSVFLFPPKPKDTLMISRTPEVTCVVVDVSH | |
| HGGA- | EDPEVKFNWYVDGVEVHNAKTKPREEQYNSTYRVVSVLTVLHQDWLNGKEYKC | |
| L_T3A- | KVSNKALAAPIEKTISKAKGQPREPQVYTLPPSRDELTKNQVSLTCLVKGFYP | |
| GGSGGGSG | SDIAVEWESNGQPENNYKTTPPVLDSDGSFFLYSKLTVDKSRWQQGNVFSCSV | |
| GGSGGGA- | MHEALHNHYTQKSLSLSPGGGSGRSDNHGGAAPASSSTKKTQLQLEHLLLDLQ | |
| R 12/15 | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGGSGGGSGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P14501951 Cleaved proteins (Complete uPA/Matriptase cleavage)

| | | |
|---|---|---|
| Chains 1 | DIVMTQTPLSLSVTPGQPASISCRASKSVSTSAYSYMHWYLQKPGQSPQLLIY | 355 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 356 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKYEPKSSDKTHTCPPCPAPEAAGGPSVFLFPPKPKDTLMISRTPE | |
| GGSGR | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGGGSGR | |
| Chains 5 | SDNHGGAAPASSSTKKTQLQLEHLLLDLQMILNGINNYKNPKLTRMLTFKFYM | 357 |
| and 6: | PKKATELKHLQCLEEELKPLEEALNLAPSKNFHLRPRDLISNINVIVLELKGS | |
| SDNHGGA- | ETTFMCEYADETATIVEFLNRWITFSQSTISTLTGGSGGGSGGGSGGGAELCD | |
| L_T3A- | DDPPEIPHATFKAMAYKEGTMLNCECKRGFRRIKSGSLYMLCTGNSSHSSWDN | |
| GGSGGGSG | QCQCTSSATRNTTKQVTPQPEEQKERKTTEMQSPMQPVDQASLPGHCREPPPW | |
| GGSGGGA- | ENEATERIYHFVVGQMVYYQCVQGYRALHRGPAESVCKMTHGKTRWTQPQLIC | |
| R | TGE | |

P14501951 Cleaved proteins (Partial uPA/Matriptase cleavage)

| | | |
|---|---|---|
| Chains 1 | DIVMTQTPLSLSVTPGQPASISCRASKSVSTSAYSYMHWYLQKPGQSPQLLIY | 358 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 359 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGRSDN | KVDKRVESKYEPKSSDKTHTCPPCPAPEAAGGPSVFLFPPKPKDTLMISRTPE | |
| HGGA- | VTCVVVDVSHEDPEVKFNWYVDGVEVHNAKTKPREEQYNSTYRVVSVLTVLHQ | |
| L_T3A- | DWLNGKEYKCKVSNKALAAPIEKTISKAKGQPREPQVYTLPPSRDELTKNQVS | |
| GGSGGGSG | LTCLVKGFYPSDIAVEWESNGQPENNYKTTPPVLDSDGSFFLYSKLTVDKSRW | |
| GGSGGGA- | QQGNVFSCSVMHEALHNHYTQKSLSLSPGGGSGRSDNHGGAAPASSSTKKTQL | |
| R | QLEHLLLDLQMILNGINNYKNPKLTRMLTFKFYMPKKATELKHLQCLEEELKP | |
| | LEEALNLAPSKNFHLRPRDLISNINVIVLELKGSETTFMCEYADETATIVEFL | |
| | NRWITFSQSTISTLTGGSGGGSGGGSGGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |
| Chain 4: | EVQLVQSGAEVKKPGESLKISCKGSGYTFTENIIHWVRQMPGKGLEWIGWFHP | 360 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGR | KVDKRVESKY | |
| | EPKSSDKTHTCPPCPAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKC | |
| | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSGR | |
| Chain 5: | SDNHGGAAPASSSTKKTQLQLEHLLLDLQMILNGINNYKNPKLTRMLTFKFYM | 361 |
| SDNHGGA- | PKKATELKHLQCLEEELKPLEEALNLAPSKNFHLRPRDLISNINVIVLELKGS | |
| L_T3A- | ETTFMCEYADETATIVEFLNRWITFSQSTISTLTGGSGGGSGGGSGGGAELCD | |
| GGSGGGSG | DDPPEIPHATFKAMAYKEGTMLNCECKRGFRRIKSGSLYMLCTGNSSHSSWDN | |
| GGSGGGA- | QCQCTSSATRNTTKQVTPQPEEQKERKTTEMQSPMQPVDQASLPGHCREPPPW | |
| R | ENEATERIYHFVVGQMVYYQCVQGYRALHRGPAESVCKMTHGKTRWTQPQLIC | |
| | TGE | |

P14501951 Cleaved proteins (Complete Legumain cleavage)

| | | |
|---|---|---|
| Chains 1 | DIVMTQTPLSLSVTPGQPASISCRASKSVSTSAYSYMHWYLQKPGQSPQLLIY | 362 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 363 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKYEPKSSDKTHTCPPCPAPEAAGGPSVFLFPPKPKDTLMISRTPE | |
| GGSGRSDN | VTCVVVDVSHEDPEVKFNWYVDGVEVHNAKTKPREEQYNSTYRVVSVLTVLHQ | |
| | DWLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGGGSGRSDN | |
| Chains 5 | HGGAAPASSSTKKTQLQLEHLLLDLQMILNGINNYKNPKLTRMLTFKFYMPKK | 364 |
| and 6: | ATELKHLQCLEEELKPLEEALNLAPSKNFHLRPRDLISNINVIVLELKGSETT | |
| HGGA- | FMCEYADETATIVEFLNRWITFSQSTISTLTGGSGGGSGGGSGGGAELCDDDP | |
| L_T3A- | PEIPHATFKAMAYKEGTMLNCECKRGFRRIKSGSLYMLCTGNSSHSSWDNQCQ | |
| GGSGGGSG | CTSSATRNTTKQVTPQPEEQKERKTTEMQSPMQPVDQASLPGHCREPPPWENE | |
| GGSGGGA- | ATERIYHFVVGQMVYYQCVQGYRALHRGPAESVCKMTHGKTRWTQPQLICTGE | |
| R | | |

P14501951 Cleaved proteins (Partial Legumain cleavage)

| | | |
|---|---|---|
| Chains 1 | DIVMTQTPLSLSVTPGQPASISCRASKSVSTSAYSYMHWYLQKPGQSPQLLIY | 365 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 366 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGRSDN | KVDKRVESKY | |
| HGGA- | EPKSSDKTHTCPPCPAPEAAGGPSVFLFPPKPKDTLMISRTPEVTCVVVDVSH | |
| L_T3A- | EDPEVKFNWYVDGVEVHNAKTKPREEQYNSTYRVVSVLTVLHQDWLNGKEYKC | |
| GGSGGGSG | KVSNKALAAPIEKTISKAKGQPREPQVYTLPPSRDELTKNQVSLTCLVKGFYP | |
| GGSGGGA- | SDIAVEWESNGQPENNYKTTPPVLDSDGSFFLYSKLTVDKSRWQQGNVFSCSV | |
| R | MHEALHNHYTQKSLSLSPGGGSGRSDNHGGAAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGGSGGGSGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |
| Chain 4: | EVQLVQSGAEVKKPGESLKISCKGSGYTFTENIIHWVRQMPGKGLEWIGWFHP | 367 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGRSDN | KVDKRVESKY | |
| | EPKSSDKTHTCPPCPAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKC | |
| | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSGRSDN | |
| Chain 5: | HGGAAPASSSTKKTQLQLEHLLLDLQMILNGINNYKNPKLTRMLTFKFYMPKK | 368 |
| HGGA- | ATELKHLQCLEEELKPLEEALNLAPSKNFHLRPRDLISNINVIVLELKGSETT | |
| L_T3A- | FMCEYADETATIVEFLNRWITFSQSTISTLTGGSGGGSGGGSGGGAELCDDDP | |
| GGSGGGSG | PEIPHATFKAMAYKEGTMLNCECKRGFRRIKSGSLYMLCTGNSSHSSWDNQCQ | |
| GGSGGGA- | CTSSATRNTTKQVTPQPEEQKERKTTEMQSPMQPVDQASLPGHCREPPPWENE | |
| R | ATERIYHFVVGQMVYYQCVQGYRALHRGPAESVCKMTHGKTRWTQPQLICTGE | |

P14501952 Activatable Proprotein

| | | |
|---|---|---|
| Chains 1 | DIVMTQTPLSLSVTPGQPASISCRASKSVSTSAYSYMHWYLQKPGQSPQLLIY | 369 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 370 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AA | KVDKRVESKYEPKSSDKTHTCPPCPAPEAAGGPSVFLFPPKPKDTLMISRTPE | |
| A_C217S) | VTCVVVDVSHEDPEVKFNWYVDGVEVHNAKTKPREEQYNSTYRVVSVLTVLHQ | |
| _delK- | DWLNGKEYKCKVSNKALAAPIEKTISKAKGQPREPQVYTLPPSRDELTKNQVS | |
| GGSGGSGG | LTCLVKGFYPSDIAVEWESNGQPENNYKTTPPVLDSDGSFFLYSKLTVDKSRW | |
| SGGS- | QQGNVFSCSVMHEALHNHYTQKSLSLSPGGGSGGSGGSGGSAPASSSTKKTQL | |
| L_T3A- | QLEHLLLDLQMILNGINNYKNPKLTRMLTFKFYMPKKATELKHLQCLEEELKP | |
| GGSGGGPL | LEEALNLAPSKNFHLRPRDLISNINVIVLELKGSETTFMCEYADETATIVEFL | |
| GLAGGGA- | NRWITFSQSTISTLTGGSGGGPLGLAGGGAELCDDDPPEIPHATFKAMAYKEG | |
| R 12/15 | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |

P14501952 Cleaved proteins (Complete MMP-2/MMP-9 cleavage)

| | | |
|---|---|---|
| Chains 1 | DIVMTQTPLSLSVTPGQPASISCRASKSVSTSAYSYMHWYLQKPGQSPQLLIY | 371 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 372 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKY | |
| GGSGGSGG | EPKSSDKTHTCPPCPAPEAAGGPSVFLFPPKPKDTLMISRTPEVTCVVVDVSH | |
| SGGS- | EDPEVKFNWYVDGVEVHNAKTKPREEQYNSTYRVVSVLTVLHQDWLNGKEYKC | |
| L_T3A- | KVSNKALAAPIEKTISKAKGQPREPQVYTLPPSRDELTKNQVSLTCLVKGFYP | |
| GGSGGGPL | SDIAVEWESNGQPENNYKTTPPVLDSDGSFFLYSKLTVDKSRWQQGNVFSCSV | |
| G | MHEALHNHYTQKSLSLSPGGGSGGSGGSGGSAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGGPLG | |
| Chains 5 | LAGGGAELCDDDPPEIPHATFKAMAYKEGTMLNCECKRGFRRIKSGSLYMLCT | 373 |
| and 6: | GNSSHSSWDNQCQCTSSATRNTTKQVTPQPEEQKERKTTEMQSPMQPVDQASL | |
| LAGGGA-R | PGHCREPPPWENEATERIYHFVVGQMVYYQCVQGYRALHRGPAESVCKMTHGK | |
| | TRWTQPQLICTGE | |

P14501952 Cleaved proteins (Partial MMP-2/MMP-9 cleavage)

| | | |
|---|---|---|
| Chains 1 | DIVMTQTPLSLSVTPGQPASISCRASKSVSTSAYSYMHWYLQKPGQSPQLLIY | 374 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 375 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AA | SWNSGALTSGVHTFPAVLQSSGLYSLSSVVTVPSSSLGTKTYTCNVDHKPSNT | |
| A_C217S) | KVDKRVESKYEPKSSDKTHTCPPCPAPEAAGGPSVFLFPPKPKDTLMISRTPE | |
| del K- | VTCVVVDVSHEDPEVKFNWYVDGVEVHNAKTKPREEQYNSTYRVVSVLTVLHQ | |
| GGSGGSGG | DWLNGKEYKCKVSNKALAAPIEKTISKAKGQPREPQVYTLPPSRDELTKNQVS | |
| SGGS- | LTCLVKGFYPSDIAVEWESNGQPENNYKTTPPVLDSDGSFFLYSKLTVDKSRW | |
| L_T3A- | QQGNVFSCSVMHEALHNHYTQKSLSLSPGGGSGGSGGSGGSAPASSSTKKTQL | |
| GGSGGGPL | QLEHLLLDLQMILNGINNYKNPKLTRMLTFKFYMPKKATELKHLQCLEEELKP | |
| GLAGGGA- | LEEALNLAPSKNFHLRPRDLISNINVIVLELKGSETTFMCEYADETATIVEFL | |
| R | NRWITFSQSTISTLTGGSGGGPLGLAGGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |
| Chain 4: | EVQLVQSGAEVKKPGESLKISCKGSGYTFTENIIHWVRQMPGKGLEWIGWFHP | 376 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGGSGG | KVDKRVESKY | |
| SGGS- | EPKSSDKTHTCPPCPAPEAAGGPSVFLFPPKPKDTLMISRTPEVTCVVVDVSH | |
| L_T3A- | EDPEVKFNWYVDGVEVHNAKTKPREEQYNSTYRVVSVLTVLHQDWLNGKEYKC | |
| GGSGGGPL | KVSNKALAAPIEKTISKAKGQPREPQVYTLPPSRDELTKNQVSLTCLVKGFYP | |
| G | SDIAVEWESNGQPENNYKTTPPVLDSDGSFFLYSKLTVDKSRWQQGNVFSCSV | |
| | MHEALHNHYTQKSLSLSPGGGSGGSGGSGGSAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGGPLG | |
| Chain 5: | LAGGGAELCDDDPPEIPHATFKAMAYKEGTMLNCECKRGFRRIKSGSLYMLCT | 377 |
| LAGGGA-R | GNSSHSSWDNQCQCTSSATRNTTKQVTPQPEEQKERKTTEMQSPMQPVDQASL | |
| | PGHCREPPPWENEATERIYHFVVGQMVYYQCVQGYRALHRGPAESVCKMTHGK | |
| | TRWTQPQLICTGE | |

P14501953 Activatable Proprotein

| | | |
|---|---|---|
| Chains 1 | DIVMTQTPLSLSVTPGQPASISCRASKSVSTSAYSYMHWYLQKPGQSPQLLIY | 378 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 379 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKYEPKSSDKTHTCPPCPAPEAAGGPSVFLFPPKPKDTLMISRTPE | |
| GGSGGSGG | VTCVVVDVSHEDPEVKFNWYVDGVEVHNAKTKPREEQYNSTYRVVSVLTVLHQ | |
| SGGS- | DWLNGKEYKCKVSNKALAAPIEKTISKAKGQPREPQVYTLPPSRDELTKNQVS | |
| L_T3A- | LTCLVKGFYPSDIAVEWESNGQPENNYKTTPPVLDSDGSFFLYSKLTVDKSRW | |
| GGSGGSGR | QQGNVFSCSVMHEALHNHYTQKSLSLSPGGGSGGSGGSGGSAPASSSTKKTQL | |
| SDNHGGA- | QLEHLLLDLQMILNGINNYKNPKLTRMLTFKFYMPKKATELKHLQCLEEELKP | |
| R 12/15 | LEEALNLAPSKNFHLRPRDLISNINVIVLELKGSETTFMCEYADETATIVEFL | |
| | NRWITFSQSTISTLTGGSGGSGRSDNHGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |

P14501953 Cleaved proteins (Complete uPA/Matriptase cleavage)

| | | |
|---|---|---|
| Chains 1 | DIVMTQTPLSLSVTPGQPASISCRASKSVSTSAYSYMHWYLQKPGQSPQLLIY | 380 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 381 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKY | |
| GGSGGSGG | EPKSSDKTHTCPPCPAPEAAGGPSVFLFPPKPKDTLMISRTPEVTCVVVDVSH | |
| SGGS- | EDPEVKFNWYVDGVEVHNAKTKPREEQYNSTYRVVSVLTVLHQDWLNGKEYKC | |
| L_T3A- | KVSNKALAAPIEKTISKAKGQPREPQVYTLPPSRDELTKNQVSLTCLVKGFYP | |
| GGSGGSGR | SDIAVEWESNGQPENNYKTTPPVLDSDGSFFLYSKLTVDKSRWQQGNVFSCSV | |
| | MHEALHNHYTQKSLSLSPGGGSGGSGGSGGSAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGSGR | |
| Chains 5 | SDNHGGAELCDDDPPEIPHATFKAMAYKEGTMLNCECKRGFRRIKSGSLYMLC | 382 |
| and 6: | TGNSSHSSWDNQCQCTSSATRNTTKQVTPQPEEQKERKTTEMQSPMQPVDQAS | |
| SDNHGGA- | LPGHCREPPPWENEATERIYHFVVGQMVYYQCVQGYRALHRGPAESVCKMTHG | |
| R | KTRWTQPQLICTGE | |

P14501953 Cleaved proteins (Partial uPA/Matriptase cleavage)

| | | |
|---|---|---|
| Chains 1 | DIVMTQTPLSLSVTPGQPASISCRASKSVSTSAYSYMHWYLQKPGQSPQLLIY | 383 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 384 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AA | SWNSGALTSGVHTFPAVLQSSGLYSLSSVVTVPSSSLGTKTYTCNVDHKPSNT | |
| A_C217S) | KVDKRVESKYEPKSSDKTHTCPPCPAPEAAGGPSVFLFPPKPKDTLMISRTPE | |
| del K- | VTCVVVDVSHEDPEVKFNWYVDGVEVHNAKTKPREEQYNSTYRVVSVLTVLHQ | |
| GGSGGSGG | DWLNGKEYKCKVSNKALAAPIEKTISKAKGQPREPQVYTLPPSRDELTKNQVS | |
| SGGS- | LTCLVKGFYPSDIAVEWESNGQPENNYKTTPPVLDSDGSFFLYSKLTVDKSRW | |
| L_T3A- | QQGNVFSCSVMHEALHNHYTQKSLSLSPGGGSGGSGGSGGSAPASSSTKKTQL | |
| GGSGGSGR | QLEHLLLDLQMILNGINNYKNPKLTRMLTFKFYMPKKATELKHLQCLEEELKP | |
| SDNHGGA- | LEEALNLAPSKNFHLRPRDLISNINVIVLELKGSETTFMCEYADETATIVEFL | |
| R | NRWITFSQSTISTLTGGSGGSGRSDNHGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |
| Chain 4: | EVQLVQSGAEVKKPGESLKISCKGSGYTFTENIIHWVRQMPGKGLEWIGWFHP | 385 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGGSGG | KVDKRVESKY | |
| SGGS- | EPKSSDKTHTCPPCPAPEAAGGPSVFLFPPKPKDTLMISRTPEVTCVVVDVSH | |
| L_T3A- | EDPEVKFNWYVDGVEVHNAKTKPREEQYNSTYRVVSVLTVLHQDWLNGKEYKC | |
| GGSGGSGR | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSGGSGGSGGSAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGSGR | |
| Chain 5: | SDNHGGAELCDDDPPEIPHATFKAMAYKEGTMLNCECKRGFRRIKSGSLYMLC | 386 |
| SDNHGGA- | TGNSSHSSWDNQCQCTSSATRNTTKQVTPQPEEQKERKTTEMQSPMQPVDQAS | |
| R | LPGHCREPPPWENEATERIYHFVVGQMVYYQCVQGYRALHRGPAESVCKMTHG | |
| | KTRWTQPQLICTGE | |

P14501953 Cleaved proteins (Complete Legumain cleavage)

| | | |
|---|---|---|
| Chains 1 | DIVMTQTPLSLSVTPGQPASISCRASKSVSTSAYSYMHWYLQKPGQSPQLLIY | 387 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chains 3 | EVQLVQSGAEVKKPGESLKISCKGSGYTFTENIIHWVRQMPGKGLEWIGWFHP | 388 |
| and 4: | GSGSIKYNEKFKDQVTISADKSISTAYLQWSSLKASDTAMYFCARHGGTGRGA | |
| FAP1- | MDYWGQGTLVTVSSASTKGPSVFPLAPCSRSTSESTAALGCLVKDYFPEPVTV | |
| G4Fd- | SWNSGALTSGVHTFPAVLQSSGLYSLSSVVTVPSSSLGTKTYTCNVDHKPSNT | |
| Fc(G1_AAA_C217S)_delK- | KVDKRVESKY | |
| GGSGGSGG | EPKSSDKTHTCPPCPAPEAAGGPSVFLFPPKPKDTLMISRTPEVTCVVVDVSH | |
| SGGS- | EDPEVKFNWYVDGVEVHNAKTKPREEQYNSTYRVVSVLTVLHQDWLNGKEYKC | |
| L_T3A- | KVSNKALAAPIEKTISKAKGQPREPQVYTLPPSRDELTKNQVSLTCLVKGFYP | |
| GGSGGSGR | SDIAVEWESNGQPENNYKTTPPVLDSDGSFFLYSKLTVDKSRWQQGNVFSCSV | |
| SDN | MHEALHNHYTQKSLSLSPGGGSGGSGGSGGSAPASSSTKKTQLQLEHLLLDLQ | |
| | MILNGINNYKNPKLTRMLTFKFYMPKKATELKHLQCLEEELKPLEEALNLAPS | |
| | KNFHLRPRDLISNINVIVLELKGSETTFMCEYADETATIVEFLNRWITFSQST | |
| | ISTLTGGSGGSGRSDN | |
| Chains 5 | HGGAELCDDDPPEIPHATFKAMAYKEGTMLNCECKRGFRRIKSGSLYMLCTGN | 389 |
| and 6: | SSHSSWDNQCQCTSSATRNTTKQVTPQPEEQKERKTTEMQSPMQPVDQASLPG | |
| HGGA-R | HCREPPPWENEATERIYHFVVGQMVYYQCVQGYRALHRGPAESVCKMTHGKTR | |
| | WTQPQLICTGE | |

P14501953 Cleaved proteins (Partial Legumain cleavage)

| | | |
|---|---|---|
| Chains 1 | DIVMTQTPLSLSVTPGQPASISCRASKSVSTSAYSYMHWYLQKPGQSPQLLIY | 390 |
| and 2: | LASNLESGVPDRFSGSGSGTDFTLKISRVEAEDVGVYYCQHSRELPYTFGQGT | |
| FAP-L1- | KLEIKRTVAAPSVFIFPPSDEQLKSGTASVVCLLNNFYPREAKVQWKVDNALQ | |
| huIgkLC | SGNSQESVTEQDSKDSTYSLSSTLTLSKADYEKHKVYACEVTHQGLSSPVTKS | |
| | FNRGEC | |
| Chain 3: | EVQLVQSGAEVKKPGESLKISCKGSGYTFTENIIHWVRQMPGKGLEWIGWFHP | 391 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGGSGG | KVDKRVESKYEPKSSDKTHTCPPCPAPEAAGGPSVFLFPPKPKDTLMISRTPE | |
| SGGS- | VTCVVVDVSHEDPEVKFNWYVDGVEVHNAKTKPREEQYNSTYRVVSVLTVLHQ | |
| L_T3A- | DWLNGKEYKCKVSNKALAAPIEKTISKAKGQPREPQVYTLPPSRDELTKNQVS | |
| GGSGGSGR | LTCLVKGFYPSDIAVEWESNGQPENNYKTTPPVLDSDGSFFLYSKLTVDKSRW | |
| SDNHGGA- | QQGNVFSCSVMHEALHNHYTQKSLSLSPGGGSGGSGGSGGSAPASSSTKKTQL | |
| R | QLEHLLLDLQMILNGINNYKNPKLTRMLTFKFYMPKKATELKHLQCLEEELKP | |
| | LEEALNLAPSKNFHLRPRDLISNINVIVLELKGSETTFMCEYADETATIVEFL | |
| | NRWITFSQSTISTLTGGSGGSGRSDNHGGAELCDDDPPEIPHATFKAMAYKEG | |
| | TMLNCECKRGFRRIKSGSLYMLCTGNSSHSSWDNQCQCTSSATRNTTKQVTPQ | |
| | PEEQKERKTTEMQSPMQPVDQASLPGHCREPPPWENEATERIYHFVVGQMVYY | |
| | QCVQGYRALHRGPAESVCKMTHGKTRWTQPQLICTGE | |
| Chain 4: | EVQLVQSGAEVKKPGESLKISCKGSGYTFTENIIHWVRQMPGKGLEWIGWFHP | 392 |
| FAP1- | GSGSIKYNEKFKDQVTISADKSISTAYLQWSSLKASDTAMYFCARHGGTGRGA | |
| G4Fd- | MDYWGQGTLVTVSSASTKGPSVFPLAPCSRSTSESTAALGCLVKDYFPEPVTV | |
| Fc(G1_AAA_C217S)_delK- | SWNSGALTSGVHTFPAVLQSSGLYSLSSVVTVPSSSLGTKTYTCNVDHKPSNT | |
| GGSGGSGG | KVDKRVESKYEPKSSDKTHTCPPCPAPEAAGGPSVFLFPPKPKDTLMISRTPE | |
| SGGS- | VTCVVVDVSHEDPEVKFNWYVDGVEVHNAKTKPREEQYNSTYRVVSVLTVLHQ | |
| L_T3A- | DWLNGKEYKCKVSNKALAAPIEKTISKAKGQPREPQVYTLPPSRDELTKNQVS | |
| GGSGGSGR | LTCLVKGFYPSDIAVEWESNGQPENNYKTTPPVLDSDGSFFLYSKLTVDKSRW | |
| SDN | QQGNVFSCSVMHEALHNHYTQKSLSLSPGGGSGGSGGSGGSAPASSSTKKTQL | |
| | QLEHLLLDLQMILNGINNYKNPKLTRMLTFKFYMPKKATELKHLQCLEEELKP | |
| | LEEALNLAPSKNFHLRPRDLISNINVIVLELKGSETTFMCEYADETATIVEFL | |
| | NRWITFSQSTISTLTGGSGGSGRSDN | |
| Chain 5: | HGGAELCDDDPPEIPHATFKAMAYKEGTMLNCECKRGFRRIKSGSLYMLCTGN | 393 |
| HGGA-R | SSHSSWDNQCQCTSSATRNTTKQVTPQPEEQKERKTTEMQSPMQPVDQASLPG | |
| | HCREPPPWENEATERIYHFVVGQMVYYQCVQGYRALHRGPAESVCKMTHGKTR | |
| | WTQPQLICTGE | |

P1972 Activatable Proprotein

| | | |
|---|---|---|
| Chains 1 | EPKSSDKTHTCPPCPAPELLGGPSVFLFPPKPKDTLMISRTPEVTCVVVDVSH | 394 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_wt_E61S_T3A- | MHEALHNHYTQKSLSLSPGKGGGGSGGGGSAPASSSTKKTQLQLEHLLLDLQM | |
| GSGSENLY | ILNGINNYKNPKLTRMLTFKFYMPKKATELKHLQCLESELKPLEEVLNLAQSK | |
| FQGGSGS- | NFHLRPRDLISNINVIVLELKGSETTFMCEYADETATIVEFLNRWITFSQSII | |
| R_K38S | STLTGSGSENLYFQGGSGSELCDDDPPEIPHATFKAMAYKEGTMLNCECKRGF | |
| | RRISSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |

P1972 Cleaved proteins (Complete TEV cleavage)

| | | |
|---|---|---|
| Chains 1 | EPKSSDKTHTCPPCPAPELLGGPSVFLFPPKPKDTLMISRTPEVTCVVVDVSH | 395 |
| and 2: | EDPEVKFNWYVDGVEVHNAKTKPREEQYNSTYRVVSVLTVLHQDWLNGKEYKC | |
| Fc- | KVSNKALPAPIEKTISKAKGQPREPQVYTLPPSREEMTKNQVSLTCLVKGFYP | |
| stableLinker- | SDIAVEWESNGQPENNYKTTPPVLDSDGSFFLYSKLTVDKSRWQQGNVFSCSV | |
| L_wt_E61S_T3A- | MHEALHNHYTQKSLSLSPGKGGGGSGGGGSAPASSSTKKTQLQLEHLLLDLQM | |
| GSGSENLY | ILNGINNYKNPKLTRMLTFKFYMPKKATELKHLQCLESELKPLEEVLNLAQSK | |
| F | NFHLRPRDLISNINVIVLELKGSETTFMCEYADETATIVEFLNRWITFSQSII | |
| | STLTGSGSENLYFQ | |
| Chains 3 | GGSGSELCDDDPPEIPHATFKAMAYKEGTMLNCECKRGFRRISSGSLYMLCTG | 396 |
| and 4: | NSSHSSWDNQCQCTSSATRNTTKQVTPQPEEQKERKTTEMQSPMQPVDQASLP | |
| QGGSGS- | GHCREPPPWENEATERIYHFVVGQMVYYQCVQGYRALHRGPAESVCKMTHGKT | |
| R_K38S | RWTQPQLICTGE | |

P1972 Cleaved proteins (Partial TEV cleavage)

| | | |
|---|---|---|
| Chain 1: | EPKSSDKTHTCPPCPAPELLGGPSVFLFPPKPKDTLMISRTPEVTCVVVDVSH | 397 |
| Fc- | EDPEVKFNWYVDGVEVHNAKTKPREEQYNSTYRVVSVLTVLHQDWLNGKEYKC | |
| stableLinker- | KVSNKALPAPIEKTISKAKGQPREPQVYTLPPSREEMTKNQVSLTCLVKGFYP | |
| L_wt_E61S_T3A- | SDIAVEWESNGQPENNYKTTPPVLDSDGSFFLYSKLTVDKSRWQQGNVFSCSV | |
| GSGSENLY | MHEALHNHYTQKSLSLSPGKGGGGSGGGGSAPASSSTKKTQLQLEHLLLDLQM | |
| FQGGSGS- | ILNGINNYKNPKLTRMLTFKFYMPKKATELKHLQCLESELKPLEEVLNLAQSK | |
| R_K38S | NFHLRPRDLISNINVIVLELKGSETTFMCEYADETATIVEFLNRWITFSQSII | |
| | STLTGSGSENLYFQGGSGSELCDDDPPEIPHATFKAMAYKEGTMLNCECKRGF | |
| | RRISSGSLYMLCTGNSSHSSWDNQCQCTSSATRNTTKQVTPQPEEQKERKTTE | |
| | MQSPMQPVDQASLPGHCREPPPWENEATERIYHFVVGQMVYYQCVQGYRALHR | |
| | GPAESVCKMTHGKTRWTQPQLICTGE | |
| Chain 2: | EPKSSDKTHTCPPCPAPELLGGPSVFLFPPKPKDTLMISRTPEVTCVVVDVSH | 398 |
| Fc- | EDPEVKFNWYVDGVEVHNAKTKPREEQYNSTYRVVSVLTVLHQDWLNGKEYKC | |
| stableLinker- | KVSNKALPAPIEKTISKAKGQPREPQVYTLPPSREEMTKNQVSLTCLVKGFYP | |
| L_wt_E61S_T3A- | SDIAVEWESNGQPENNYKTTPPVLDSDGSFFLYSKLTVDKSRWQQGNVFSCSV | |
| GSGSENLY | MHEALHNHYTQKSLSLSPGKGGGGSGGGGSAPASSSTKKTQLQLEHLLLDLQM | |
| F | ILNGINNYKNPKLTRMLTFKFYMPKKATELKHLQCLESELKPLEEVLNLAQSK | |
| | NFHLRPRDLISNINVIVLELKGSETTFMCEYADETATIVEFLNRWITFSQSII | |
| | STLTGSGSENLYFQ | |
| Chain 3: | GGSGSELCDDDPPEIPHATFKAMAYKEGTMLNCECKRGFRRISSGSLYMLCTG | 399 |
| QGGSGS- | NSSHSSWDNQCQCTSSATRNTTKQVTPQPEEQKERKTTEMQSPMQPVDQASLP | |
| R_K38S | GHCREPPPWENEATERIYHFVVGQMVYYQCVQGYRALHRGPAESVCKMTHGKT | |
| | RWTQPQLICTGE | |

P2008 Activatable Proprotein

| | | |
|---|---|---|
| Chains 1 | EPKSSDKTHTCPPCPAPEAAGGPSVFLFPPKPKDTLMISRTPEVTCVVVDVSH | 400 |
| and 2: | EDPEVKFNWYVDGVEVHNAKTKPREEQYNSTYRVVSVLTVLHQDWLNGKEYKC | |
| 90_Fc(G1_AAA_C217S)_delK- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGS- | MHEALHNHYTQKSLSLSPGGGSPLGLAGGGSAAASSSTKKTQLQLEHLLLDLQ | |
| L_F42A_P2A_T3A- | MILNGINNYKNPKLTRMLTAKFYMPKKATELKHLQCLEEELKPLEEVLNLAQS | |
| GGSGGSGG | KNFHLRPRDLISNINVIVLELKGSETTFMCEYADETATIVEFLNRWITFSQSI | |
| SGGSGGA- | ISTLTGGSGGSGGSGGSGGAELCDDDPPEIPHATFKAMAYKEGTMLNCECKRG | |
| R | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P2009 Activatable Proprotein

| | | |
|---|---|---|
| Chains 1 | EPKSSDKTHTCPPCPAPEAAGGPSVFLFPPKPKDTLMISRTPEVTCVVVDVSH | 401 |
| and 2: | EDPEVKFNWYVDGVEVHNAKTKPREEQYNSTYRVVSVLTVLHQDWLNGKEYKC | |
| 91_Fc(G1_AAA_C217S)_delK- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGS- | MHEALHNHYTQKSLSLSPGGGSPLGLAGGGSAAASSSTKKTQLQLEHLLLDLQ | |
| L_wt_P2A_T3A- | MILNGINNYKNPKLTRMLTFKFYMPKKATELKHLQCLEEELKPLEEVLNLAQS | |
| GGSGGSGG | KNFHLRPRDLISNINVIVLELKGSETTFMCEYADETATIVEFLNRWITFSQSI | |
| SGGSGGA- | ISTLTGGSGGSGGSGGSGGAELCDDDPPEIPHATFKAMAYKEGTMLNCECKRG | |
| R | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P2010 Activatable Proprotein

| | | |
|---|---|---|
| Chains 1 | EPKSSDKTHTCPPCPAPEAAGGPSVFLFPPKPKDTLMISRTPEVTCVVVDVSH | 402 |
| and 2: | EDPEVKFNWYVDGVEVHNAKTKPREEQYNSTYRVVSVLTVLHQDWLNGKEYKC | |
| 92_Fc(G1_AAA_C217S)_delK- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGS- | MHEALHNHYTQKSLSLSPGGGSPLGLAGGGSAAASSSTKKTQLQLEHLLLDLQ | |
| L_tm_P2A_T3A- | MILNGINNYKNPKLTRMLTFKFYMPKKATELKHLQCLEEELKPLEEALNLAPS | |
| GGSGGSGG | KNFHLRPRDLISNINVIVLELKGSETTFMCEYADETATIVEFLNRWITFSQST | |
| SGGSGGA- | ISTLTGGSGGSGGSGGSGGAELCDDDPPEIPHATFKAMAYKEGTMLNCECKRG | |
| R | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P2011 Activatable Proprotein

| | | |
|---|---|---|
| Chains 1 | EPKSSDKTHTCPPCPAPEAAGGPSVFLFPPKPKDTLMISRTPEVTCVVVDVSH | 403 |
| and 2: | EDPEVKFNWYVDGVEVHNAKTKPREEQYNSTYRVVSVLTVLHQDWLNGKEYKC | |
| 93_Fc(G1_AAA_C217S)_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS- | MHEALHNHYTQKSLSLSPGGGSGGSGGSGGSAAASSSTKKTQLQLEHLLLDLQ | |
| L_tm_P2A_T3A- | MILNGINNYKNPKLTRMLTFKFYMPKKATELKHLQCLEEELKPLEEALNLAPS | |
| GGSGGSPL | KNFHLRPRDLISNINVIVLELKGSETTFMCEYADETATIVEFLNRWITFSQST | |
| GLAGGGA- | ISTLTGGSGGSPLGLAGGGAELCDDDPPEIPHATFKAMAYKEGTMLNCECKRG | |
| R | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P2012 Activatable Proprotein

| | | |
|---|---|---|
| Chains 1 | EPKSSDKTHTCPPCPAPEAAGGPSVFLFPPKPKDTLMISRTPEVTCVVVDVSH | 404 |
| and 2: | EDPEVKFNWYVDGVEVHNAKTKPREEQYNSTYRVVSVLTVLHQDWLNGKEYKC | |
| 94_Fc(G1_AAA_C217S)_delK | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGS- | MHEALHNHYTQKSLSLSPGGGSPLGLAGGGSAAASSSTKKTQLQLEHLLLDLQ | |
| L_tm_P2A_T3A- | MILNGINNYKNPKLTRMLTFKFYMPKKATELKHLQCLEEELKPLEEALNLAPS | |
| GGSGGSPL | KNFHLRPRDLISNINVIVLELKGSETTFMCEYADETATIVEFLNRWITFSQST | |
| GLAGGGA- | ISTLTGGSGGSPLGLAGGGAELCDDDPPEIPHATFKAMAYKEGTMLNCECKRG | |
| R | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |

P2013 Activatable Proprotein

| | | |
|---|---|---|
| Chains 1 | EPKSSDKTHTCPPCPAPEAAGGPSVFLFPPKPKDTLYITREPEVTCVVVDVSH | 405 |
| and 2: | EDPEVKFNWYVDGVEVHNAKTKPREEQYNSTYRVVSVLTVLHQDWLNGKEYKC | |
| 95_Fc(G1 | KVSNKALAAPIEKTISKAKGQPREPQVYTLPPSRDELTKNQVSLTCLVKGFYP | |
| _AAA_C21 | SDIAVEWESNGQPENNYKTTPPVLDSDGSFFLYSKLTVDKSRWQQGNVFSCSV | |
| 7S)_delK | MHEALHNHYTQKSLSLSPGGGSPLGLAGGGSAAASSSTKKTQLQLEHLLLDLQ | |
| _YTE- | MILNGINNYKNPKLTRMLTFKFYMPKKATELKHLQCLEEELKPLEEALNLAPS | |
| GGSPLGLA | KNFHLRPRDLISNINVIVLELKGSETTFMCEYADETATIVEFLNRWITFSQST | |
| GGGS- | ISTLTGGSGGSGGSGGSGGAELCDDDPPEIPHATFKAMAYKEGTMLNCECKRG | |
| L_tm_P2A | FRRIKSGSLYMLCTGNSSHSSWDNQCQCTSSATRNTTKQVTPQPEEQKERKTT | |
| _T3A- | EMQSPMQPVDQASLPGHCREPPPWENEATERIYHFVVGQMVYYQCVQGYRALH | |
| GGSGGSGG | RGPAESVCKMTHGKTRWTQPQLICTGE | |
| SGGSGGA- | | |
| R | | |

P2236 Activatable Proprotein

| | | |
|---|---|---|
| Chains 1 | EPKSSDKTHTCPPCPAPEAAGGPSVFLFPPKPKDTLYITREPEVTCVVVDVSH | 406 |
| and 2: | EDPEVKFNWYVDGVEVHNAKTKPREEQYNSTYRVVSVLTVLHQDWLNGKEYKC | |
| 248_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_wt_del_APASSS_N88R- | LNGINNYKNPKLTRMLTFKFYMPKKATELKHLQCLEEELKPLEEVLNLAQSKN | |
| GGSGGSGG | FHLRPRDLISRINVIVLELKGSETTFMCEYADETATIVEFLNRWITFSQSIIS | |
| SGGSGGA- | TLTGGSGGSGGSGGSGGAELCDDAPPEIPHATFKAMAYKEGTMLNCECKRGFR | |
| R_D6A | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2237 Activatable Proprotein

| | | |
|---|---|---|
| Chains 1 | EPKSSDKTHTCPPCPAPEAAGGPSVFLFPPKPKDTLYITREPEVTCVVVDVSH | 407 |
| and 2: | EDPEVKFNWYVDGVEVHNAKTKPREEQYNSTYRVVSVLTVLHQDWLNGKEYKC | |
| 249_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R- | LNGINNYKNPKLTRMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDAPPEIPHATFKAMAYKEGTMLNCECKRGFR | |
| R_D6A | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2238 Activatable Proprotein

| | | |
|---|---|---|
| Chains 1 | EPKSSDKTHTCPPCPAPEAAGGPSVFLFPPKPKDTLYITREPEVTCVVVDVSH | 408 |
| and 2: | EDPEVKFNWYVDGVEVHNAKTKPREEQYNSTYRVVSVLTVLHQDWLNGKEYKC | |
| 250_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R- | LNGINNYKNPKLTRMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDKPPEIPHATFKAMAYKEGTMLNCECKRGFR | |
| R_D6K | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2239 Activatable Proprotein

| | | |
|---|---|---|
| Chains 1 | EPKSSDKTHTCPPCPAPEAAGGPSVFLFPPKPKDTLYITREPEVTCVVVDVSH | 409 |
| and 2: | EDPEVKFNWYVDGVEVHNAKTKPREEQYNSTYRVVSVLTVLHQDWLNGKEYKC | |
| 251_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R_R38E- | LNGINNYKNPKLTEMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDKPPEIPHATFKAMAYKEGTMLNCECKRGFR | |
| R_D6K | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2240 Activatable Proprotein

| | | |
|---|---|---|
| Chains 1 | EPKSSDKTHTCPPCPAPEAAGGPSVFLFPPKPKDTLYITREPEVTCVVVDVSH | 410 |
| and 2: | EDPEVKFNWYVDGVEVHNAKTKPREEQYNSTYRVVSVLTVLHQDWLNGKEYKC | |
| 252_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R_R38E- | LNGINNYKNPKLTEMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDKPPEIPHATFKAMAYKEGTMLNCACKRGFR | |
| R_D6K_E29A | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2241 Activatable Proprotein

| | | |
|---|---|---|
| Chains 1 | EPKSSDKTHTCPPCPAPEAAGGPSVFLFPPKPKDTLYITREPEVTCVVVDVSH | 411 |
| and 2: | EDPEVKFNWYVDGVEVHNAKTKPREEQYNSTYRVVSVLTVLHQDWLNGKEYKC | |
| 253_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R_R38E- | LNGINNYKNPKLTEMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDKPPEIPHATFKAMAYKEGTMLNCECKRGFR | |
| R_D6K_R3EA | AIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2242 Activatable Proprotein

| | | |
|---|---|---|
| Chains 1 | EPKSSDKTHTCPPCPAPEAAGGPSVFLFPPKPKDTLYITREPEVTCVVVDVSH | 412 |
| and 2: | EDPEVKFNWYVDGVEVHNAKTKPREEQYNSTYRVVSVLTVLHQDWLNGKEYKC | |
| 254_Fc(G1_AAA_C217S)_YTE- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGSGGS- | MHEALHNHYTQKSLSLSPGKGGSPLGLAGGGSGGSTKKTQLQLEHLLLDLQMI | |
| L_tm_del_APASSS_N88R_R38E- | LNGINNYKNPKLTEMLTFKFYMPKKATELKHLQCLEEELKPLEEALNLAPSKN | |
| GGSGGSGG | FHLRPRDLISRINVIVLELKGSETTFMCEYADETATIVEFLNRWITFSQSTIS | |
| SGGSGGA- | TLTGGSGGSGGSGGSGGAELCDDKPPEIPHATFKAMAYKEGTMLNCECKRGFR | |
| R_D6K_K38A | RIASGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |

P2280 Activatable Proprotein

| | | |
|---|---|---|
| Chains 1 | EPKSSDKTHTCPPCPAPEAAGGPSVFLFPPKPKDTLMISRTPEVTCVVVDVSH | 413 |
| and 2: | EDPEVKFNWYVDGVEVHNAKTKPREEQYNSTYRVVSVLTVLHQDWLNGKEYKC | |
| 273_Fc(G1_AAA_C217S)_delK- | KVSNKALAAPIEKTISKAKGQPREPQVYTLPPSRDELTKNQVSLTCLVKGFYP | |
| GGSPLGLA | SDIAVEWESNGQPENNYKTTPPVLDSDGSFFLYSKLTVDKSRWQQGNVFSCSV | |
| GGGS-GG- | MHEALHNHYTQKSLSLSPGGGSPLGLAGGGSGGSTKKTQLQLEHLLLDLQMIL | |
| (STKK)- | NGINNYKNPKLTEMLTFKFYMPKKATELKHLQCLEEELKPLEEVLNLAQSKNF | |
| L_wt_del_APASS_N88R_R38E- | HLRPRDLISRINVIVLELKGSETTFMCEYADETATIVEFLNRWITFSQSIIST | |
| GGSGGSGG | LTGGSGGSGGSGGSGGAELCDDKPPEIPHATFKAMAYKEGTMLNCECKRGFRR | |
| SGGSGGA- | IKSGSLYMLCTGNSSHSSWDNQCQCTSSATRNTTKQVTPQPEEQKERKTTEMQ | |
| R_D6K | SPMQPVDQASLPGHCREPPPWENEATERIYHFVVGQMVYYQCVQGYRALHRGP | |
| | AESVCKMTHGKTRWTQPQLICTGE | |

P2281 Protein

| | | |
|---|---|---|
| Chains 1 | EPKSSDKTHTCPPCPAPEAAGGPSVFLFPPKPKDTLMISRTPEVTCVVVDVSH | 414 |
| and 2: | EDPEVKFNWYVDGVEVHNAKTKPREEQYNSTYRVVSVLTVLHQDWLNGKEYKC | |
| 274_Fc(G1_AAA_C217S)_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| (STKK)- | NGINNYKNPKLTEMLTFKFYMPKKATELKHLQCLEEELKPLEEVLNLAQSKNF | |
| L_wt_del_APASS_N88R_R38E- | HLRPRDLISRINVIVLELKGSETTFMCEYADETATIVEFLNRWITFSQSIIST | |
| GGSGGSGG | LTGGSGGSGGSGGSGGAELCDDKPPEIPHATFKAMAYKEGTMLNCECKRGFRR | |
| SGGSGGA- | IKSGSLYMLCTGNSSHSSWDNQCQCTSSATRNTTKQVTPQPEEQKERKTTEMQ | |
| R_D6K | SPMQPVDQASLPGHCREPPPWENEATERIYHFVVGQMVYYQCVQGYRALHRGP | |
| | AESVCKMTHGKTRWTQPQLICTGE | |

P2282 Activatable Proprotein

| | | |
|---|---|---|
| Chains 1 | EPKSSDKTHTCPPCPAPEAAGGPSVFLFPPKPKDTLMISRTPEVTCVVVDVSH | 415 |
| and 2: | EDPEVKFNWYVDGVEVHNAKTKPREEQYNSTYRVVSVLTVLHQDWLNGKEYKC | |
| 275_Fc(G1_AAA_C217S)_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| (STKK)- | NGINNYKNPKLTEMLTFKFYMPKKATELKHLQCLEEELKPLEEALNLAPSKNF | |
| L_tm_del_APASS_N88R_R38E- | HLRPRDLISRINVIVLELKGSETTFMCEYADETATIVEFLNRWITFSQSTIST | |
| GGSPLGLA | LTGGSPLGLAGGGSGGAELCDDKPPEIPHATFKAMAYKEGTMLNCECKRGFRR | |
| GGGSGGA- | IKSGSLYMLCTGNSSHSSWDNQCQCTSSATRNTTKQVTPQPEEQKERKTTEMQ | |
| R_D6K | SPMQPVDQASLPGHCREPPPWENEATERIYHFVVGQMVYYQCVQGYRALHRGP | |
| | AESVCKMTHGKTRWTQPQLICTGE | |

P2283 Protein

| | | |
|---|---|---|
| Chains 1 | EPKSSDKTHTCPPCPAPEAAGGPSVFLFPPKPKDTLMISRTPEVTCVVVDVSH | 416 |
| and 2: | EDPEVKFNWYVDGVEVHNAKTKPREEQYNSTYRVVSVLTVLHQDWLNGKEYKC | |
| 276_Fc(G1_AAA_C217S)_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| (STKK)- | NGINNYKNPKLTEMLTFKFYMPKKATELKHLQCLEEELKPLEEALNLAPSKNF | |
| L_tm_del_APASS_N88R_R38E- | HLRPRDLISRINVIVLELKGSETTFMCEYADETATIVEFLNRWITFSQSTIST | |
| GGSGGSGG | LTGGSGGSGGSGGSGGAELCDDKPPEIPHATFKAMAYKEGTMLNCECKRGFRR | |
| SGGSGGA- | IKSGSLYMLCTGNSSHSSWDNQCQCTSSATRNTTKQVTPQPEEQKERKTTEMQ | |
| R_D6K | SPMQPVDQASLPGHCREPPPWENEATERIYHFVVGQMVYYQCVQGYRALHRGP | |
| | AESVCKMTHGKTRWTQPQLICTGE | |

P2314 Activatable Proprotein

| | | |
|---|---|---|
| Chains 1 | EPKSSDKTHTCPPCPAPEAAGGPSVFLFPPKPKDTLYITREPEVTCVVVDVSH | 417 |
| and 2: | EDPEVKFNWYVDGVEVHNAKTKPREEQYNSTYRVVSVLTVLHQDWLNGKEYKC | |
| 301_Fc(G1_AAA_C217S)_YTE_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| L_tm_del_APASS_N88R_- | NGINNYKNPKLTRMLTFKFYMPKKATELKHLQCLEEELKPLEEALNLAPSKNF | |
| GGSPLGLA | HLRPRDLISRINVIVLELKGSETTFMCEYADETATIVEFLNRWITFSQSTIST | |
| GGGSGGA- | LTGGSPLGLAGGGSGGAELCDDDPPEIPHATFKAMAYKEGTMLNCECKRGFRR | |
| R | IKSGSLYMLCTGNSSHSSWDNQCQCTSSATRNTTKQVTPQPEEQKERKTTEMQ | |
| | SPMQPVDQASLPGHCREPPPWENEATERIYHFVVGQMVYYQCVQGYRALHRGP | |
| | AESVCKMTHGKTRWTQPQLICTGE | |

P2315 Activatable Proprotein

| | | |
|---|---|---|
| Chains 1 | EPKSSDKTHTCPPCPAPEAAGGPSVFLFPPKPKDTLYITREPEVTCVVVDVSH | 418 |
| and 2: | EDPEVKFNWYVDGVEVHNAKTKPREEQYNSTYRVVSVLTVLHQDWLNGKEYKC | |
| 302_Fc(G1_AAA_C217S)_YTE_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| L_tm_del_APASS_N88R_- | NGINNYKNPKLTRMLTFKFYMPKKATELKHLQCLEEELKPLEEALNLAPSKNF | |
| GGSPLGLA | HLRPRDLISRINVIVLELKGSETTFMCEYADETATIVEFLNRWITFSQSTIST | |
| GGGSGGA- | LTGGSPLGLAGGGSGGAELCDDDPPEIPHATFKAMAYKEGTMLNCECKRGFRR | |
| R K38A | IASGSLYMLCTGNSSHSSWDNQCQCTSSATRNTTKQVTPQPEEQKERKTTEMQ | |
| | SPMQPVDQASLPGHCREPPPWENEATERIYHFVVGQMVYYQCVQGYRALHRGP | |
| | AESVCKMTHGKTRWTQPQLICTGE | |

P2316 Protein

| | | |
|---|---|---|
| Chains 1 | EPKSSDKTHTCPPCPAPEAAGGPSVFLFPPKPKDTLYITREPEVTCVVVDVSH | 419 |
| and 2: | EDPEVKFNWYVDGVEVHNAKTKPREEQYNSTYRVVSVLTVLHQDWLNGKEYKC | |
| 303_Fc(G1_AAA_C217S)_YTE_delK- | KVSNKALAAPIEKTISKAKGQPREPQVYTLPPSRDELTKNQVSLTCLVKGFYP | |
| GGSGGSGG | SDIAVEWESNGQPENNYKTTPPVLDSDGSFFLYSKLTVDKSRWQQGNVFSCSV | |
| SGGS-GG- | MHEALHNHYTQKSLSLSPGGGSGGSGGSGGSGGSTKKTQLQLEHLLLDLQMIL | |
| L_tm_del_APASS_N88R_- | NGINNYKNPKLTRMLTFKFYMPKKATELKHLQCLEEELKPLEEALNLAPSKNF | |
| GGSGGSGG | HLRPRDLISRINVIVLELKGSETTFMCEYADETATIVEFLNRWITFSQSTIST | |
| SGGSGGA- | LTGGSGGSGGSGGSGGAELCDDDPPEIPHATFKAMAYKEGTMLNCECKRGFRR | |
| R | IKSGSLYMLCTGNSSHSSWDNQCQCTSSATRNTTKQVTPQPEEQKERKTTEMQ | |
| | SPMQPVDQASLPGHCREPPPWENEATERIYHFVVGQMVYYQCVQGYRALHRGP | |
| | AESVCKMTHGKTRWTQPQLICTGE | |

P2236 Cleaved proteins (Complete MMP-2 cleavage)

| | | |
|---|---|---|
| Chains 1 | EPKSSDKTHTCPPCPAPEAAGGPSVFLFPPKPKDTLYITREPEVTCVVVDVSH | 420 |
| and 2: | EDPEVKFNWYVDGVEVHNAKTKPREEQYNSTYRVVSVLTVLHQDWLNGKEYKC | |
| Fc(G1_AAA_C217S)_YTE- | KVSNKALAAPIEKTISKAKGQPREPQVYTLPPSRDELTKNQVSLTCLVKGFYP | |
| GGSPLG | SDIAVEWESNGQPENNYKTTPPVLDSDGSFFLYSKLTVDKSRWQQGNVFSCSV | |
| | MHEALHNHYTQKSLSLSPGKGGSPLG | |
| Chains 3 | LAGGGSGGSTKKTQLQLEHLLLDLQMILNGINNYKNPKLTRMLTFKFYMPKKA | 421 |
| and 4: | TELKHLQCLEEELKPLEEVLNLAQSKNFHLRPRDLISRINVIVLELKGSETTF | |
| LAGGGSGG | MCEYADETATIVEFLNRWITFSQSIISTLTGGSGGSGGSGGSGGAELCDDAPP | |
| S- | EIPHATFKAMAYKEGTMLNCECKRGFRRIKSGSLYMLCTGNSSHSSWDNQCQC | |
| L_wt_del_APASSS_N88R- | TSSATRNTTKQVTPQPEEQKERKTTEMQSPMQPVDQASLPGHCREPPPWENEA | |
| GGSGGSGG | TERIYHFVVGQMVYYQCVQGYRALHRGPAESVCKMTHGKTRWTQPQLICTGE | |
| SGGSGGA- | | |
| R_D6A | | |

P2236 Cleaved proteins (Partial MMP-2 cleavage)

| | | |
|---|---|---|
| Chain 1: | EPKSSDKTHTCPPCPAPEAAGGPSVFLFPPKPKDTLYITREPEVTCVVVDVSH | 422 |
| 248_Fc(G1_AAA_C217S)_YTE- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGSGGS- | SDIAVEWESNGQPENNYKTTPPVLDSDGSFFLYSKLTVDKSRWQQGNVFSCSV | |
| L_wt_del_APASSS_N88R- | MHEALHNHYTQKSLSLSPGKGGSPLGLAGGGSGGSTKKTQLQLEHLLLDLQMI | |
| GGSGGSGG | LNGINNYKNPKLTRMLTFKFYMPKKATELKHLQCLEEELKPLEEVLNLAQSKN | |
| SGGSGGA- | FHLRPRDLISRINVIVLELKGSETTFMCEYADETATIVEFLNRWITFSQSIIS | |
| R_D6A | TLTGGSGGSGGSGGSGGAELCDDAPPEIPHATFKAMAYKEGTMLNCECKRGFR | |
| | RIKSGSLYMLCTGNSSHSSWDNQCQCTSSATRNTTKQVTPQPEEQKERKTTEM | |
| | QSPMQPVDQASLPGHCREPPPWENEATERIYHFVVGQMVYYQCVQGYRALHRG | |
| | PAESVCKMTHGKTRWTQPQLICTGE | |
| Chain 2: | EPKSSDKTHTCPPCPAPEAAGGPSVFLFPPKPKDTLYITREPEVTCVVVDVSH | 423 |
| Fc(G1_AAA_C217S)_YTE- | EDPEVKFNWYVDGVEVHNAKTKPREEQYNSTYRVVSVLTVLHQDWLNGKEYKC | |
| GGSPLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGKGGSPLG | |
| Chain 3: | LAGGGSGGSTKKTQLQLEHLLLDLQMILNGINNYKNPKLTRMLTFKFYMPKKA | 424 |
| LAGGGSGG | TELKHLQCLEEELKPLEEVLNLAQSKNFHLRPRDLISRINVIVLELKGSETTF | |
| S- | MCEYADETATIVEFLNRWITFSQSIISTLTGGSGGSGGSGGSGGAELCDDAPP | |
| L_wt_del_APASSS_N88R- | EIPHATFKAMAYKEGTMLNCECKRGFRRIKSGSLYMLCTGNSSHSSWDNQCQC | |
| GGSGGSGG | TSSATRNTTKQVTPQPEEQKERKTTEMQSPMQPVDQASLPGHCREPPPWENEA | |
| SGGSGGA- | TERIYHFVVGQMVYYQCVQGYRALHRGPAESVCKMTHGKTRWTQPQLICTGE | |
| R_D6A | | |

P2012 Cleaved proteins (Complete MMP-2 cleavage)

| | | |
|---|---|---|
| Chains 1 | EPKSSDKTHTCPPCPAPEAAGGPSVFLFPPKPKDTLMISRTPEVTCVVVDVSH | 425 |
| and 2: | EDPEVKFNWYVDGVEVHNAKTKPREEQYNSTYRVVSVLTVLHQDWLNGKEYKC | |
| Fc(G1_AAA_C217S)_delK- | KVSNKALAAPIEKTISKAKGQPREPQVYTLPPSRDELTKNQVSLTCLVKGFYP | |
| GGSPLG | SDIAVEWESNGQPENNYKTTPPVLDSDGSFFLYSKLTVDKSRWQQGNVFSCSV | |
| | MHEALHNHYTQKSLSLSPGGGSPLG | |
| Chains 3 | LAGGGSAAASSSTKKTQLQLEHLLLDLQMILNGINNYKNPKLTRMLTFKFYMP | 426 |
| and 4: | KKATELKHLQCLEEELKPLEEALNLAPSKNFHLRPRDLISNINVIVLELKGSE | |
| LAGGGS-L_tm_P2A_T3A- | TTFMCEYADETATIVEFLNRWITFSQSTISTLTGGSGGSPLG | |
| GGSGGSPL | | |
| G | | |
| Chains 5 | LAGGGAELCDDDPPEIPHATFKAMAYKEGTMLNCECKRGFRRIKSGSLYMLCT | 427 |
| and 6: | GNSSHSSWDNQCQCTSSATRNTTKQVTPQPEEQKERKTTEMQSPMQPVDQASL | |
| LAGGGA-R | PGHCREPPPWENEATERIYHFVVGQMVYYQCVQGYRALHRGPAESVCKMTHGK | |
| | TRWTQPQLICTGE | |

P2012 Cleaved proteins (Product-1 from partial MMP-2 cleavage)

| | | |
|---|---|---|
| Chain 1: | EPKSSDKTHTCPPCPAPEAAGGPSVFLFPPKPKDTLMISRTPEVTCVVVDVSH | 428 |
| Fc(G1_AAA_C217S)_delK- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| L_tm_P2A_T3A- | MHEALHNHYTQKSLSLSPGGGSPLGLAGGGSAAASSSTKKTQLQLEHLLLDLQ | |
| GGSGGSPL | MILNGINNYKNPKLTRMLTFKFYMPKKATELKHLQCLEEELKPLEEALNLAPS | |
| GLAGGGA- | KNFHLRPRDLISNINVIVLELKGSETTFMCEYADETATIVEFLNRWITFSQST | |
| R | ISTLTGGSGGSPLGLAGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |
| Chain 2: | EPKSSDKTHTCPPCPAPEAAGGPSVFLFPPKPKDTLMISRTPEVTCVVVDVSH | 429 |
| Fc(G1_AAA_C217S)_delK- | EDPEVKFNWYVDGVEVHNAKTKPREEQYNSTYRVVSVLTVLHQDWLNGKEYKC | |
| GGSPLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSPLG | |
| Chain 3: | LAGGGSAAASSSTKKTQLQLEHLLLDLQMILNGINNYKNPKLTRMLTFKFYMP | 430 |
| LAGGGS- | KKATELKHLQCLEEELKPLEEALNLAPSKNFHLRPRDLISNINVIVLELKGSE | |
| L_tm_P2A_T3A- | TTFMCEYADETATIVEFLNRWITFSQSTISTLTGGSGGSPLGLAGGGAELCDD | |
| GGSGGSPL | DPPEIPHATFKAMAYKEGTMLNCECKRGFRRIKSGSLYMLCTGNSSHSSWDNQ | |
| GLAGGGA- | CQCTSSATRNTTKQVTPQPEEQKERKTTEMQSPMQPVDQASLPGHCREPPPWE | |
| R | NEATERIYHFVVGQMVYYQCVQGYRALHRGPAESVCKMTHGKTRWTQPQLICT | |
| | GE | |

P2012 Cleaved proteins (Product-2 from partial MMP-2 cleavage)

| | | |
|---|---|---|
| Chain 1: | EPKSSDKTHTCPPCPAPEAAGGPSVFLFPPKPKDTLMISRTPEVTCVVVDVSH | 431 |
| Fc(G1_AAA_C217S)_delK- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| L_tm_P2A_T3A- | MHEALHNHYTQKSLSLSPGGGSPLGLAGGGSAAASSSTKKTQLQLEHLLLDLQ | |
| GGSGGSPL | MILNGINNYKNPKLTRMLTFKFYMPKKATELKHLQCLEEELKPLEEALNLAPS | |
| GLAGGGA- | KNFHLRPRDLISNINVIVLELKGSETTFMCEYADETATIVEFLNRWITFSQST | |
| R | ISTLTGGSGGSPLGLAGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |
| Chain 2: | EPKSSDKTHTCPPCPAPEAAGGPSVFLFPPKPKDTLMISRTPEVTCVVVDVSH | 432 |
| Fc(G1_AAA_C217S)_delK- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| L_tm_P2A_T3A- | MHEALHNHYTQKSLSLSPGGGSPLGLAGGGSAAASSSTKKTQLQLEHLLLDLQ | |
| GGSGGSPL | MILNGINNYKNPKLTRMLTFKFYMPKKATELKHLQCLEEELKPLEEALNLAPS | |
| G | KNFHLRPRDLISNINVIVLELKGSETTFMCEYADETATIVEFLNRWITFSQST | |
| | ISTLTGGSGGSPLG | |
| Chain 3: | LAGGGAELCDDDPPEIPHATFKAMAYKEGTMLNCECKRGFRRIKSGSLYMLCT | 433 |
| LAGGGA-R | GNSSHSSWDNQCQCTSSATRNTTKQVTPQPEEQKERKTTEMQSPMQPVDQASL | |
| | PGHCREPPPWENEATERIYHFVVGQMVYYQCVQGYRALHRGPAESVCKMTHGK | |
| | TRWTQPQLICTGE | |

P2012 Cleaved proteins (Product-3 from partial MMP-2 cleavage)

| | | |
|---|---|---|
| Chain 1: | EPKSSDKTHTCPPCPAPEAAGGPSVFLFPPKPKDTLMISRTPEVTCVVVDVSH | 434 |
| Fc(G1_AAA_C217S)_delK- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| L_tm_P2A_T3A- | MHEALHNHYTQKSLSLSPGGGSPLGLAGGGSAAASSSTKKTQLQLEHLLLDLQ | |
| GGSGGSPL | MILNGINNYKNPKLTRMLTFKFYMPKKATELKHLQCLEEELKPLEEALNLAPS | |
| GLAGGGA- | KNFHLRPRDLISNINVIVLELKGSETTFMCEYADETATIVEFLNRWITFSQST | |
| R | ISTLTGGSGGSPLGLAGGGAELCDDDPPEIPHATFKAMAYKEGTMLNCECKRG | |
| | FRRIKSGSLYMLCTGNSSHSSWDNQCQCTSSATRNTTKQVTPQPEEQKERKTT | |
| | EMQSPMQPVDQASLPGHCREPPPWENEATERIYHFVVGQMVYYQCVQGYRALH | |
| | RGPAESVCKMTHGKTRWTQPQLICTGE | |
| Chain 2: | EPKSSDKTHTCPPCPAPEAAGGPSVFLFPPKPKDTLMISRTPEVTCVVVDVSH | 435 |
| Fc(G1_AAA_C217S)_delK- | EDPEVKFNWYVDGVEVHNAKTKPREEQYNSTYRVVSVLTVLHQDWLNGKEYKC | |
| GGSPLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSPLG | |
| Chain 3: | LAGGGSAAASSSTKKTQLQLEHLLLDLQMILNGINNYKNPKLTRMLTFKFYMP | 436 |
| LAGGGS- | KKATELKHLQCLEEELKPLEEALNLAPSKNFHLRPRDLISNINVIVLELKGSE | |
| L_tm_P2A_T3A- | TTFMCEYADETATIVEFLNRWITFSQSTISTLTGGSGGSPLG | |
| GGSGGSPL | | |
| G | | |
| Chain 4: | LAGGGAELCDDDPPEIPHATFKAMAYKEGTMLNCECKRGFRRIKSGSLYMLCT | 437 |
| LAGGGA-R | GNSSHSSWDNQCQCTSSATRNTTKQVTPQPEEQKERKTTEMQSPMQPVDQASL | |
| | PGHCREPPPWENEATERIYHFVVGQMVYYQCVQGYRALHRGPAESVCKMTHGK | |
| | TRWTQPQLICTGE | |

P2012 Cleaved proteins (Product-4 from partial MMP-2 cleavage)

| | | |
|---|---|---|
| Chain 1: | EPKSSDKTHTCPPCPAPEAAGGPSVFLFPPKPKDTLMISRTPEVTCVVVDVSH | 438 |
| Fc(G1_AAA_C217S)_delK- | EDPEVKFNWYVDGVEVHNAKTKPREEQYNSTYRVVSVLTVLHQDWLNGKEYKC | |
| GGSPLG | KVSNKALAAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSV | |
| | MHEALHNHYTQKSLSLSPGGGSPLG | |
| Chain 2: | LAGGGSAAASSSTKKTQLQLEHLLLDLQMILNGINNYKNPKLTRMLTFKFYMP | 439 |
| LAGGGS- | KKATELKHLQCLEEELKPLEEALNLAPSKNFHLRPRDLISNINVIVLELKGSE | |
| L_tm_P2A_T3A- | TTFMCEYADETATIVEFLNRWITFSQSTISTLTGGSGGSPLGLAGGGAELCDD | |
| GGSGGSPL | DPPEIPHATFKAMAYKEGTMLNCECKRGFRRIKSGSLYMLCTGNSSHSSWDNQ | |
| GLAGGGA- | CQCTSSATRNTTKQVTPQPEEQKERKTTEMQSPMQPVDQASLPGHCREPPPWE | |
| R | NEATERIYHFVVGQMVYYQCVQGYRALHRGPAESVCKMTHGKTRWTQPQLICT | |
| | GE | |
| Chain 3: | EPKSSDKTHTCPPCPAPEAAGGPSVFLFPPKPKDTLMISRTPEVTCVVVDVSH | 440 |
| Fc(G1_AAA_C217S)_delK- | EDPEVKFNWYVDGVEVHNAKTKPREEQYNSTYRVVSVLTVLHQDWLNGKEYKC | |
| GGSPLGLA | KVSNKALAAPIEKTISKAKGQPREPQVYTLPPSRDELTKNQVSLTCLVKGFYP | |
| GGGS- | SDIAVEWESNGQPENNYKTTPPVLDSDGSFFLYSKLTVDKSRWQQGNVFSCSV | |
| L_tm_P2A_T3A- | MHEALHNHYTQKSLSLSPGGGSPLGLAGGGSAAASSSTKKTQLQLEHLLLDLQ | |
| GGSGGSPL | MILNGINNYKNPKLTRMLTFKFYMPKKATELKHLQCLEEELKPLEEALNLAPS | |
| G | KNFHLRPRDLISNINVIVLELKGSETTFMCEYADETATIVEFLNRWITFSQST | |
| | ISTLTGGSGGSPLG | |
| Chain 4: | LAGGGAELCDDDPPEIPHATFKAMAYKEGTMLNCECKRGFRRIKSGSLYMLCT | 441 |
| LAGGGA-R | GNSSHSSWDNQCQCTSSATRNTTKQVTPQPEEQKERKTTEMQSPMQPVDQASL | |
| | PGHCREPPPWENEATERIYHFVVGQMVYYQCVQGYRALHRGPAESVCKMTHGK | |
| | TRWTQPQLICTGE | |

Thus, in certain embodiments, an activatable proprotein comprises a first polypeptide that comprises, consists, or consists essentially of an amino acid sequence that is at least 80, 85, 90, 95, 98, or 100% identical to a sequence selected from Table S4. In certain embodiments, the proteast cleavage site (e.g., the TEV protease cleavage site) of any one or more of the foregoing sequences (from Table S4) is replaced with a human protease cleavage site, that is, a cleavage site cleavable by a human protease, for example, a human protease expressed in a cancer tissue or cancer cell (see, for example, Table S3 for exemplary cleavable linkers).

### Methods of Use and Pharmaceutical Compositions

Certain embodiments include methods of treating, ameliorating the symptoms of, and/or reducing the progression of, a disease or condition in a subject in need thereof, comprising administering to the subject at least one activatable proprotein, as described herein. Also included are methods of enhancing an immune response in a subject comprising administering to the subject at least one activatable proprotein, as described herein. In particular embodiments, the disease is selected from one or more of a cancer, a viral infection, and an immune disorder.

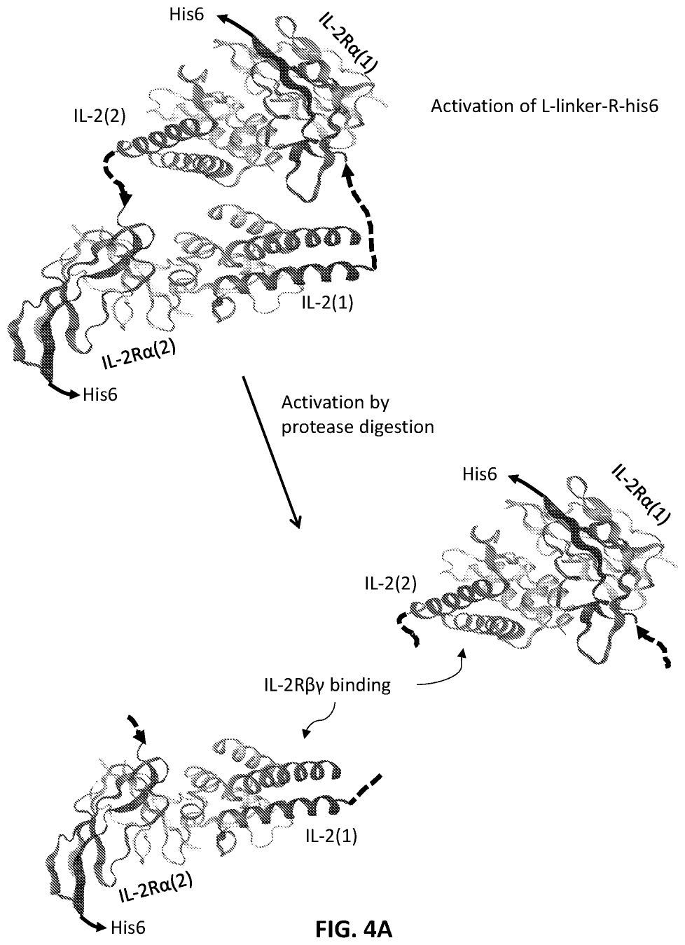

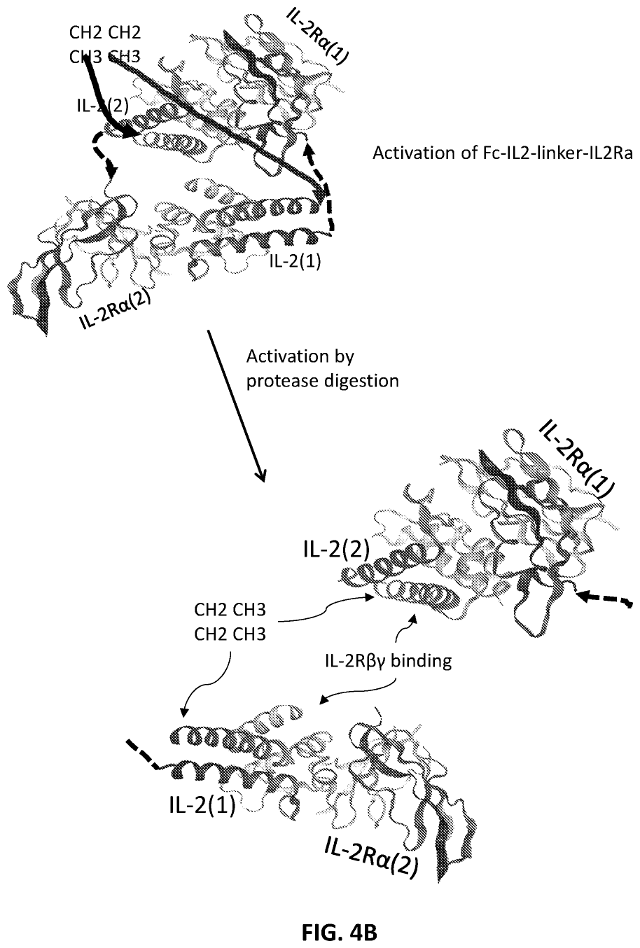

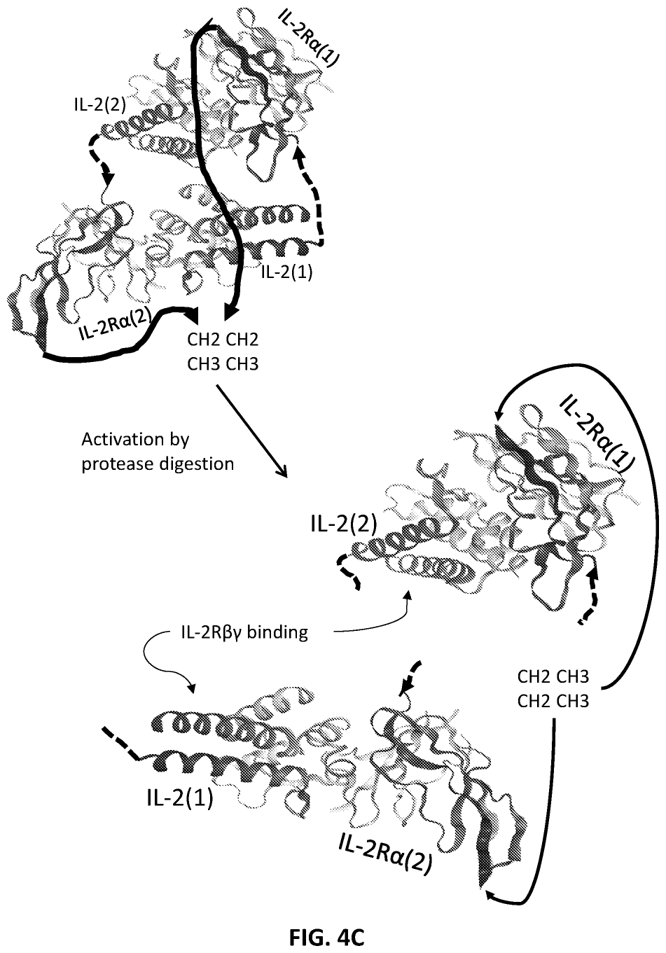

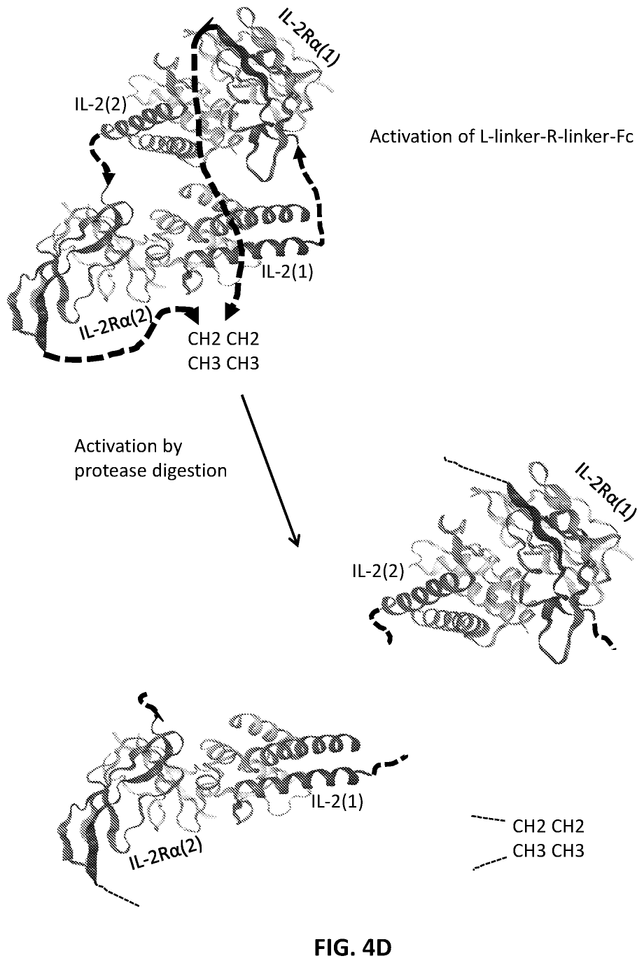

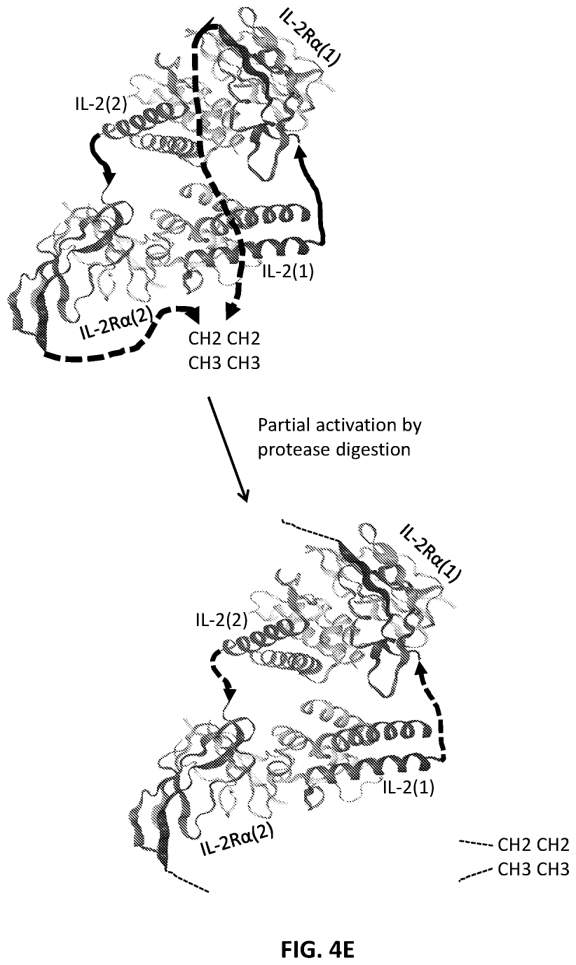

In some embodiments, following administration, the activatable proprotein is activated through protease cleavage in a cell or tissue, which releases or opens the homodimer, exposes the binding site of the IL-2 proteins that binds to the IL-2Rβ/γc chain present on the surface of the immune cell in vitro or in vivo, and thereby generates an activated protein (see, for example, FIGS. 4A-4D). In particular embodiments, the protease cleavage occurs in a cancer cell or cancer tissue, or a virally-infected cell or virally-infected tissue. Typically, the activated protein has at least one immune-stimulating IL-2 activity, for example, by binding to the IL-2Rβ/γc chain present on the surface of an immune cell in vivo, and thereby stimulating the immune cell. In particular embodiments, the immune cell is selected from one or more of a T cell, a B cell, a natural killer cell, a monocyte, and a macrophage.

In some embodiments, administration and activation of the activatable proprotein, to generate an activated protein, increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some instances, the immune response is an anti-cancer or anti-viral immune response. In some embodiments, administration and activation of the activatable proprotein, to generate an activated protein, increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, wherein the cell-killing is cancer cell-killing or virally-infected cell-killing.

In some embodiments, administration and activation of the activatable proprotein, to generate an activated protein, does not significantly increase binding of the activated protein to the IL-2Rα/β/γc chain expressed on regulatory T cells (T_{regs}). For example, in certain activated proteins, the binding between the IL-2 protein and the IL-2 binding protein (for example, disulfide binding between the IL-2 protein and the IL-2Rα protein) is maintained following linker cleavage, masks the binding site of the IL-2 protein that binds to the IL-2Rα/β/γc chain expressed on T_{regs}, and thereby interferes with binding of the activated protein to T_{regs}. Thus, in certain embodiments, the activated protein does not significantly stimulate or enhance the proliferation and/or activation of (T_{regs}), relative to the activatable proprotein.

In some embodiments, the disease is a cancer, that is, the subject in need thereof has or is suspected of having a cancer. Certain embodiments thus include methods of treating, ameliorating the symptoms of, or inhibiting the progression of, a cancer in a subject in need thereof, comprising administering to the subject at least one activatable proprotein, as described herein. In particular embodiments, the cancer is a primary cancer or a metastatic cancer. In specific embodiments, the cancer is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject at least one activatable proprotein described herein in combination with at least one additional agent, for example, a chemotherapeutic agent, a hormonal therapeutic agent, and/or a kinase inhibitor. In some embodiments, administering the at least one activatable proprotein enhances the susceptibility of the cancer to the additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide , and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine)

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The skilled artisan will appreciate that the various chemotherapeutic agents described herein can be combined with any one or more of the activatable proproteins described herein, and used according to any one or more of the methods or compositions described herein.

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab, ixadotin, and bemarituzumab; and inhibitors of the PDGF receptor such as olaratumab and tovetumab.

The skilled artisan will appreciate that the various hormonal therapeutic agents described herein can be combined with any one or more of the various activatable proproteins described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The skilled artisan will appreciate that the various kinase inhibitors described herein can be combined with any one or more of the various activatable proproteins described herein, and used according to any one or more of the methods or compositions described herein.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and pharmaceutical compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In some embodiments, the disease is a viral disease or viral infection. In certain embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis. In specific embodiments, the subject is HIV-positive. In some embodiments, the methods and pharmaceutical compositions described herein increase an anti-viral immune response by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the immune disorder is selected from one or more of type 1 diabetes, vasculitis, and an immunodeficiency. In some embodiments, the methods and pharmaceutical compositions described herein improve immune function in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In certain embodiments, the methods and therapeutic compositions described herein are sufficient to result in clinically relevant reduction in symptoms of a particular disease indication known to the skilled clinician.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to pharmaceutical or therapeutic compositions that comprise at least one activatable proprotein, as described herein. In some instances, a pharmaceutical or therapeutic composition comprises one or more of the activatable proproteins described herein in combination with a pharmaceutically- or physiologically-acceptable carrier or excipient. Certain pharmaceutical or therapeutic compositions further comprise at least one additional agent, for example, a chemotherapeutic agent, a hormonal therapeutic agent, and/or a kinase inhibitor as described herein.

Some therapeutic compositions comprise (and certain methods utilize) only one activatable proprotein. Certain therapeutic compositions comprise (and certain methods utilize) a mixture of at least two, three, four, or five different activatable proproteins.

In particular embodiments, the pharmaceutical or therapeutic compositions comprising at least one activatable proprotein is substantially pure on a protein basis or a weight-weight basis, for example, the composition has a purity of at least about 80%, 85%, 90%, 95%, 98%, or 99% on a protein basis or a weight-weight basis.

In certain embodiments, the first and second polypeptides, prior to cleavage, are substantially in homodimeric form in a composition or other physiological solution, or under physiological conditions, for example, in vivo conditions.

In some embodiments, the activatable proproteins described herein do not form aggregates, have a desired solubility, and/or have an immunogenicity profile that is suitable for use in humans, as known in the art. Thus, in some embodiments, the therapeutic composition comprising an activatable proprotein is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise an activatable proprotein that is at least about 50%, about 60%, about 70%, about 80%, about 90% or about 95% monodisperse with respect to its apparent molecular mass.

In some embodiments, the activatable proprotein are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an activatable proprotein and an additional therapeutic agent (e.g., chemotherapeutic agent, hormonal therapeutic agent, kinase inhibitor), as well as administration of compositions comprising an activatable proprotein and an additional therapeutic agent in its own separate pharmaceutical dosage formulation. For example, an activatable proprotein and additional therapeutic agent can be administered to the subject together in a single oral dosage composition such as a tablet or capsule, or each agent administered in separate oral dosage formulations Similarly, an activatable proprotein and additional therapeutic agent can be administered to the subject together in a single parenteral dosage composition such as in a saline solution or other physiologically acceptable solution, or each agent administered in separate parenteral dosage formulations. As another example, for cell-based therapies, an activatable proprotein can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one activatable proprotein, as described herein; and optionally (b) at least one additional therapeutic agent (e.g., chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an activatable proprotein and optionally at least one additional therapeutic agent. For example, the kit includes a plurality of syringes, ampules, foil packets, or blister packs, each containing a single unit dose of an activatable proprotein and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

Expression and Purification Systems

Certain embodiments include methods and related compositions for expressing and purifying an activatable proprotein described herein. Such recombinant activatable proproteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, activatable proproteins may be prepared by a procedure including one or more of the steps of: (a) preparing one or more vectors or constructs comprising one or more polynucleotide sequences that encode an individual polypeptide chain of the homodimer, which are operably linked to one or more regulatory elements; (b) introducing the one or more vectors or constructs into one or more host cells; (c) culturing the one or more host cell to express the polypeptides, which bind together to form an activatable proprotein homodimer; and (d) isolating the activatable proprotein homodimer from the host cell. Alternatively, the polypeptide chain can be first isolated and produced in a host cell, and then incubated under suitable conditions to form an activatable proprotein homodimer.

To express a desired polypeptide, a nucleotide sequence encoding a first and/or second polypeptide chain of an activatable proprotein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS⋅TAG® fusions), followed by immobilized metal affinity chromatography (IMAC) purification, or related techniques. In certain aspects, however, clinical grade proteins can be isolated from E. coli inclusion bodies, without or without the use of affinity tags (see, e g , Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others. In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL 51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K.C Lo, ed., Humana Press, Totowa, N.J., 2003), pp. 255-268. Certain preferred mammalian cell expression systems include CHO and HEK293-cell based expression systems Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt- cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif. 40:237-43, 2005; and U.S. Pat. No. 6,210,922).

An activatable proprotein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6× His), cMyc tags, VS-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., Coomassie, silver stain), immunoblot, Bradford, and ELISA, which may be utilized during any step of the production or purification process, typically to measure the purity of the protein composition.

Also included are methods of concentrating activatable proproteins, and composition comprising concentrated soluble activatable proproteins. In some aspects, such concentrated solutions of at least one activatable proprotein comprise proteins at a concentration of about or at least about 5 mg/mL, 8 mg/mL, 10 mg/mL, 15 mg/mL, 20 mg/mL, or more.

In some aspects, such compositions may be substantially monodisperse, meaning that an activatable proprotein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, an activatable proprotein in a composition has a purity of at least about 90%, as measured according to routine techniques in the art. In certain embodiments, such as diagnostic compositions or certain pharmaceutical or therapeutic compositions, an activatable proprotein composition has a purity of at least about 95%, or at least about 97% or 98% or 99%. In some embodiments, such as when being used as reference or research reagents, activatable proproteins can be of lesser purity, and may have a purity of at least about 50%, 60%, 70%, or 80%. Purity can be measured overall or in relation to selected components, such as other proteins, e.g., purity on a protein basis.

Purified activatable proproteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to cell-based assays, including those that utilize at least one IL-2 receptor, which is optionally functionally coupled to a readout or indicator, such as a fluorescent or luminescent indicator of biological activity, as described herein.

In certain embodiments, as noted above, an activatable proprotein composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, an activatable proprotein composition is made from a eukaryotic cell such as a mammalian or human cell in substantially serum free media. In certain embodiments, as noted herein, an activatable proprotein composition has an endotoxin content of less than about 10 EU/mg of activatable proprotein, or less than about 5 EU/mg of activatable proprotein, less than about 3 EU/mg of activatable proprotein, or less than about 1 EU/mg of activatable proprotein.

In certain embodiments, an activatable proprotein composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade activatable proproteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents (e.g., activatable proprotein) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of activatable proproteins and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Activatable proprotein with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an activatable proprotein of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### ENGINEERING OF “IL-2-LINKER-IL-2RA” AND “IL-2RA-LINKER-IL-2” ACTIVATABLE PROPROTEINS

In order to reduce the toxicity of IL-2 related therapeutic drugs, IL-2-linker-IL-2Rα (hereon referred to as ILR fusion proteins) and IL-2Rα-linker-IL-2 fusion proteins (hereon referred to as RLI fusion proteins) were generated as prodrugs, or activatable proproteins. The prodrugs have very low activity in their activatable form. Full or nearly full activities can be restored upon protease cleavage of the designed protease specific linker sequence within the prodrugs (see, for example, FIGS. 4A-4E).

Human IL-2-T3 with triple mutation of V69A, Q74P, and I128T, which has higher binding affinity towards IL-2Rα, was used in the exemplary fusion proteins. In order to restore the activity of IL-2, the TEV protease cleavage site was used to provide proof of concept.

Plasmids coding for single chain IL-2-linker-IL-2Rα (ILR) and IL-2Rα-linker-IL-2 (RLI) with or without Fc fusion were constructed by standard gene synthesis, followed by sub-cloning into pTT5 expression vector. Schematics of illustrative ILR fusion protein formats are depicted in FIGS. 2A, 2B, 2D, 2E, 2G, and 2H. Schematics of illustrative RLI fusion protein formats are depicted in FIGS. 3A, 3B, 3D, 3E, 3G, and 3H.

Illustrative proteins of IL-2-stable linker-IL-2Rα format (FIG. 2A) with C-terminal His-tag include P1522 and P1525. Illustrative proteins of IL-2-TEV-IL-2Rα format (FIG. 2B) with C-terminal His-tag include P1630, P1664, and P1667. Illustrative proteins of Fc-TEV-IL-2-stable linker-IL-2Rα format (FIG. 2D) include P1523 and P1526. Illustrative proteins of Fc-stable liner-IL-2-TEV-IL-2Rα format (FIG. 2E) include P1631, P1665, and P1668. Illustrative proteins of IL-2-stable linker-IL-2Rα-TEV-Fc format (FIG. 2G) include P1524 and P1527. Illustrative proteins of IL-2-TEV-IL-2Rα-stable linker-Fc format (FIG. 2H) include P1632, P1666, and P1669.

Illustrative proteins of IL-2Rα-stable linker-IL-2 format (FIG. 3A) with C-terminal His-tag include P1528 and P1531. Illustrative proteins of IL-2Rα-TEV-IL-2 format (FIG. 3B) with C-terminal His-tag include P1633, P1773, and P1776. Illustrative proteins of Fc-TEV-IL-2Rα-stable linker-IL-2 format (FIG. 3D) include P1529 and P1532. Illustrative proteins of Fc-stable liner-IL-2Rα-TEV-IL-2 format (FIG. 3E) include P1634, P1774, and P1777. Illustrative proteins of IL-2Rα-stable linker-IL-2-TEV-Fc format (FIG. 3G) include P1530 and P1533. Illustrative proteins of IL-2Rα-TEV-IL-2-stable linker-Fc format (FIG. 3H) include P1635, P1775, and P1778.

In P1522, P1523, P1524, P1528, P1529, and P1530, the linker length is 5 amino acid between IL-2 and IL-2Rα. In P1525, P1526, P1527, P1531, P1532, and P1533, the linker length is 10 amino acid between IL-2 and IL-2Rα. In P1630, P1631, P1632, P1633, P1634, and P1635, the linker length is 11 amino acid between IL-2 and IL-2Rα. In P1664, P1665, P1666, P1773, P1774, and P1775, the linker length is 15 amino acid between IL-2 and IL-2Rα. In P1667, P1668, P1669, P1776, P1777, and P1778, the linker length is 19 amino acid between IL-2 and IL-2Rα.

For ILR format, real protease cleavage sites (PSs) were introduced into the linker between IL-2 and IL-2Rα. Potential O-glycosylation site in IL-2 was substituted with alanine (T3A). Disulfide bond was introduced between IL-2 and IL-2Rα by introducing E61C into IL-2 and K38C into IL-2Rα. Illustrative proteins include P1719 (IL-2-PS-IL-2Rα-His6), P1720 (Fc-stable linker-IL-2-PS-IL-2Rα), P1721 (IL-2-PS-IL-2Rα-stable linker-Fc), P1722 (IL-2-PS-IL-2Rα-His6), P1723 (Fc-stable linker-IL-2-PS-IL-2Rα), and P1724 (IL-2-PS-IL-2Rα-stable linker-Fc).

For Fc-stable linker-IL-2Rα-PS-IL-2 format, real protease cleavage sites (PSs) were introduced into the linker between IL-2Rα and IL-2. A potential O-glycosylation site in IL-2 was substituted with alanine (T3A). A disulfide bond was introduced between IL-2 and IL-2Rα. At least one cysteine mutation was introduced in IL-2 at K35, R38, or E61, and in IL-2Rα at D04, H120, K38, or S39. Illustrative proteins include P1725, P1726, P1727, P1728, P1729 and P1730.

For activatable proprotein design, real protease cleavage sites (PSs) were introduced into the linker between IL-2 and IL-2Rα for IL-2-PS-IL-2Rα-stable linker-Fc format. A potential O-glycosylation site in IL-2 was substituted with alanine (T3A). Plasmids coding for IL-2-PSs-IL-2Rα-stable linker-Fc were constructed by standard gene synthesis and sub-cloned into pTT5 expression vector with linker flanking protease cleavage sites. Illustrative proteins include P1779, P1780, P1781, P1782, P1783, P1784, and P1785. P1786 was generated as a control protein without a cleavage site between IL-2 and IL-2Rα.

For activatable proprotein design, different real protease cleavage sites (PSs) were introduced into the linker between Fc/IL-2 and IL-2/IL-2Rα, respectively. A potential O-glycosylation site in IL-2 was substituted with alanine (T3A). Potential N-glycosylation sites in IL-2R were substituted with alanine (N49A and N68A) in one construct. IL-2-D10 was also tested in one construct. Plasmids coding for Fc-PS1-IL-2-PS2-IL2Rα were constructed by standard gene synthesis and sub-cloned into pTT5 expression vector with linker flanking protease cleavage sites. Illustrative proteins include P1834, P1835, P1836, P1837, P1838, P1839, P1840, P1841, P1842, P1843, P1844, P1845, P1846, P1847, P1848, P1849, and P1850.

Wild type IL-2 and IL-2 muteins with lower binding affinity towards IL-2Rα were also tested in the Fc-stable linker-IL-2-TEV-IL-2Rα format. A potential O-glycosylation site was substituted with alanine (T3A). IL-2 muteins tested include IL-2-F42A, IL-2-Y45A, and IL-2-F42A-Y45A. Illustrative proteins include P1946, P1947, P1948, and P1949. The IL-2-E61S/IL-2Rα-K38S combination was also tested. An illustrative protein includes P1972.

ILR format was also tested in antibody fusion format. ILR was fused to the C-terminal of an antibody heavy chain with a protease cleavage site between the heavy chain and IL-2, or between IL-2 and IL-2Rα. A potential O-glycosylation site in IL-2 was substituted with alanine (T3A), and the C-terminal lysine (K) on the heavy chain was deleted. The cysteine 217 on heavy chain was substituted with serine since IgG4-Fd was used. Illustrative proteins include P14501950, P14501951, P14501952, and P14501953.

Production, purification and characterization. Fc fusion proteins were produced by transient transfection in Expi293 cells and purified by a two-step purification process comprising Mab Select SuRe chromatography (GE Healthcare) and size exclusion chromatography (Superdex 200, GE Healthcare). His-tagged proteins were produced by transient transfection in Expi293 cells and purified by a two-step purification process comprising nickel affinity chromatography (GE Healthcare) and size exclusion chromatography (Superdex 200, GE Healthcare).

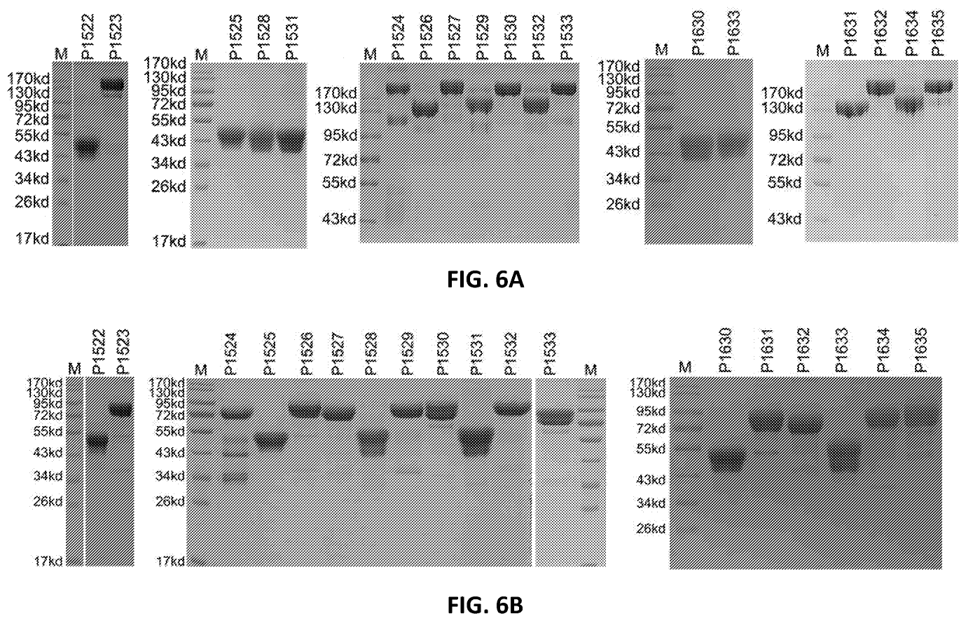

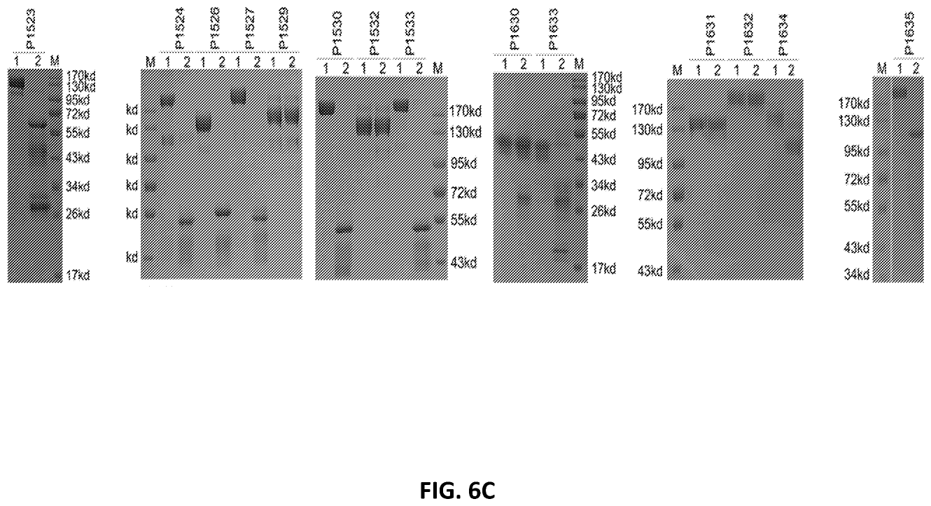

Purified proteins were characterized by SDS-PAGE for purity assessment and showed good purity as shown, for example, in FIGS. 6A, 6B, 10A, 10B, 13A, 13B, 16A, 16B, 19A, 19B, 22A, 22B, 25A, 25B, 27A, and 27B.

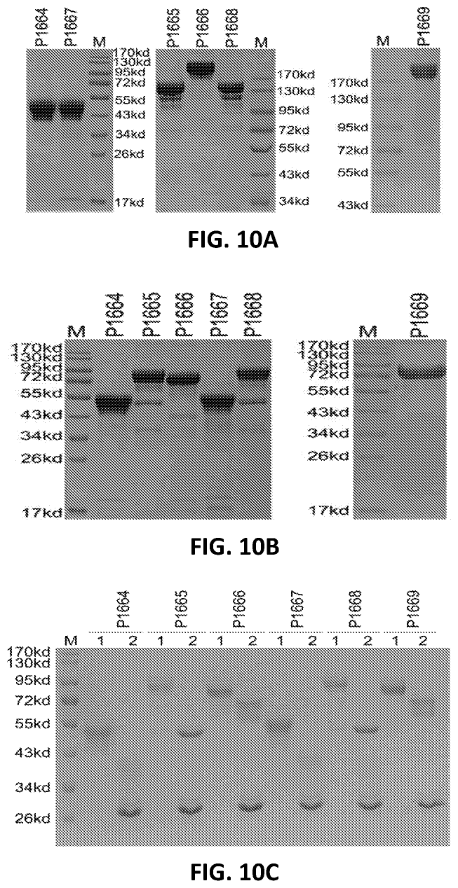

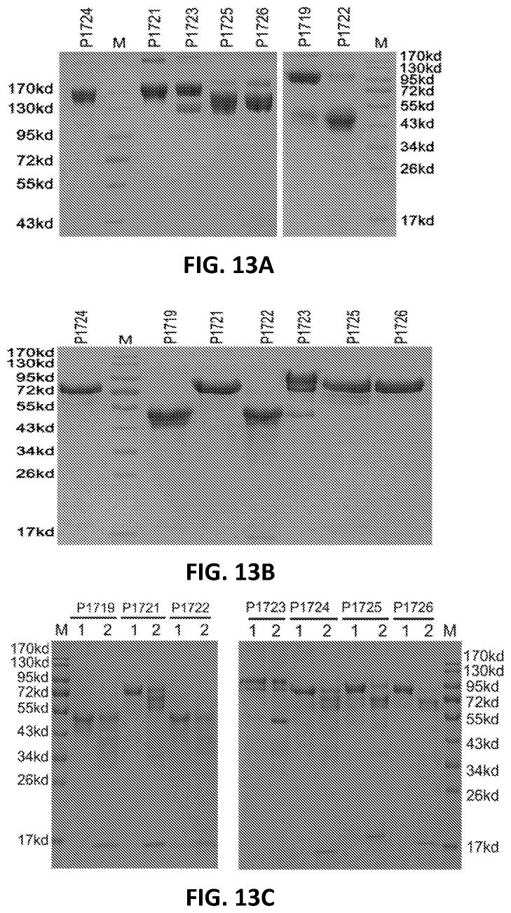

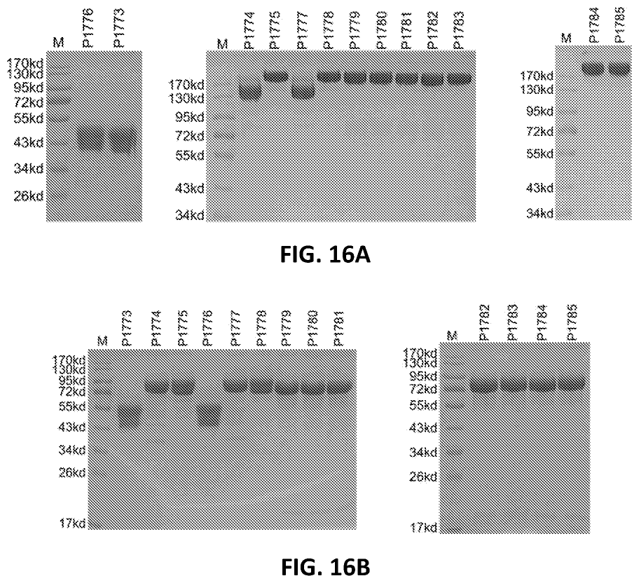

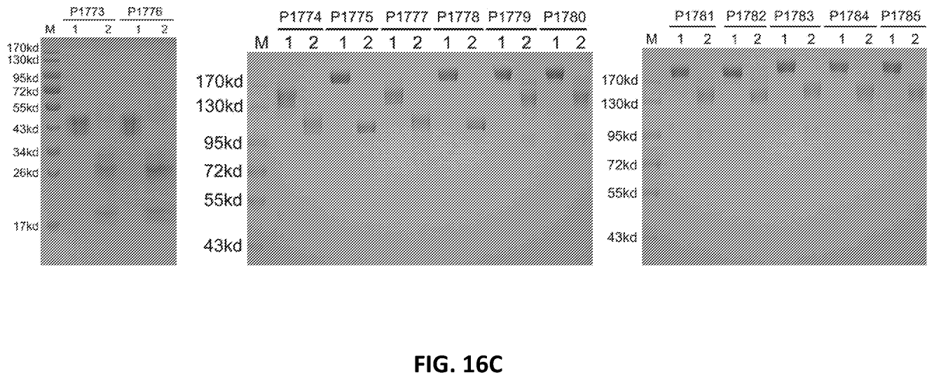

Protease cleavage was performed for purified proteins with the corresponding cleavage site. The proteases tested were as follows: TEV, uPA(R&D, Cat #1310-SE-010), matriptase (R&D, Cat #3946-SEB-010) and MMP-2 (R&D, Cat #902-MP-010). P1529, P1532, P1630, P1631, and P1632 could not be cleaved by TEV and the other proteins could be cleaved by TEV as shown in FIG. 6C. P1664, P1665, P1666, P1667, P1668, and P1669 could be cleaved by TEV as shown in FIG. 10C. P1719, P1721, P1722, P1723, P1724, P1725, and P1726 could be cleaved by uPA protease partially as shown in FIG. 13C. P1773, P1774, P1775, P1776, P1777, and P1778 could be cleaved by TEV, and P1779, P1780, P1781, P1782, P1783, P1784, and P1785 could be cleaved by uPA protease, as shown in FIG. 16C.

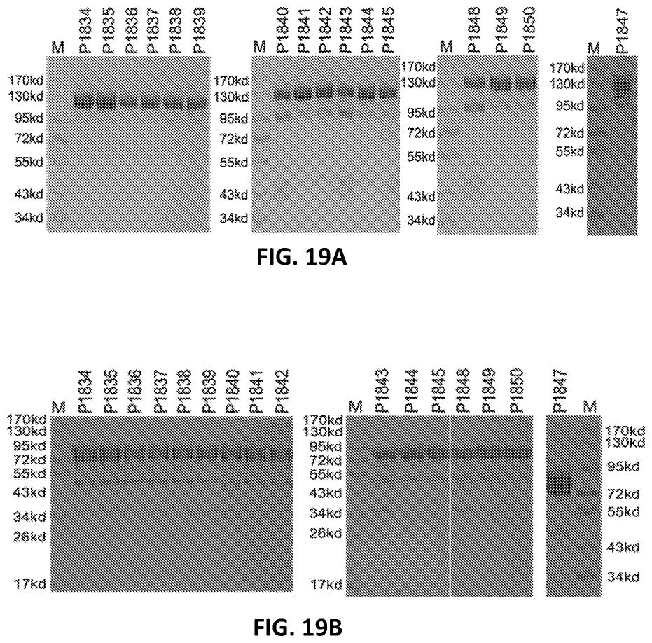

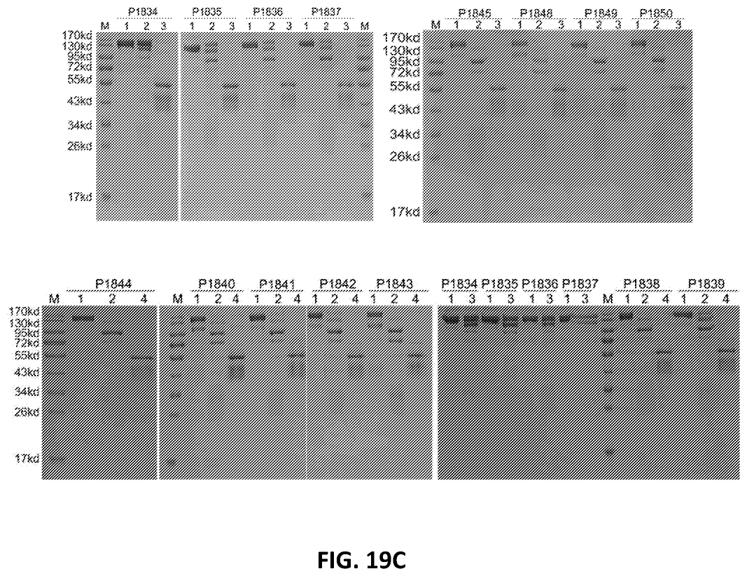

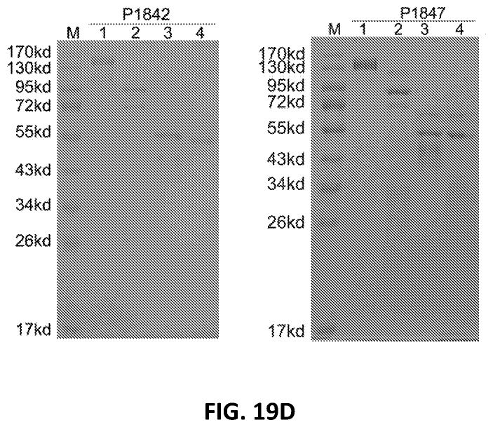

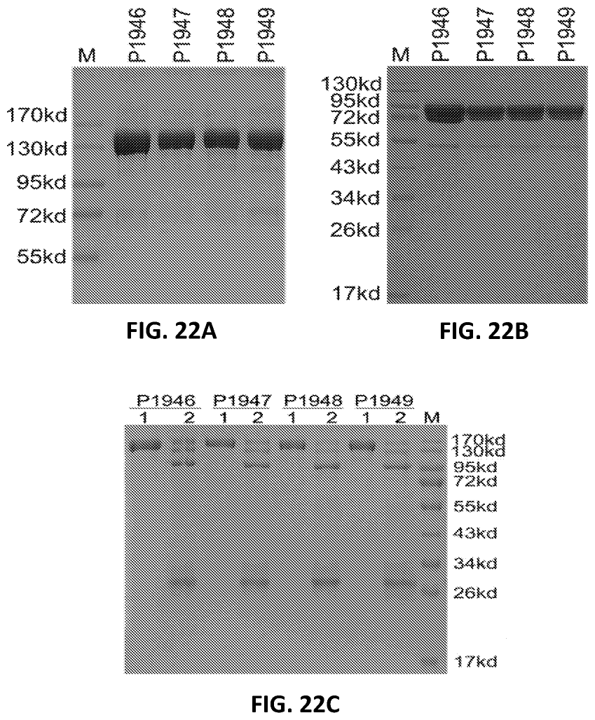

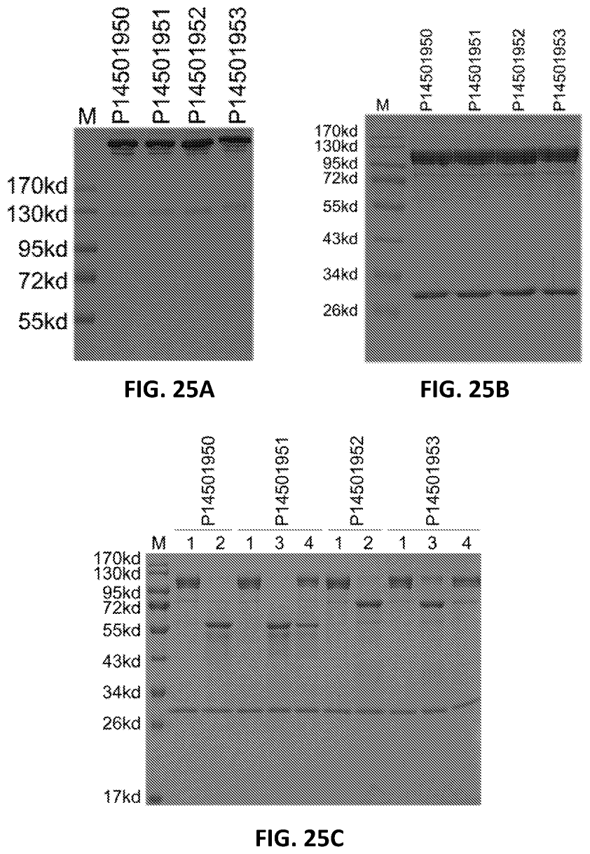

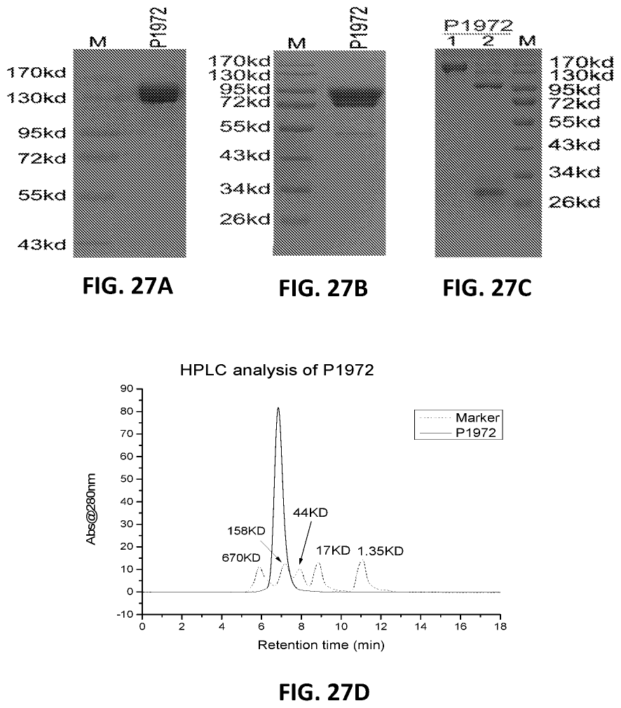

As shown in FIG. 19C, the purified proteins could be cleaved by uPA, matriptase, or MMP-2 partially or completely. As shown in FIG. 19D, P1842 and P1847 could be cleaved by uPA and MMP-2 at the same time. P1946, P1947, P1948, and P1949 could be cleaved by TEV partially as shown in FIG. 22C. P14501950, P14501951, P14501952, and P14501953 could be cleaved by MMP-2, uPA, or matriptase completely or partially as shown in FIG. 25C. P1972 could be cleaved by TEV partially as shown in FIG. 27C.

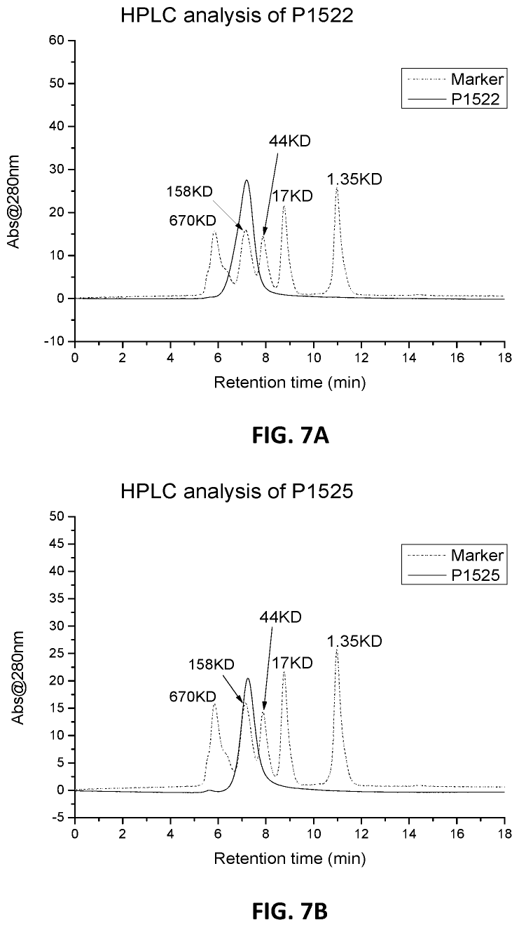

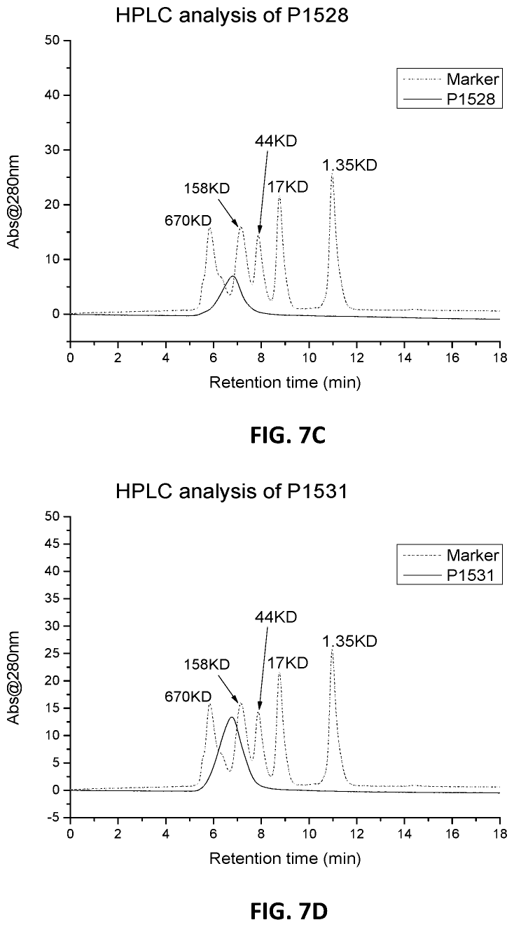

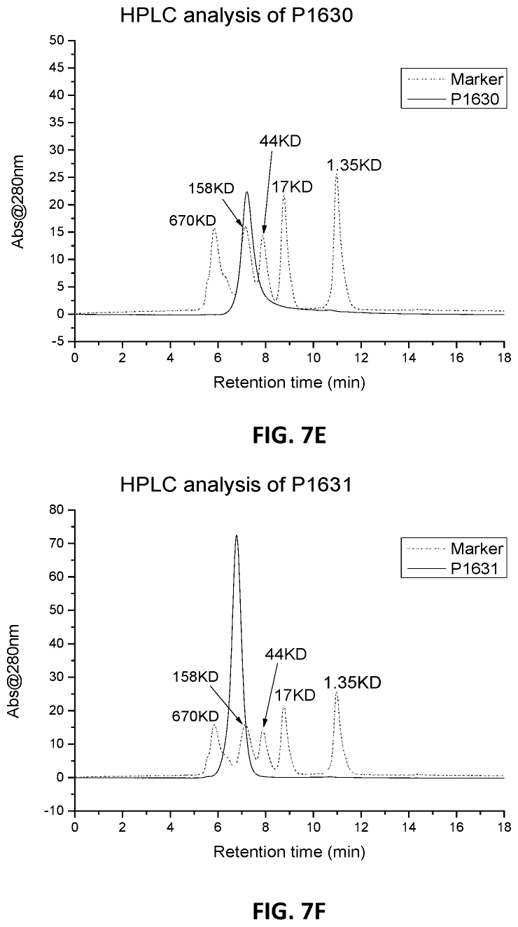

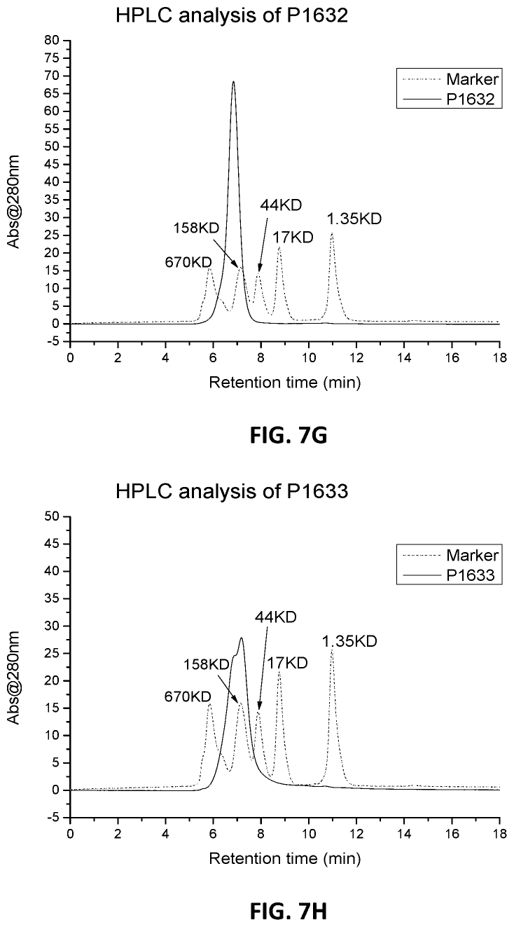

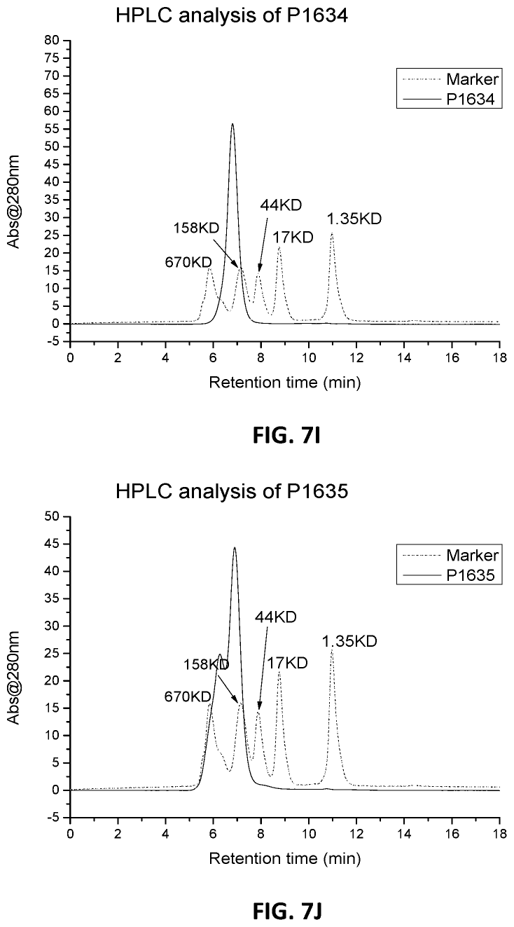

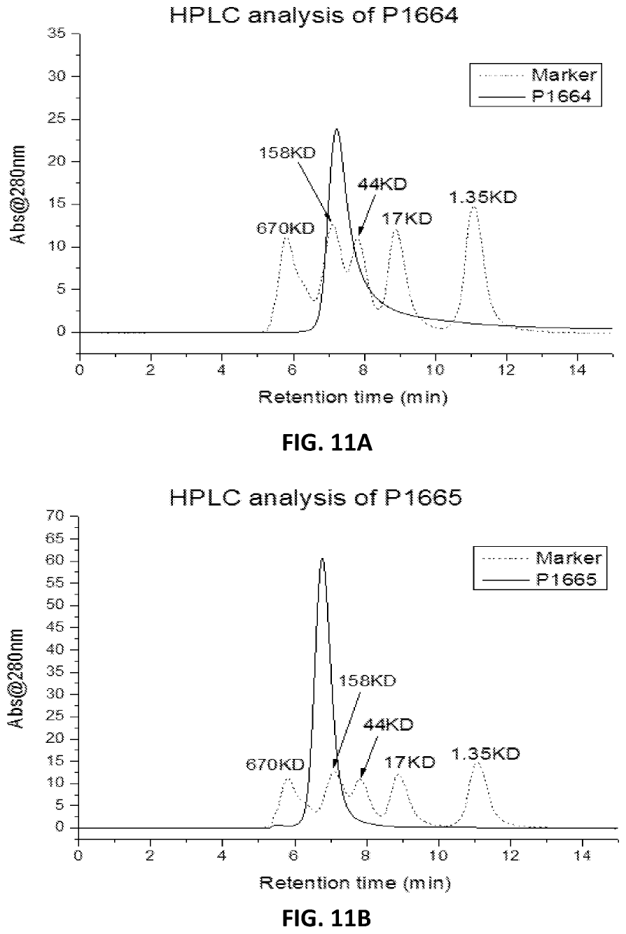

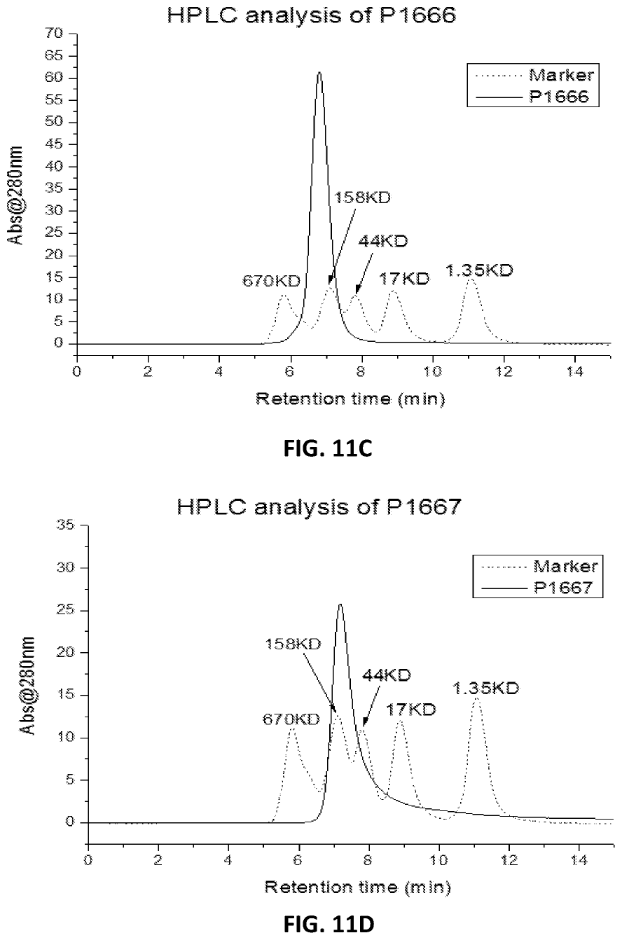

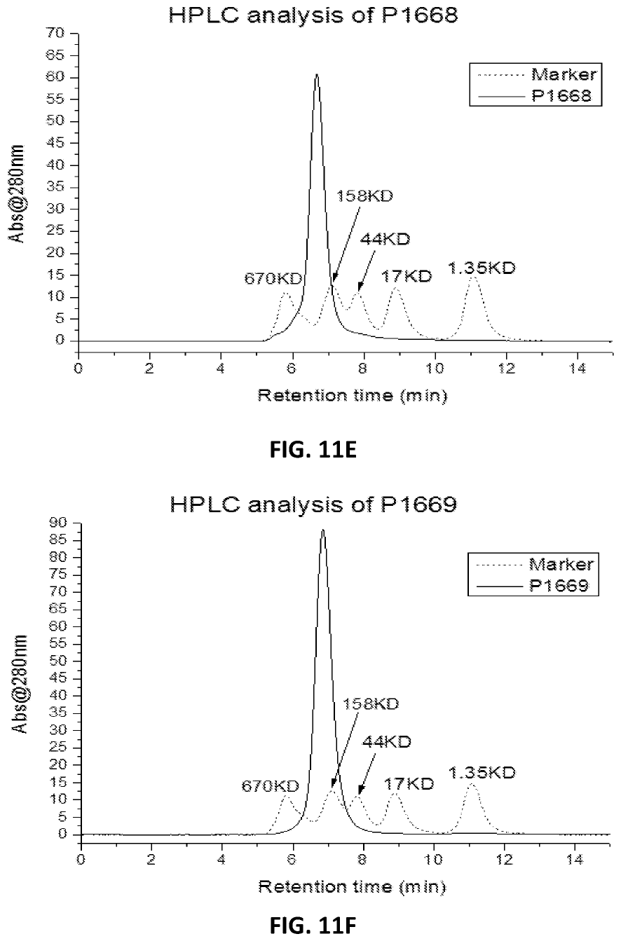

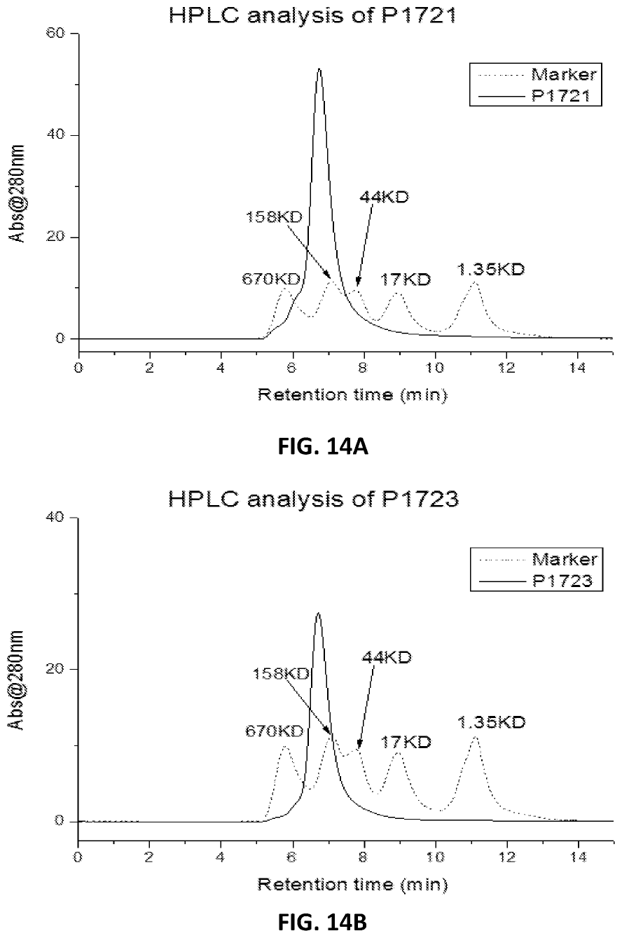

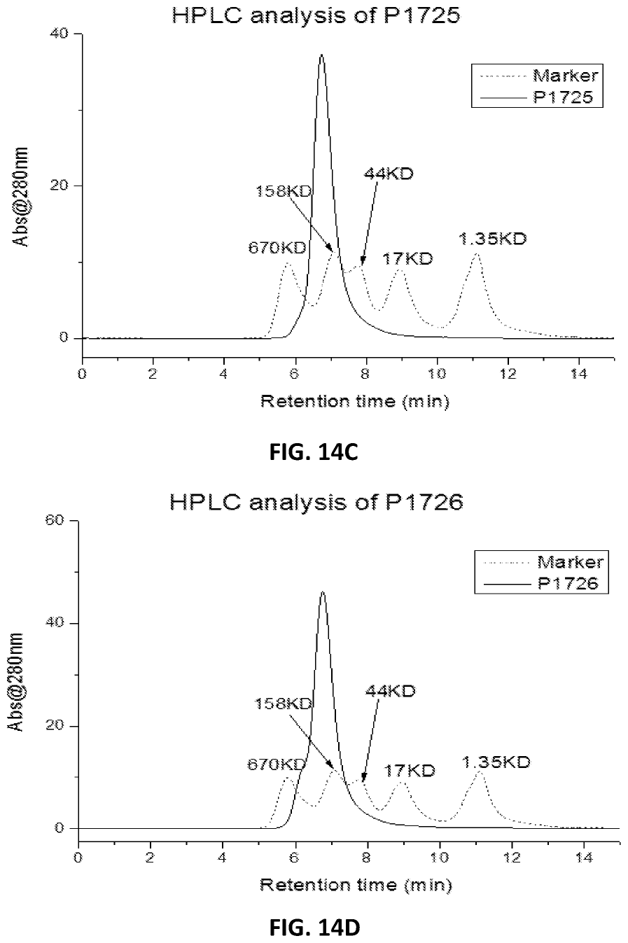

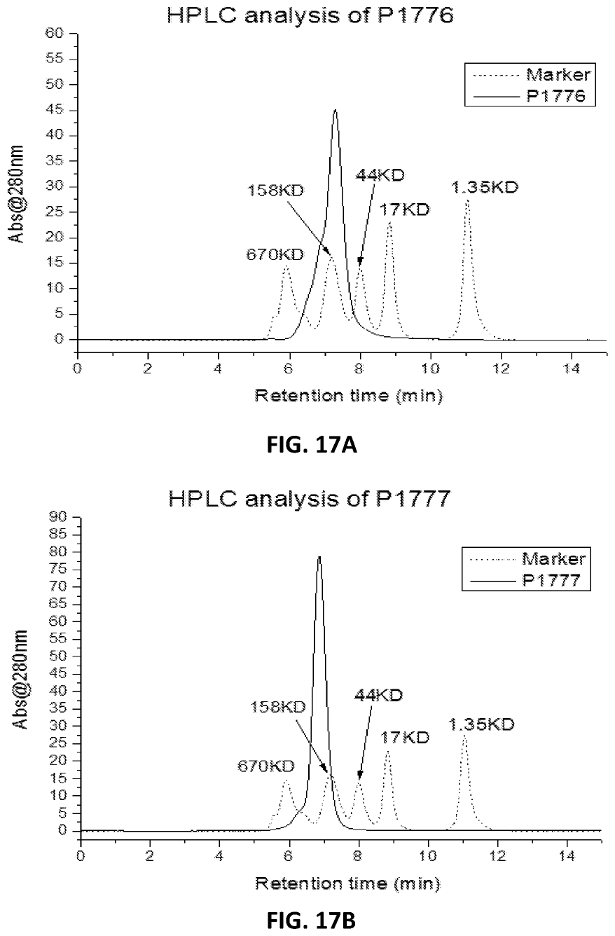

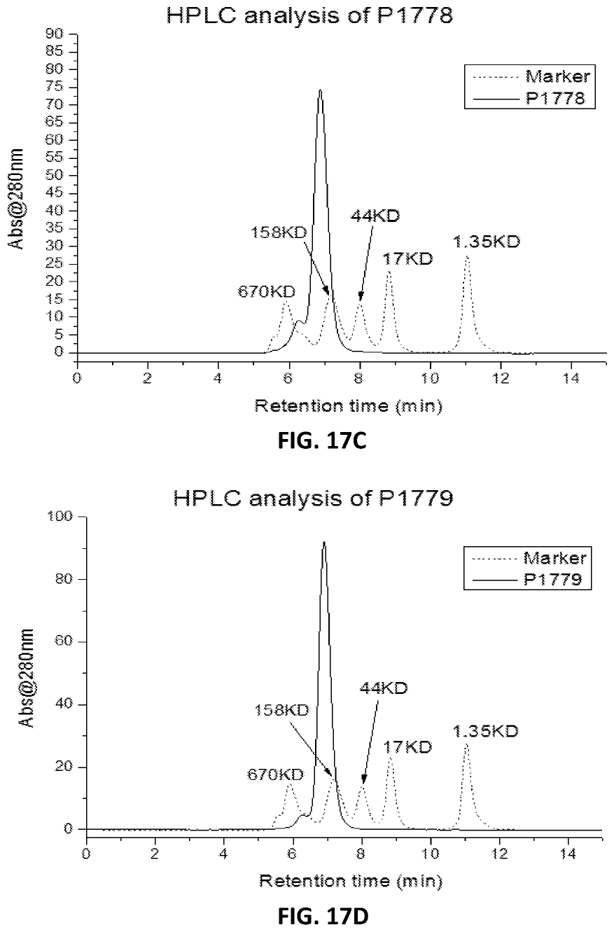

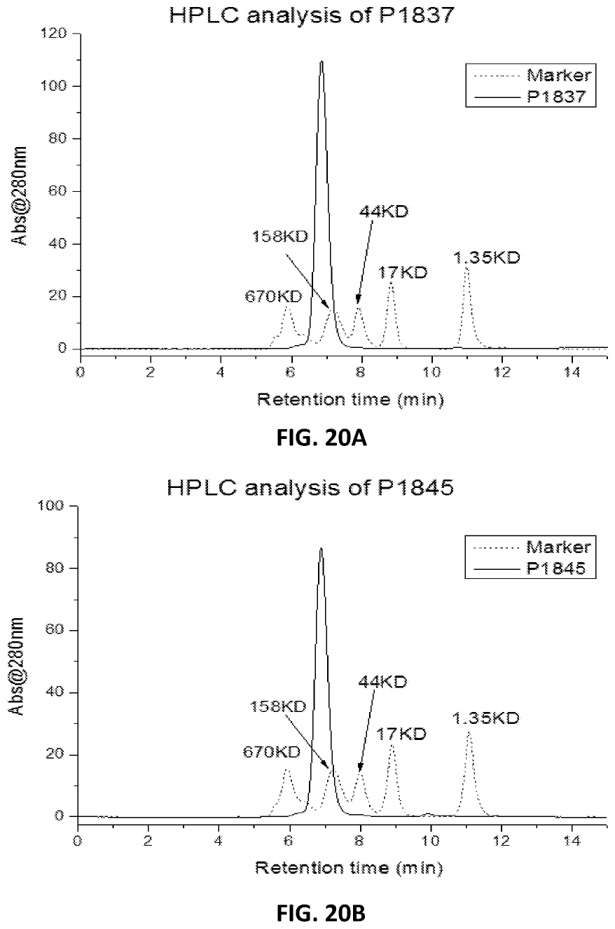

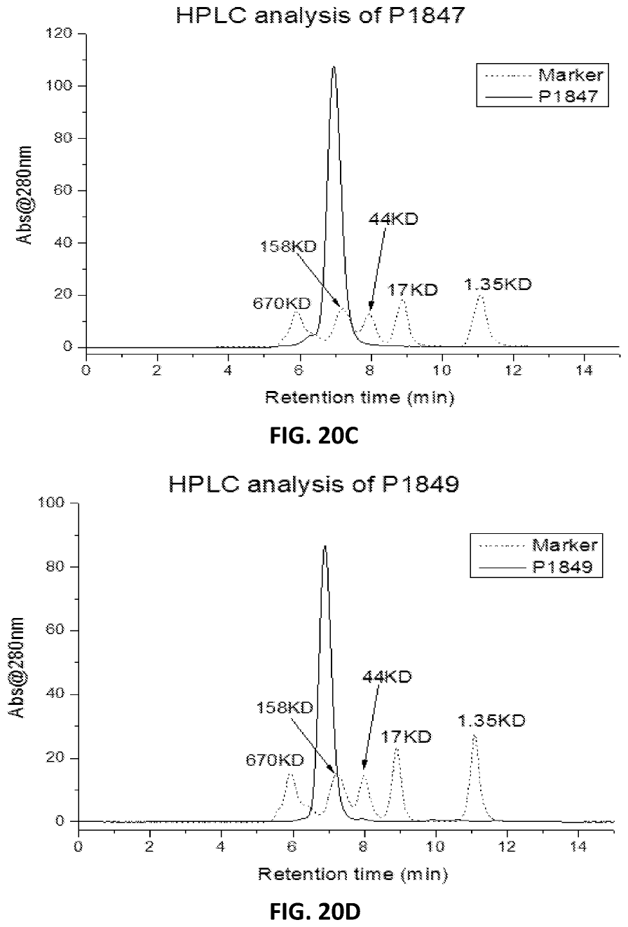

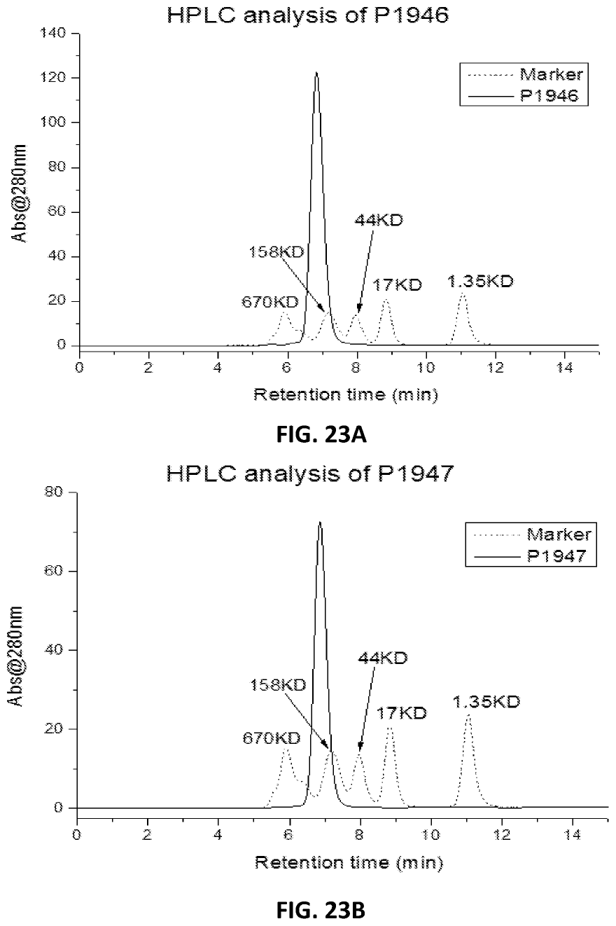

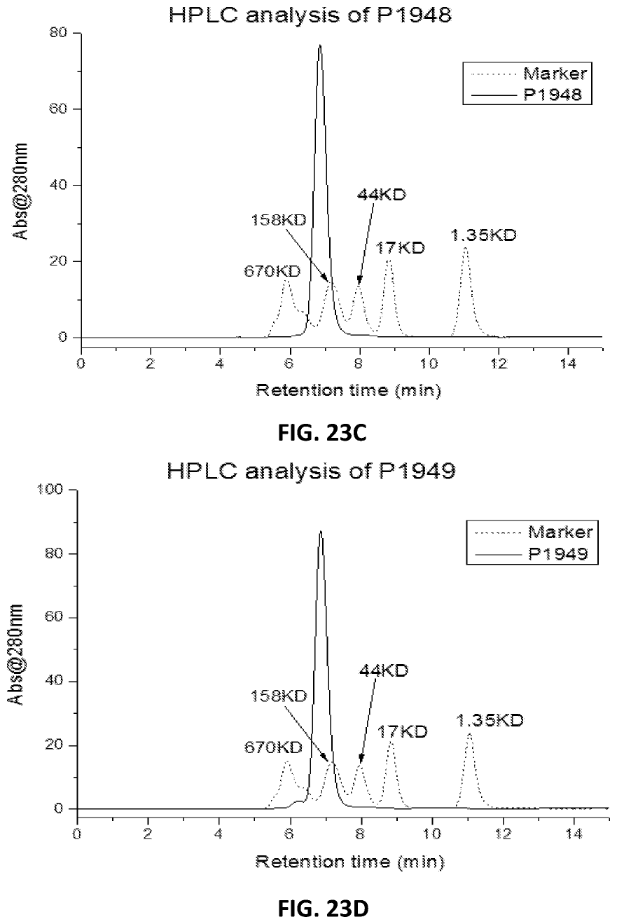

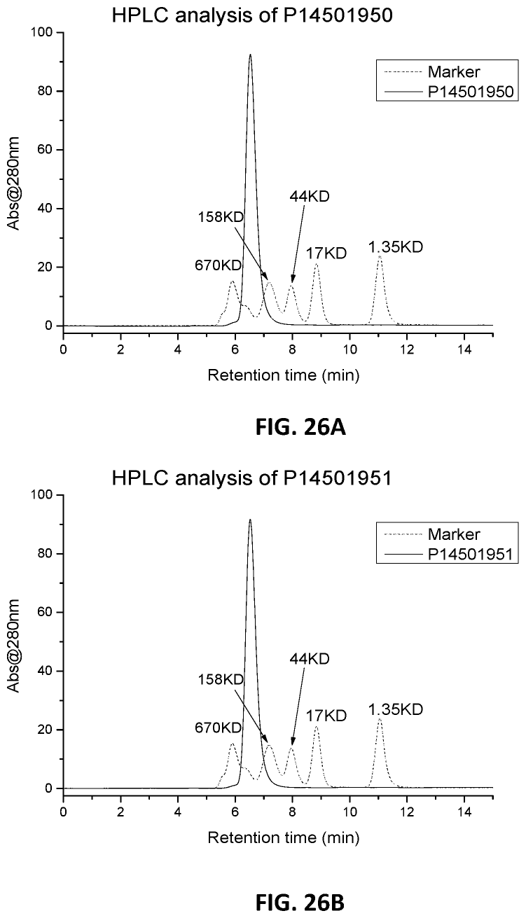

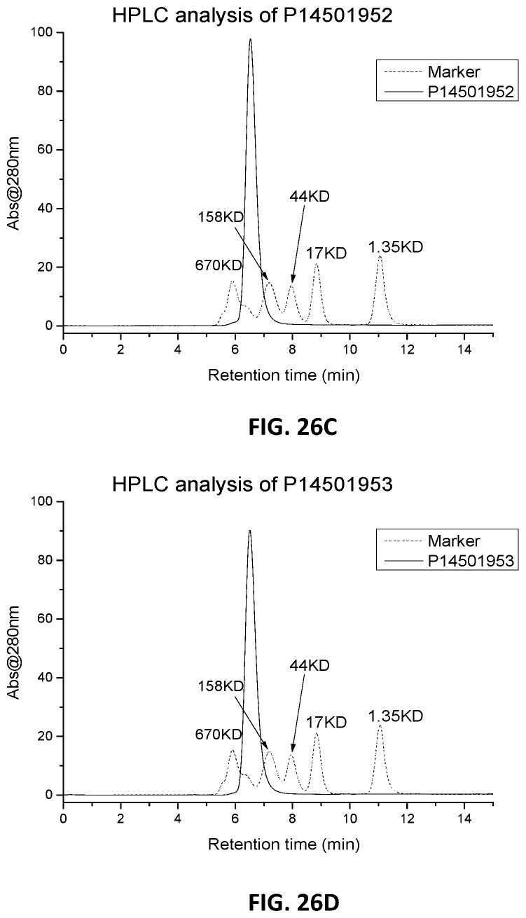

Purified proteins were also characterized by high performance liquid chromatography (HPLC) for homogeneity assessment. HPLC analysis was performed using Nanofilm SEC-250 column (Sepax) and Agilent 1260 according to the manufacturer's instructions. Representative HPLC results are shown in FIGS. 7A-7J, 11A-11F, 14A-14D, 17A-17D, 20A-20D, 23A-23D, 26A-26D, and 27D. Most of the proteins showed one single peak, indicating good homogeneity.

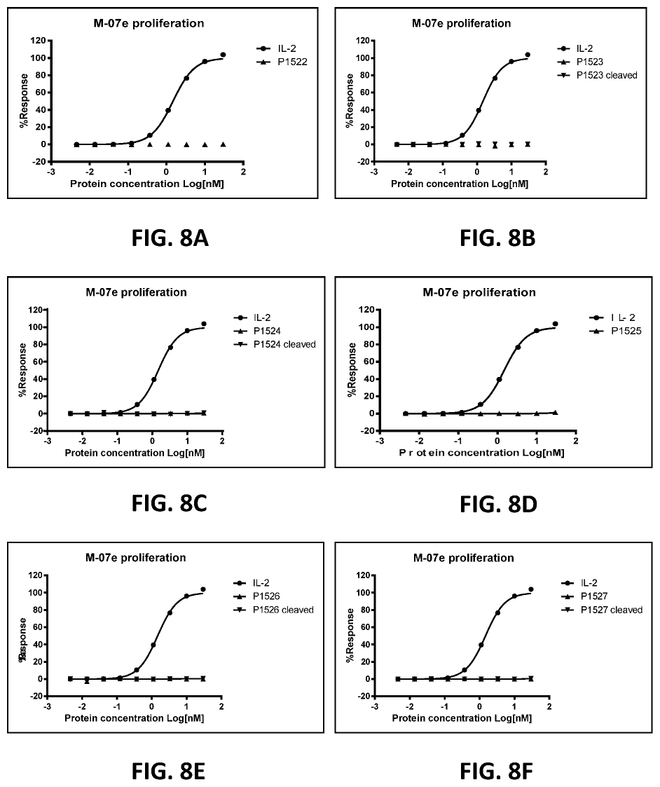

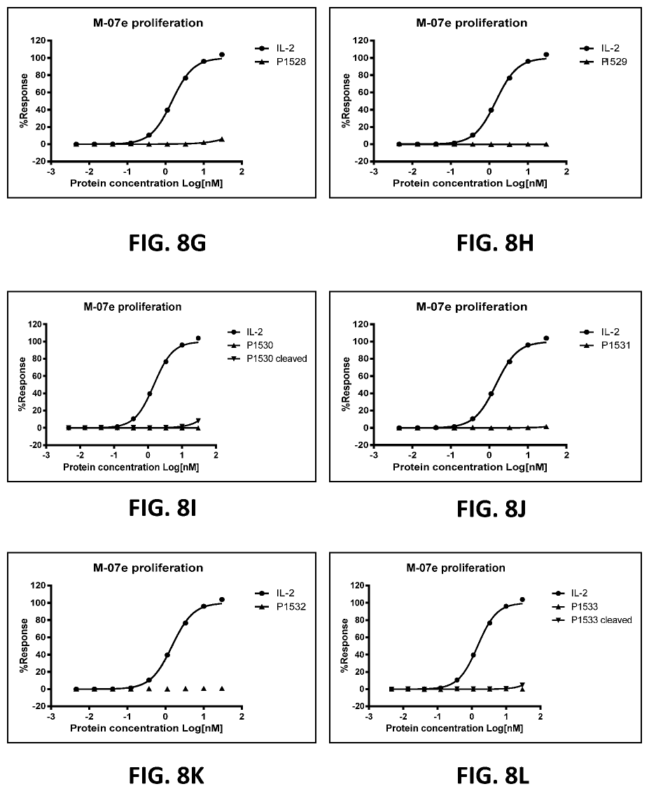

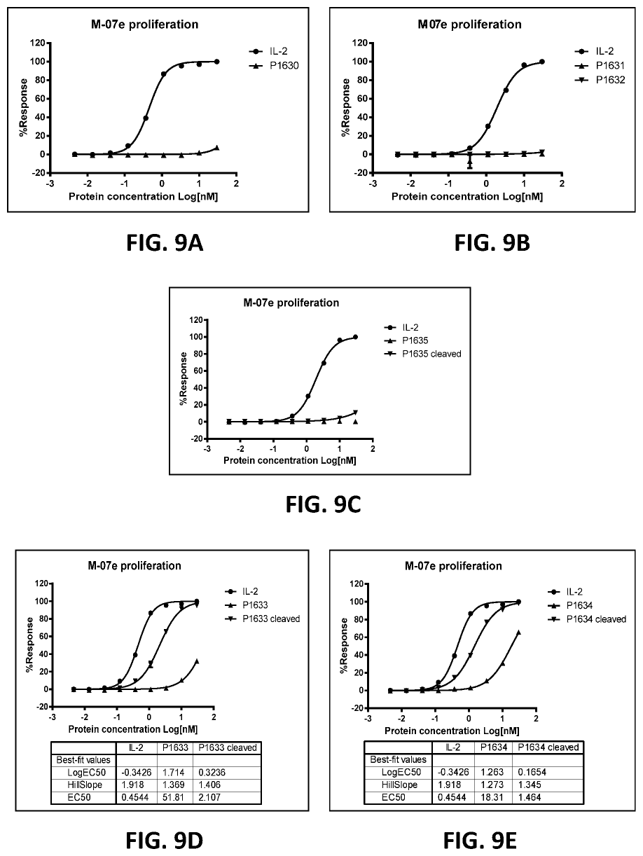

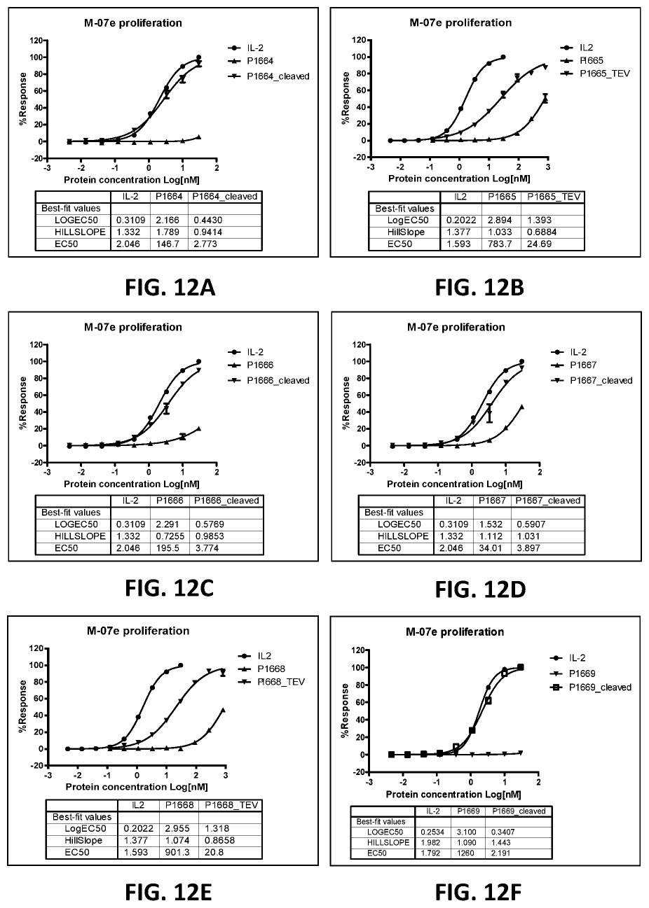

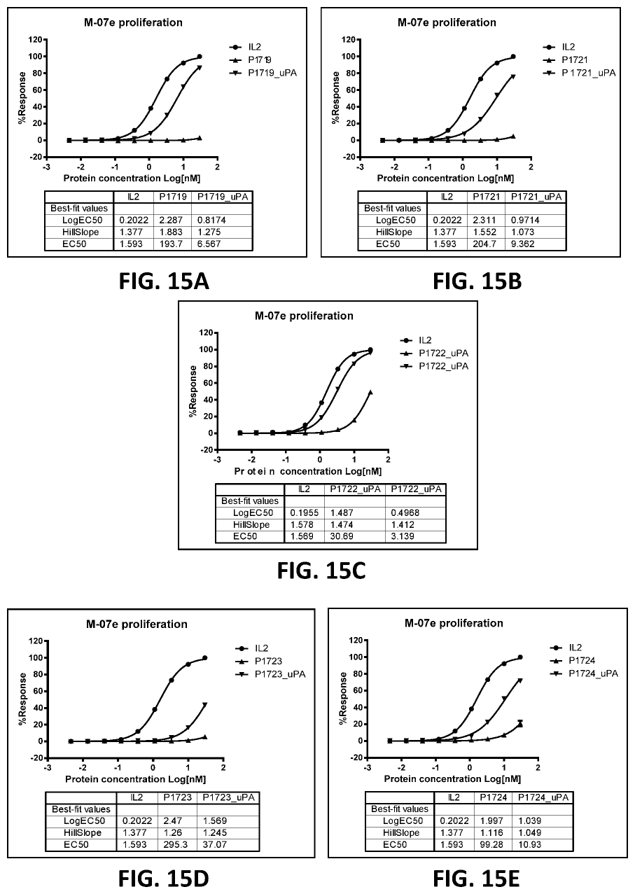

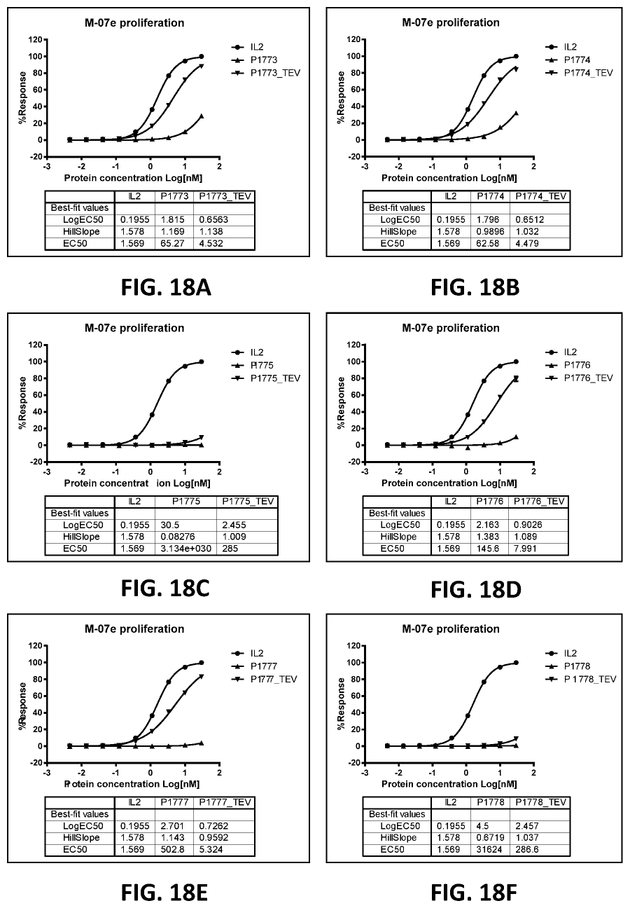

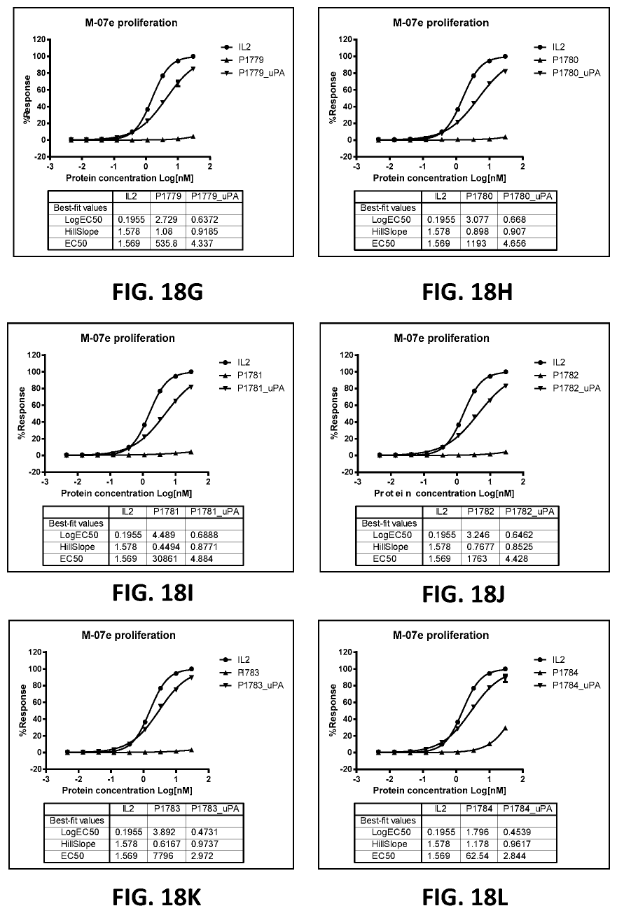

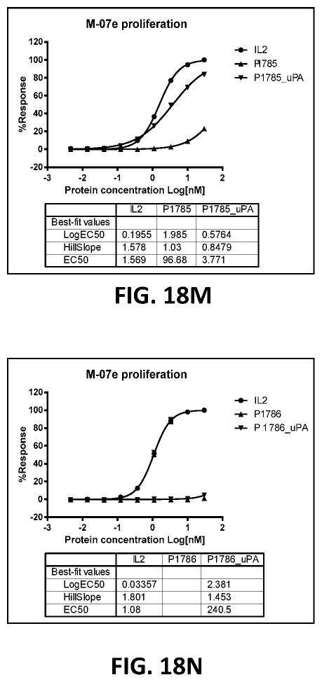

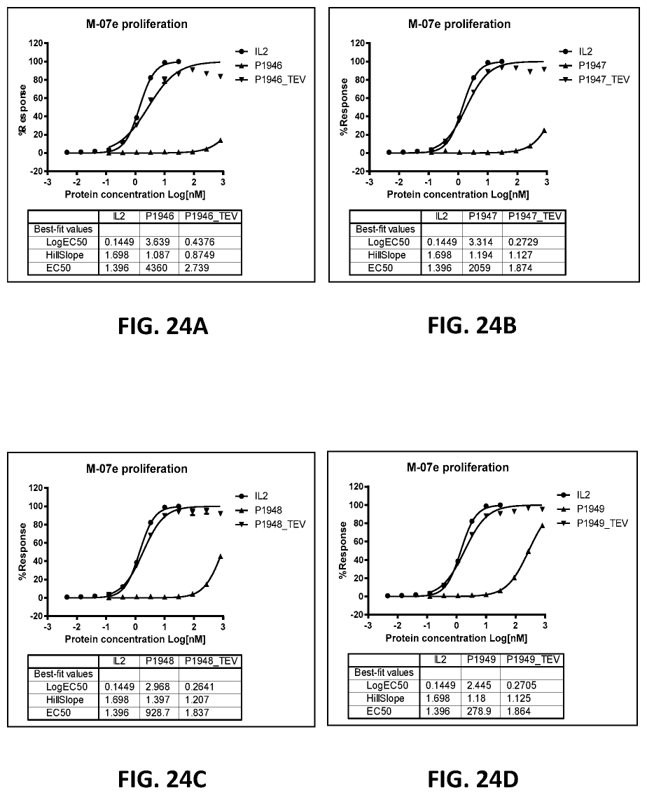

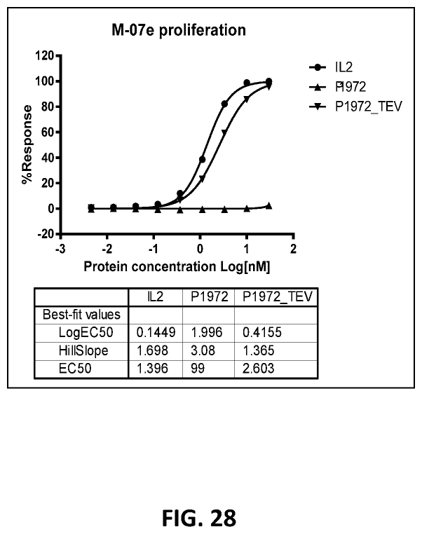

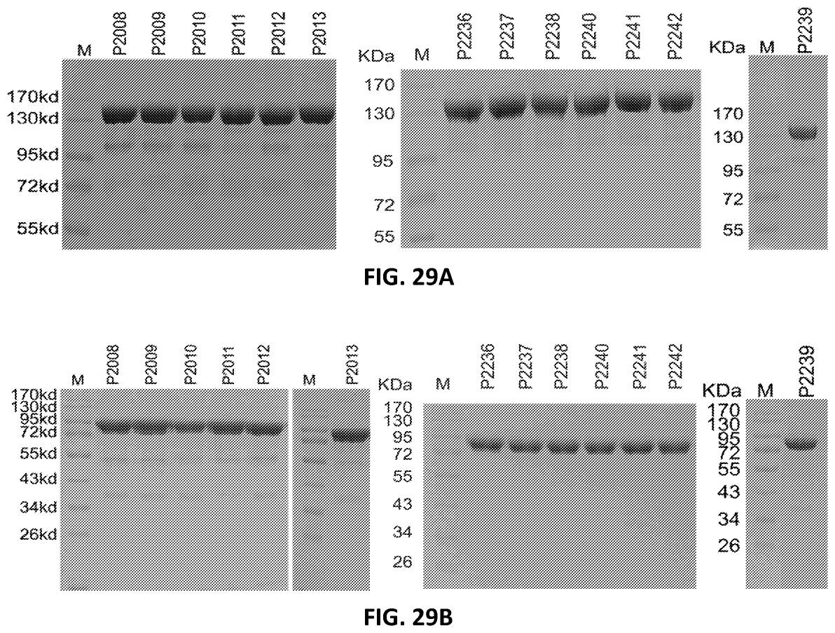

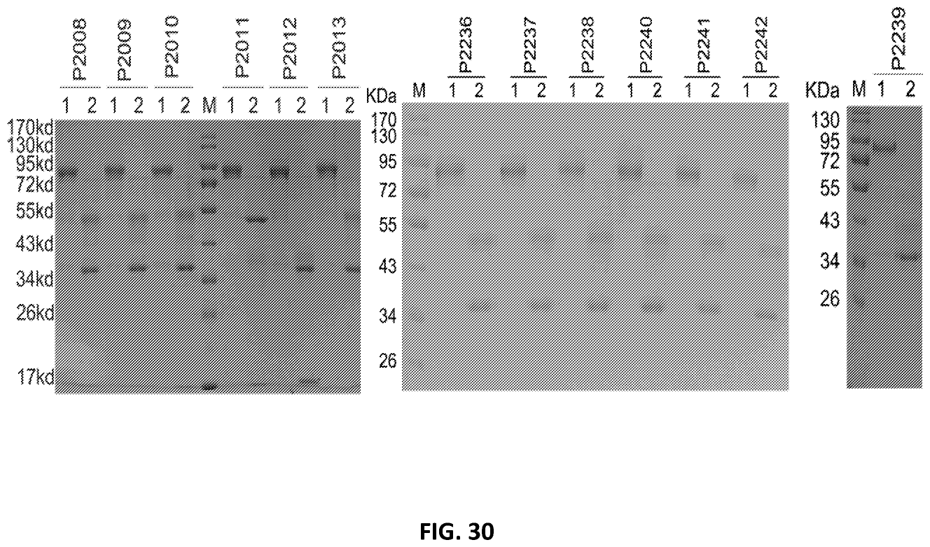

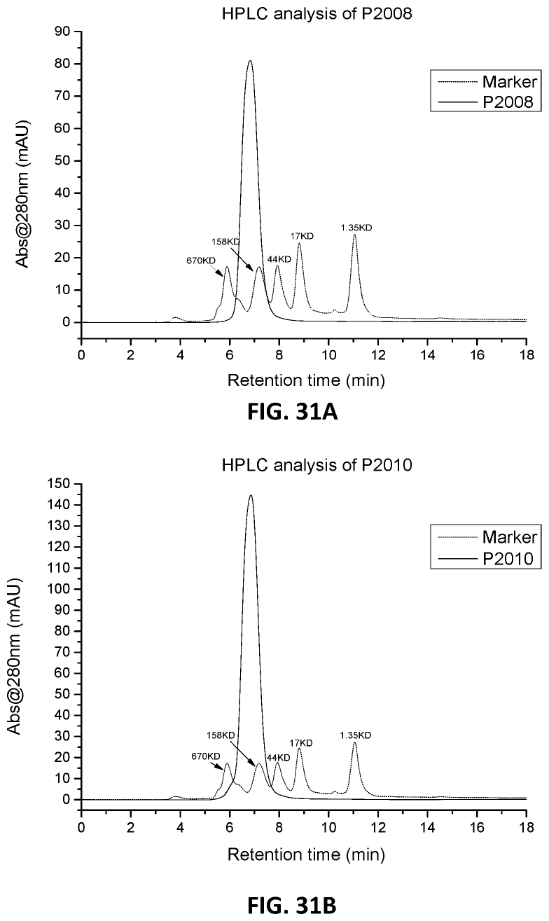

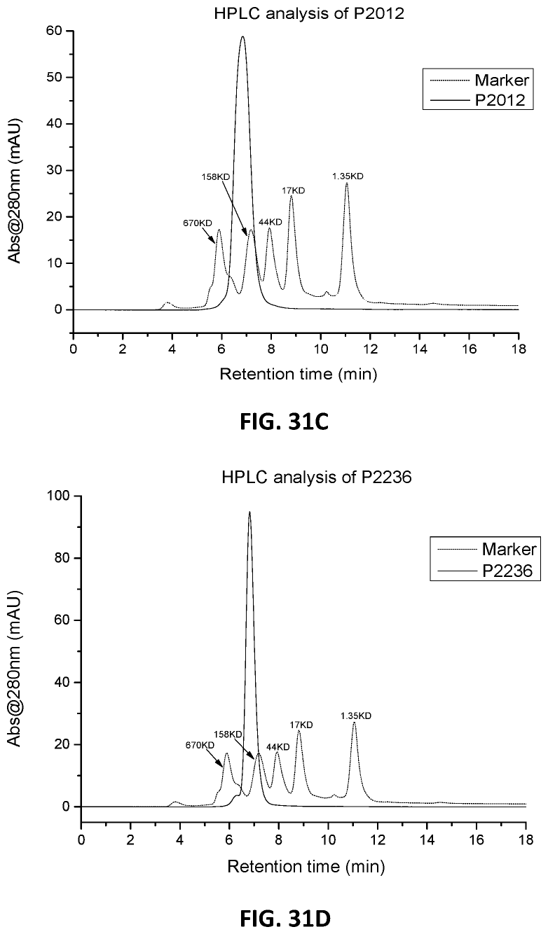

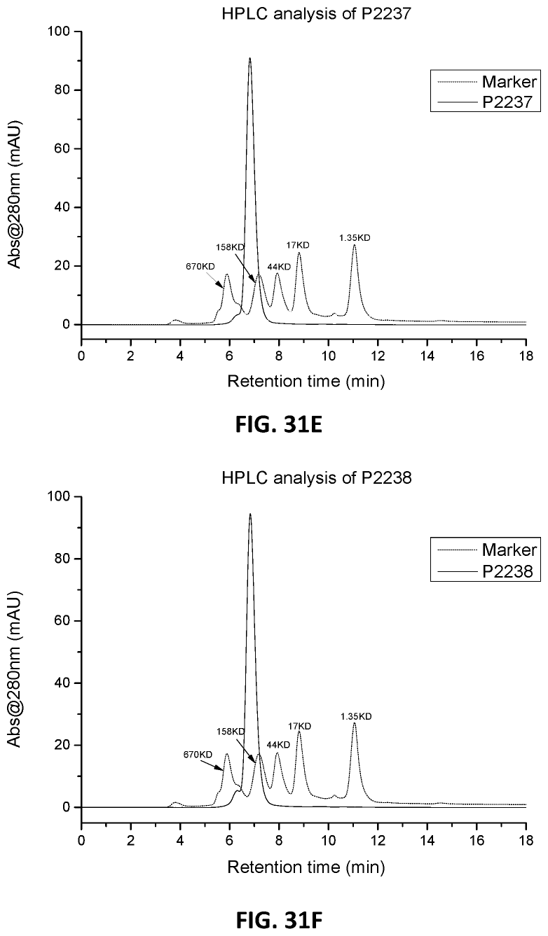

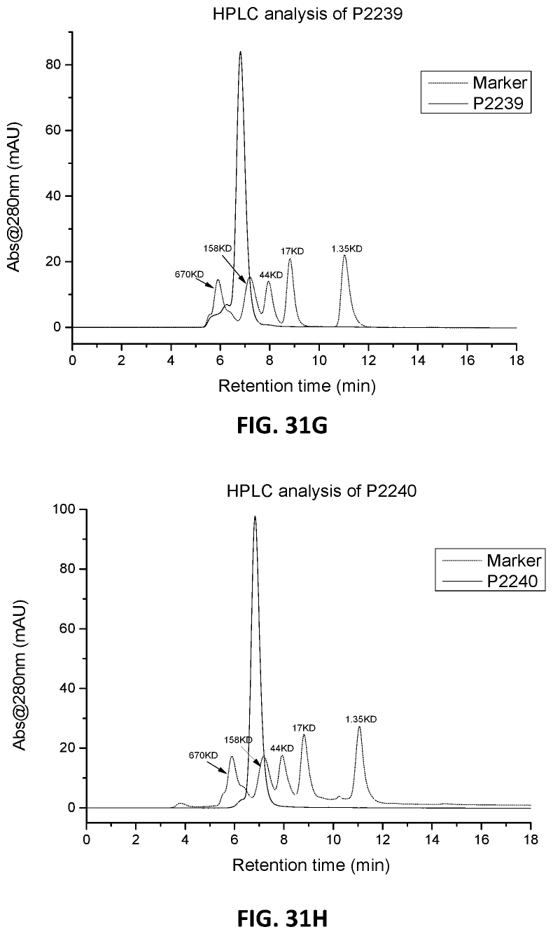

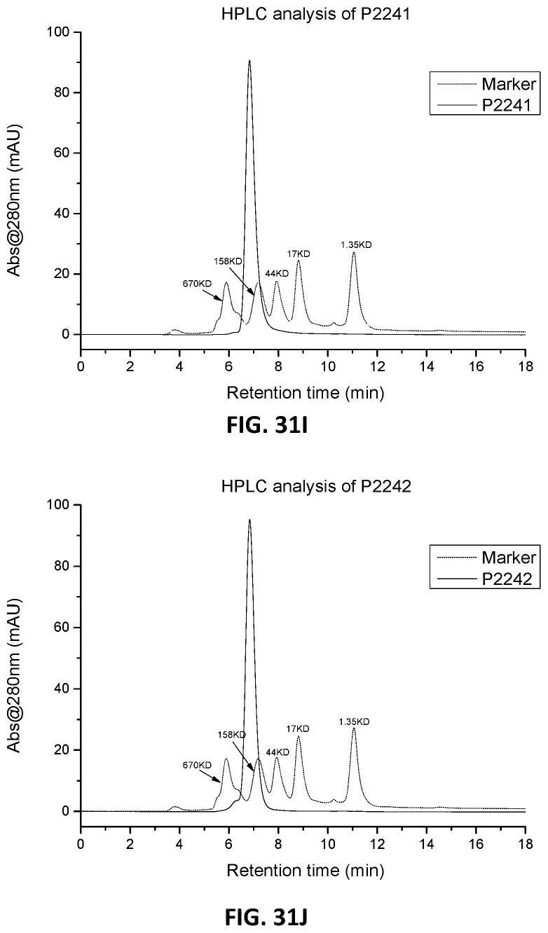

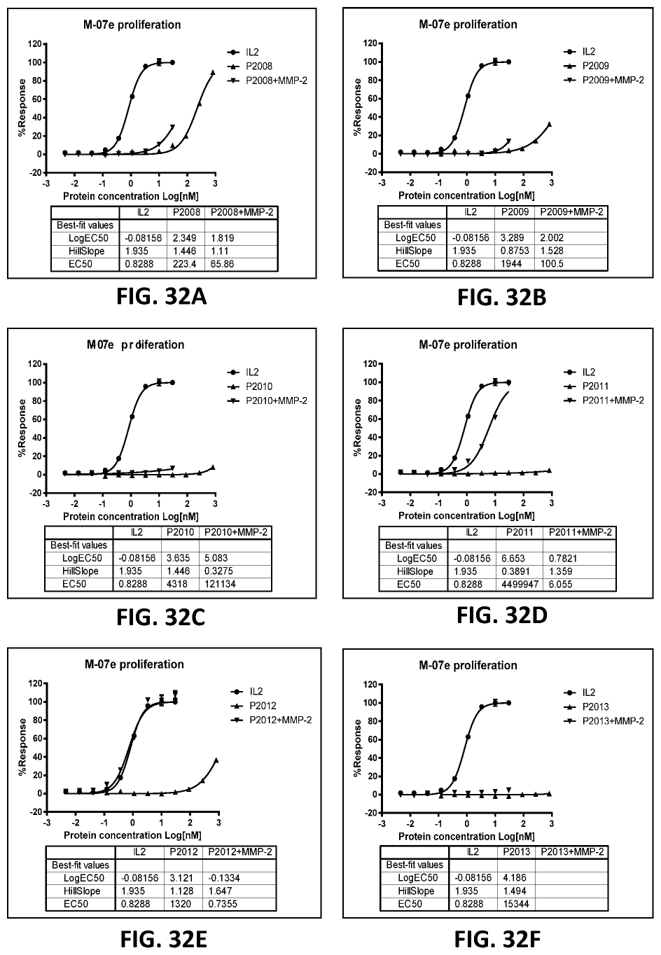

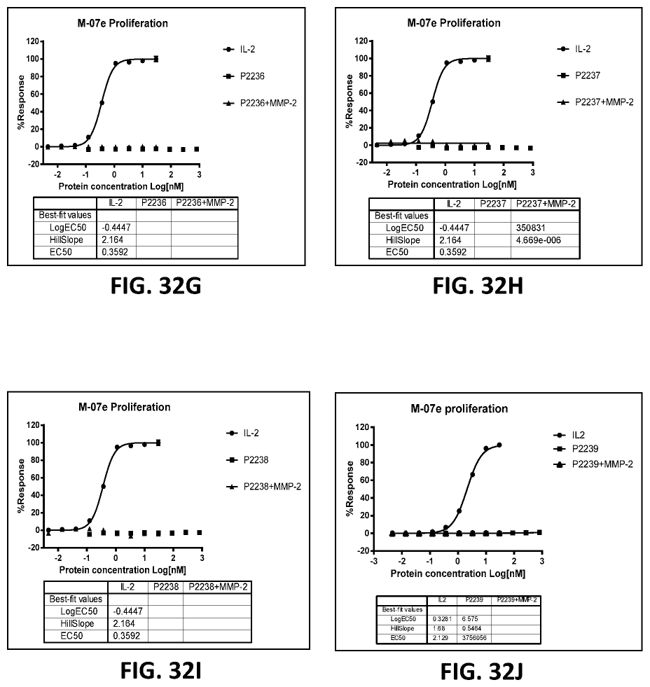

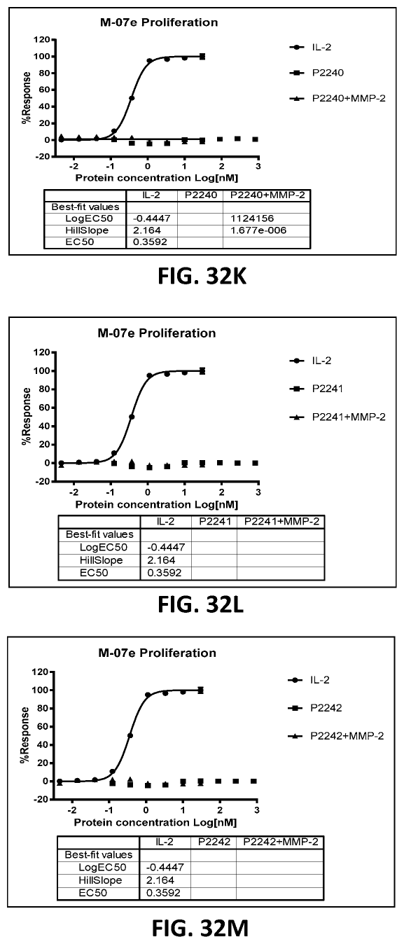

Functional assays—Proliferation. Proliferation assays were performed for purified proteins before and after cleavage. M-07e (IL-2Rβ/γc) cells were cultured in RPMI 1640 supplemented with 20% fetal bovine serum (FBS), 1% non-essential amino acids (NEAA), and 10% of 5637 cell culture supernatant. To measure cytokine-dependent cell proliferation, Mole cells were harvested in their logarithmic growth phase and washed twice with PBS. 90 μl of cell suspension (2×10⁴ cells/well) was seeded into 96-well plate and incubated for 4 hours in assay medium (RPMI 1640 supplemented with 10% FBS and 1% NEAA) for cytokine starvation at 37° C. and 5% CO2. IL-2 control and purified proteins samples used in the assays were prepared in assay medium to an initial concentration of 300 nM, followed by 1/3 serial dilutions. 10 μl of diluted protein was added into corresponding wells and incubated at 37° C. and 5% CO2 for 72 hours. Colorimetric assays using a Cell Counting Kit-8 (CCK-8, Dojindo, CK04) were performed to measure the amount of live cells. The results are shown in FIGS. 8A-8L, 9A-9E, 12A-12F, 15A-15E, 18A-18N, 21A-21Q, 24A-24D, and 28.

No activity was detected for fusion proteins without TEV cleavage site from IL-2-stable linker-IL-2Rα format (P1522 and P1525) and IL-2Rα-stable linker-IL-2 format (P1528 and P1531). No activity was detected for fusion proteins before TEV cleavage from Fc-TEV-IL-2-stable linker-IL-2Rα format (P1523 and P1526), IL-2-stable linker-IL-2Rα-TEV-Fc format (P1524 and P1527), Fc-TEV-IL-2Rα-stable linker-IL-2 format (P1529 and P1532) and IL-2Rα-stable linker-IL-2-TEV-Fc format (P1530 and P1533). IL-2 activity was not restored for these fusion proteins after TEV cleavage.

For the formats with TEV cleavage site between IL-2 and IL-2Rα, P1630, P1631, and P1632 showed no activity before TEV cleavage and could not be cleaved by TEV; P1635 showed no activity before and after TEV cleavage; P1633 and P1634 showed low activity before TEV cleavage and restored full or partial activity after TEV cleavage.

For the fusion proteins with longer cleavable linker (TEV cleavage site) between IL-2 and IL-2Rα, P1664, P1665, P1666, P1667, P1668, P1669, P1773, P1774, P1776, and P1777 showed very low activity before TEV cleavage and restored full or partial activity after TEV cleavage. P1775 and P1778 showed no or very low activity before TEV cleavage and activity could not be recovered after TEV cleavage.

For the fusion proteins with real protease cleavage site in the linker between IL-2 and IL-2Rα, P1779, P1780, P1781, P1782, P1783, and P1785 showed no or very low activity before protease cleavage and activity was recovered after protease cleavage. P1786, as a negative control, showed no activity before and after protease cleavage.

For ILR format with disulfide bond between IL-2 and IL-2Rα, P1719 P1721, P1722, P1723, and P1724 showed no or low activity before protease cleavage and activity was recovered partially or fully after protease cleavage.

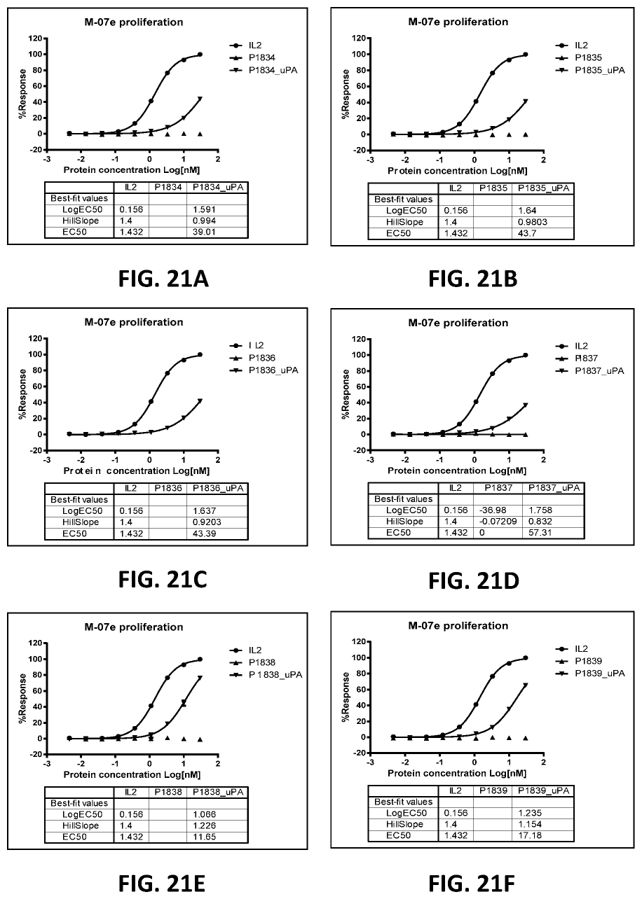

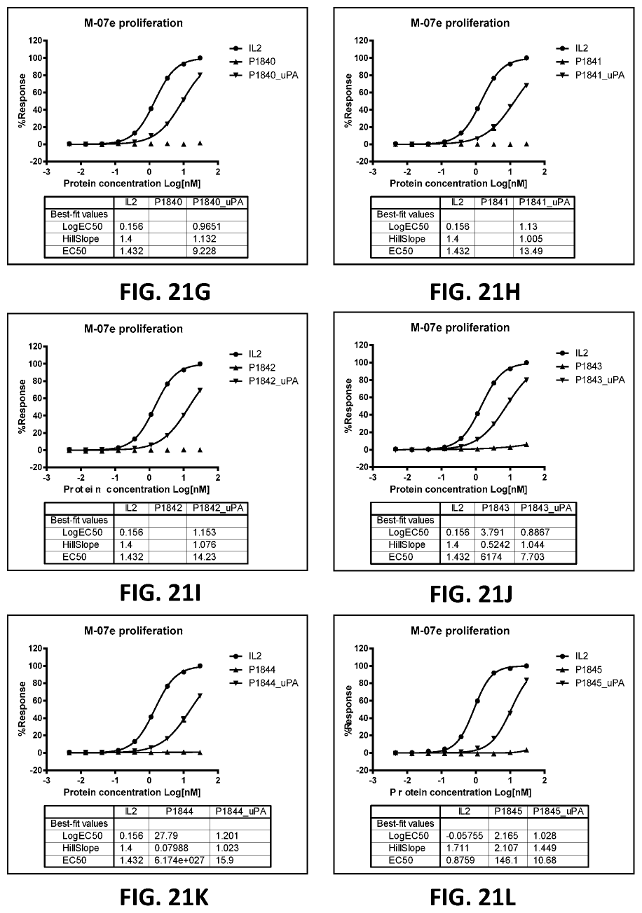

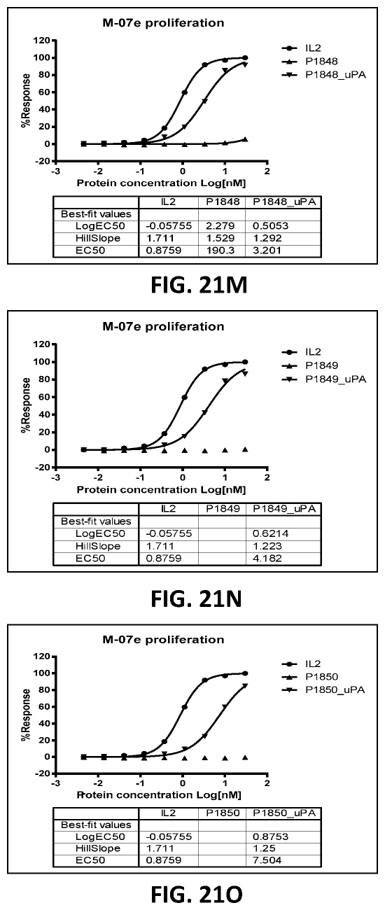

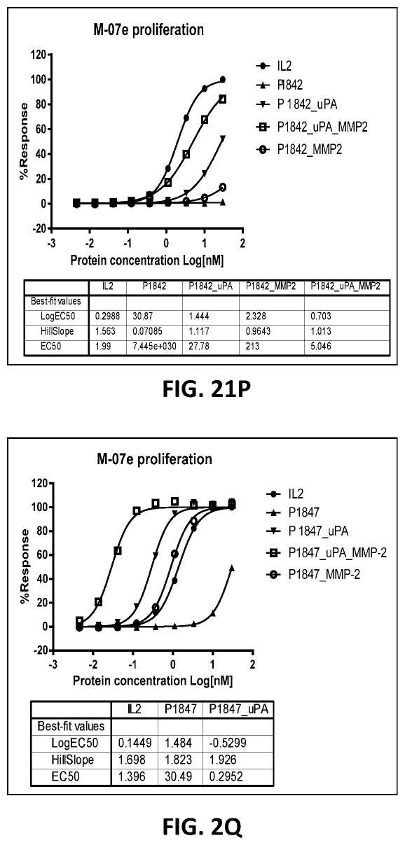

For Fc-PS1-IL-2-PS2-IL2Rα format, fusion proteins showed no or very low activity before protease cleavage, and activity was recovered partially after protease cleavage at PS2, as shown in FIGS. 21A-21O. For P1842 and P1847, different cleavage combinations were tested: single cleavage at PS1, single cleavage at PS2, and double cleavage at both PS1 and PS2. For P1842, low activity was recovered after single cleavage at PS1 or PS2, and full activity was restored after double cleavage at both PS1 and PS2. For P1847 with superkine D10, low activity was detected before protease cleavage and full activity was recovered after single cleavage at PS1 or PS2, and after double cleavage at both PS1 and PS2; indeed, after double cleavable this construct showed higher activity than wild-type IL-2.

For the fusion proteins with wild type IL-2 or IL-2 muteins with lower binding affinity towards IL-2Rα, P1946, P1947, P1948, and P1949 showed low activity before protease cleavage and restored full activity after cleavage. P1947, P1948, and P1949 with IL-2 muteins showed higher activity than P1946 before protease cleavage.

P1972, with IL-2-E61S and IL-2Rα-K38S, showed low activity before TEV cleavage and recovered full activity after cleavage.

1. An activatable proprotein homodimer, comprising a first polypeptide and a second polypeptide, wherein:
(a) the first polypeptide and the second polypeptide comprise, in an N- to C-terminal orientation, or a C- to N-terminal orientation, a binding moiety, a first linker, an IL-2 protein, a second linker, and an IL-2 binding protein; or
(b) the first polypeptide and the second polypeptide comprise, in an N- to C-terminal orientation, or a C- to N-terminal orientation, a binding moiety, a first linker, an IL-2 binding protein, a second linker, and an IL-2 protein,
wherein the binding moiety of the first polypeptide binds to the binding moiety of the second polypeptide, wherein the IL-2 protein of the first polypeptide binds to the IL-2 binding protein of the second polypeptide, and wherein the IL-2 binding protein of the first polypeptide binds to the IL-2 protein of the second polypeptide, wherein said binding masks a binding site of IL-2 protein(s) that otherwise binds to an IL-2Rβ/γc and/or IL-2Rα/β/γc chain present on the surface of an immune cell in vitro or in vivo, and wherein at least one of the first or the second linker is a cleavable linker; or
(c) the first and the second polypeptide comprise, in an N- to C-terminal orientation, or a C- to N-terminal orientation, an IL-2 protein, a first linker, an IL-2 binding protein, a second linker, and an affinity purification tag; or
(d) the first and the second polypeptide comprise, in an N- to C-terminal orientation, or a C- to N-terminal orientation, an IL-2 binding protein, a first linker, an IL-2 protein, a second linker, and an affinity purification tag,
wherein the IL-2 protein of the first polypeptide binds to the IL-2 binding protein of the second polypeptide, and wherein the IL-2 binding protein of the first polypeptide binds to the IL-2 protein of the second polypeptide, wherein said binding masks a binding site of IL-2 protein(s) that otherwise binds to an IL-2Rβ/γc and/or IL-2Rα/β/γc chain present on the surface of an immune cell in vitro or in vivo, and wherein the first linker is a cleavable linker.

2. The activatable proprotein homodimer of claim 1, wherein the first and second IL-2 proteins comprise, consist, or consist essentially of an amino acid sequence that is at least 80, 85, 90, 95, 98, or 100% identical to an amino acid sequence selected from Table S1, optionally amino acids 21-153 of SEQ ID NO: 1 (full-length wild-type human IL-2), optionally comprising a C145X (X is any amino acid) or a C145S substitution as defined by SEQ ID NO: 1.

3. The activatable proprotein homodimer of claim 1 or 2, wherein the first and second IL-2 proteins comprise, consist, or consist essentially of an amino acid sequence that is at least 80, 85, 90, 95, 98, or 100% identical to SEQ ID NO: 2 (mature human IL-2 with C125S substitution), optionally wherein the IL-2 protein retains the S5125 residue as defined by SEQ ID NO: 2.

4. The activatable proprotein homodimer of any one of claims 1-3, wherein the first and second IL-2 proteins comprise one or more substitutions selected from K35C, R38C, T41C, F42C, E61C, and V69C as defined by SEQ ID NO: 2.

5. The activatable proprotein homodimer of claim 4, wherein the first IL-2 protein forms a disulfide bond with the second IL-2 binding protein, and wherein the second IL-2 protein forms a disulfide bond with the first IL-2 binding protein, optionally via one or more of the cysteines in claim 4 and one or more cysteines in the first and second IL-2 binding proteins(s).

6. The activatable proprotein of any one of claims 1-5, wherein the first and second IL-2 proteins comprise one or more amino acid substitutions at position 69, 74, and/or 128 as defined by SEQ ID NO: 2, optionally wherein the one or more amino acid substitutions are selected from V69A, Q74P, and I128T as defined by SEQ ID NO: 2.

7. The activatable proprotein homodimer of any one of claims 1-6, wherein the first and second IL-2 proteins comprise one or more amino acid substitutions at position T3, R38, F42, Y45, E61, E62, E68, and/or L72 as defined by SEQ ID NO: 2, optionally wherein the one or more amino acid substitutions are selected from T3A; R38A and R38K; F42A, F42G, F42S, F42T, F42Q, F42E, F42N, F42D, F42R, F42K, and F42I; Y45A, Y45G, Y45S, Y45T, Y45Q, Y45E, Y45N, Y45D, Y45R, and Y45K; E61S; E62A and E62L; E68A and E68V; and L72A, L72G, L72S, L72T, L72Q, L72E, L72N, L72D, L72R, and L72K, including combinations thereof, optionally a combination selected from F42A, Y45A, and L72G; R38K, F42Q, Y45N, E62L, and E68V; R38K, F42Q, Y45E, and E68V; R38A, F42I, Y45N, E62L, and E68V; R38K, F42K, Y45R, E62L, and E68V; R38K, F42I, Y45E, and E68V; and R38A, F42A, Y45A, and E62A.

8. The activatable proprotein homodimer of any one of claims 1-7, wherein the first and second IL-2 proteins comprise, consist, or consist essentially of an amino acid sequence that is at least 80, 85, 90, 95, 98, or 100% identical to SEQ ID NO: 3 (mature human IL-2 “D10” variant), optionally wherein the IL-2 protein retains any one or more of the Q74H, L80F, R81D, L85V, I86V, and/or I92F substitutions as defined by SEQ ID NO: 3.

9. The activatable proprotein homodimer of any one of 1-8, wherein the first and second IL-2 binding proteins comprise a first and second IL-2Rα protein, or a first and second antibody or antigen binding fragment thereof that specifically binds to the IL-2 protein(s), optionally a bi-specific antibody or antigen binding fragment thereof.

10. The activatable proprotein homodimer of claim 9, wherein the first and second IL-2Rα proteins comprise, consist, or consist essentially of an amino acid sequence that is at least 80, 85, 90, 95, 98, or 100% to an amino acid sequence selected from Table S2, optionally amino acids 22-187 of SEQ ID NO: 4 (full-length wild-type human IL-2Rα).

11. The activatable proprotein homodimer of claim 9 or 10, wherein the first and second IL-2Rα proteins comprise one or more cysteine substitutions selected from D4C, D6C, N27C, K38C, S39C, L42C, Y43C, I118C, and H120C as defined by SEQ ID NO: 6 (human IL-2Rα Sushi 1 to Sushi 2 domain), and/or a K38S substitution.

12. The activatable proprotein homodimer of any one of claims 9-11, wherein the first IL-2Rα protein forms a disulfide bond with the second IL-2 protein, and wherein the second IL-2Rα protein forms a disulfide bond with the first IL-2 protein, optionally via one or more of the cysteines in claim 11 and one or more cysteines in the IL-2 protein, optionally one or more of the cysteines in claim 4, optionally one or more cysteine pairs selected from IL2-K35C and IL2Rα-D4C, IL2-R38C and IL2Rα-D6C, IL2-R38C and IL2Rα-H120C, IL2-T41C and IL2Rα-I118C, IL2-F42C and IL2Rα-N27C, IL2-E61C and IL2Rα-K38C, IL2-E61C and IL2Rα-S39C, and IL2-V69C and IL2Rα-L42C,
wherein disulfide binding between the IL-2 protein and the IL-2Rα protein masks the binding site of the IL-2 protein that preferentially binds to the IL-2Rα/β/γc chain expressed on T_{regs}.

13. The activatable proprotein homodimer of any one of claims 9-12, wherein first and second IL-2Rα proteins comprise an alanine substitution at position 49 and/or 68 as defined by SEQ ID NO: 6.

14. The activatable proprotein homodimer of claim 9, wherein the first and second antibody or antigen binding fragment thereof that specifically binds to the IL-2 protein is selected from one or more of a whole antibody, Fab, Fab′, F(ab′)2, monospecific Fab2, bispecific Fab2, FV, single chain Fv (scFv), scFV-Fc, nanobody, diabody, camelid, and a minibody, optionally wherein the antibody is NARA1 or an antigen binding fragment thereof.

15. The activatable proprotein homodimer of any one of claims 1-14, wherein the binding moieties of (a) and/or (b) do not bind to the IL-2 protein or the IL-2 binding protein.

16. The activatable proprotein homodimer of any one of claims 1-14, wherein the binding moieties of (a) and/or (b) bind to the IL-2 protein.

17. The activatable proprotein homodimer of any one of claims 1-16, wherein the binding moieties of the first polypeptide and the second polypeptide of (a) and/or (b) bind together, optionally homodimerize, via at least one non-covalent interaction.

18. The activatable proprotein homodimer of any one of claims 1-17, wherein the binding moieties of the first polypeptide and the second polypeptide of (a) and/or (b) bind together, optionally homodimerize, via at least one covalent bond.

19. The activatable proprotein homodimer of claim 18, wherein the at least one covalent bond comprises at least one disulfide bond.

20. The activatable proprotein homodimer of any one of claims 1-19, wherein the binding moieties of the first polypeptide and the second polypeptide of (a) and/or (b) are selected from Table M1.

21. The activatable proprotein homodimer of any one of claims 1-20, wherein the binding moieties of the first polypeptide and the second polypeptide of (a) or (b) comprise an antigen binding domain of an immunoglobulin, including antigen binding fragments and variants thereof.

22. The activatable proprotein of any one of claims 1-21, wherein the binding moieties of the first polypeptide and the second polypeptide of (a) and/or (b) comprise a CH1, CH2, CH3, CH1CH3, CH2CH3, CH1CH2CH3, and/or CL domain of an immunoglobulin, including fragments and variants thereof.

23. The activatable proprotein homodimer of claim 21 or 22, wherein the binding moieties of the first polypeptide and the second polypeptide of (a) and/or (b) comprise, in an N- to C-terminal orientation: (1) an antigen binding domain of an immunoglobulin, including antigen binding fragments and variants thereof; and (2) a CH1, CH2, CH3, CH1CH3, CH2CH3, CH1CH2CH3, and/or CL domain of an immunoglobulin, including fragments and variants thereof.

24. The activatable proprotein homodimer of any one of claims 21-23, wherein the antigen binding domain comprises a VH or VL domain of an immunoglobulin, including antigen binding fragments and variants thereof.

25. The activatable proprotein homodimer of any one of claims 1-24, wherein the binding moieties of the first polypeptide and the second polypeptide of (a) and/or (b) do not bind to an antigen.

26. The activatable proprotein homodimer of any one of claims 1-25, wherein the binding moieties of the first polypeptide and the second polypeptide of (a) and/or (b) comprise a CH2CH3 domain of an immunoglobulin.

27. The activatable proprotein homodimer of any one of claims 21-26, wherein the immunoglobulin is from an immunoglobulin class selected from IgG1, IgG2, IgG3, IgG4, IgA, IgD, IgE, and IgM.

29. The activatable proprotein homodimer of any one of claims 1-28, wherein the binding moieties of the first polypeptide and the second polypeptide of (a) and/or (b) comprise a leucine zipper peptide.

30. The activatable proprotein homodimer of any one of claims 1-29, wherein the affinity purification tag of (c) and/or (d) is selected from a polyhistidine tag (optionally hexahistidine tag), a VSV-G tag, a universal tag, a Strep-tag, an S-tag, an S1-tag, a Phe-tag, a Cys-tag, an Asp-tag, an Arg-tag, a Myc epitope tag, a KT3 epitope tag, an HSV epitope tag, a histidine affinity tag, a hemagglutinin (HA) tag, a FLAG epitope tag, an E2 epitope tag, a V5-tag, a T7-tag, an AU5 epitope tag, and an AU1 epitope tag.

31. The activatable proprotein homodimer of any one of claims 1-30, wherein the cleavable linker comprises a protease cleavage site, optionally wherein the cleavable linker is selected from Table S3.

32. The activatable proprotein homodimer of claim 31, wherein the protease cleavage site is cleavable by a protease selected from one or more of a metalloprotease, a serine protease, a cysteine protease, and an aspartic acid protease.

33. The activatable proprotein homodimer of claim 31 or 32, wherein protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

34. The activatable proprotein homodimer of any one of claims 1-33, wherein the first linker and/or the second linker are about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16 ,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

35. The activatable proprotein homodimer of any one of claims 1-34, wherein the first linker of (a) and/or (b) is a cleavable linker, and wherein the second linker of (a) and/or (b) is a non-cleavable linker.

36. The activatable proprotein homodimer of claim 35, wherein cleavage, optionally protease cleavage, of the first linker of (a) and/or (b) exposes the binding site(s) of the first and/or second IL-2 proteins that bind to the IL-2Rβ/γc chain present on the surface of the immune cell in vitro or in vivo.

37. The activatable proprotein homodimer of any one of claims 1-34, wherein the first linker of (a) and/or (b) is a non-cleavable linker, and wherein the second linker of (a) and/or (b) is a cleavable linker.

38. The activatable proprotein homodimer of claim 37, wherein cleavage, optionally protease cleavage, of the second linker of (a) and/or (b) exposes the binding site(s) of the first and/or second IL-2 proteins that bind to the IL-2Rβ/γc chain present on the surface of the immune cell in vitro or in vivo.

39. The activatable proprotein homodimer of any one of claims 1-34, wherein cleavage, optionally protease cleavage, of the first linker of (c) and/or (d) exposes the binding site(s) of the first and/or second IL-2 proteins that bind to the IL-2Rβ/γc chain present on the surface of the immune cell in vitro or in vivo.

40. The activatable proprotein homodimer of any one of claims 1-39, wherein the immune cell is selected from one or more of a T cell, a B cell, a natural killer cell, a monocyte, and a macrophage.

41. The activatable proprotein homodimer of any one of claims 1-40, wherein the first polypeptide and the second polypeptide of (a) comprise, in an N- to C-terminal orientation, the binding moiety, the first linker, the IL-2 protein, the second linker, and the IL-2 binding protein.

42. The activatable proprotein homodimer of any one of claims 1-40, wherein the first polypeptide and the second polypeptide of (a) comprise, in an N- to C-terminal orientation, the IL-2 binding protein, the first linker, the IL-2 protein, the second linker, and the binding moiety.

43. The activatable proprotein homodimer of any one of claims 1-40, wherein the first polypeptide and the second polypeptide of (b) comprise, in an N- to C-terminal orientation, the binding moiety, the first linker, the IL-2 binding protein, the second linker, and the IL-2 protein.

44. The activatable proprotein homodimer of any one of claims 1-40, wherein the first polypeptide and the second polypeptide of (b) comprise, in an N- to C-terminal orientation, the IL-2 protein, the first linker, the IL-2 binding protein, the second linker, and the binding moiety.

45. The activatable proprotein homodimer of any one of claims 1-40, wherein the first polypeptide and the second polypeptide of (c) comprise, in an N- to C-terminal orientation, the IL-2 protein, the first linker, the IL-2 binding protein, the second linker, and the affinity purification tag.

46. The activatable proprotein homodimer of any one of claims 1-40, wherein the first polypeptide and the second polypeptide of (d) comprise, in an N- to C-terminal orientation, the IL-2 binding protein, the first linker, the IL-2 protein, the second linker, and the affinity purification tag.

47. The activatable proprotein homodimer of any one of claims 1-46, wherein the first polypeptide and the second polypeptide comprise, consist, or consist essentially of an amino acid sequence that is at least 80, 85, 90, 95, 98, or 100% identical to a sequence selected from Table S4, optionally wherein the TEV protease cleavage site is replaced with cleavage site cleavable by a human protease, optionally a cleavable linker selected from Table S3.

48. The activatable proprotein homodimer of any one of claims 1-47, which is substantially in homodimeric form in a physiological solution, or under physiological conditions, optionally in vivo conditions.

49. A recombinant nucleic acid molecule encoding the activatable proprotein homodimer of any one of claims 1-48.

50. A vector comprising the recombinant nucleic acid molecule of claim 49.

51. A host cell comprising the recombinant nucleic acid molecule of claim 44 or the vector of claim 50.

52. A method of producing an activatable proprotein, comprising culturing the host cell of claim 51 under culture conditions suitable for the expression of the activatable proprotein homodimer, and isolating the activatable proprotein from the culture.

53. A pharmaceutical composition, comprising the activatable proprotein homodimer of any one of claims 1-48, and a pharmaceutically acceptable carrier.

54. A method of treating disease in a subject, and/or a method of enhancing an immune response in a subject, comprising administering to the subject a therapeutically effective amount of the pharmaceutical composition of claim 53.

55. The method of claim 54, wherein the disease is selected from one or more of a cancer, a viral infection, and an immune disorder.

56. The method of claim 55, wherein the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

57. The method of any one of claims 54-56, wherein following administration, the activatable proprotein homodimer is activated through protease cleavage in a cell or tissue, optionally a cancer cell or cancer tissue, which exposes the binding site(s) of the first and/or second IL-2 proteins that bind to the IL-2Rβ/γc chain present on the surface of the immune cell in vitro or in vivo, and thereby generates an activated protein.

58. The method of claim 57, wherein the activated protein binds via the IL-2 protein to the IL-2Rβ/γc chain present on the surface of an immune cell in vitro or in vivo.

59. The method of claim 58, wherein the immune cell is selected from one or more of a T cell, a B cell, a natural killer cell, a monocyte, and a macrophage.

60. The method of any one of claims 57-59, wherein binding between the IL-2 protein(s) and the IL-2 binding protein(s) (optionally disulfide binding between the IL-2 protein(s) and the IL-2Rα protein(s)) in the activated protein masks the binding site of the IL-2 protein(s) that binds to the IL-2Rα/β/γc chain expressed on T_{regs}, and thereby interferes with binding of the activated protein to T_{regs}.

61. The method of any one of claims 54-60, wherein administration and activation of the activatable proprotein increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response.

62. The method of any one of claims 54-61, wherein administration and activation of the activatable proprotein increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

63. The method of claim 55, wherein the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

64. The method of claim 55, wherein the immune disorder is selected from one or more of type 1 diabetes, vasculitis, and an immunodeficiency.

65. The method of any one of claims 54-64, wherein the pharmaceutical composition is administered to the subject by parenteral administration.

66. The method of claim 65, wherein the parenteral administration is intravenous administration.

67. Use of a pharmaceutical composition of claim 53 in the preparation of a medicament for treating a disease in a subject, and/or for enhancing an immune response in a subject.

68. A pharmaceutical composition of claim 53 for use in treating a disease in a subject, and/or for enhancing an immune response in a subject.
